(19) United States
(12) Patent Application Publication
(10) Pub. No.: US 2024/0299062 A1
(43) Pub. Date: Sep. 12, 2024

(54) CARDIAC LEAD EXTRACTION DEVICE

(71) Applicant: MDSG Innovation Ltd., Rehovot (IL)

(72) Inventors: Benny ROUSSO, Rishon-LeZion (IL); Naama WINETRAUB, Holon (IL); Rodny ZARINI, Doar-Na Shimshon (IL); Or ZIGELBOIM, Ness Ziona (IL)

(73) Assignee: MDSG Innovation Ltd., Rehovot (IL)

(21) Appl. No.: 18/292,350

(22) PCT Filed: Jul. 27, 2022
(86) PCT No.: PCT/IL2022/050819

§ 371 Date: Jan. 26, 2024

Related U.S. Application Data
(60) Provisional application No. 63/226,001, filed on Jul. 27, 2021.

(51) Int. Cl. A61B 17/50; A61B 17/00; A61B 17/3201

(52) U.S. Cl. CPC A61B 17/50; A61B 17/3201; A61B 2017/00243; A61B 2017/00398; A61B 2017/00734

(57) ABSTRACT

The invention relates to a cardiac lead extraction system, comprising: a handle; an elongated body in communication with said handle; a bendable flexible portion in communication with said elongated body, said bendable flexible portion comprising a first lumen sized and shaped to fit over a cardiac lead; said bendable flexible portion being more flexible than said elongated body; an operational distal end in communication with said bendable flexible portion; where said bendable portion is configured to bend to a bending radius of less than 4 cm while keeping said first lumen open; and where said operational distal end comprises at least one lead extraction assistive tool, said operational distal end comprising a second lumen sized and shaped to fit over a cardiac lead, said second lumen being in communication with said first lumen, and said first lumen comprises an inner diameter of from about 1 mm to about 5 mm.

### RELATED APPLICATION/S

This application claims the benefit of priority of U.S. Provisional Patent Application No. 63/226,001 filed on 27 Jul. 2021, the contents of which are incorporated herein by reference in their entirety.

### FIELD AND BACKGROUND OF THE INVENTION

The indications and populations requiring the removal of pacemaker and defibrillator leads appears to be growing and may be expected to continue to grow. The removal of cardiac leads may be complicated by, for example, the development of encapsulating fibrous tissue around the leads at certain locations within the veins and the heart, and the removal procedure highly depends on the experience of the physician. In some cases, removing the lead includes separating the tissue from either the lead and/or the vein. For example, tissue may be cut and/or ablated in order to remove the lead. A device for removing such leads may include a telescoping sheath. For example, the telescoping sheath may be used to manually dilate the fibrous tissue. Some solutions disclose, for example, a mechanical rotating sheath used to dilate the tissue more aggressively than the manual sheaths. Some solutions disclose, for example, a laser and/or an RF ablation sheath used to ablate the fibrous tissue. Sometimes serious complications may occur due to removal of leads. For example, forces that are exerted on the lead, the vein, and/or the heart tissue in order to free the lead from the fibrous tissue may occasionally cause serious damage to the walls of the veins and the heart.

### SUMMARY OF THE INVENTION

The following describe some examples of embodiments of the invention. Other embodiments are within the scope of the description, including embodiments in which only some of the features from one example are used and embodiments in which one or more features are selected from two or more examples.

Example 1. A cardiac lead extraction device, comprising:
a. a handle;
b. an elongated body having a first proximal end, a first distal end, and a first lumen extending from said first proximal end toward said first distal end, said lumen sized and shaped to fit over a cardiac lead;
c. a controllable bendable flexible portion more flexible that said elongated body; said flexible portion having a second proximal end, a second distal end and a second lumen extending from said second proximal end toward said second distal end, said lumen sized and shaped to fit over a cardiac lead; said second proximal end interconnected to said first distal end; and said second distal end interconnected to an operational distal end;
wherein said operational distal end comprises at least one lead extraction assistive tool, said lead extraction helping tool is activated by a motor located at said handle or proximally to said handle.

Example 2. The device of example 1, wherein the inner diameter of said cardiac lead extraction device is from about 3 mm to about 7 mm.

Example 3. The device of examples 1 or 2, wherein the outer diameter of said cardiac lead extraction device is from about 6 mm to about 8 mm.

Example 4. The device of examples 1-3, wherein said controllable bendable flexible portion bends to a maximal angle of from about 35 degrees to about 150 degrees.

Example 5. The device of example 4, wherein an inner diameter of said controllable bendable flexible portion changes from about 0% to about 10% during said maximal angle.

Example 6. The device of examples 1-5, wherein said controllable bendable flexible portion comprises an articulated structure having multiple non-flexible components.

Example 7. The device of examples 4-6, wherein said controllable bendable flexible portion is capable of bending to said maximal angle while withstanding forces from about 300 gr to about 1000 gr.

Example 8. The device of example 1, wherein said lead extraction assistive tool comprises a tissue cutter.

Example 9. The device of example 8, wherein said tissue cutter comprises at least one movable blade.

Example 10. The device of examples 8 or 9, wherein said tissue cutter comprises at least one transmission attached to said motor; said transmission adapted to transfer motion from said motor to said at least one movable blade.

Example 11. The device of example 10, wherein said motion of said at least one movable blade is linear.

Example 12. The device of examples 10 or 11, wherein said motion of said at least one movable blade is circular.

Example 13. The device of examples 10-12, wherein said movement of said transmission is adapted to provide said at least one movable blade with a linear movement comprising impact force.

Example 14. The device of examples 10-13, wherein said motion of said at least one movable blade is a combination of linear movement and circular movement.

Example 15. The device of examples 10-14, wherein said motion of said at least one movable blade is characterized by a frequency from about 0.5 Hz to about 100 Hz.

Example 16. The device of example 10-15, wherein said motion of said at least one movable blade is characterized by a frequency from about 1 Hz to about 15 Hz.

Example 17. The device of examples 10-16, wherein said at least one movable blade comprises a retracted state where said at least one movable blade is not exposed thereby avoiding said at least one movable blade from cutting.

Example 18. The device of examples 10-17, wherein said at least one movable blade exits distally said operational distal end from about 0.15 mm to about 2 mm.

Example 19. The device of example 8, wherein said tissue cutter comprises at least two movable blades.

Example 20. The device of example 19, wherein the movement of said at least two movable blades is towards each therefore allowing cutting by shearing.

Example 21. The device of example 1, wherein said elongated body comprises an inner bending shaft.

Example 22. The device of example 21, wherein said inner bending shaft is as long as said elongated body.

Example 23. The device of examples 21 or 22, wherein said inner bending shaft transmits motion from said handle to said operational distal end through said elongated body.

Example 24. The device of example 1, wherein said controllable bendable flexible portion comprises an inner shaft.

Example 25. The device of example 1, wherein said lead extraction assistive tool comprises a lead cutter.

Example 26. The device of example 25, wherein said lead cutter comprises at least one blade and at least one movable part.

Example 27. The device of examples 25 or 26, wherein said lead cutter engages a cardiac lead within said lumen with said at least one movable part and moves said cardiac lead against said at least one blade.

Example 28. The device of examples 25-27, wherein said lead cutter comprises a groove, not aligned with the general direction of said lumen, where said cardiac lead is cut.

Example 29. The device of examples 25-28, wherein at least one blade is located in said movable part.

Example 30. The device of examples 25-29, wherein at least one blade is not located in said movable part.

Example 31. The device of examples 25-30, wherein the movement of said at least one movable part is a linear movement.

Example 32. The device of examples 25-31, wherein the movement of said at least one movable part comprises a screw rotating mechanism.

Example 33. The device of example 1, wherein said lead extraction assistive tool comprises a tissue identification tool.

Example 34. The device of example 33, wherein said tissue identification tool comprises at least one light emitting component, which is mechanically positioned to radiate in a direction aligned in front with the distal head of said device.

Example 35. The device of examples 33 or 34, wherein said tissue identification tool comprises an electronic phased array of transducers stationary placed around the distal end of said operational distal end.

Example 36. The device of example 1, wherein said lead extraction assistive tool comprises a steering tool.

Example 37. The device of example 36, wherein said steering tool comprises at least one wire that runs from said handle to said operational distal end.

Example 38. The device of examples 36 or 37, wherein said at least one wire runs inside a counter sleeve on said elongated sheath.

Example 39. The device of example 1, wherein said lead extraction assistive tool comprises a tissue separator.

Example 40. The device of example 39, wherein said tissue separator vibrates said operational distal end.

Example 41. The device of examples 39 or 40, wherein said vibration is generated by said steering tool.

Example 42. The device of examples 39-41, wherein said vibration comprises at least two-axis movement.

Example 43. The device of examples 39-42, wherein said vibration is in the range from about 1 Hz to about 100 Hz.

Example 44. The device of examples 39-43, wherein said tissue separator comprises fixed protrusions from the distal end of said operational distal end.

Example 45. The device of examples 39-44, wherein said tissue separator comprises movable protrusions, which extend radially and outwardly.

Example 46. The device of example 1, wherein said lead extraction assistive tool comprises a force analysis tool.

Example 47. The device of example 46, wherein said force analysis tool provides indication of the forces applied between said device and the tissue surrounding said device.

Example 48. The device of examples 46 or 47, wherein said force analysis tool provides indication of the forces applied between said device and said lead.

Example 49. A lead extraction accessory, comprising:
a. a handle;
b. an elongated body having a first proximal end, a first distal end, and a first lumen extending from said first proximal end toward said first distal end, said lumen sized and shaped to fit over a cardiac lead extraction device;
c. said elongated body comprising a controllable bendable flexible portion having a second proximal end and a second distal end, said second distal end interconnected to an operational distal end;
wherein said operational distal end comprises at least one lead extraction assistive tool.

Example 50. The lead extraction accessory of example 49, wherein a motor located at said handle activates said at least one lead extraction assistive tool.

Example 51. A cardiac lead cutter device, comprising:
a. an elongated body having a proximal end, a distal end and a lumen extending from said proximal end toward said distal end, said lumen sized and shaped to fit over a cardiac lead; and
b. a lead cutter tool located at said distal end of said device, said lead cutter component comprises at least one blade and at least one movable part;
wherein said movable part engages said cardiac lead and moves said cardiac lead against said at least one blade.

Example 52. The device of example 51, wherein said lead cutter comprises a groove, not aligned with the general direction of said lumen, where said cardiac lead is cut.

Example 53. The device of examples 51 or 52, wherein at least one blade is located in said movable part.

Example 54. The device of examples 51-53, wherein at least one blade is not located in said movable part.

Example 55. The device of examples 51-54, wherein the movement of said at least one movable part is a linear movement.

Example 56. The device of examples 51-55, wherein the movement of said at least one movable part comprises a screw rotating mechanism.

Example 57. A tissue cutter device for a cardiac lead, comprising:
a. an elongated body having a proximal end, a distal end and a lumen extending from said proximal end toward said distal end, said lumen sized and shaped to fit over a cardiac lead; and
b. a tissue cutter located at said distal end of said device, said tissue cutter comprises at least two separate movable blades.

Example 58. The device of example 57, wherein said tissue cutter comprises at least one transmission attached to a motor; said transmission adapted to transfer motion from said motor to said at least two movable blades.

Example 59. The device of examples 57 or 58, wherein the motion of at least one of said at least two movable blades is linear.

Example 60. The device of examples 57-59, wherein the motion of at least one of said at least two movable blades is circular.

Example 61. The device of examples 58-60, wherein said movement of said transmission is adapted to provide to at least one of said at least two movables blade with a linear movement comprising impact force.

Example 62. The device of examples 57-61, wherein said at least two movable blades comprise a retracted state where said at least two movable blades are not exposed thereby avoiding said at least two movable blades from cutting.

Example 63. The device of examples 58-62, wherein said motion is characterized by a frequency from about 1 Hz to about 100 Hz.

Example 64. The device of examples 57-63, wherein said at least two movable blade exit distally said distal end from about 0.15 mm to about 2 mm.

Example 65. The device of examples 57-64, wherein the movement of said at least two movable blades is towards each therefore allowing cutting by shearing.

Example 66. A lead extraction accessory, comprising:
a. a handle;
b. an elongated body having a first proximal end, a first distal end, and a first lumen extending from said first proximal end toward said first distal end, said lumen sized and shaped to fit over a cardiac lead extraction device;
c. said first distal end of said elongated body comprising an operational portion comprising having a second proximal end and a second distal end;
   wherein said operational portion comprises a controllable bendable flexible portion.

Example 77. A cardiac lead extraction system, comprising:
a. a handle;
b. an elongated body in communication with said handle;
c. a bendable flexible portion in communication with said elongated body, said bendable flexible portion comprising a first lumen sized and shaped to fit over a cardiac lead; said bendable flexible portion being more flexible than said elongated body;
d. an operational distal end in communication with said bendable flexible portion;
wherein said bendable portion is configured to bend to a bending radius of less than 4 cm while keeping said first lumen open; and
wherein said operational distal end comprises at least one lead extraction assistive tool, said operational distal end comprising a second lumen sized and shaped to fit over a cardiac lead, said second lumen being in communication with said first lumen, and said first lumen comprises an inner diameter of from about 1 mm to about 5 mm.

Example 78. The system of example 77, wherein said system further comprises a controllable steering mechanism configured to orient said operational distal end.

Example 79. The system of example 77, wherein said bendable portion is configured to bend to a minimum bending radius of from about 2 mm to about 15 mm.

Example 80. The system of example 77, wherein said bendable portion comprises at least one articulated structure configured to maintain said first lumen open.

Example 81. The system of example 77, wherein a size of said inner diameter is selected from the group consisting of:
a. from about 2 mm to about 8 mm; and
b. from about 4 mm to about 6 mm.

Example 82. The system of example 77, wherein the outer diameter of said cardiac lead extraction system is from about 5 mm to about 8 mm.

Example 83. The system of example 77, wherein said bendable flexible portion bends to a maximal angle of from about 35 degrees to about 150 degrees.

Example 84. The system of example 83, wherein an inner diameter of said bendable flexible portion changes in length from about 0% to about 10% during said maximal angle.

Example 85. The system of example 77, wherein said bendable flexible portion is configured to perform a movement from 0 degrees to about 180 degrees.

Example 86. The system of example 83, wherein one or more of the following is true:
a. said bendable flexible portion is capable of bending to said maximal angle during active deflection of the system while withstanding forces up to 3000 gf;
b. said bendable flexible portion is capable of bending to said maximal angle during passive deflection of the system while withstanding forces up to 500 gf.

Example 87. The system of example 77, wherein:
a. said elongated body comprises a first proximal end, a first distal end, and a third lumen extending from said first proximal end toward said first distal end, said third lumen sized and shaped to fit over a cardiac lead; and
b. said bendable flexible portion comprises a second proximal end, a second distal end and said first lumen extending from said second proximal end toward said second distal end, said second lumen sized and shaped to fit over a cardiac lead.

Example 88. The system of example 77, further comprising a motor.

Example 89. The system of example 88, wherein said motor is located at said handle.

Example 90. The system of example 77, further comprising a pedal in communication with said handle.

Example 91. The system of example 88, wherein said motor is located at said pedal.

Example 92. The system of example 90, wherein said pedal is used to activate and control said at least one lead extraction assistive tool.

Example 92a. The system of example 90, wherein said pedal comprises one or more of a rechargeable battery, a reusable battery and a replaceable battery.

Example 92b. The system of example 77, wherein said system comprises one or more of a rechargeable battery, a reusable battery and a replaceable battery.

Example 93. The system of example 77, wherein said handle is used to activate and control said at least one lead extraction assistive tool.

Example 94. The system of example 77, wherein at least one lead extraction assistive tool comprises one or more components configured to perform repeatable movement at a repetition rate of from about 1 Hz to about 100 Hz.

Example 95. The system of example 94, wherein said repetition rate is from about 5 Hz to about 60 Hz.

Example 96. The system of example 77, wherein said lead extraction assistive tool comprises a tissue cutter.

Example 97. The system of example 96, wherein said tissue cutter comprises at least one movable blade.

Example 98. The system of example 96, wherein said tissue cutter comprises at least one transmission attached to said motor; said transmission adapted to transfer motion from said motor to said at least one movable blade.

Example 99. The system of example 98, wherein said motion of said at least one movable blade is linear.

Example 100. The system of example 98, wherein said motion of said at least one movable blade is circular.

Example 101. The system of example 98, wherein said movement of said transmission is configured to provide said at least one movable blade with a linear movement comprising an impact force to apply on the tissue.

Example 102. The system of example 98, wherein said motion of said at least one movable blade is a combination of linear movement and circular movement.

Example 103. The system of example 98, wherein said motion of said at least one movable blade is characterized by a frequency from about 0.5 Hz to about 100 Hz.

Example 104. The system of example 98, wherein said motion of said at least one movable blade is characterized by a frequency from about 1 Hz to about 15 Hz.

Example 105. The system of example 98, wherein said at least one movable blade comprises a retracted state where said at least one movable blade is not exposed thereby minimizing said at least one movable blade from damaging tissue.

Example 106. The system of example 98, wherein said at least one movable blade exits distally said operational distal end from about 0.15 mm to about 2 mm.

Example 107. The system of example 96, wherein said tissue cutter comprises at least two movable blades.

Example 108. The system of example 107, wherein a relative movement of said at least two movable blades provides cutting by shearing.

Example 109. The system of example 77, wherein said bendable portion comprises at least one internal structure configured to transmit motion from said handle to said operational distal end through said elongated body.

Example 110. The system of example 77, wherein said lead extraction assistive tool comprises a lead cutter.

Example 111. The system of example 78, wherein said controllable steering mechanism comprises at least one wire that runs from said handle to said operational distal end, and wherein said at least one wire runs inside a counter sleeve on said elongated body.

Example 112. A cardiac lead extraction system, comprising:
a. a handle;
b. an elongated body in communication with said handle;
c. a bendable flexible portion in communication with said elongated body, said bendable portion being more flexible that said elongated body;
d. an operational distal end in communication with said bendable flexible portion;
   wherein said operational distal end comprises at least one lead extraction assistive tool comprising one or more components configured to perform repeatable movement at a repetition rate of from about 1 Hz to about 100 Hz.

Example 113. The system of example 112, further comprising a controllable steering mechanism configured to orient said operational distal end.

Example 114. The system of example 112, further comprising a motor configured to actuate said at least one lead extraction assistive tool.

Example 115. The system of example 112, further comprising one or more internal components configured to perform repeatable linear movement.

Example 116. The system of example 112, wherein said repetition rate is from about 5 Hz to about 60 Hz.

Example 117. A cardiac lead extraction system configured to be operated by a single operator, comprising:
a. a handle;
b. an elongated body in communication with said handle;
c. a bendable flexible portion in communication with said elongated body, said bendable portion being more flexible that said elongated body;
d. an operational distal end in communication with said bendable flexible portion, said operational distal end comprises at least one lead extraction assistive tool;
   wherein said system comprises at least one selected from the group consisting of:
e. an automatic lead tensioning mechanism configured to automatically pull said lead, thereby allowing a single operator to operate said system;
f. a controllable steering mechanism configured to orient said operational distal end;
g. a motor configured to actuate said at least one lead extraction assistive tool;
h. a lead cutter assistive component;
i. an operational distal end accessory, instead of said operational distal end, said operational distal end accessory comprising:
   I. a body configured to be mounted on a distal end of said elongated body;
   II. said at least one lead extraction assistive tool; and
   III. a hand controller configured to control said at least one lead extraction assistive tool.

An aspect of some embodiments of the invention relates to a method/device/system for substantially separating between the linear/longitudinal pushing force being applied by the physician through a lead extraction catheter and the pushing force being applied to the tissue, the method comprises bringing a lead extraction catheter through a vessel into contact with the adhesion site, and activating the device such that the tip of the device locally and/or temporally generates the majority of the longitudinal/linear impact/force applied to the tissue. In some embodiments, the device is characterized by having a mechanism at the distal end (the “tip”/the “head”) comprising a lumen with a radius from about 1 mm to about 7 mm. In some embodiments, the device is characterized by having a mechanism at the distal end (the “tip”/the “head”) comprising a lumen with a radius of at least 2.5 mm. For example, a radius from about 1.5 mm to about 3 mm; optionally from about 0.5 mm to about 5 mm; optionally from about 0.3 mm to about 10 mm; optionally from about 3 mm to about 10 mm; optionally from about 3 mm to about 15 mm; optionally above 5 mm. In some embodiments, the lumen is configured for passage of the lead. In some embodiments, the distal end of the device comprises one or more of the following mechanisms: (i) a flexible component for transferring linear (longitudinal) force forward along the catheter to the distal tip to provide longitudinal impact to the target tissue; (ii) a mechanism (e.g. a spring) for storing and abruptly releasing of energy wherein the storing of energy is internally within the tip and the abrupt releasing of energy has part of the motion being accelerating internally within the tip (without direct friction with external tissue) and part of the motion being extending outside the device to generate substantially longitudinal impact on the target tissue; and (iii) a tip orientation control mechanism to steer the tip and forces/impact application toward the desired direction, with bending of at least 20 degrees (from about 10 degrees to about 45 degrees, optionally from about 5 degrees to about 90 degrees, optionally from about 1 degree to about 180 degrees) with a radius of less than about 4 cm (optionally less than 3 cm, optionally less than 2 cm) while effectively transferring the forces through a flexible shaft towards the tissue and maintaining an open lumen of at least 2.5 mm for passage of the lead.

In some embodiments, the method/device/system, further comprising applying rotational motion to the target tissue. In some embodiments, the method/device/system, characterized by that it reduces the magnitude of pushing force required to penetrate and/or separate an adhesion site compared with the pushing force required when the device is not activated. In some embodiments, the method/device/system, being effective in penetrating adhesive tissue with pushing force being less than 800 gr. In some embodiments, the method/device/system, being effective in penetrating adhesive tissue with pushing force being less than 500 gr. In some embodiments, the method/device/system, being effective in penetrating adhesive tissue with pushing force being less than 300 gr. In some embodiments, the method/device/system, being effective in penetrating adhesive tissue with pushing force being less than 1300 gr. For example, from about 0 gr to about 1300 gr, optionally from about 50 gr to about 800 gr, optionally from about 100 gr to about 600 gr, optionally from about 450 gr to about 750 gr.

In some embodiments, the method/device/system, characterized by that it reduces the magnitude of lead pulling force required to penetrate and/or separate an adhesion site compared with the lead pulling force required when the device is not activated. In some embodiments, the method/device/system, being effective in penetrating adhesive tissue with lead pulling force being less than 800 gr. In some embodiments, the method/device/system, being effective in penetrating adhesive tissue with lead pulling force being less than 500 gr. In some embodiments, the method/device/system, being effective in penetrating adhesive tissue with lead pulling force being less than 300 gr. In some embodiments, the method/device/system, being effective in penetrating adhesive tissue with pushing force being less than 1300 gr. For example, from about 0 gr to about 1300 gr, optionally from about 50 gr to about 800 gr, optionally from about 100 gr to about 600 gr, optionally from about 450 gr to about 750 gr.

An aspect of some embodiments of the invention relates to a lead extraction catheter comprising one or more mechanisms selected from a group consisting of: (i) a steerable sheath; (ii) a lead cutter; (iii) a lead bending and/or fixating mechanism for firmly holding the lead against the catheter to enable cutting of the lead by the catheter (iv) a mechanism for indicating catheter pushing force; (v) a catheter gripping handle with catheter pushing force sensor/evaluation/indication; (vi) a handle for gripping the one or more lead pulling wires and/or lead locking stylets; (vii) a handle for gripping the one or more lead pulling wires and/or lead locking stylets with lead pulling force sensor/evaluation/indication/control and/or limiter; and (viii) a mechanism for gripping the one or more lead pulling wires and/or lead locking stylet with lead pulling distance and/or velocity control and/or indicator and/or limiter. (ix) a mechanism (as part of the catheter or as an independent device) that can be pushed ahead of the catheter main body, capable of inflating a balloon inside the blood vessel, to form a firm resistance for the catheter to push against, and/or to generate an anchoring point for the device within the vessel, and/or to assist in directing the device by centralizing/positioning the device within the vessel using the balloon as pushers for assisting in separation of the lead from surrounding tissue, and/or to use the balloon as a rescue balloon, for example, in case of bleeding and/or vein rupture, and/or to use the balloon as a blood blocker to allow intervention in case of bleeding; (x) modularity of the system, where the device may be composed of two separable parts, for example a hand-held part and a pedal part, with the electronics and potentially also the motor are part of the pedal component; (xi) a mechanism for sensing the position of the lead inside the catheter, i.e. who well the lead is centered in the catheter, and an indicator reporting this information to the user by visual or audio signals; (xii) a steering limiter configured to prevent excessive steering force to be applied, either to the device, to the tissue or to the lead.

In some embodiments, said lead extraction catheter incorporates/utilizes one or more of said mechanisms, and is characterized by that it reduces the magnitude of pushing force required to penetrate and/or separate an adhesion site compared with the pushing force required when the one or more mechanisms is not utilized. In some embodiments, said lead extraction catheter being effective in penetrating adhesive tissue with pushing force being less than 800 gr. In some embodiments, said lead extraction catheter being effective in penetrating adhesive tissue with pushing force being less than 500 gr. In some embodiments, said lead extraction catheter being effective in penetrating adhesive tissue with pushing force being less than 300 gr. For example, from about 0 gr to about 1300 gr, optionally from about 50 gr to about 800 gr, optionally from about 100 gr to about 600 gr, optionally from about 50 gr to about 300 gr, optionally from about 450 gr to about 750 gr.

In some embodiments, said lead extraction catheter incorporates/utilizes one or more of said mechanisms, and is characterized by that it reduces the magnitude of lead pulling force required to penetrate and/or separate an adhesion site compared with the lead pulling force required when the one or more mechanisms is not utilized. In some embodiments, said lead extraction catheter, being effective in penetrating adhesive tissue with pushing force being less than 800 gr. In some embodiments, said lead extraction catheter, being effective in penetrating adhesive tissue with pushing force being less than 500 gr. In some embodiments, said lead extraction catheter, being effective in penetrating adhesive tissue with pushing force being less than 300 gr. For example, from about 0 gr to about 1300 gr, optionally from about 50 gr to about 800 gr, optionally from about 100 gr to about 600 gr, optionally from about 50 gr to about 300 gr, optionally from about 450 gr to about 750 gr.

An aspect of some embodiments of the invention relates to an add-on device being used to control and/or augment and/or modulate the function of a lead extraction catheter (e.g. either laser based and/or mechanical based and/or thermal based and/or ablation based and/or combination thereof), the add-on device comprises one or more of: (i) a steerable outer sheath; (ii) a lead cutter; (iii) a lead bending and/or fixating mechanism for firmly holding the lead against the catheter to enable cutting of the lead by the catheter (iv) a mechanism for indicating catheter pushing force; (v) a catheter gripping handle with catheter pushing force sensor/evaluation/indication; (vi) a handle for gripping the one or more lead pulling wires and/or lead locking stylets; (vii) a handle for gripping the one or more lead pulling wires and/or lead locking stylets with lead pulling force sensor/evaluation/indication/control and/or limiter; and (viii) a mechanism for gripping the one or more lead pulling wires and/or lead locking stylet with lead pulling distance and/or velocity control and/or indicator and/or limiter.

In some embodiments, said add-on device is coaxial with the lead extraction catheter. In some embodiments, said add-on device is mounted around the lead extraction catheter with an overlapping length of at least along majority of the length of the catheter. In some embodiments, said add-on device is mounted around the lead extraction catheter with an overlapping length of no more than a third of the length of the catheter. In some embodiments, said add-on device having a side opening for being mounted on the lead extraction catheter from the side of the catheter.

In some embodiments, said add-on device characterized by that it reduces the magnitude of pushing force required to penetrate and/or separate an adhesion site compared with the pushing force required when the add-on device is not utilized. In some embodiments, said add-on device, being effective in penetrating adhesive tissue with pushing force being less than 800 gr. In some embodiments, said add-on device, being effective in penetrating adhesive tissue with pushing force being less than 500 gr. In some embodiments, said add-on device, being effective in penetrating adhesive tissue with pushing force being less than 300 gr. For example, from about 0 gr to about 1300 gr, optionally from about 50 gr to about 800 gr, optionally from about 100 gr to about 600 gr, optionally from about 50 gr to about 300 gr, optionally from about 450 gr to about 750 gr.

In some embodiments, said add-on device, characterized by that it reduces the magnitude of lead pulling force required to penetrate and/or separate an adhesion site compared with the lead pulling force required when the add-on device is not utilized. In some embodiments, said add-on device, being effective in penetrating adhesive tissue with lead pulling force being less than 800 gr. For example, from about 0 gr to about 1300 gr, optionally from about 50 gr to about 800 gr, optionally from about 100 gr to about 600 gr, optionally from about 50 gr to about 300 gr, optionally from about 450 gr to about 750 gr.

In some embodiments, said add-on device, being effective in penetrating adhesive tissue with lead pulling force being less than 500 gr. In some embodiments, said add-on device, being effective in penetrating adhesive tissue with lead pulling force being less than 300 gr. For example, from about 0 gr to about 1300 gr, optionally from about 50 gr to about 800 gr, optionally from about 100 gr to about 600 gr, optionally from about 50 gr to about 300 gr, optionally from about 450 gr to about 750 gr.

An aspect of some embodiments of the invention relates to a lead extraction catheter comprising one or more of: (i) a sensor for tissue contact force; and (ii) a sensor for tissue classification.

An aspect of some embodiments of the invention relates to an add-on device being used to control and/or augment and/or modulate the function of a lead extraction catheter (e.g. either laser based and/or mechanical based and/or thermal based and/or ablation based and/or combination thereof), the add-on device comprises one or more of: (i) a sensor for tissue contact force; and (ii) a sensor for tissue classification.

Following is a non-exclusive list including some examples of embodiments of the invention. The invention also includes embodiments, which include fewer than all the features in an example and embodiments using features from multiple examples, also if not expressly listed below.

Example 1. A cardiac lead extraction system, comprising:
a. a handle;
b. an elongated body in communication with said handle;
c. a bendable flexible portion in communication with said elongated body, said bendable flexible portion comprising a first lumen sized and shaped to fit over a cardiac lead; said bendable flexible portion being more flexible than said elongated body;
d. an operational distal end in communication with said bendable flexible portion;
wherein said bendable portion is configured to bend to a bending radius of less than 4 cm while keeping said first lumen open; and
wherein said operational distal end comprises at least one lead extraction assistive tool, said operational distal end comprising a second lumen sized and shaped to fit over a cardiac lead, said second lumen being in communication with said first lumen, and said first lumen comprises an inner diameter of from about 1 mm to about 8 mm.

Example 2. The system of example 1, wherein said system further comprises a controllable steering mechanism configured to orient said operational distal end.

Example 3. The system of example 1, wherein said bendable portion is configured to bend to a minimum bending radius of from about 2 mm to about 15 mm.

Example 4. The system of example 1, wherein said bendable portion comprises at least one articulated structure configured to maintain said first lumen open.

Example 5. The system of example 1, wherein a size of said inner diameter is selected from the group consisting of:
a. from about 2 mm to about 8 mm;
a. from about 2 mm to about 5 mm; and
b. from about 4 mm to about 6 mm.

Example 6. The system of example 1, wherein the outer diameter of said cardiac lead extraction system is from about 5 mm to about 8 mm.

Example 7. The system of example 1, wherein said bendable flexible portion bends to a maximal angle of from about 35 degrees to about 150 degrees.

Example 8. The system of example 7, wherein an inner diameter of said bendable flexible portion changes in length from about 0% to about 10% during said maximal angle.

Example 9. The system of example 1, wherein said bendable flexible portion is configured to perform a movement from 0 degrees to about 180 degrees.

Example 10. The system of example 7, wherein one or more of the following is true:
a. said bendable flexible portion is capable of bending to said maximal angle during active deflection of the system while withstanding forces up to 3000 gf;
b. said bendable flexible portion is capable of bending to said maximal angle during passive deflection of the system while withstanding forces up to 500 gf.

Example 11. The system of example 1, wherein:
a. said elongated body comprises a first proximal end, a first distal end, and a third lumen extending from said first proximal end toward said first distal end, said third lumen sized and shaped to fit over a cardiac lead; and
b. said bendable flexible portion comprises a second proximal end, a second distal end and said first lumen extending from said second proximal end toward said second distal end, said second lumen sized and shaped to fit over a cardiac lead.

Example 12. The system of example 1, further comprising a motor.

Example 13. The system of example 12, wherein said motor is located at said handle.

Example 14. The system of example 1, further comprising a pedal in communication with said handle.

Example 15. The system of example 12, wherein said motor is located at said pedal.

Example 16. The system of example 14, wherein said pedal is used to activate and control said at least one lead extraction assistive tool.

Example 17. The system of example 1, wherein said handle is used to activate and control said at least one lead extraction assistive tool.

Example 18. The system of example 1, wherein at least one lead extraction assistive tool comprises one or more components configured to perform repeatable movement at a repetition rate of from about 1 Hz to about 100 Hz.

Example 19. The system of example 18, wherein said repetition rate is from about 5 Hz to about 60 Hz.

Example 20. The system of example 1, wherein said lead extraction assistive tool comprises a tissue cutter.

Example 21. The system of example 20, wherein said tissue cutter comprises at least one movable blade.

Example 22. The system of example 20, wherein said tissue cutter comprises at least one transmission attached to said motor; said transmission adapted to transfer motion from said motor to said at least one movable blade.

Example 23. The system of example 22, wherein said motion of said at least one movable blade is linear.

Example 24. The system of example 22, wherein said motion of said at least one movable blade is circular.

Example 25. The system of example 22, wherein said movement of said transmission is configured to provide said at least one movable blade with a linear movement comprising an impact force to apply on the tissue.

Example 26. The system of example 22, wherein said motion of said at least one movable blade is a combination of linear movement and circular movement.

Example 27. The system of example 22, wherein said motion of said at least one movable blade is characterized by a frequency from about 0.5 Hz to about 100 Hz.

Example 28. The system of example 22, wherein said motion of said at least one movable blade is characterized by a frequency from about 1 Hz to about 15 Hz.

Example 29. The system of example 22, wherein said at least one movable blade comprises a retracted state where said at least one movable blade is not exposed thereby minimizing said at least one movable blade from damaging tissue.

Example 30. The system of example 22, wherein said at least one movable blade exits distally said operational distal end from about 0.15 mm to about 2 mm.

Example 31. The system of example 20, wherein said tissue cutter comprises at least two movable blades.

Example 32. The system of example 31, wherein a relative movement of said at least two movable blades provides cutting by shearing.

Example 33. The system of example 1, wherein said bendable portion comprises at least one internal structure configured to transmit motion from said handle to said operational distal end through said elongated body.

Example 34. The system of example 1, wherein said lead extraction assistive tool comprises a lead cutter.

Example 35. The system of example 2, wherein said controllable steering mechanism comprises at least one wire that runs from said handle to said operational distal end, and wherein said at least one wire runs inside a counter sleeve on said elongated body.

Example 36. A cardiac lead extraction system, comprising:
a. a handle;
b. an elongated body in communication with said handle;
c. a bendable flexible portion in communication with said elongated body, said bendable portion being more flexible that said elongated body;
d. an operational distal end in communication with said bendable flexible portion;
wherein said operational distal end comprises at least one lead extraction assistive tool comprising one or more components configured to perform repeatable movement at a repetition rate of from about 1 Hz to about 100 Hz.

Example 37. The system of example 36, further comprising a controllable steering mechanism configured to orient said operational distal end.

Example 38. The system of example 36, further comprising a motor.

Example 39. The system of example 36, further comprising one or more internal components configured to perform repeatable linear movement.

Example 40. The system of example 36, wherein said repetition rate is from about 5 Hz to about 60 Hz.

Example 41. A cardiac lead extraction system configured to be operated by a single operator, comprising:
a. a handle;
b. an elongated body in communication with said handle;
c. a bendable flexible portion in communication with said elongated body, said bendable portion being more flexible that said elongated body;
d. an operational distal end in communication with said bendable flexible portion, said operational distal end comprises at least one lead extraction assistive tool;
wherein said system comprises at least one selected from the group consisting of:
e. an automatic lead tensioning mechanism configured to automatically pull said lead, thereby allowing a single operator to operate said system;
f. a controllable steering mechanism configured to orient said operational distal end;
g. a motor;
h. a lead cutter assistive component;
i. an operational distal end accessory, instead of said operational distal end, said operational distal end accessory comprising:
I. a body configured to be mounted on a distal end of said elongated body;
II. said at least one lead extraction assistive tool; and
III. a hand controller configured to control said at least one lead extraction assistive tool.

Example 42. A model for evaluating performance of a lead extraction system, comprising:
a. at least one first tube segment comprising a long arc tube segment or a long straight tube segment;
b. at least one second segment comprising a curved segment comprising an angle of from about 80 degrees to about 100 degrees, said at least one second segment in communication with said at least one first tube segment.

Example 43. The model of example 42, comprising a lead or a lead mimicking wire attached to an inner curve at the adjacent end of the curved segment.

Example 44. The model of example 42, comprising a lead or a lead mimicking wire attached to the outer curve at the adjacent end of the curved segment.

Example 45. A method of lead tracking, comprising,
a. advancing a sheath along a lead, said sheath comprises at least a lumen suitable for passage of the lead and a flexible distal segment;
wherein said flexible distal segment is configured in accordance with one or more of a group consisting of:
b. allowing said flexible distal segment of said sheath to passively bend along a curve in said lead;
c. actively bending said flexible distal segment of said sheath; and
d. separating said lead from tissue using said bent sheath.

Example 46. The method of example 45, wherein actively bending is performed to match a lead curvature.

Example 47. The method of example 45, wherein actively bending is performed to bend said lead.

Example 48. The method of example 45, wherein separating comprises rotating a distal end of said bent flex dist segment.

Example 49. The method of example 45, wherein said sheath is actively bent based on feedback from imaging input.

Example 50. The method of example 45, wherein said sheath comprises an active component at the tip used for tissue separation

Example 51. The method of example 45, wherein said sheath is suitable for passage of a lead extraction tool within its lumen.

Example 52. An apparatus for lead separation, comprising:
a. a hollow elongated body sized to fit in a blood vessel and having a lumen sized to fit a lead of an electrical stimulator, said body having a hollow distal tip and said elongated body defining an axis;
b. an impactor having a first axial position and a second axial position, said first axial position being distal than said second axial position;
c. a local energy store coupled to said impactor and localized at said tip; and
d. an trigger which selectively releases said energy into said impactor, causing said impactor to move distally and apply axial force to tissue adjacent said tip.

Example 53. The apparatus of example 52, wherein said impactor moves within a lumen/cover/outer tube of the apparatus.

Example 54. The apparatus of example 52, wherein said local energy store is a spring.

Example 55. The apparatus of example 52, wherein a distance between said first axial position and said second axial position is from about 2 mm to about 2.5 mm.

Example 56. The apparatus of example 52, wherein said impactor comprises a blade.

Example 57. The apparatus of example 52, wherein said axial force is applied directly by a head of said impactor.

Example 58. The apparatus of example 52, wherein said axial force is applied indirectly via the tip of said apparatus.

Example 59. The apparatus of example 52, wherein said local energy store is charged by rotating said impactor in relation to said tip.

Example 60. The apparatus of example 52, wherein a blade located at said tip cuts when said impactor is periodically activated.

Example 61. The apparatus of example 52, wherein said impactor comprises a non-uniform proximal side matching a non-uniform geometry at said tip, said non-uniform comprises different circumferential locations at different axial positions.

Example 62. The apparatus of example 52, wherein said impactor impacts between one time to three times per rotation.

Example 63. A sheath for a lead extraction (LE) device, comprising at least one sensor.

Example 64. The sheath of example 63, wherein said sheath is an external sheath to be mounted in a LE device.

Example 65. The sheath of example 63, wherein said sheath is a built-in sheath of a LE device.

Example 66. The sheath of example 63, wherein said at least one sensor is a tissue characterization sensor.

Example 67. The sheath of example 63, wherein said sheath further comprises a connection to a 3D mapping system.

Example 68. The sheath of example 63, wherein said sheath further comprises a connection to at least one imaging system.

Example 69. The sheath of example 63, wherein said sheath is further connected to an ultrasound measurement system.

Example 70. The sheath of example 63, wherein said sensors are located at the tip of said device.

Example 71. The sheath of example 63, wherein said sensors are around a interaction area between said device and a tissue to be separated.

Example 72. The sheath of example 63, wherein said sensor comprises a temperature sensor.

Example 73. The sheath of example 63, wherein said sensor measures the lead location.

Example 74. The sheath of example 63, wherein said sensor provides an image of a vessel wall.

Example 75. The sheath of example 63, wherein said sensor detects a distance to a vessel wall.

Example 76. The sheath of example 63, wherein said sensor allows the detection of the 3D position and/or orientation of a tip of said LE device.

Example 77. The sheath of example 63, wherein said sensor allows the detection of the 3D position and/or orientation of a vessel wall.

Example 78. The sheath of example 63, wherein said sensor allows the detection of the 3D position and/or orientation of the lead.

Example 79. The sheath of example 63, wherein said sensor allows the detection of the relative 3D position and/or relative orientation between the tip of the device and the lead.

Example 80. The sheath of example 63, wherein said sensor allows the detection of the relative 3D position and/or relative orientation and/or relative distance between the tip of the device and a vessel wall.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example, and for purposes of illustrative discussion of embodiments of the invention. In this regard, the description taken with the drawings makes apparent to those skilled in the art how embodiments of the invention may be practiced.

In the drawings:

FIG. 1a is a schematic view of the kit, according to some embodiments of the present invention;

FIG. 1b is a schematic view of the lead extraction device, according to some embodiments of the present invention;

FIG. 2a is a schematic representation of exemplary components, exemplary tools and exemplary mechanisms according to their exemplary location on the device and/or outside the device, according to some embodiments of the present invention;

FIG. 2b is a schematic representation of the system, according to some embodiments of the invention;

FIG. 2c is a schematic representation of an exemplary shaft, according to some embodiments of the invention;

FIGS. 2d, 2e, 2f, 2g, 2h, 2i are schematic representations of the shaft and torque transmission mechanism, according to some embodiments of the present invention;

FIG. 2j is a schematic representation of an exemplary shaft made using different techniques, according to some embodiments of the invention;

FIGS. 3a, 3b, 3c, 3d, 3e are schematic views of exemplary incorporated steering mechanisms, according to some embodiments of the present invention;

FIGS. 3f, 3g, 3h, 3i, 3j, 3k, 3l, 3m, 3n, 3o, 3p, 3q, 3r, 3s, 3t, 3u, 3v, 3w, 3x, 3y, 3z, 4a and 4b are schematic views of exemplary handles and mechanisms located in the handle, according to some embodiments of the present invention;

FIG. 4c is a schematic representation of another configuration of an exemplary breaking-prevention mechanism, according to some embodiments of the invention;

FIGS. 4d, 4e and 4f are schematic representations of exemplary handles with ergonomic features, according to some embodiments of the invention;

FIGS. 4g, 4h, 4i, 4j, 4k, 4l, 4m, 4n, 4o, 4p, 4q, 4r, 4s, 4t, 4u, 4v, 4w, 4x, 4y, 4z, 5a and 5b are schematic representations of structures of hinges and distal tips, according to some embodiments, of the present invention.

FIGS. 5c, 5d, 5e, 5f, 5g, 5h, 5i, 5j, 5k, 5l, 5m and 5n are schematic views of exemplary embodiments of some components in the flexible region, according to some embodiments of the present invention;

FIGS. 5o, 5p, 5q, 5r, 5s, 5t are schematic views of exemplary embodiments of some operational components located at the distal head, according to some embodiments of the present invention;

FIGS. 6a, 6b, 6c, 6d, 6e, 6f, 6g are schematic views of exemplary embodiments of some exemplary mechanisms of action performed by some operational components located at the distal head, according to some embodiments of the present invention;

FIG. 6h is a schematic representation of an impact element, according to some embodiments of the invention;

FIGS. 7a, 7b are schematic views of exemplary embodiments of some exemplary mechanisms of movement performed at the distal head, according to some embodiments of the present invention;

FIGS. 8a, 8b, 8c, 8d, 8e, 8f are schematic views of exemplary embodiments of exemplary activation movements of the distal head, according to some embodiments of the present invention;

FIGS. 9a, 9b are schematic views of exemplary embodiments of vibration of the outer tube, according to some embodiments of the present invention;

FIGS. 10a, 10b, 10c, 10d, 10e are schematic views of exemplary eccentric rings, according to some embodiments of the present invention;

FIGS. 11a, 11b, 11c, 11d, 11e, 11f, 11g, 11h, 11i are schematic views of exemplary tissue spreaders, according to some embodiments of the present invention;

FIGS. 12a, 12b, 12c, 12d are schematic views of exemplary lead wire grasping tool, according to some embodiments of the present invention;

FIG. 13 is a schematic view of an exemplary integration of spectroscopy components with ablation components at the distal head, according to some embodiments of the present invention;

FIGS. 14a, 14b are schematic views of exemplary lead cutter tool, according to some embodiments of the present invention;

FIGS. 14c, 14d, 14e, 14f, 14g are schematic views of exemplary wire lead cutter tool, according to some embodiments of the present invention;

FIGS. 15a, 15b, 15c, 15d, 15e are schematic views of exemplary tension control and movement limiting mechanism, according to some embodiments of the present invention;

FIGS. 15f, 15g are schematic views of exemplary articulated structures intended for catheter steering, according to some embodiments of the present invention;

FIG. 16 is a schematic view of an exemplary opto-mechanical tool located at the distal head, according to some embodiments of the present invention;

FIGS. 17a, 17b are schematic views of an exemplary opto-mechanical tool located at the distal head, according to some embodiments of the present invention;

FIGS. 18a, 18b are schematic views of an exemplary opto-mechanical tool located at the distal head, according to some embodiments of the present invention;

FIGS. 19a, 19b are schematic views of an exemplary electro-mechanical tool located at the distal head, according to some embodiments of the present invention;

FIG. 20a, 20b are schematic figures related to capacitive-inductive force sensing, according to some embodiments of the present invention;

FIG. 21 is a schematic view of an exemplary LC based force sensor, according to some embodiments of the present invention;

FIGS. 22a, 22b are schematic figures related to exemplary sensors, according to some embodiments of the present invention;

FIGS. 23a, 23b, 23c, 23d, 23e are schematic views of an exemplary mechanism located at the handle, according to some embodiments of the present invention;

FIGS. 24a, 24b, 24c, 24d are schematic views of exemplary mechanisms located at the handle, according to some embodiments of the present invention;

FIGS. 24e, 24f, 24g, 24h, 24i, 24j, 24k, 24l, 24m, 24n, 24o, 24p, 24q 24r, 24s, 24t, 24u 24v, 24w, 24x, 24y, 24z, 25a, 25b, 25c and 25d are schematic views of exemplary mechanisms located at the distal head, according to some embodiments of the present invention;

FIG. 25e is a schematic representation of an exemplary blade for engaging calcifications during the lead extraction procedure, according to some embodiments of the invention;

FIG. 25f is a schematic representation of an internal tapered configuration of an exemplary inner shaft, according to some embodiments of the invention;

FIG. 25g is a schematic representation of an exemplary handle of the lead extraction device, according to some embodiments of the present invention;

FIGS. 25h, 25i, 25j, 25k, 251 are schematic views of exemplary balloon embodiment, according to some embodiments of the present invention;

FIG. 25m is a schematic representation of an exemplary additional outer sheath, according to some embodiments of the invention;

FIGS. 26a, 26b, 26c, 26d, 26e are schematic views of exemplary pulling/grapping device, according to some embodiments of the present invention;

FIG. 27 are schematic views of exemplary pulling device, according to some embodiments of the present invention;

FIG. 28 is a schematic view of exemplary steerable sheath embodiment, according to some embodiments of the present invention;

FIG. 29 is a schematic view of exemplary steerable sheath embodiment, according to some embodiments of the present invention;

FIGS. 30a, 30b are schematic views of exemplary steerable sheath embodiment, according to some embodiments of the present invention;

FIG. 31 is a schematic view of exemplary steerable sheath embodiment, according to some embodiments of the present invention;

FIG. 32 is a schematic view of exemplary steerable sheath embodiment, according to some embodiments of the present invention;

FIGS. 33a, 33b, 33c are schematic views of exemplary attachment ring for LE device embodiment, according to some embodiments of the present invention;

FIG. 34 are schematic views of exemplary pulling/grapping accessory device, according to some embodiments of the present invention;

FIG. 35 are schematic views of exemplary pulling accessory device, according to some embodiments of the present invention;

FIGS. 36a, 36b, 36c, 36d, 36e, 36f, 36g, 36h are schematic views of exemplary lead cutting accessory device, according to some embodiments of the present invention;

FIGS. 37, 38, 39 and 40 are schematic flowcharts of exemplary methods, according to some embodiments of the present invention;

FIG. 41 is a schematic representation of a femoral approach, according to some embodiments of the invention;

FIG. 42 is X-ray pictures of defibrillator leads implanted in sheep;

FIG. 43 is pictures showing leads covered with fibrotic, possibly calcified, tissue extracted from sheep;

FIG. 44 is an exemplary model for lead extraction system evaluation, according to some embodiments of the invention;

FIG. 45 is an exemplary element of the model which allow the mimicking of adhesion and positioning of a lead or lead mimicking wire in a lateral or medial path along the vein model;

FIGS. 46a, 46b, 46c, 46d are schematic representations of exemplary scenarios where lead extraction devices might cut the lead or damage the vessel's wall;

FIGS. 47a and 47b are graphs showing exemplary High and Low Motor speed control, respectively, according to some embodiments of the invention.

FIGS. 48a, 48b, 48c, 48d, 48e, 48f, and 48g are exemplary distal end diameter configurations, according to some embodiments of the invention;

FIG. 49 is a schematic representation of an exemplary lead extraction device with two balloons and drainage system, according to some embodiments of the invention;

FIGS. 50a and 50b schematically show an exemplary double blade configuration, according to some embodiments of the invention; and

FIGS. 51a, 51b, 51c, 51d, 51e, 51f, 51g and 51h are schematic representations of exemplary leadless pacemaker extraction, according to some embodiments of the invention.

### CONTENTS

1. Overview
2. Exemplary lead extraction kit
3. Exemplary sheath
   3.1 Exemplary incorporated steering mechanism
   3.2 Exemplary reinforced central lumen
4. Exemplary distal head
   4.1 Exemplary steering tool of the distal head
      4.1.1 Exemplary inner bending shaft
   4.2 Exemplary tissue cutting tool
      4.2.1 Exemplary concentric rotating blades
      4.2.2 Exemplary circumferential rotating blades
      4.2.3 Exemplary impact tip
   4.3 Exemplary motion mechanisms
   4.4 Exemplary vibration of the distal head
   4.5 Exemplary eccentric rings
   4.6 Exemplary tissue spreaders
   4.7 Exemplary lead wire grasping
   4.8 Exemplary tissue and binding site assessment
   4.9 Exemplary IR Spectroscopic classification of matter distally to the device head
   4.10 Exemplary ultrasonic classification of matter distally to the device head
   4.11 Exemplary lead cutter
5. Exemplary general mechanisms/characteristics of the device
   5.1 Exemplary motion repetition
   5.2 Exemplary modifiable mechanical properties
   5.3 Exemplary combinatorial use of components/embodiments
   5.4 Exemplary characteristics of the pull-wires of the device
   5.5 Exemplary tension control and movement limiting mechanism
6. Exemplary characteristics of force measurements in the device
   6.1 Exemplary force transducer in the distal portion of the device
   6.2 Exemplary Model and Shape Based Force Estimation
   6.3 Exemplary opto-mechanical methods
      6.3.1 Exemplary optical methods based on reflective intensity of light
      6.3.2 Exemplary Fiber Bragg Grating methods based on wavelength shift
   6.4 Exemplary electro-mechanical methods
      6.4.1 Exemplary PVDF force sensing
      6.4.2 Exemplary capacitive-inductive force sensing
   6.5 Force analysis unit—Exemplary feature
   6.6 Lead centering detection unit
7. Handle of the device and motion
   7.1 Exemplary linear impact element/ring motion of a LE device
   7.2 Exemplary dual motion cutting mechanism—rotating impact element/ring
   7.3 Exemplary fluid dynamics and forces
   7.4 Exemplary general description of the handle assembly
8. Exemplary balloon embodiment
9. Additional information
10. Exemplary Pulling/Grapping device
11. Exemplary Pulling device
12. Exemplary Accessories
   12.1 Steerable sheath (for any LE device)
   12.2 Exemplary attachment ring for LE device
   12.3 Exemplary pulling/grapping accessory device
   12.4 Exemplary pulling device accessory
   12.5 Exemplary tissue and binding site assessment accessory
   12.6 Exemplary lead cutter accessory
   12.7 Exemplary pulling rod accessory
13. Exemplary methods

### DESCRIPTION OF SPECIFIC EMBODIMENTS OF THE INVENTION

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In the following paragraphs, the term “force” is used. In some embodiments, force is defined by using the unit “gram(s)—gr”. It should be understood that the references to forces refer to “gram force” (defined as a unit of force defined as a force that produces an acceleration equal to the acceleration of gravity when acting on a mass of one gram, where one gram-force is equal to 9.80665E-3 Newton), or Newton, or Kg*m/s², etc. The use of the term “gr” or “Kg” refer to those force units, and a person having skills in the art would know the meaning and applicable conversions of such units.

### 1. Overview

An aspect of some embodiments of the invention relates to a device for facilitating pacemaker/defibrillator lead extraction comprising of an elongated shaft having a length greater than 60 cm (optionally greater than 80 cm, optionally greater than 100 cm), said shaft configured to have pushability and flexibility suitable for passage through iliac and femoral veins towards the heart to perform dilation and removal of tissue. In some embodiments, said shaft comprises a lumen for passage of a tool and/or a lead with inner diameter of at least 4 mm (for example in a 13Fr device model), and/or at least 3.5 mm (for example in an 11Fr model) and/or at least 2.8 mm (for example in a 9Fr device model). In some embodiments, said shaft had a steerable distal end controller by a handle connected to the shaft. In some embodiments, said steerable distal end comprises a bending segment with a length of at least 5 cm (from about 1 cm and about 10 cm, optionally from about 0.1 cm to about 15 cm, for example from about 2 cm to about 8 cm, for example, 4 cm, 6 cm, 8 cm) and in some options up to 140 mm or more. In some embodiments, the bending segment reaches a steering to at least 75 degrees and up to 110 degrees or more, while sustaining side forces of at least 200 gr and up to 400 gr or more, and maintaining substantially circular open lumen along the bending segment. In some embodiments, the device comprises a lumen with a radius of from about 1 mm to about 7 mm. In some embodiments, the inner diameter is at least 5 mm (for example, from about 4 mm to about 7 mm, optionally from about 5 mm to about 10 mm, optionally from about 5 mm to about 25 mm). In some embodiments, the shaft comprises two portions, an inner portion (inner shaft) and an outer portion (outer shaft) wherein said inner shaft rotates within the outer shaft while maintaining said open inner lumen. In some embodiments, said shaft comprises as operable tip comprising components for facilitating lead extraction. In some embodiments, the components comprise at least one dilation blade. In some embodiments, said components comprise at least one capturing element useful for capturing a lead. In some embodiments, said components comprise at least one impact element for facilitating tissue separation. In some embodiments, said components include at least one cutting tool. In some embodiments, said components comprise at least one pushing and/or clearance tool for clearing blockade. In some embodiments, said device includes a hemostatic valve. In some embodiments, said devise includes a kit comprising at least one of: dilators, introducer, ports, hemostatic valve, a syringe, guide wire, external sheath. In some embodiments, said devise includes a kit with an additional internal sheath having a smaller diameter that can be inserted into the inner shaft, all the way from the handle to the distal side, tip side. In some embodiments, the device can be implemented also for left side approach and particularly for right side approach. For example, having the pushability and flexibility to pass through the vein without damaging the vein wall through a right side (subclavian) entry towards the heart without a need to pull the electrode to align the vein and the device with more than 500 gr., optionally without any pulling at all.

In some embodiments, entry from the right subclavian vein into the SVC is considered complex due to small radius of curvature. Often, stiff devices, or devices with external sheath have difficulties to pass such a curve, or alternatively, a device which might be too soft has difficulty to pass through the vein and take the turn through the friction and towards an adhesion site without substantial pilling of the wires and leads. In some embodiments, the device's proximal part is relatively less flexible and aimed to provide more push-ability and torque-ability, and the shaft gradually becomes softer and more flexible towards its distal end. In some embodiments, the distal segment has a substantially more flexible structure that enables the device to bend in about 90 degrees (for example from about 45 degrees to about 135 degrees, optionally from about 1 degree to about 180 degrees) along a path of less than 10 cm (for example 8 cm, 6 cm, 4 cm). For example along a path of about 7 cm or 5 cm while maintaining the functionality of the device in terms of passing torque and activation of the tip and while maintaining the lumen open for passage of the leads and pulling wire, while keeping away from the vein walls. In some embodiments, such bending is achieved without any pulling of a wire or lead, or with minimal pulling of less than 300-500 gr. In some embodiments, such turning is assisted by steering, thus reduces the need for pulling a wire or lead. In some embodiments, the device fits itself to the vein structure, since it is flexible, pushable and since the tip bends when it “feels” contra-force from the vein walls.

An aspect of some embodiments of the invention relates to a lead extraction device having an inner diameter of more than or equal to 9Fr, more than or equal to 11Fr, more than or equal to 13Fr, more than or equal to 15Fr. In some embodiments, the lead extraction device is configured to have an inner lumen that is substantially circular and it is kept open during the lead extraction procedure. In some embodiments, the lead extraction device is configured to keep its steering capabilities against forces higher than 300 gr, optionally higher than 400 gr. In some embodiments, the lead extraction device is steered using wires configured to withstand forces higher than 4 kg, and withstanding pulling forces of higher than 5 kg. In some embodiments, the lead extraction device comprises a breaking-prevention mechanism, optionally at the handle, configured to prevent breaking of the wires or peripherals responsible for the steering. In some embodiments, the lead extraction device is used a steering device for other devices. In some embodiments, the lead extraction device allows the insertion of other devices in the inner lumen, for example, a snare, workstation, pulling wires, bulldog, biopsy devices, needles and any other required tools.

An aspect of some embodiments of the present invention relates to a lead extraction device that can be operated by one user alone and does not require the assistance of a second operator to perform the lead extraction procedure. In some embodiments, a user can operate all the mechanical features of the lead extraction device using two hands only, without the need of assistance from a second person. In some embodiments, a pedal is optionally used to activate mechanical features of the lead extraction device.

An aspect of some embodiments of the present invention relates to reducing forces exerted on the leads, the veins, and/or heart tissue during a procedure for removal of cardiac leads from the body. In some embodiments, reduction of forces includes reducing pressure between a dilating tip of the device and the tissue while dilating the fibrous tissue encapsulating the lead. In some embodiments, a lead extraction device provides feedback about the pressure applied between the tip of the device and the tissue. In some embodiments, a deflectable sheath follows the bends of the lead within the veins while the lead is under reduced tension and/or while reducing a force applied to the sheath. In some embodiments, a lead extraction device provides controlled tension and/or limited pulling distance on the lead. In some embodiments, for example, limiting tension and/or pulling distance on the lead may prevent accidental pulling of the lead harder than and/or further than intended. In some embodiments, a lead extraction device provides feedback about the position and/or bending and/or the curvature of the tip of the device during the lead extraction procedure. In some embodiments, the device include ability to cut the lead itself so that the distal component of the lead may be abandoned in the body, while the proximal part is extracted without forcefully puling and tearing the lead.

In some embodiments, the lead extraction device separates the lead from the encapsulating fibrous tissue with reduced force exertion on the leads, veins, and/or heart tissue. In some embodiments, the device is easy to use, even for inexperienced users, which provide a potential advantage over similar prior art devices. In some embodiments, examples of the factors that enable ease of use of a device: a highly effective device in relation to the actions performed by the tip mechanism of the device (for example, separating the lead from tissue), which potentially reduces the efforts performed by the user during the procedure, a device that is configured to balance between pushability and flexibility and torquability in a manner that fits the mechanical properties and curvatures of the venous path, a device that is ergonomic, a device that comprises electrical motorized control to ease the activation process and reduce hand fatigue, a device the enables maneuvering the shaft through the curvature of venous path along the lead orientation, including providing steering control of the distal end of the device, a device that can be operated by a single user (only one hand advances/actuates the device while the other hand applies traction to the lead), a mechanical extraction device that comprises a pedal that allows the user to actuate the device in an easy and comfortable manner. All these and others would be apparent to a person having skills in the art after reading the present invention.

An aspect of some embodiments of the invention relates to provide a lead extraction device that requires from the user to apply fewer forces during the procedure. In some embodiments, reduction of required force from the user is performed by actively targeting and orienting the forces towards the adhesive tissue while avoiding the vessels. In some embodiments, reduction of required force from the user is performed by translocating the generation of forces to the distal end of the device. In some embodiments, translocating the generation of forces to the distal end comprises providing an active lead extraction accessory at the distal end of the device. In some embodiments, the device weights less than 300 grams (optionally is more than 300 gr, for example from about 300 gr to about 400 gr without the alimentation cord. In some embodiments the handle weights about 600 gr with the alimentation cord), which enable the user to have one hand on the shaft of the device and the other on the lead that is being extracted, while leaving the handle in the air.

An aspect of some embodiments of the invention relates to provide a lead extraction device that is flexible enough to steer inside the blood vessel without the need for active steering, but stiff enough so the user can push the device during the procedure. In some embodiments, the device is flexible enough to adapt itself to the blood vessel. In some embodiments, the force applied by the user on pulling the lead is less than the force applied on pushing the device. In some embodiments, the force applied by the distal head in less than a 1 Kg force. In some embodiments, the user does not pull the lead during the lead extraction procedure, just hold it to keep it from tangling inside the vessel during the extraction. In some embodiments, the user pushes the device as much as the user finds suitable, while the force applied by the distal tip will still be less than 1 Kg force.

An aspect of some embodiments of the invention relates to provide a lead extraction device comprising an internal element having a linear movement and another internal element having a rotational movement, the device comprising an additional external sheath, where the thickness of the device, including all the internal mechanisms, from the outer diameter of the most external part to the internal diameter of the most internal part is less than 8 mm.

An aspect of some embodiments of the invention relates to a LE device having a lumen configured to receive a lead and comprising a tissue cutter at the distal end where the device comprises a minimal distance between the outer diameter and the inner diameter to enable easy passage of the device through the tissue.

An aspect of some embodiments of the invention relates to a ratchet mechanism for a tissue cutter mechanism of a lead extraction device configured to allow the device to activate two different actions by changing the direction of the rotation of the actuation mechanism of the tissue cutter mechanism. In some embodiments, rotation to one direction causes a double action of the tissue cutter mechanism, for example cutting and providing impact, while rotation to the other direction causes the tissue cutter mechanism to cut only. In some embodiments, impact actions are used to separate/cut hard tissue, while rotation actions are used to separate/cut soft tissue (see below paragraph related to “differential interaction with tissue”).

An aspect of some embodiments of the invention relates to flexibility and steerability of a catheter segment comprising a lumen wide enough for passage of a pacemaker/defibrillator lead, said catheter segment configured to bend towards radius of curvature of less than 40 mm without failing and/or without substantially reducing the diameter of the lumen. In some embodiments, the ID of said lumen of the flexible segment and/or active end is greater than 8 F, for example in a range of from about 8 F to about 15 F, for example, in the rage of from about 9 F to about 13 F, for example about 9 F or about 11 F or about 13 F. In some embodiments, said failing comprises a break of the shaft and/or a kink and/or a stuck in the cardiac lead extraction mechanism. In some embodiments, said without substantially reducing the diameter of the lumen include reduction of about greater than 10% in the diameter, or reduction of more than 20% in the diameter of the lumen. In some embodiments, said capable of bending comprises bending towards radius of curvature of less than 25 mm, without failing or without substantially reducing the diameter of the lumen. In some embodiments, said capability of bending is supported by at least one structure, for example a hypo-tube structure, in at least a part of said catheter segment. In some embodiments, said structure is part of an outer shaft of said catheter segment. In some embodiments, said capability of bending is supported by at least one metal spring having a configuration as shown in the table below in the section “Exemplary dimensions of parts of the system”.

In some embodiments, the OD of the tip at the distal side is less than 7.2 mm or less than 7.1 mm or less than 7 mm or less than 6.9 mm, for example from about 3 mm to about 7.2 mm, optionally from about 3.5 mm to about 7 mm, optionally from about 4 mm to about 6.5 mm, for example 7.2 mm, 7.1 mm, 7 mm, 6.9 mm, 6.8 mm, 6.5 mm, 6 mm, 5.5 mm, 5 mm, 4.5 mm, 4 mm, 3.5 mm, 3 mm. Optionally less than 3 mm, for example 2.5 mm, 2 mm, 1.5 mm.

In some embodiments, the ID of the tip at the distal end is more than 4.2 mm or 4.3 mm or 4.4 mm or 13 F (−/+0.05 mm). Optionally more or equal to 1.5 mm. Optionally in the range of from about 1.5 mm to about 6 mm. For example from about 1 mm to about 6 mm, optionally from about 2 mm to about 7 mm, optionally from about 3 mm to about 5 mm. For example, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm.

In some embodiments, the length of the distal end, not including the hinge, is for example less than 25 mm, preferably less than 20 mm, preferably less than 18 mm, preferably less than 15 mm. Optionally from about 5 mm to about 10 mm. Optionally from about 9 mm to about 16 mm.

In some embodiments, the thickness at the distal side of the tip from the ID of the tip to the OD of the tip, is less than 1.2 mm, or less than 1.25 mm or less than 1.3 mm or less than 1.35 mm or less than 1.4 mm. For example, from about 0.5 mm to about 2 mm, optionally form about 0.3 mm to about 2.5 mm, optionally from about 0.1 mm to about 3 mm, for example 0.1 mm, 0.2 mm, 0.3 mm, 0.4 mm, 0.5 mm, 0.6 mm, 0.7 mm, 0.8 mm, 0.9 mm, 1 mm, 1.5 mm.

An aspect of some embodiments of the invention relates to passive and active methods of lead tracking, comprising, advancing a sheath along a lead; allowing a tip of said sheath to bend with a bend in the lead; actively bending said tip of said sheath using an actuator; and separating said lead from tissue using said bent sheath. In some embodiments, bending is performed actively to match lead curvature. In some embodiments, bending is performed actively to bend the lead. In some embodiments, separating comprises rotating a distal end of said bent tip. In some embodiments, the user bends the device based on feedback from imaging. In some embodiments, separating the lead from the tissue comprises sideways steering the tip to assist in the separation of the lead from the tissue and/or from other leads, rather than only advancing and/or dilating.

An aspect of some embodiments of the invention relates to providing an impact mechanism that requires little travel distance to be loaded, little travel impact distance and generates a strong impact. In some embodiments, little travel distance is from about 1.5 mm to about 3 mm. In some embodiments, strong impact is from about 2.5 gm*m/s to about 10 gm*m/s.

An aspect of some embodiments of the invention relates to providing a LE device that comprises a flexible portion that is more flexible than the lead that is being extracted.

An aspect of some embodiments of the invention relates to methods of extracting a lead using a lead extraction device comprising a flexible element and at least one lead extraction assistive tool at the distal end. In some embodiments, the method comprises actively steering the distal end of the device. In some embodiments, the method comprises passively allowing the device to follow the lead. In some embodiments, the method comprises activating said at least one lead extraction assistive tool utilizing a pedal, thereby allowing a single user to perform the method of extraction. In some embodiments, the method comprises utilizing imaging means to monitor the advancement of the LE device. In some embodiments, the method comprises actively holding the lead while not pulling the lead.

An aspect of some embodiments of the invention relates to a system that comprises multi-function (cutting and/or impact) for a composite interaction with the adhesive tissue, said system provides a powerful interaction for hard tissue while softer interaction for soft tissue. In some embodiments, the composite interaction comprises both interacting with soft tissue and with hard tissue, optionally in the same manner or in a different manner.

An aspect of some embodiments of the invention relates to a system that comprises multi-function (cutting and/or impact) for a differential interaction with the adhesive tissue, said system provides a powerful interaction for hard tissue while softer interaction for soft tissue. In some embodiments, the differential interaction comprises both interacting with soft tissue and with hard tissue, but in different manner.

While some of the examples refer specifically to cardiac leads, cardiac lead extraction devices and methods, it is clear that the devices and methods disclosed herein are useful for extracting other leads in blood vessels (or other lumens) where the leads may stick. It should be also noted that the device can be mounted on anything embedded in the heart tissue (e.g. sensors, pacemakers) and remove them.

In the following disclosure the term “distal” refers to the general direction further from a user (e.g. a physician), while the term “proximal” refers to the general direction closer to the user; for example, something located distally may be in the body (e.g. towards the heart), and proximal may be, for example, outside the body or towards a handle, if any.

Some embodiments of the invention relate to an improved lead extraction device where the user may choose a suitable size of the device for the extraction. This means that a same or similar device design may be provided in a plurality of sizes (e.g. inner diameter (ID)). For example, a physician may choose the suitable ID according to the lead. In some embodiments, the minimum distance between the ID of the LE device and the OD of the lead is from about 0.2 mm to about 1.5 mm; optionally from about 0.5 mm to 1 mm; optionally from about 0.7 mm to about 0.9 mm. Alternatively, an operator may choose a suitable outer diameter (OD) and/or other physical attribute. For example, an ID may range between 2 mm to 8 mm, or optionally between 3 mm to 7 mm, or optionally, between 4 mm to 6 mm.

### 2. Exemplary Lead Extraction Kit

In some embodiments, the kit includes all necessary mechanisms and accessories needed in order to perform a safe and quick extraction of a cardiac lead while minimizing the chance of damaging the tissue surrounding the lead and minimizing the physical efforts required by the user to perform the extraction. In some embodiments, the kit comprises one device. In some embodiments, the kit comprises more than one device.

In some embodiments, the kit is an add-on/accessories kit for existing lead extraction devices. See below for further explanation on accessories. In some embodiments, the kit comprises one accessory. In some embodiments, the kit comprises more than one accessory.

In some embodiments, the lead extraction device comprises at least one of the following characteristics: highly maneuverability at the distal end of the lead extraction device (i.e.: steering mechanism); easy control of the different mechanisms of the lead extraction device at the proximal end of the lead extraction device (by using a handle, a pedal and/or a combination thereof); high precision of the mechanisms responsible of separating the lead from the surrounding tissue; effective lead cutting mechanisms; or any combination thereof. In some embodiments, separating the lead from the tissue comprises sideways steering the tip to assist in the separation of the lead from the tissue and/or from other leads, rather than only advancing and/or dilating.

### 2.1. Exemplary General Characteristics of a Lead Extraction Kit

In some embodiments, the mechanisms/tools/accessories described below are powered from outside the body. For example, they can be powered using manual or motorized means.

In some embodiments, the LE device includes one or more of the components/tools/accessories described below, as integral parts of the device. In some embodiments, the components/tools/accessories are independent parts that are configured and adapted to be used as accessories to LE devices to enhance their capabilities (e.g. steerability, manipulation, etc.).

In some embodiments, the components described below are connected to at least one indicator located at the handle of the device and/or other device outside the body of the patient, which enables the user to be aware of actions related to the lead extraction procedure. In some embodiments, the indicator is an interface that provides clear and simple feedback to the user, for example, about power level, steering direction, rotation direction and impact mode. In some embodiments, the indicator is a force indicator, which provides real-time information regarding the force applied when pushing/pulling. In some embodiments, the indicator is a meter, a screen showing colors, a sound, or any other suitable indicator device (e.g. to be shown on displays, on the device itself, etc.). In some embodiments, steering and/or bend direction relative to handle is notable to the user by means of at least one indicator. In some embodiments, indication and/or marking of the steering direction is located over the body of the handle. In some embodiments, indication and/or marking of the cutting direction is located over the body of the handle.

In some embodiments, the force indicator (or force gauge) is used for training and/or education purposes for users, in addition to the feedback provided to a user during a procedure, as described above.

In some embodiments, the lead extraction (LE) device is steerable while maintaining the integrity of the LE device, which may allow it to sustain the high forces that may be required for the procedure, for example, as will be described below with regards of the structure of the device.

In some embodiments, the lead extraction (LE) device will have a one directional valve in the handle to prevent blood from flowing out of the shaft in the proximal side. In some embodiments, the LE device comprises one or more valves configured to allow introduction and/or extraction of liquids from the shaft. In some embodiments, the one or more valves are hemostatic valves. In some embodiments, the valves are integrated with the lead extraction device and optionally located at the proximal end of the device. In some embodiments, the valves are located at the additional external sheath. In some embodiments, the valves are additions to the device, optionally configured to be plugged-in, for example, at the proximal end of the handle at the location of the opening where the lead that is being extracted exits the handle. In some embodiments, hemostatic valves are optionally used independently of the approach type performed by the user (for example, femoral approach).

In some embodiments, irrigation of the system is performed using the one or more valves in order to allow cleaning the device and optionally allow for a better performance of the system.

In some embodiments, the LE device may include one or more mechanisms to separate the surrounding tissue from the lead, for example blades and/or lasers and/or spreaders. In some embodiments, each mechanism is activated independently. In some embodiments, the mechanisms are activated in synchronization. In some embodiments, a pedal activates one or more mechanisms. In some embodiments, separating the lead from the tissue comprises sideways steering the tip to assist in the separation of the lead from the tissue and/or from other leads, rather than only advancing and/or dilating.

In some embodiments, the LE steerable device is configured to align itself to the lead and thereby potentially decreasing the force necessary for the extraction. In some embodiments, the LE steerable device is configured to align itself to the vein and thereby potentially avoiding damaging the vein. In some embodiments, alignment is done through activation of a steering mechanism controlled by the user by bending a section of the device at a desired angle, as will be further described below. This is contrary to prior art LE devices, which are configured to align the lead and the vein to the LE device by use of force, therefore potentially damaging the vein and/or encouraging undesirable force application directions. In some embodiments, the device comprises one or more balloons that are used to assist in directing the device towards a desired direction.

In some embodiments, the movement for the cutting tools is delivered by linear and\or circular (e.g. HHS and/or multi-lumen) motion mechanism, driven from outside the body of the patient and transmitted to the distal end of the device. In some embodiments, movement generated outside the body is converted from linear to circular (or vice versa) at the distal end of the device. These mechanisms will be further explained below.

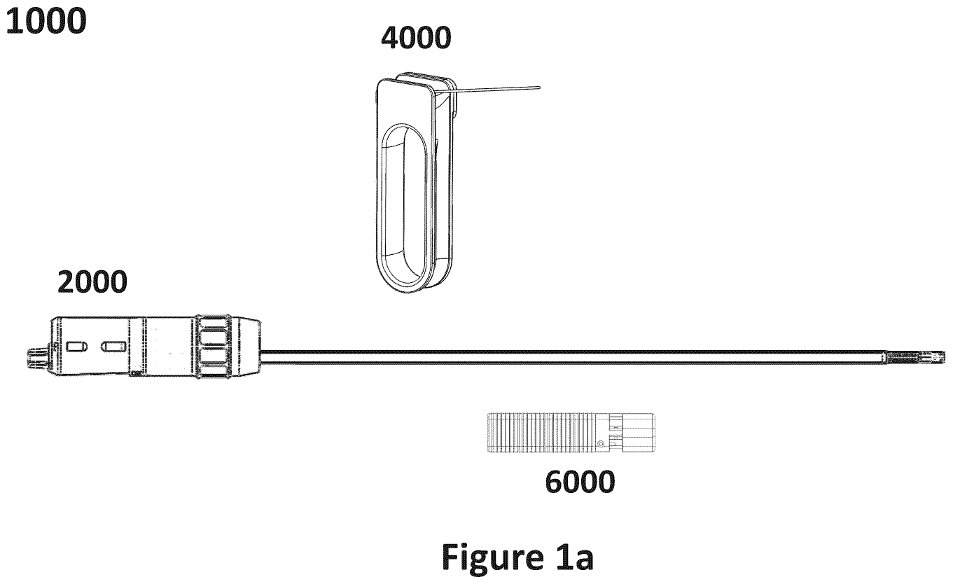

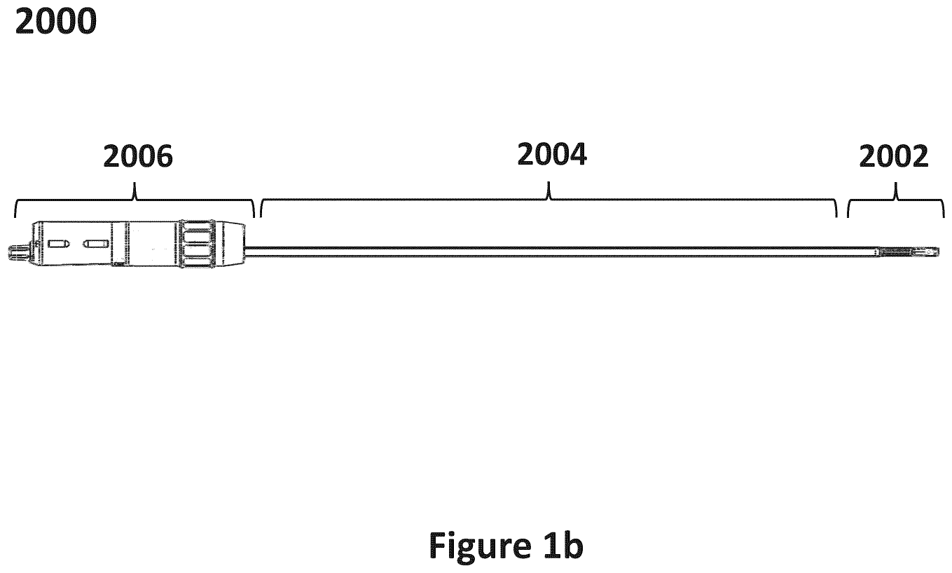

In some embodiments, the kit 1000 comprises a lead extraction device 2000, a sheath handle 4000 and a lead puller 6000, as shown for example in FIG. 1a. To facilitate the explanation of different embodiments of the invention, three general zones of the lead extraction device 2000 are identified: the distal head 2002, the sheath 2004 and the handle 2006, as shown for example in FIG. 1b.

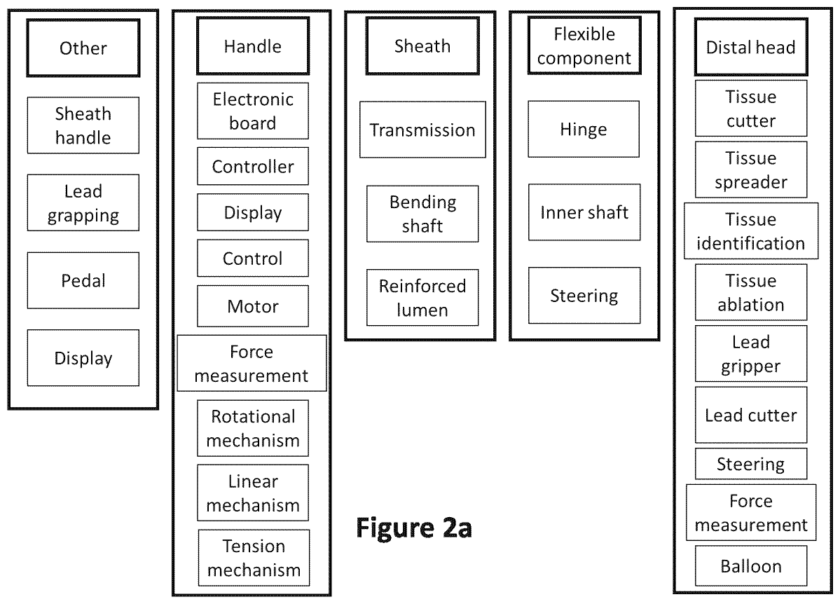

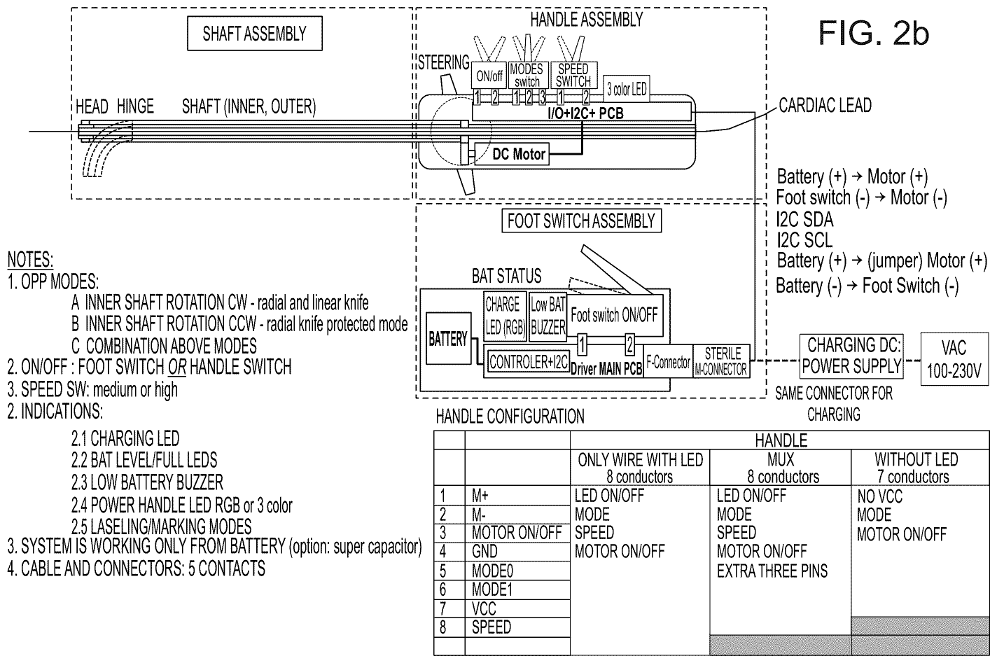

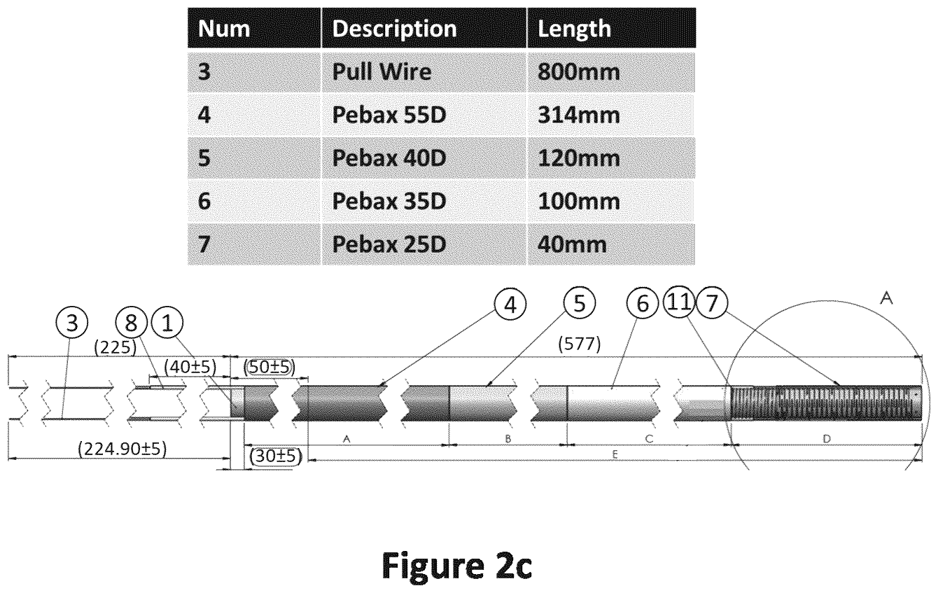

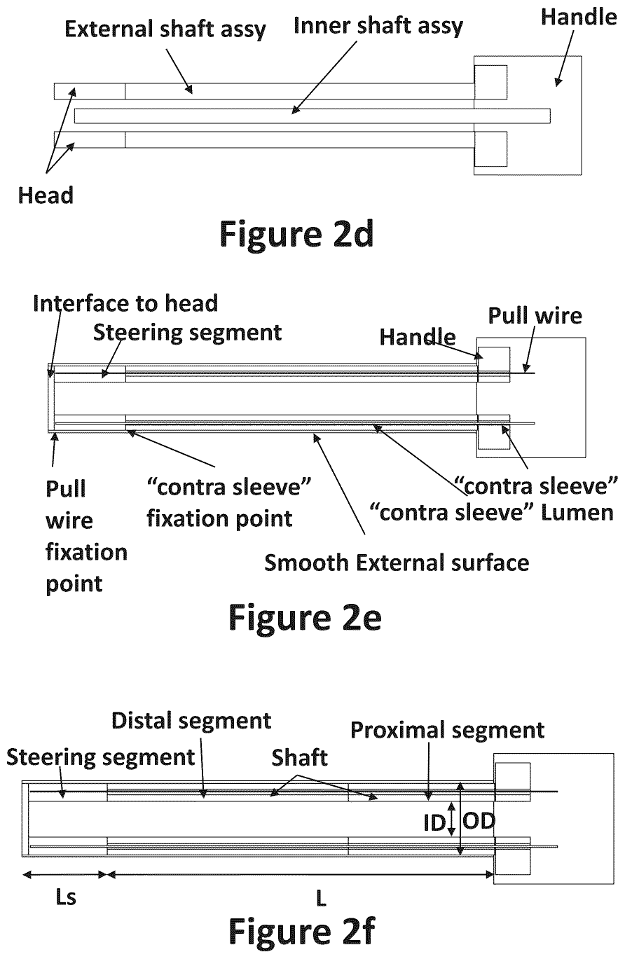

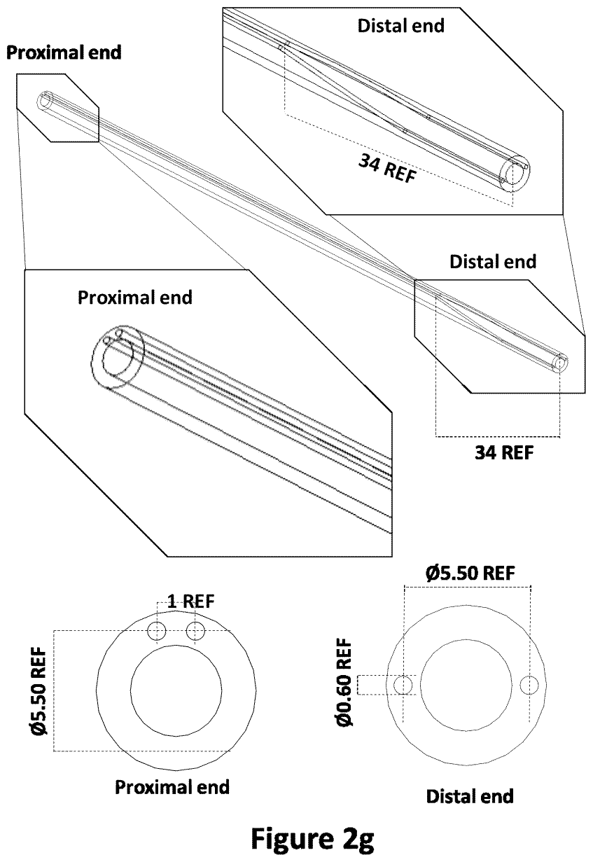

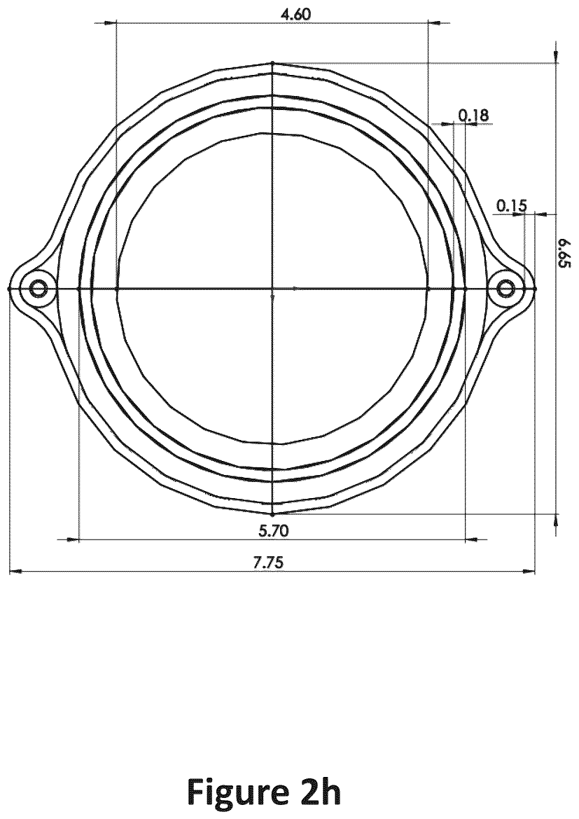

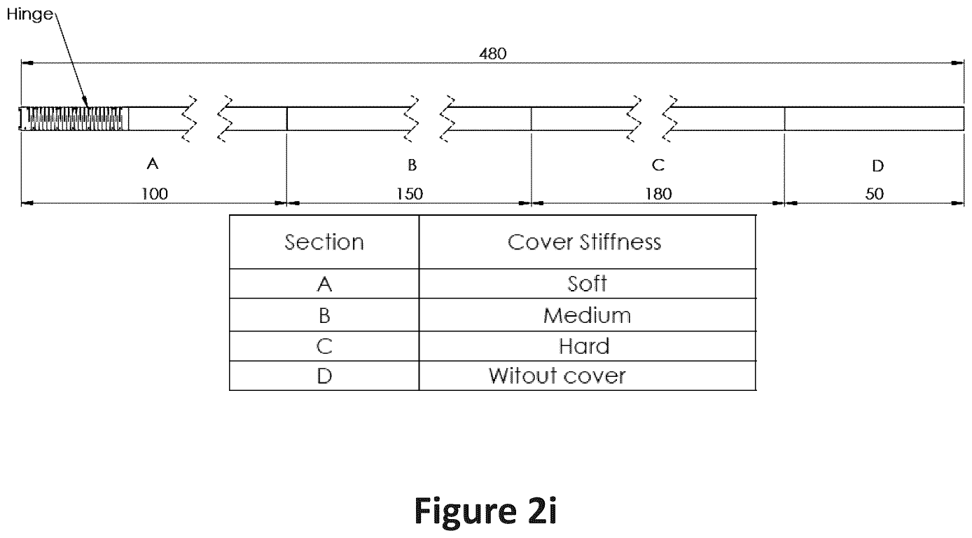

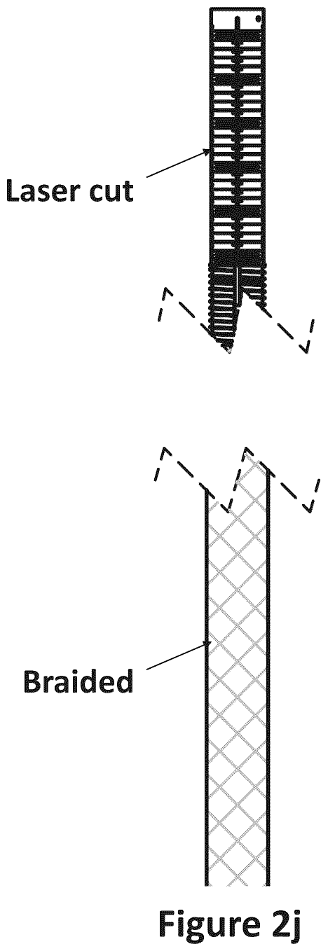

Referring now to FIG. 2a, showing a schematic representation of exemplary components, exemplary tools and exemplary mechanisms according to their exemplary location on the device and/or outside the device.

In some embodiments, components at the distal head comprise at least one of the following: a tissue cutter tool, a tissue spreader tool, a tissue identification tool, a tissue ablation tool, a lead gripper tool, a lead cutter tool, a steering mechanism, a force measurement tool, a balloon device. In some embodiments, the tissue cutter tool removes tissue surrounding the lead. In some embodiments, a tissue spreader tool separates the tissue surrounding the lead. In some embodiments, the tissue identification tool identifies the tissue (e.g. blood, blood vessel, calcified tissue, etc.) and/or the lead located distally or adjacent to the distal head. In some embodiments, the tissue ablation tool removes tissue from the lead by erosive means (e.g. laser). In some embodiments, the lead gripper tool physically holds the lead allowing the user to pull the lead proximally. In some embodiments, the lead cutter tool cuts the lead at the user's desired location. In some embodiments, the steering mechanism specifically moves the distal head to any direction desired by the user. In some embodiments, the force, friction and/or load measurement mechanism(s) provide indication of the forces, frictions and/or loads applied to the distal end and/or along the shaft and/or on the steering mechanism and/or on the motor. In some embodiments, one or more measurement mechanism(s) are used for providing feedback during the procedure and/or for educational purposes during training. In some embodiments, one or more balloon components are used as a tissue separator. In some embodiments, one or more balloon components are used for isolating specific zones from the blood flow. In some embodiments, one or more balloon components are used as anchorage for the LE device. In some embodiments, one or more balloon components is used to form a support (relative to one or more of a vein, muscle, tissue and/or lead) for assisting the catheter to be pushed against, and/or to generate an anchoring point for the device within the vessel, and/or to assist in directing the device by centralizing/positioning the device within the vessel using balloons as a pushing mechanism, and/or to achieve dilation by separating tissue from and/or surrounding the lead, and/or to use the balloon as a rescue balloon, for example, in case of bleeding and/or vein rupture, and/or to use the balloon as a blood blocker to allow the medical personnel to intervene in case of bleeding.

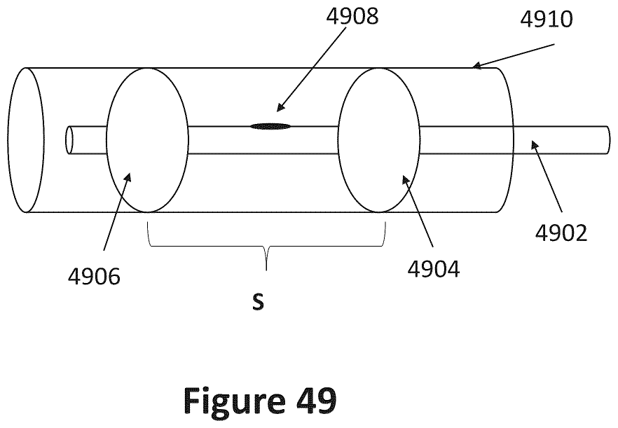

In some embodiments, the device comprises, for example, two balloons located at a distal end of the device (it should be understood that more than two balloons can be used in the device), with a certain separation from each other. For example, with a separation of from about 1 cm to about 5 cm. Optionally from about 0.5 cm to about 7 cm. Optionally from about 0.1 cm to about 10 cm. In some embodiments, the lead extraction device comprises a drainage system located between the two balloons. In some embodiments, a user inflates the two balloons, thereby isolating a specific portion of the vessel from the rest, and then the user activated the drainage system, thereby evacuating the contents within the isolated portion (see below explanations related to FIG. 49).

Referring now to FIG. 2b, showing a schematic representation of the system, according to some embodiments of the invention. Also shown in FIG. 2b, a table describing 3 optional connections to the handle and more functions related to the handle and for the pedal, for example, connection, buzzer, LED light, battery and charging and some alerts and feedbacks for the user. In some embodiments, a buzzer is located in the handle, the pedal or both. In some embodiments, LED indicators are located on the handle, the pedal or both. In some embodiments, the system comprises a dedicated software configured to monitor the correct functioning of the parts of the device, for example, monitoring the motor current and the predetermined motor activation limits.

For example, the software monitors and measures for one or more of the following exemplary performance conditions:
Too low current: might indicate a broken shaft that does not transmit torque to the tip;
Too high current: might indicate overload or high friction or excessive linear or bending forces applied which might not translate the force to the tip, and/or might indicate break or lock of the shaft;
Abnormal current (possibly high) or unique current pattern: which might be associated with material applying friction while passing through the device;
High current due to possible high pulling force applied on the lead/stylet.

In some embodiments, upon repeated measurements (performed for example in order to reduce the chances of a temporary false alarm), which indicate a too high or a too low current consumption, an alert may be triggered and the motor may be stopped for allowing the user to evaluate the situation.

Referring now to FIG. 2c, showing a schematic representation of an exemplary shaft according to some embodiments of the invention. In some embodiments, exemplary different parts of the shaft are as shown for example in FIG. 2c. Explanations about each of the different parts can be found below. It should be understood that the numbers of the sizes and features disclosed in FIG. 2c are exemplary only, and are provided to allow a person having skills in the art to understand the invention and should not be limiting in any way.

In some embodiments, components of the flexible component comprise at least one of the following: at least one hinge, an inner shaft, a steering mechanism. In some embodiments, the at least one hinge is the bending location of the device, usually located at or in the vicinity of the distal end of the device, which is used, for example for steering. In some embodiments, the inner shaft holds/absorbs/transmits the forces of push/torque/rotation/speed rotating/hammering/bending radius of the flexible component. In some embodiments, the steering mechanism bends the flexible component to the desired direction. In some embodiments, the shaft is designed to follow the anatomy of veins and heart sites where the cardiac lead is positioned and needed to be extracted. In some embodiments, the shaft allows transfer of torque from the handle to the distal tip. Referring now to FIGS. 2d-2f, showing exemplary diagrams of shaft and the torque transmission mechanism. In some embodiments, the shaft structure is built from two shafts one over the other. In some embodiments, the inner shaft delivers torque from the handle to the distal tip at a rotation speed in the range of about 0 RPM to about 600 RPM. Optionally, in the range of about 20 RPM to about 500 RPM. Optionally, in the range of about 100 RPM to about 400 RPM, for example 300 RPM, 350 RPM, 400 RPM, 420 RPM. A potential advantage of having a velocity of 400±50 RPM is that it allows the system to work optimally. In some embodiments, the external shaft supports the internal shaft, holds and controls and steers the distal tip. In some embodiments, the external shaft, and optionally any external surface of the device, is coated with a hydrophilic coating to decrease the friction between the device and the organic surrounding (for example the blood vessel) where the device is used. In some embodiments, the shaft's distal tip contains a cutting blade and/or an impact mechanism. In some embodiments, the impact mechanism is a tube and/or a ring. In some embodiments, the impact mechanism comprise a blade. In some embodiments, the impact mechanism is adapted to perform other functions, other than impact function, for example provide cover for other blades when it is not necessary to give an impact. In some embodiments, the impact mechanism comprises a distal end that is perfectly perpendicular to a longitudinal axis of the device. In some embodiments, the impact mechanism comprises a distal end that is angled and/or beveled in relation to the longitudinal axis of the device. In some embodiments, impact actions are used to separate/cut hard tissue, while rotation actions are used to separate/cut soft tissue (see below paragraph regarding “differential interaction with tissue”). In some embodiments, the impact mechanism is activated in other modes, other than impact mode, for example, the impact mechanism is activated as a vibrating mechanism. In some embodiments, the device comprise a plurality of operating modes. In some embodiments, examples of the plurality of modes are:
a. activating contemporarily the cutting mechanism and the impact mechanism;
b. activating contemporarily rotation of the distal end in vibrating mode and inner cutting blade;
c. activating contemporarily cutting and impact mechanism for 1 second, then change directionality of rotation for 60 seconds (and vice versa after 1 sec-60 sec), while inner cutting with the blade while vibrating mechanism is active.
d. activating as mentioned in (c) with the modalities mentioned in (a), then changing modalities as mentioned in (b), each activation is for 1 sec then change for 60 sec. Optionally for 2 sec, then change for 40 sec. Optionally, for 3 sec, then change for 30 sec. Optionally, for 4 sec and then change for 20 sec or 3 sec or 4 sec or 5 sec or 6 sec. In some embodiments, the changes between directions and modes and duration of activation change between changes. In some embodiments, the changes are random and/or in a repetitive manner.

In some embodiments, due to the use of a ratchet mechanism, the activation modality can be symmetric or asymmetric per each direction, for example, 2 seconds CW and 2 seconds CCW when symmetric, and 2 seconds CW and 1 second CCW when asymmetric, both versions when working in fast mode. In some embodiments, due to the use of the ratchet mechanism, the activation modality can have a slow mode, using for example the following pattern of activation: 2 seconds CW and 1.5 seconds CCW. Optionally or additionally from 0.3 sec to 1 sec CW and 0.3 sec to 1 sec CCW, optionally from 0.8 to 2 sec CW and 0.8 sec to 2 sec CCW, optionally from 2 sec to 5 sec CW and 2 sec to 5 sec CCW, or any number of seconds can be used in any modality. In some embodiments, the activation is continuous. In some embodiments, the activation order is the opposite, first CCW and then CW, for example 1.5 seconds CW and 2 second CCW. In some embodiments, other measurements of activation can be used, for example number of cycles per CW or CCW for example 1 to 3 cycles, 2 to 5 cycles, 4 to 10 cycles, 8 to 15 cycles, 10-20 cycles,

In some embodiments, any activation of the mechanisms is provided first at slow velocity and then the velocity is gradually increased and in some options, the speed is gradually decreased when changing the direction from CCW to CW and/or from CW to CCW and/or from CCW to Stop and/or from CW to STOP. See below section “Motor Profile”. In some embodiments, a potential advantage of this is that it provides a security measure against mistaken activation of the device during the procedure. In some embodiments, a potential advantage of this is that it potentially improves the life cycle of the system. In some embodiments, it improves functionality.

In some embodiments, when there is no fixed protocol of activation, the device includes a plurality of software instructions that are executed while in operation, which allow changing modality of activation, time of activation and directionality of activation automatically, according, for example, to changes in the torque of the shaft as sensed by dedicated sensors in the device.

Referring now to FIG. 2f, showing some dimensions of an exemplary external shaft, according to some embodiments of the invention. In some embodiments, the external shaft is characterized by a length (L), an external diameter (OD), an internal diameter (ID) and a bending section length (LS), for example, the dimensions of the external shaft comprise a length (L) of 450 mm±10 mm, an external diameter (OD) of 7 mm±0.5 mm, an internal diameter (ID) of 5.5 mm±0.5 mm, a bending section length (LS) of 30 mm±1.0 mm. Optionally, the dimensions follow the known French catheter scale, for example 9 French, 11 French, 13 French. In some embodiments, the dimensions are different of those known in the French catheter scale. In some embodiments, the dimensions change according to the French gauge number according to the scale. In some embodiments, the dimensions of the internal shaft comprise a length of 450 mm±10 mm, an external diameter of 5 mm±0.5 mm, an internal diameter (ID) of 4.3 mm±0.5 mm. In some embodiments, the external diameter allows the rotation, the bending and the steering according to the requirements. In some embodiments the contra sleeve lumen will go in parallel to the shaft main lumen and, in some embodiments, the contra sleeve lumen will have a 90 degrees twist next to the distal tip and go at the same side of the shaft as in FIG. 2g.

In some embodiments, the contra sleeve slightly protrudes from the outer diameter of the shaft, as shown in FIG. 2h. In some embodiments, a potential advantage of doing this is to avoid increasing the overall OD of the device by filling the areas around the contra sleeve, as shown for example in FIG. 2g.

In some embodiments, components of the sheath comprise at least one of the following: a transmission, a bending shaft (Hinge), a reinforced lumen. In some embodiments, the transmission delivers the mechanical movements from the handle to the distal head and vice versa. In some embodiments, the bending shaft (Hinge) holds the sheath from being affected by high torque forces and from the activation of the transmission. In some embodiments, the reinforced lumen preserves preserve the cross section of the sheath.

In some embodiments, components of the handle comprise one or more of the following: at least one electronic board, at least one controller, at least one display, at least one control, at least one motor, a rotation mechanism, a linear mechanism, at least one force measurement tool and a tension tool. In some embodiments, the electronic board is responsible for receiving and delivering commands from the user to the different components in the handle. In some embodiments, the controller is responsible for activating the different components in the handle according to the commands received by the user through the electronic board. In some embodiments, the display provides visual information to the user regarding the different components of the device. In some embodiments, the display is an interface that provides clear and simple feedback to the user, for example, about power level, steering direction and angulation, rotation direction and impact mode. In some embodiments, an on\off switch is located next to the shaft on the handle, so the user can find and easily push the button by sliding the hand on the shaft and pushing the button on the handle with the hand. In some embodiments, an emergency stop mechanism of the device includes: pulling the handle backwards (proximally) stops the mechanism of the device; pulling the shaft backwards (proximally) stops the mechanism of the device; and pressing the button located next to the shaft on the handle. In some embodiments, the control what the user presses and/or moves in order to actuate the components of the device. In some embodiments, the control is connected to the electronic board. In some embodiments, the control is connected directly to movement mechanism (e.g. motor, springs, rings). In some embodiments, the motor provides the necessary force to actuate the components of the device. In some embodiments, the rotation mechanism provides rotational movement to the components of the device. In some embodiments, the rotation mechanism receives the force from the motor. In some embodiments, the motor is located at the handle. In some embodiments, the motor is located at the pedal. In some embodiments, the linear mechanism provides linear movement to the components of the device. In some embodiments, the linear mechanism receives the force from the motor. In some embodiments, linear movement is converted into circular movement at the handle. In some embodiments, circular movement is converted into linear movement at the handle. In some embodiments, linear movement is converted into circular movement at the distal head. In some embodiments, circular movement is converted into linear movement at the distal head. In some embodiments, the force measurement tool provides indication to the user on the forces applied on the handle (generally forces in the distal direction. In some embodiments, the tension tool keeps the tension on the lead at a fixed chosen level, for example by pulling the lead. In some embodiments, the tension tool releases the lead if the tension increases over the set parameter. In some embodiments, the tension tool pulls the lead if the tension decreases under the set parameter.

In some embodiments, components and/or elements reside outside the LE device. In some embodiments, external components and/or elements comprise at least one of the following: a sheath handle, a lead grapping tool, at least one pedal, at least one display. In some embodiments, the sheath handle allows the user to hold the sheath. In some embodiments, the lead grapping tool allows the user to pull and hold the lead not with his/her own hand. In some embodiments, the at least one pedal is used to activate one or more components through the handle. In some embodiments, components can be activated at the handle. In some embodiments, components can be activated at the pedal. In some embodiments, components can be activated at the handle and at the pedal. In some embodiments, the display provides visual information to the user regarding the different components of the device. In some embodiments, the display is an interface that provides clear and simple feedback to the user, for example, about power level, steering direction and angulation, rotation direction and impact mode.

In some embodiments, the handle contains at least one knob and/or lever to support the steering, angulation and shifting in-betweens. In some embodiments, the pedal contains a main On/Off button switch. In some embodiments, a pedal controls the cutting velocity. In some embodiments, a pedal comprise controls to choose working modes.

In some embodiments, the pedal is configured to have one or more modes of activation, for example: mode A: pushing the pedal activates a mechanism, pushing the pedal again and it deactivates a mechanism; mode B: as long as the pedal is pressed the mechanism is activated, when leaving the pedal, the mechanism deactivates; mode C: the pedal work as an accelerator, the more it is pressed the faster the mechanism is activated.

In some embodiments, the pedal system is integrated with the control, for example, of imaging systems (e.g. fluoroscopy, or ultrasound) in the operating room. For example, same pedal, or optionally two pedals, are in the pedal unit for controlling multiple systems. For another example, same leg can be used to control x-ray or imaging as well as CLE device operations. For example, one pedal press activates the imaging, and a stronger pedal press activates the imaging and the CLE. For example, when the pedal is pressed then the imaging starts, optionally immediately, optionally with a delay (e.g. 0 sec, 0.5 sec, 1 sec, 2 sec, 3 sec or any configurable seconds) the CLE starts, and then when the leg releases the pedal then the CLE stops and at a delay of, for example, 0 sec, 0.5 sec, 1 sec, 2 sec, 3 sec, or any configurable seconds, the imaging stops. In some embodiments, a potential advantage of this is that it can potentially save the user of another operator. It can also potentially enable the synchronization between device and imaging, and, if desired, it can potentially make sure that the device does not necessarily work without an imaging being present while device is active.

In some embodiments, more than one mechanism is activated from the pedal, for example, the motor is activated by the pedal and also imaging means are activated by the pedal. In some embodiments, on the handle there is provided one or more buttons that can be either activated or deactivated which perform the same actions as the pedal. In some embodiments, the user can choose to use either the pedal or the buttons or both.

In some embodiments, the pedal comprises a battery, optionally a rechargeable battery. In some embodiments, the pedal comprises a reusable battery. In some embodiments, the pedal comprises an easily accessible battery that can be replaced. In some embodiments, the battery is configured to work for a plurality of hours of use, for example for 6 hours of use, 12 hours of use, 24 hours of use, or more or less hours of use and intervals. In some embodiments, the pedal is not connected to the electricity during procedures. In some embodiments, the battery is configured to work for at least 10 min. In some embodiments, the battery is configured to work for more than 20 min. In some embodiments, the battery is configured to work for more than 1.5 hours. In some embodiments, the battery is configured to work for a period of time from about 7 hours to about 8 hours, optionally from about 6 hours to about 10 hours, optionally from about 5 hours to about 15 hours.

In some embodiments, the pedal further comprises a data storage unit for recording performing data during the lead extraction process, for example: the current required to rotate the blade at certain locations during the lead extraction process. In some embodiments, procedure data is used for evaluating the forces applied during the procedure and the reason for using more and/or less forces during the procedure. For example, the device encountered a calcification on the lead and, in order to remove it, the device applied 10% more current to the blades, meaning it required 10% more force to deal with the calcification. In some embodiments, the data storage unit is located at the pedal or at the handle. In some embodiments, the data storage unit comprises space to record data (for example: current and/or forces and/or time and/or reconnecting handle to pedal, during working time and/or activation and/or motor on) from about 8 hours to about 12 hours of working procedures, optionally from about 15 hours to about 100 hours, optionally from about 20 hours to about 150 hours, optionally from about 15 hours to about 300 hours. In some embodiments from about 30 minutes to 60 minutes of working motor time of the handle.

### 3. Exemplary Sheath 2004

In some cases, the stiffness of a sheath may significantly cause complications in the lead extraction procedure. In some occasions, in order to induce the stiff sheath to bend and/or to follow the curved path of a lead through a vein, the lead may be pulled taut. In some cases, this tension in the lead may cause the lead to break resulting in a more complicated extraction procedure, or the tension in the lead may result in the lead tearing a vein and/or the heart's wall. For example, this may occur when the lead is attached to the vein and/or the heart wall by fibrous tissue. Such tearing may result in a serious bleeding complication. In other occasions, the stiffness of the sheath may contribute to complications as a result of the forces applied to the vein walls by the sheath after it has been bent. For example, when force is applied to the sheath in an attempt to move it forwards along the lead, the sheath may apply forces on the walls of the vein, for example at a bend. In some cases, a stiffer sheath may exert more force on the walls of the vein when compared to a less stiff sheath.

In some embodiments, the sheath and/or the distal head include a region, which is significantly more flexible, than other parts of the sheath. Optionally, the sheath and/or the distal head include multiple highly flexible regions along their length. In some embodiments, these characteristics can provide one or more of the following potential advantages: reduced tension over the lead and/or the blood vessels; enhanced control of the LE device; easier extraction procedures for the user; and more. For example, the flexible region may support a bending radius (without kink, under a given force) 3 times smaller than the other parts support, for example 5 times smaller, for example 10 times smaller, for example 20 times smaller, or any ratio in between those mentioned. In some embodiments, the device comprises the flexible region and is adapted to withstand the internal forces from the actuation mechanisms occurring in the distal tip. In some embodiments, the distance between the flexible region and the location where the actuation in the distal tip occurs is from about 0 mm to about 15 mm; optionally from about 2 mm to about 10 mm; optionally from about 4 mm to about 8 mm.

In some embodiments, the outer shaft comprises different zones comprising different stiffness, as shown for example in FIG. 2i. In some embodiments, the more proximal zones are stiffer than the distal zones. In some embodiments, Zone D is inside the handle and optionally does not comprise an outer shaft.

### 3.1 Exemplary Incorporated Steering Mechanism

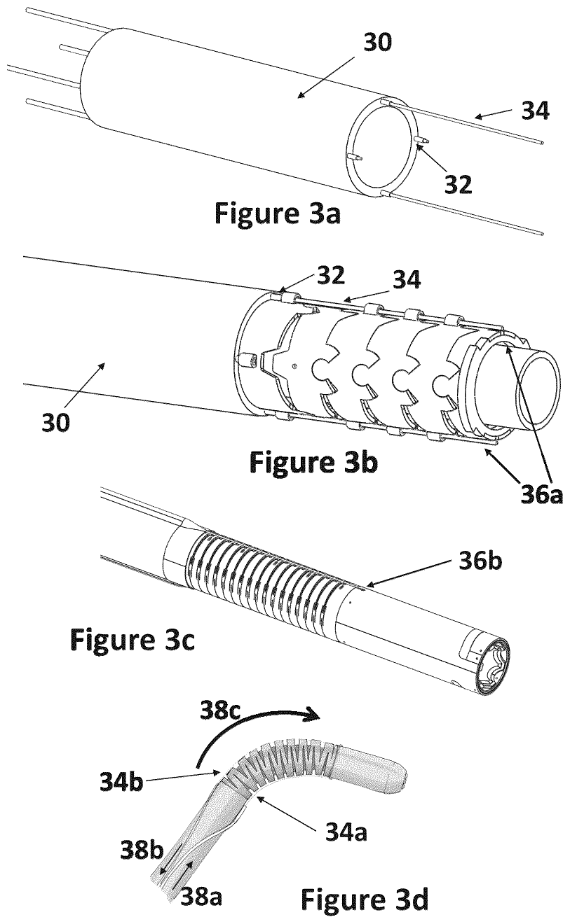

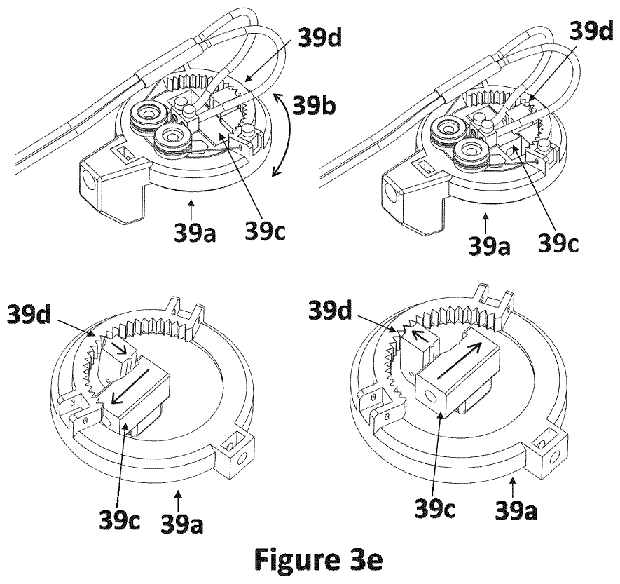

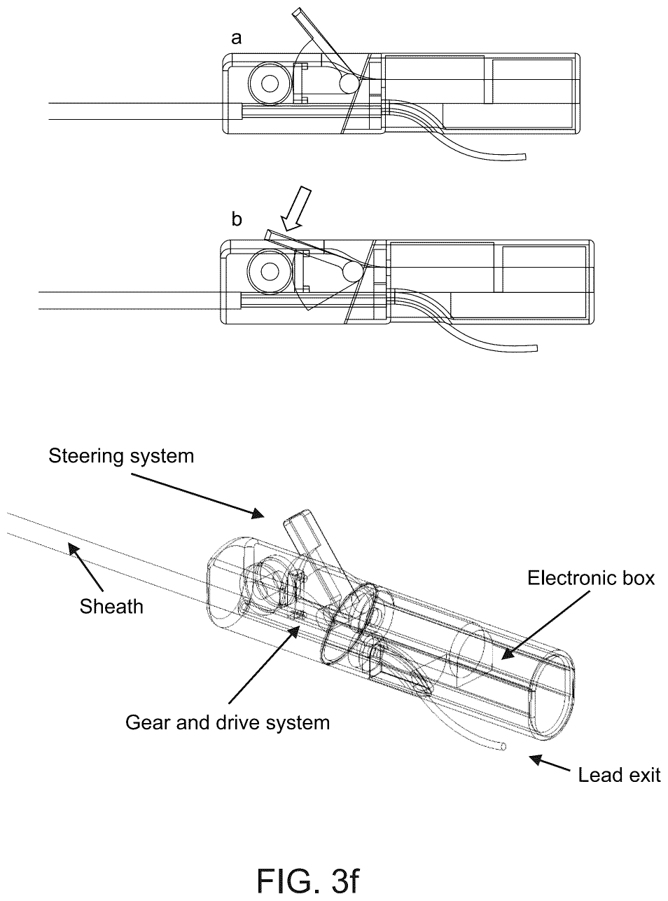

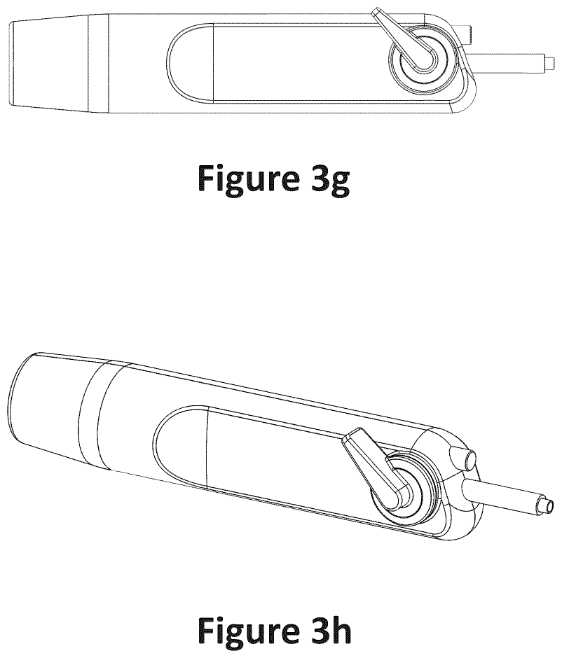

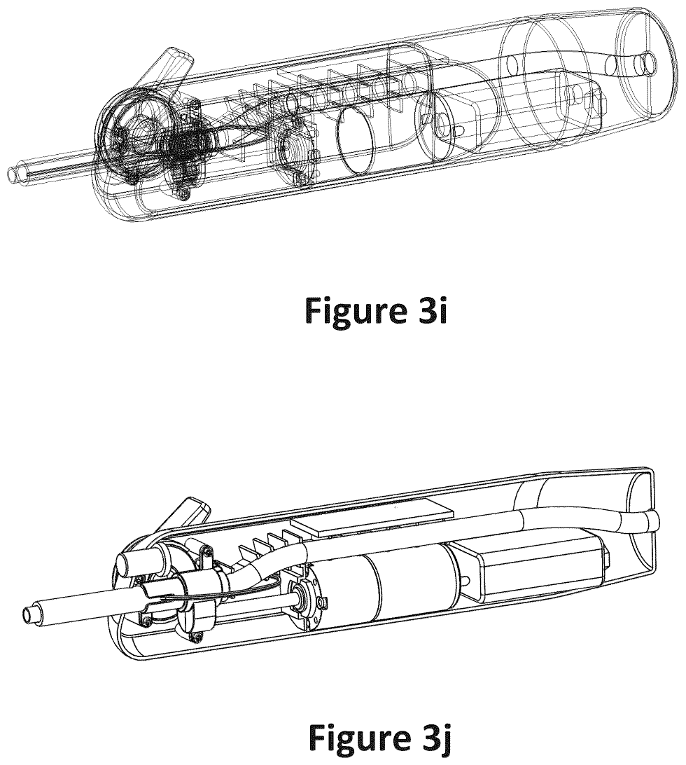

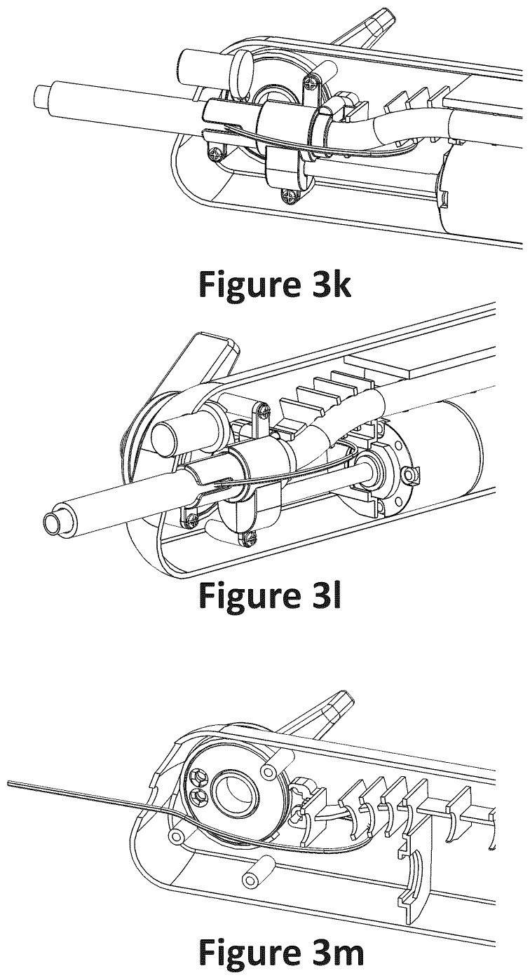

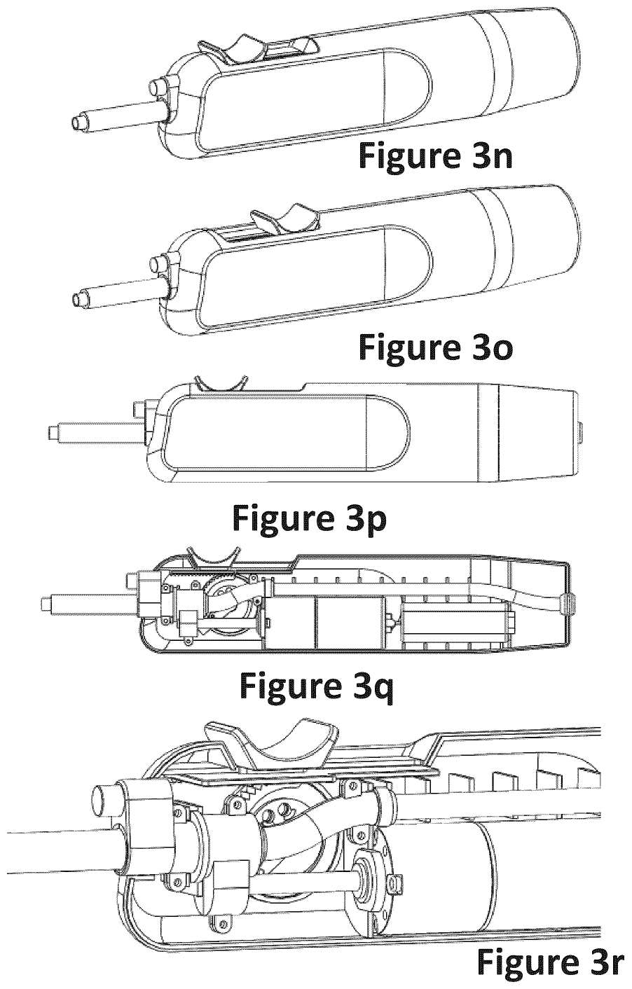

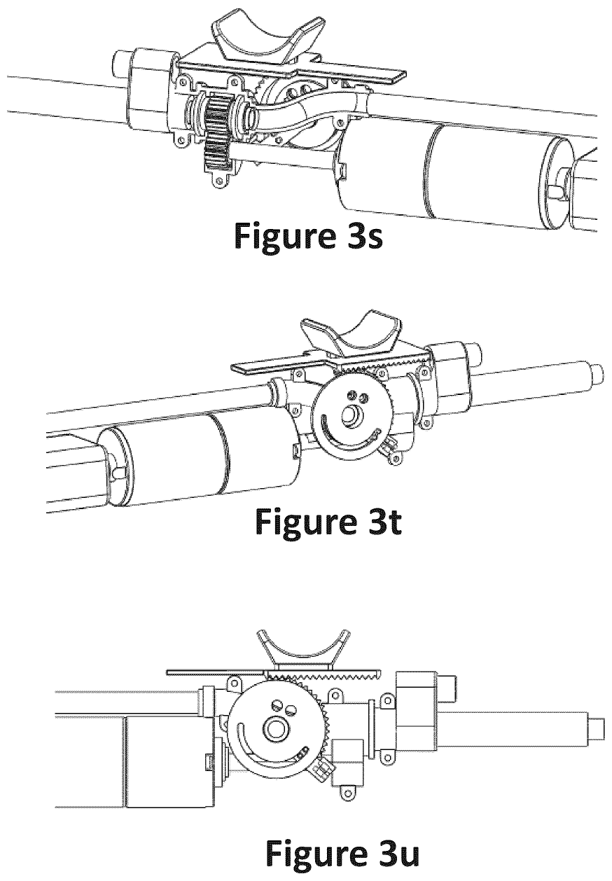

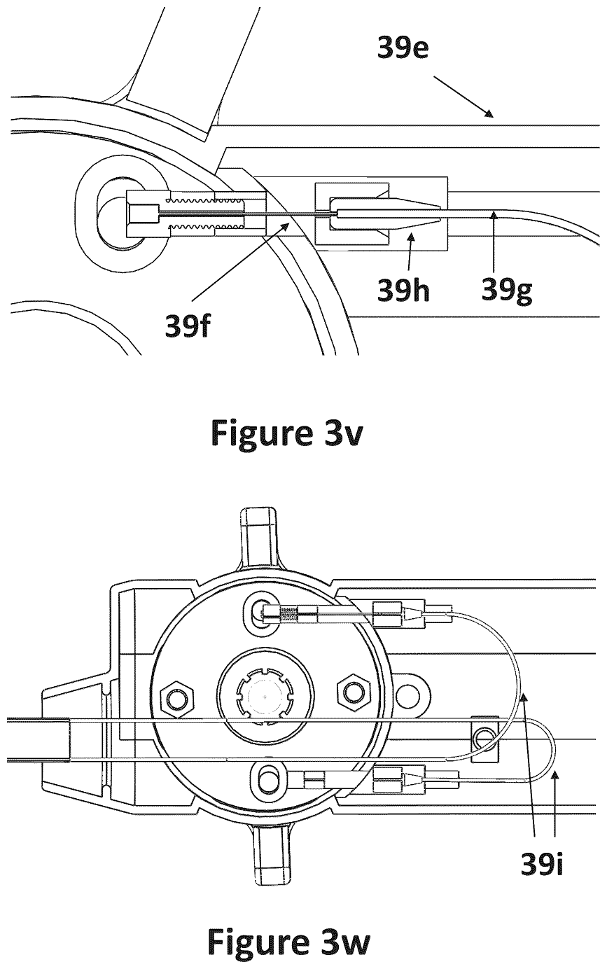

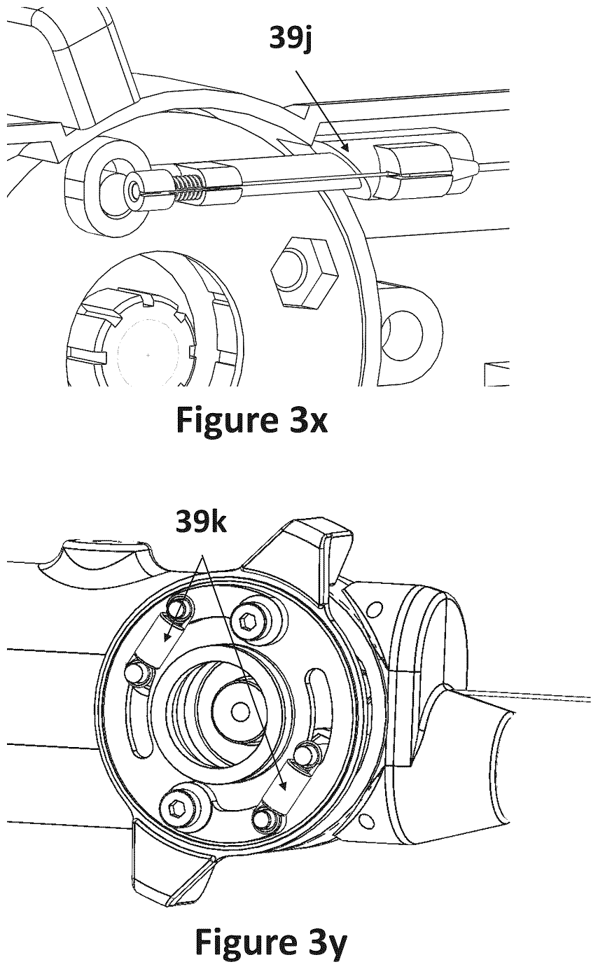

In some embodiments, the steering mechanism is incorporated in a dedicated sheath that covers the catheter, as shown for example in FIG. 3a. In some embodiments, the dedicated sheath comprises an outer envelope 30, at least one wire guide 32, and at least one wire 34. In some embodiments, the wire guide provides a suitable free path of actuation to the wire. In some embodiments, the wire guide enables the wires to move distally and proximally without affecting or causing distortions in the sheath. In some embodiments, the sheath is an integral part of the LE device. In some embodiments, the sheath is an add-on to an existing LE device (see accessories in section 12). In some embodiments, the steering mechanisms comprise one wire guide with one wire. In some embodiments, the steering mechanisms comprise two wire guides with two wires. In some embodiments, the steering mechanisms comprise three wire guides with three wires. In some embodiments, the steering mechanisms comprise four wire guides with four wires, as shown for example in FIG. 3a. In some embodiments, the number of wire guides/wires dictate the number of directions to which the steering mechanism can direct the distal end of the device.

In some embodiments, the steering mechanism is connected to a hinge as shown, for example, in FIG. 3b. In some embodiments, the location where the steering mechanism meets the hinge and/or the distal head comprises a cap (36b), which protects the wire from disconnecting from the hinge and/or the distal head. In some embodiments, from the outer envelope 30, over the wire guides 32, the wires 34 are connected to dedicated slots 36a located on the hinges. In some embodiments, the wires 44 are connected to dedicated slots 36b on the distal head, as shown for example in FIG. 3c. In some embodiments, the position of the wires, either more distal or more proximal, provides the distal head with the variable mobility. In some embodiments, the user pulls or pushes the wires to control the movement of the distal head, as shown for example in FIG. 3d. In some embodiments, when the user pulls wire 34a, following arrow 38a while pushing wire 34b, following arrow 38b, it causes the distal head to bend as shown by arrow 38c.

In some embodiments, the steering wire that runs from the handle 106 to the tip 102 runs outside and along the sheath 104, as shown for example in FIG. 3d.

In some embodiments, the steering wire that runs from the handle 106 to the tip 102 runs in a braided reinforced coil sleeve (extension coil with PTFE cover or a tube that is braided reinforced coil), for example: Vention Medical 142-0011 or 142-0008 or “Microlumen”—Pure PTFE ID with Braid—0.0005″×0.0025″ @ 80 PIC. In some embodiments, the dimensions are for example: ID: 0.0104″±20%, OD: 0.0234″±20%, Wall: 0.0065″±20%. In some embodiments, the wire is inserted in a PTFE tube. In some embodiments, the braided reinforced coil sleeve is connected to the handle or/and to the hinge to maintain length of the wires that runs in it.

In some embodiments, the reinforced coil sleeve comprise welded ends having a length of from about 0.1 mm to about 5 mm. Optionally from about 0.3 mm to about 4 mm. Optionally from about 0.5 mm to about 3 mm. For example 0.1 mm, 0.2 mm, 0.3 mm, 0.4 mm, 0.5 mm, 1 mm. 1.5 mm, 2 mm, 2.5 mm, 3 mm. In some embodiments, the reinforced coil sleeve does not comprise wielded ends.

In some embodiments, the steering wires that run from the handle to the tip need to hold forces up to 8 kg at the maximum bending radius of the hinge. In some embodiments, the same forces are applied to the braid-reinforced coil, for example: at 45 degrees, the force can be 400-700 g on free air; and at 100 degrees, the force can be 1100 g-1800 g. In some embodiments, these forces include the accumulated friction forces between the wire and the reinforced tube, along the full path to the handle.

In some embodiments, the user sets the steering mechanism to bend the distal end at any angle or position, for example at 10 degrees, 0 degrees, 90 degrees, 30 degrees, etc. In some embodiments, the user can “lock” the position by activating a locking mechanism (example shown in FIG. 3e) on the handle and then the hinge will stay in this position due to the wires and the braid reinforced coil, which are holding the hinge at the chosen position. In some embodiments, a lever operates the locking mechanism. In some embodiments, a button operates the locking mechanism. In some embodiments, the steering mechanism is activated by a ring 39a around the handle that can be rotated 39b to control the degree of bending, and a lever 39c is pushed to engage a lock 39d, as shown for example in FIG. 3e. In some embodiments, the user works with the cutting blades at the chosen position. In some embodiments, the steering wires that run from the handle to the tip comprise a slack of the braided reinforced tube. In some embodiments, the slack compensates for the about ±12 mm deflection of external shaft and the bended hinge. In some embodiments, the slack is located at the handle. In some embodiments, the slack is located along the shaft under the outer cover of the shaft. In some embodiments, the length of the slack is from about 2 cm to about 7 cm; optionally from about 3 cm to about 6 cm; optionally from about 4 cm to about 5 cm. In some embodiments, there is minimum slack or no slack at all. In some embodiments, there is no braided reinforced tube.

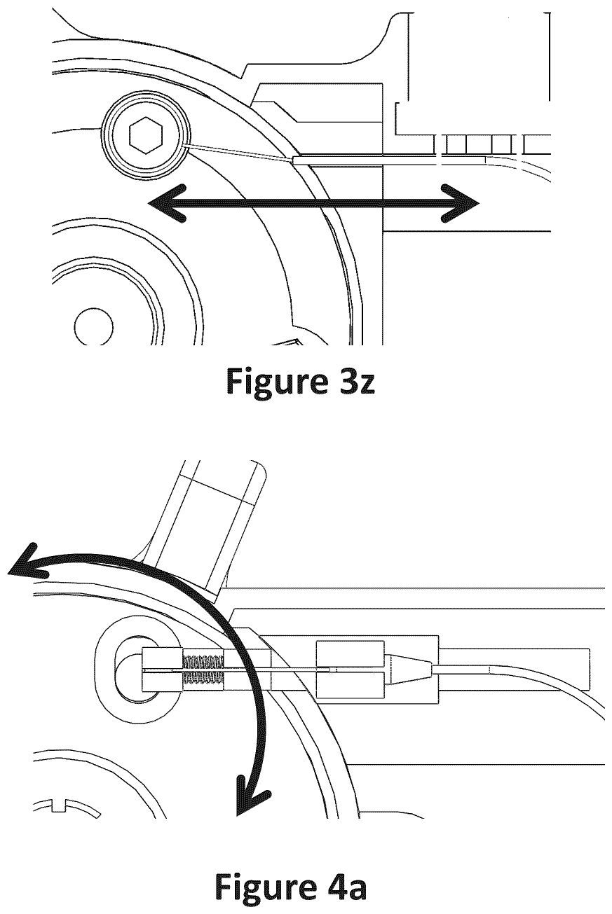

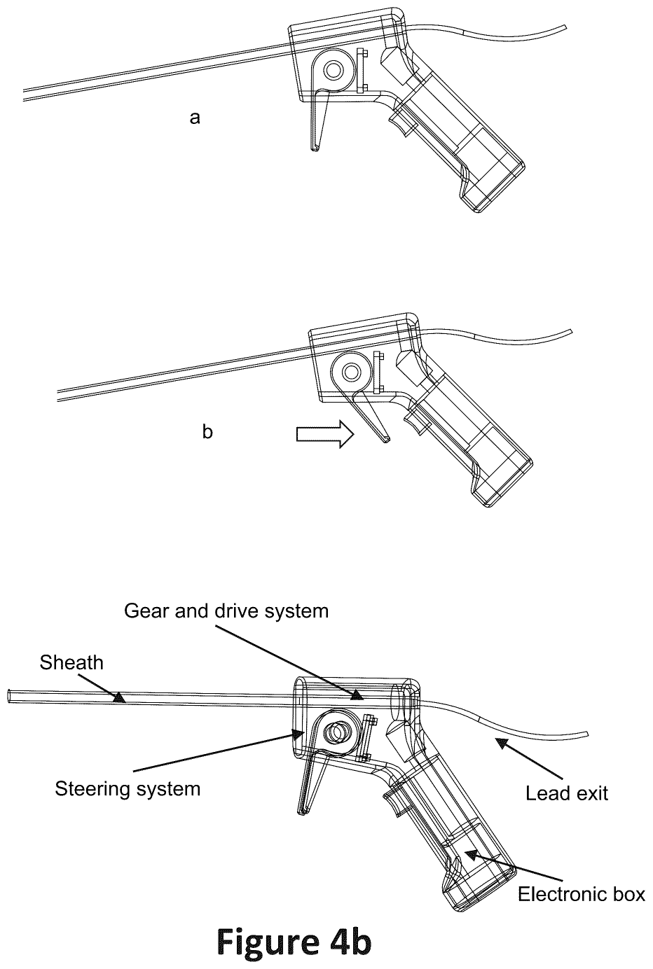

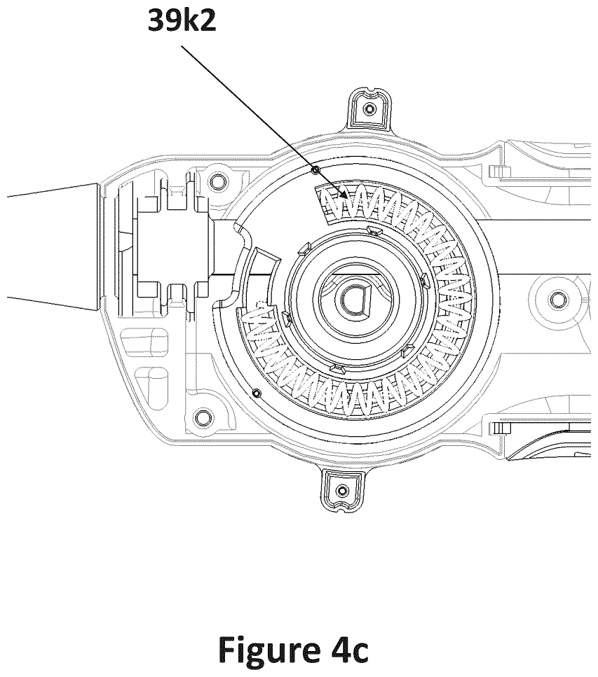

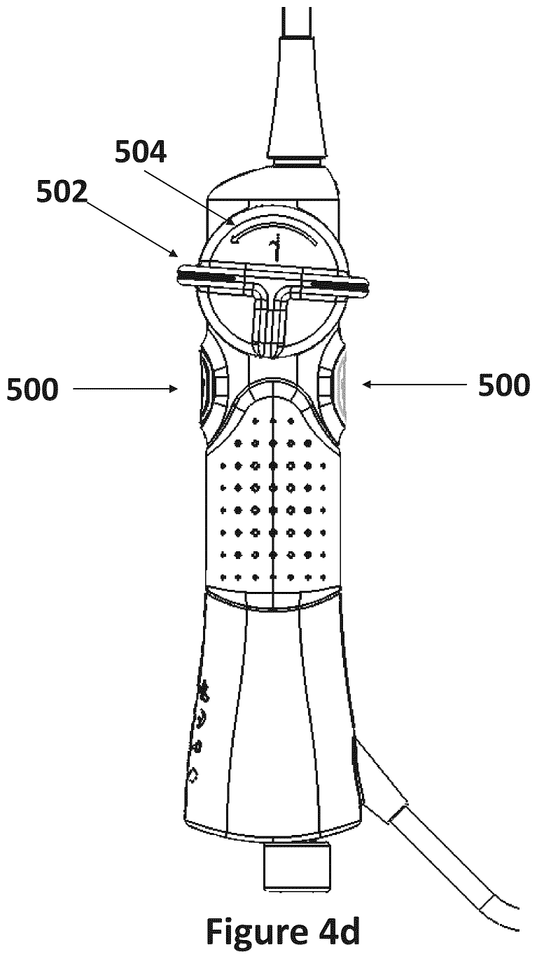

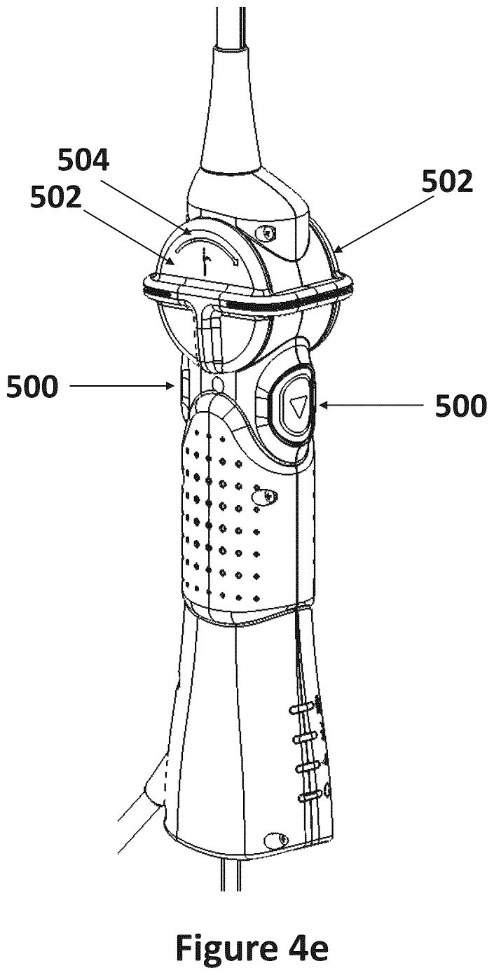

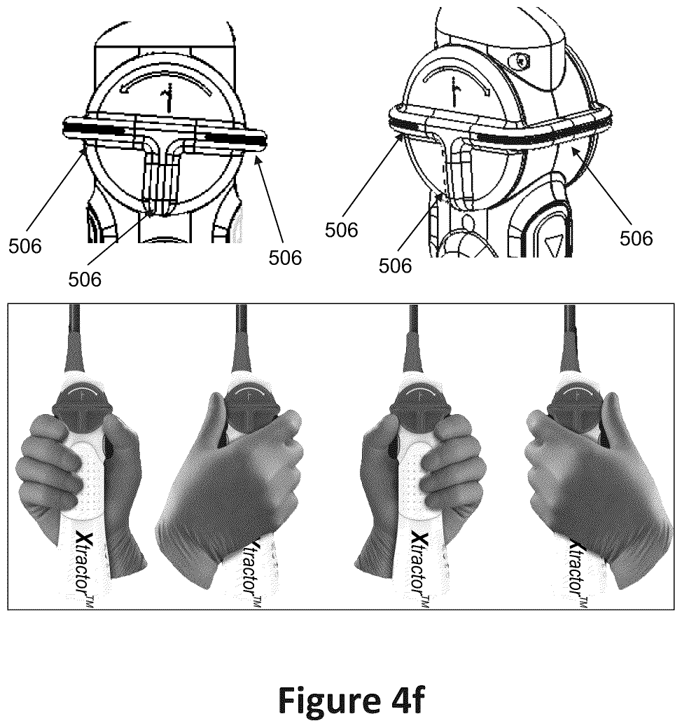

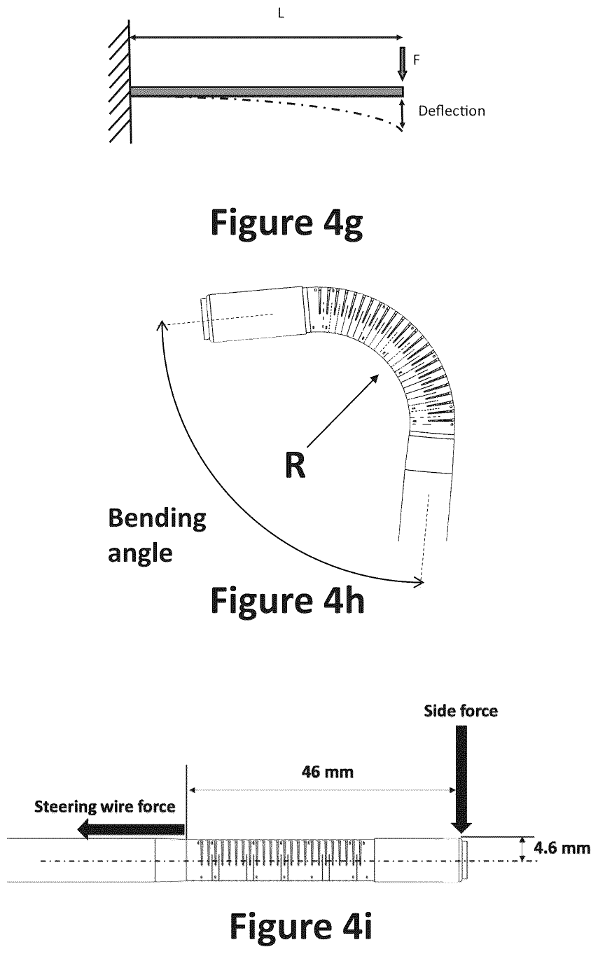

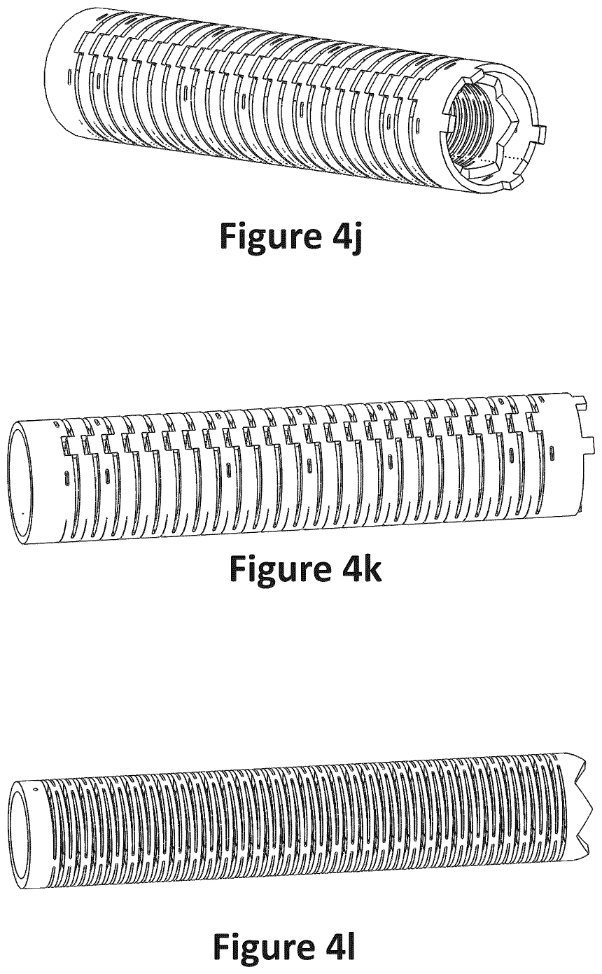

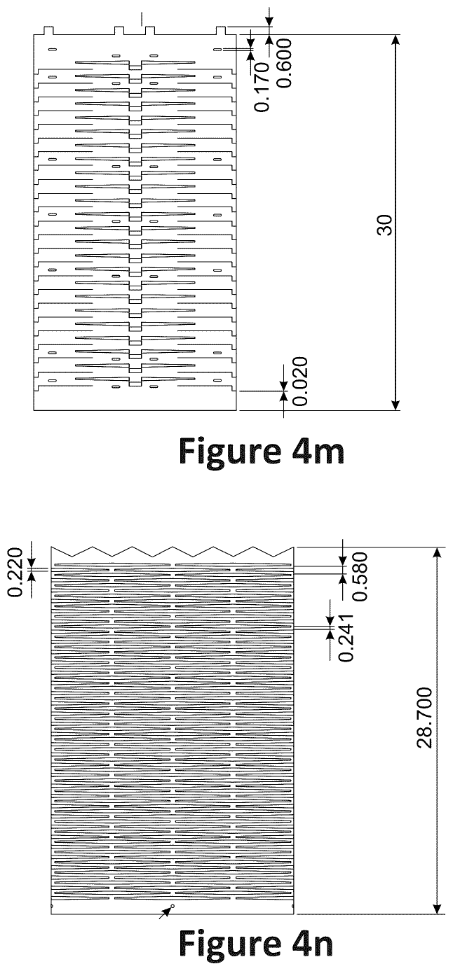

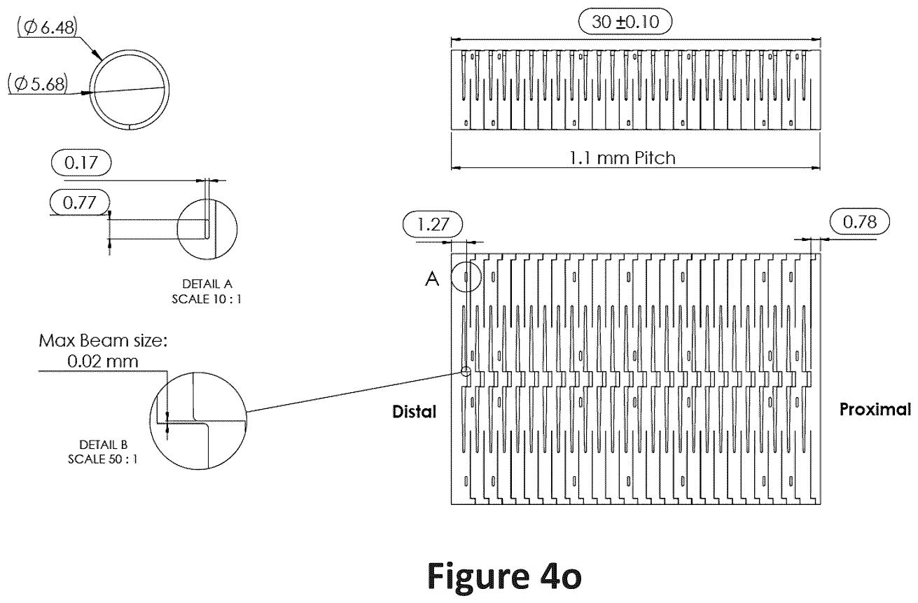

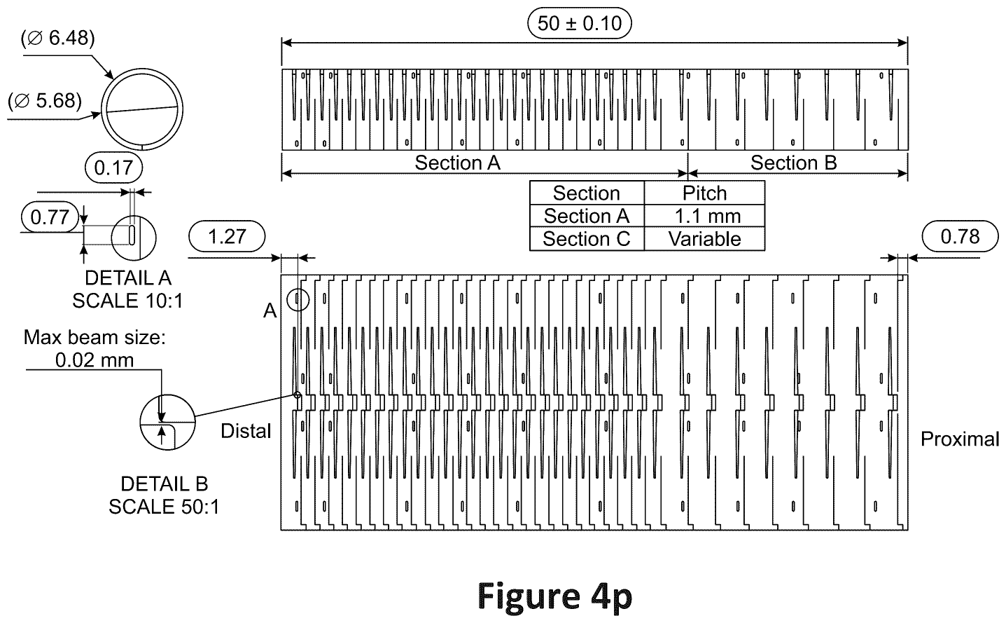

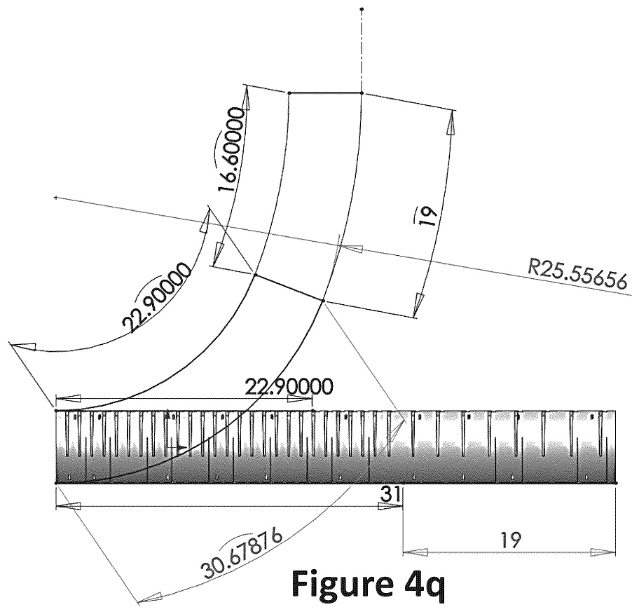

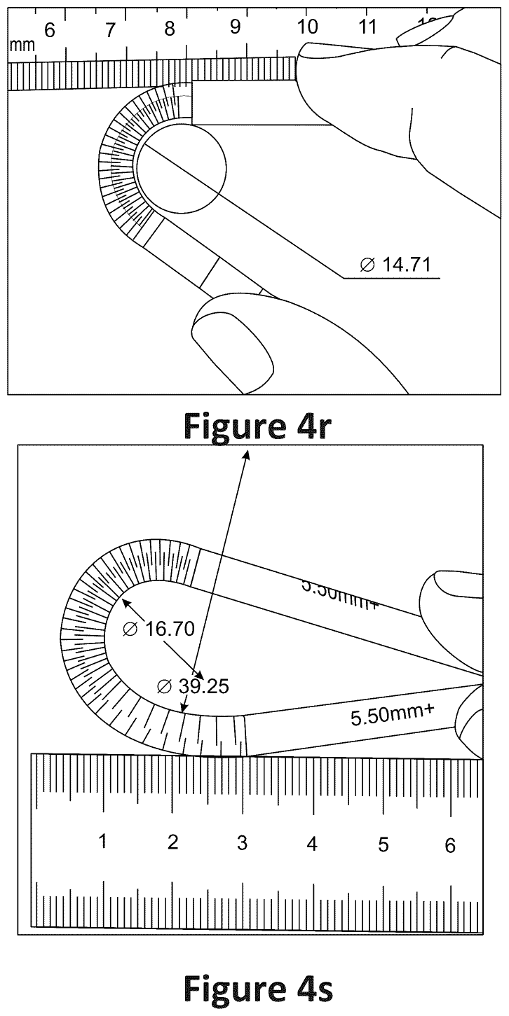

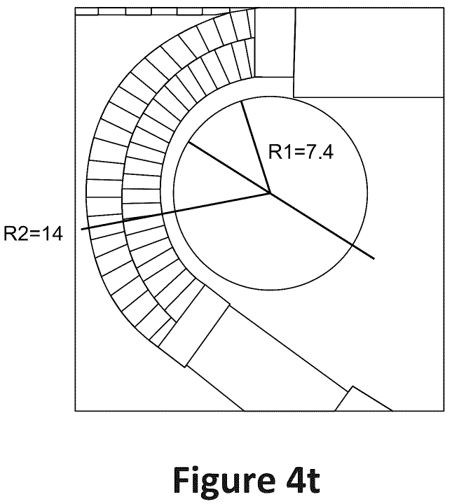

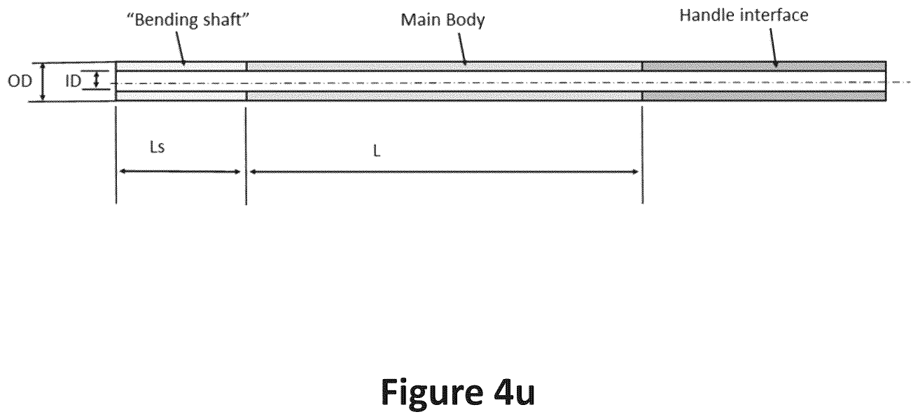

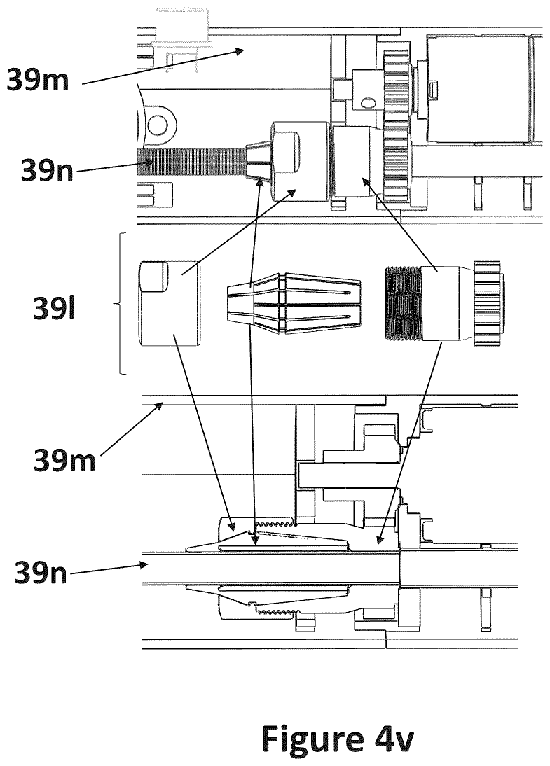

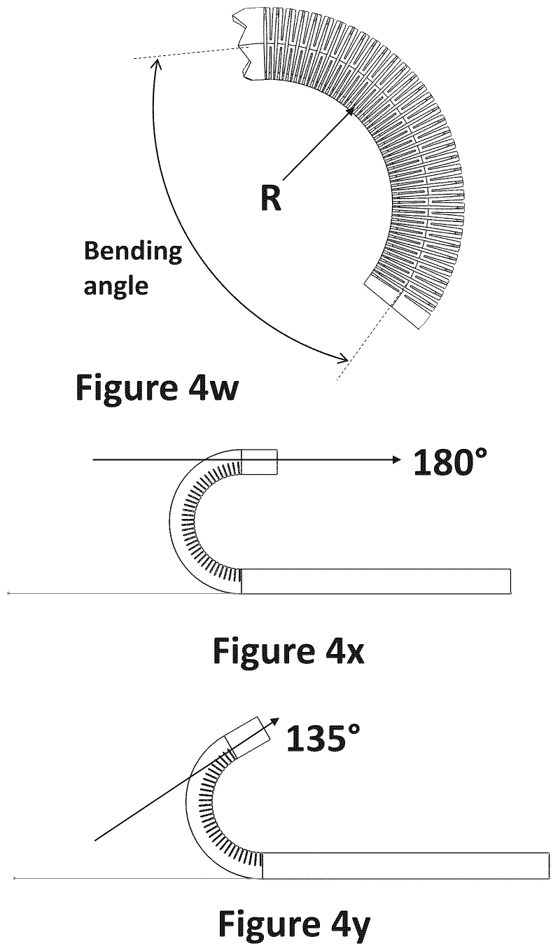

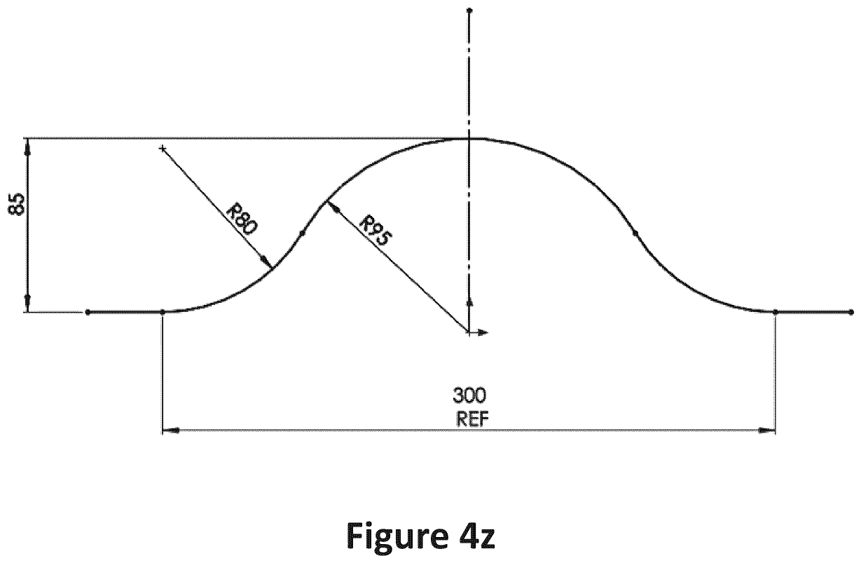

Referring now to FIGS. 3f, 3g, 3h, 3i, 3j, 3k, 3l, 3m, 3n, 3o, 3p, 3q, 3r, 3s, 3t and FIG. 4b, showing embodiments of exemplary handles, according to some embodiments of the invention. In some embodiments, the handle comprises a handle shape, as shown for example in FIG. 3f. In some embodiments, the handle comprises a handle shape, as shown for example in FIG. 4d. In some embodiments, the handle comprises a pistol shape, as shown for example in FIG. 4b. In some embodiments, the handle, comprising a handle shape, comprise the mechanical mechanism as shown in FIGS. 3g-3t.

In some embodiments, the “center of gravity” of the handle will be located at the center of the handle. In some embodiments, the configuration of the center of gravity minimizes the rotation of the handle when the user holds the unit with one hand on the lead or locking stylet and the other hand on the shaft or elsewhere (see below more information regarding “Exemplary ergonomic handle”). In some embodiments, the lead or locking stylet is passed through the shaft to the pulling motors located in the handle. In some embodiments, the path of the lead or locking stylet inside the handle is not aligned with the shaft, for example, slightly goes over the motor and back to the center of the handle. In some embodiments, this shape of the shaft and handle add friction to prevent the rotation of the shaft.

### 3.2 Exemplary Reinforced Central Lumen

In some embodiments, a lumen of the flexible device shaft is reinforced. Optionally, the reinforcement is designed to preserve the cross section of the lumen. For example, a circular cross section of the inner lumen may be retained during bending. In some embodiments, maintaining the cross section of the lumen may reduce friction on the lead wire due to shaft bending. Optionally, the reinforcement may include a coil and/or a braid and/or a ring.

In some embodiments, the shaft is reinforced with one or more coils, braids, wires, or other components in order to achieve the desired combination of mechanical properties, for example, flexibility and pushability. In some embodiments, multiple reinforcements of the shaft provide the desired properties, which effectively transmit the distal force applied to the device handle outside the patient's body to a distal section located within the vasculature and/or the heart.

In some embodiments, any of the shafts (outer, inner, external, etc.) is made of a combination of techniques, for example, part of the shaft is braided, while another part is hypo-tube, as shown for example in FIG. 2j. In some embodiments, the techniques used are one or more of coils, braids, hypo-tube, made of for example metal, plastic, reinforced plastic, and any combination thereof. In some embodiments, the division of how much of the whole device is made of different techniques is from about 10% to about 90% that is made of one material and the rest is made of at least one another material. In some embodiments, the whole device is made of 100% of a technique. For example 50% length of inner shaft is made from Braid and the other 50% of distal length is made of hypo-tube or any other combination of techniques.

In some embodiments, the inner lumen of the lead extraction device is smooth. In some embodiments, the inner lumen of the lead extraction device does not comprise steps and/or interferences within the inner lumen. In some embodiments, the inner lumen of the lead extraction device is substantially straight. In some embodiments, this potentially helps avoiding problems of passage of the lead and/or lead with tissue within the lead extraction device and ease the extraction procedure. In some embodiments, the inner lumen and/or an element within the inner lumen is configured to avoid passage of liquids from the distal end of the device towards the handle/proximal end of the device. In some embodiments, the inner lumen of the lead extraction device has different inner diameter along the inner shaft. For example, the distal side at the tip comprises a smaller diameter than the rest of the shaft or, for example, the knife area comprises a slightly smaller diameter than other parts of the inner shaft in order to potentially reduce the inner friction.

### 3.3 Exemplary Structural Characteristics of the Shaft

In some embodiments, two segments of rigidity characterize the shaft: a distal segment, which is softer, comprising a length of about 300 mm, and a proximal segment, which is rigid, comprising a length of about 150 mm. In some embodiments, the segments lengths are, for example: Distal soft: 300 mm, Proximal semi-rigid: 150 mm or 200 mm or 250 mm. In some embodiments, the shaft comprises a gradual change in rigidity along the shaft. In some embodiments, the minimal bending radius of the distal segment is about R<50 mm, and of the proximal segment is about R<100 mm. Optionally, the minimal bending radius of the distal segment is less than 20 mm, optionally less than 15 mm, optionally less than 10 mm. In some embodiments, the torque rigidity, for CCW rotation: a torque of about 5 [N*cm] twists the shaft by 45 Degrees, for the full shaft length; and for CW rotation: a torque of about 10 [N*cm] twists the shaft by 45 Degrees, for full shaft length. In some embodiments, the shaft comprises a torque strength of 50[N*cm] and more preferred more than 75 [N*cm].

In some embodiments, the shaft is characterized by a Flexural Rigidity (Bending), as schematically shown in FIG. 4g. In some embodiments, for the distal segment:_a setup of L=130 [mm] and F=0.25 [N], results in a deflection of about 16 [mm]; for the proximal segment: a setup of L=130 [mm] and F=0.5 [N] [or >0.5N], results in a deflection of about 16 [mm]. Optionally, for the distal segment:_a setup of L=130 [mm] and F=0.3 [N], results in a deflection of about 14 [mm]. Optionally, for the distal segment:_a setup of L=130 [mm] and F=0.35 [N], results in a deflection of about 12 [mm].

In some embodiments, the shaft of the device is characterized by having a measured rigidity. In some embodiments, the device comprises an additional accessory, a dilator sheath (sheath over the shaft), which increases the rigidity of the shaft. In some embodiments, the dilator sheath is between 10 cm to 40 cm or between 10 cm to 30 cm or between 10 cm to 20 cm or 13 cm or 15 cm or 17 cm. In some embodiments, the dilator sheath will be mounted and/or removed as the user choice by sliding it on and/or off the distal tip. In some embodiments, the dilator sheath is removed by a “pill way” method. In some embodiments, the dilator sheath can be deployed in the vein. In some embodiments, the dilator sheath stays on the shaft but externally to the body.

### Compression and Tensile Resistance

In some embodiments, the pull-wire is supported and/or covered by a contra sleeve (i.e. a tension coil or torque coil or a “Bowden cable”—Straight flexible coil produced from round wire to specified dimensions. Close wound, meaning that there is no space between adjacent coils within the length) which prevents applying and/or transmitting forces to the external shaft. In some embodiments, the contra sleeve comprises a bending radius of about R<4 [mm]. In some embodiments, the contra sleeve holds a compression force of about 54 [N]. In some embodiments, the contra sleeve comprises an external diameter (OD) of about 0.3 [mm] and up to 0.9 [mm] preferred OD of 0.5 [mm]. In some embodiments, the contra sleeve is fixed at its distal end and protrude (proximal to the external shaft) by about 60 mm. In some embodiments, the external shaft is characterized by a tensile rigidity of about 20 [N/mm]. In some embodiments, the external shaft is characterized by a compression rigidity of about 40 [N/mm]. In some embodiments, the external holds a compression and tensile force of about 160 [N].

In some embodiments, the wire runs freely inside the contra sleeve. In some embodiments, this means that when the distal end of the device bends, the wires can run freely inside the contra sleeve without applying any kind of resistance on the contra sleeve. In some embodiments, the movement of the distal end, the steering mechanism at the handle follows the movement of the distal end, and this is allowed due to the fact that the wire runs free inside the contra sleeve. In some embodiments, the contra sleeve is fixed at certain points on the device, for example, at the connection point between the shaft and the hinge. For example, inside the handle 39e before the wire 39f exits the contra sleeve 39g, the contra sleeve is fixed 39h, as shown for example in FIG. 3v. In some embodiments, the slack (39i in FIG. 3v) mentioned above, allow the wire to be freely pulled or pushed along the contra sleeve, when the distal end is bent.

In some embodiments, the force applied on the wire necessary to move and/or straighten the distal head is from about 5 Kg to about 25 Kg, for example, 5 Kg, 10 Kg, 15 Kg, 20 Kg.

In some embodiments, the handle comprises a breaking-prevention mechanism configured to keep the wire from breaking, for example, as shown in FIGS. 3x and 3y. In FIG. 3x, the part 39j, which holds the contra sleeve and the wire can be seen exiting from it, is configured to move longitudinally if high forces are applied on the wire. Another example, in FIG. 3y, the steering mechanism comprises one or more springs 39k configured to absorb the extra pressure coming from the wires, meaning that before the wires can break, the wires will pull the springs.

In some embodiments, the wire is pulled/pushed using a linear motion, as shown in FIG. 3z, or a circular motion as shown in FIG. 4a.

Referring now to FIG. 4c showing a schematic representation of another configuration of an exemplary breaking-prevention mechanism, according to some embodiments of the invention. In some embodiments, instead of having two springs 39k, as shown for example in FIG. 3y, the breaking-prevention mechanism comprises one long spring 39k2 for absorbing the extra pressure coming from the wires. In some embodiments, this means that even when the user actuates the knob 502 (see below information about the knob 502), and therefore pulling the wires to bend the distal end of the device, and there is something at the distal end that counters the bending action, the extra-pressure on the wire will be absorbed by the spring. In some embodiments, this mechanism provides a breaking-prevention mechanism against breakage of the wire and its peripherals.

In some embodiments, additionally and/or alternatively, the mechanical mechanisms (components) in the handle are separated from the electronic mechanism (components) in the handle. In some embodiments, a potential advantage of this is that is potentially avoids from liquids to enter the electronic parts of the handle.

### Exemplary Ergonomic Handle

Referring now to FIGS. 4d, 4e and 4f, showing exemplary ergonomic handles according to some embodiments of the invention. In some embodiments, the handle is provided with a plurality of ergonomic features that enhance the ease of use of the user. In some embodiments, the handle comprises a symmetrical allocation of actuation elements, as shown for example in FIGS. 4d and 4e. In some embodiments, the handle comprises two activation buttons 500, one on each side of the handle, for activating/deactivating the operational distal end of the device. In some embodiments, the handle comprises a knob 502 for actuating the bending of the distal end of the device. In some embodiments, the knob 502 comprises an arrow 504 showing the direction of the bending of the distal end of the device. In some embodiments, knob 502 comprises a plurality of actuation points 506, on both sides of the handle, that allow the user to reach and actuate the knob 502 from a plurality of directions and to use either finger that the user desires, as shown for example in FIG. 4f. In some embodiments, the steering knob is configured to be intuitive with the movement of the shaft. In some embodiments, this means that a user holding the handle will find intuitive to move the knob to a desired direction to perform the desired bending of the distal end of the device. In some embodiments, the ergonomic features allow for this intuitive configuration.

In some embodiments, the handle is balanced. In some embodiments, the elements/components inside the handle were weighted and distributed along the internal space of the handle to achieve a handle that is balanced. In some embodiments, a handle that is balanced means, for example, that if the user releases the hold on the handle, the handle will not tend to perform a rotation around the longitudinal axis of the handle due to imbalances in the distribution of the components inside the handle. For example, if the device were to be raised in the air by holding, on one side, an end of a lead that passes through the distal end of the device, inside of the device and exits through the proximal end of the device, and, on the other side, the other end of the same lead (practically lifting the device by means of the lead that passes through), the device will not tend to rotate on its longitudinal axis due to the specific distribution of the components inside the handle. Another example, the handle is balanced by allocating part of the electronics and motor on one side of the lumen of the handle, and part of the electronics and the cable on the other side of the main lumen of the handle.

### Hinge and Pull-Wire

In some embodiments, the hinge and the pull-wire are integrated in the lead extraction device. In some embodiments, the hinge and the pull-wire are separate components from the lead extraction device and can be assembled into a lead extraction device. In some embodiments, the hinge is operated by two wires. In some embodiments, the hinge is operated by one wire. In some embodiments, the hinge is operated with no wires just by pushing it over the lead and rotating the handle cording to the path of the lead or the vain. In some embodiments, the hinge can be bent from about 0 degrees to about 90 degrees, optionally from about 0 degrees to about 180 degrees. In some embodiments, while one wire is being pulled (straighten), the other one is released. Referring now to FIG. 4h, showing an exemplary bent hinge. In some embodiments, the bend radius is R<8 [mm], with a bending angle >90 degrees. In some embodiments, the bending radius of the hinge is from about 1 mm to about 50 mm, for example, 5 mm, 10 mm, 15 mm, 20 mm, 25 mm, 30 mm, 35 mm, 40 mm, 45 mm, and any millimeter in between. In some embodiments, the pull-wire holding force is of about 54 [N]. In some embodiments, the hinge/pull wire enables applying force of about 3[N] or about 4[N] at its distal end (“Side force” shown in the picture is the reaction for such a force), as shown for example in FIG. 4i.

In some embodiments, the handle's steering mechanism comprises an “idle mode” where the wires and steering mechanism do not prevent from the hinge to move when forces are applied on the tip. In some embodiments, the overall force resistance (applied at distal end) required to bend the hinge (and the “inner bending shaft” inside it) is of about a maximum of 0.5 [N] for 45 degrees bend and of about a maximum of 1 [N] for 90 degrees bend. In some embodiments, the pull-wire, or pull-cable 7×1, comprises a total diameter of about 0.21 mm, optionally it can be in the range of 0.14 mm to 0.32 mm. In some embodiments, the pull-wire is made of smaller wires. In some embodiments, the number of smaller wires of the pull-wire cable is 7 (called 1×7 stainless steel) or from 5 to 21 wires. In some embodiments, the user can easily move from one mode to another using the handle or pedal.

Referring now to FIGS. 4j, 4k and 4l, showing schematic representations of exemplary interfaces to shaft and head. In some embodiments, the external shaft assay contains and supports the inner shaft while the former is turning at velocity of up to 600 RPM CW and CCW, or at velocities as disclosed above.

In some embodiments, the hinge will be laser cut from a tube OD 6.5 mm ID 5.7 mm and have the parameters as in FIG. 4m.

In some embodiments, the hinge is configured to be bent using forces in the range of 30 gr to 200 gr or in the range of 40 gr to 150 gr, for example, 50 gr, 100 gr, 150 gr, and any force in between, when no active steering is used.

In some embodiments, the head is integrated into the external shaft by a “key feature” and fixed by laser welding. In some embodiments, the handle is fixed to external shaft fixture through a “pre-defined interface feature”. In some embodiments, strain relief should be located at the interface between handle and external shaft to eliminate sharp curve. In some embodiments, the pull-wires are connected to steering mechanism locker inside handle.

In some embodiments, the inner shaft delivers torque from the handle to the head at a velocity range of 0-600 RPM. Preferred velocity is in the range of about 180 RPM to about 450 RPM for example 420 RPM or 380 RPM or 270 RPM. Referring now to FIG. 4u, showing a schematic representation of the inner shaft. In some embodiments, the inner shaft is characterized by a length (L) of from about 280 mm to about 490 mm. In some embodiments, the inner shaft is characterized by a bending shaft (Hinge) length (Ls) of about 23 [mm]. In some embodiments, the inner shaft is characterized by an internal diameter (ID) from about 4.3 mm to about 4.7 mm for the 13 F device model (other alternative size of ID can be 11 F or 9 F or any other between 6 F to 30 F). In some embodiments, the inner shaft is characterized by an external diameter (OD) of from about 5 mm to about 5.5 mm, for example 5.3 mm. In some embodiments, the internal diameter of the device is from about 2 mm to about 7 mm, optionally form about 3 mm to about 5 mm. In some embodiments, the outer diameter of the device is from about 5 mm to about 9 mm, optionally form about 6 mm to about 8 mm.

### Structural Characteristics of the Inner Shaft:

In some embodiments, the inner shaft is characterized by a minimal bending radius of about R<30 [mm]. In some embodiments, the inner shaft is characterized by a torque rigidity: CCW—torque of 5 [N*cm] will twist shaft by 45 [Deg] (for full shaft length); and CW—torque of 10 [N*cm] will twist shaft by 45 [Deg] (for full shaft length). In some embodiments, the inner shaft is characterized by a torque strength of at least 75 [N*cm], for example between about 30 and about 80. In some embodiments, the inner shaft is characterized by a flexural rigidity (Bending) of: For setup of L=70 [mm], F=0.1 [N], which results in a deflection=16 [mm] (see FIG. 4g).

### Bending Shaft (Hinge)

In some embodiments, the bending shaft (Hinge) functions as torque transmission element (capable of turning a full cycle around its axis). In some embodiments, the structure comprises minimum gaps and max shock absorption due to the blade impacts. In some embodiments, minimum bending forces are required. In some embodiments, the interface between a rotating inner bending shaft and the rigid hinge is carefully selected when choosing the material, in order to reduce friction and to minimalize hazards, for example, material and lead grinding, and collapsing of the center on the lead. Referring now to FIG. 4w, showing an exemplary bending shaft. In some embodiments, the bending shaft transmits torque of 75 [N*cm] at 0 to 600 RPM. In some embodiments, the bending shaft comprises a bending radius of R<4 [mm]. In some embodiments, the bending shaft comprises a bending angle >90 [deg].

In some embodiments, bending radius, which is measured to the inside curvature, means the minimum radius one can bend the device without kinking it, damaging it, or shortening its life. Usually, the smaller the bend radius, the greater is the material flexibility (as the radius of curvature decreases, the curvature increases). FIG. 4x illustrates a bending that bends 180 degrees, in relation to zero (or straight), while FIG. 4y illustrates a bending that bends 135 degrees in relation to zero (or straight). In some embodiments, the hinge bends to a maximal angle of from about 35 degrees to about 150 degrees over a bending segment/length of less than 10 cm, optionally over a bending segment/length of less than 6 cm.

In some embodiments, the inner bending shaft will be laser cut from a tube OD 5.35 mm ID 4.35 mm (or OD 5.4 mm ID 4.4 mm) and have the parameters as in FIG. 4n.

In some embodiments, the head length with all rotating and impact mechanism is between 11 mm to 22 mm, or between 15 mm to 18 mm.

In some embodiments, the bending shaft (Hinge) will comprise the dimensions as shown for example in FIG. 4o. In some embodiments, the length of the bending shaft (Hinge) is from about 20 mm to about 60 mm, for example 25 mm, 30 mm, 40 mm, 50 mm. In some embodiments, the bending shaft (Hinge) will have an increasing length of links, as shown for example in FIG. 4p. In some embodiments, for example, a length of the bending shaft (Hinge) comprises a certain distance between links, for example, the first 30 mm (from proximal to distal) the bending shaft (Hinge) comprises links having a large length, while the following 20 mm comprise links having shorter length. It should be understood that the dimensions disclosed herein are examples only, which are provided to allow a person having skill in the art to understand the invention. While some of the examples are suitable for a 13 French scale catheter, for example, it is the scope of the invention to include either bigger or smaller sizes, for example 15 French, 11 French, 9 French, by reducing or rescale the size of the parts.

FIG. 4q shows the bending angles of the exemplary bending shaft (Hinge) showed in FIG. 4p. In some embodiments, the bending shaft (Hinge) in section “A” (30 mm) can have a bending angle of more than 110 degrees (0-120 deg) with a radius of 6 mm to 20 mm for example about 8 mm or about 10 mm or about 12 mm or about 14 mm or about 16 mm or about 18 mm. In some embodiments, the bending shaft (Hinge) for the section “B” can have bending radius in a rage of 12 mm to 50 mm or 20 mm-40 mm for example, about 14 mm or about 16 mm or about 18 mm or about 20 mm or about 22 mm or about 25 mm; and can have bending angle of about 30 deg or about 40 deg or about 50 deg or about 60 deg or from about 0 deg to about 70 deg. In some embodiments, the bending shaft (Hinge) in section “A” or in section “B” can have equal or increasing gaps between the laser cut ribs, that will give the bending shaft (Hinge) to bend and get to the radius as described above or to bend in an increasing radius due to increasing gaps of the cuts as showed in FIG. 4p section “B”. FIGS. 4r and 4s show photographs of the bending angles of the exemplary bending shaft (Hinge) showed in FIG. 4p. FIG. 4r, shows a photograph of an exemplary 30 mm hinge, while FIG. 4s, shows a photograph of an exemplary 50 mm hinge.

In exemplary embodiments of the present invention, the system comprises a shaft (or any tube) that has a very flexible component which keeps the lumen open while bending, with a wall thickness of 0.2-1 mm, for example 0.3-0.6 mm thickness, to withstand the forces and torques are required in a lead extraction system, and an outer diameter of about 5-9 mm, for example about 6-8 mm. In some embodiments, the shaft is able to bend at curvatures having radius of less than 15 mm, for example about R1=7.4 mm (as shown in FIG. 4t), and the radius of the lateral part of the shaft is about R2=14 mm. Accordingly, the ratio between the radius of the outer curve (R2) and the radius of the inner curve (R1, the bending radius) is 1.89, which means that the path along the outer curve is nearly 90% longer than the path along the inner curve. This is due to the wide diameter of the tube vs the small bending radius. In an example, for such shaft to be able to bend up to about 85-110 degrees (about 1.5-1.9 radians, say about 1.7 radians), it means that the lateral side (outer curve) needs to stretch and become about 1.7 rad×7.4 mm=about 12.5 mm longer than the other (inner) curve. Plastic/polymer type of tubes, having similar wall thickness of up to about 1 mm to be suitable for shaft entrance and pushability into the blood vessels and for proper force and torque transmission as required in lead extraction, even with commonly used braiding, are not suitable for repeatedly elastically stretching by more than 50% on one end (lateral curve) relative to the other end (inner bending curve) while keeping the lumen open (for example, avoiding radius reduction of the lumen by 10% or by 20%). They can stretch and tear, they can collapse, break or kink, but are not suitable for maintaining the lumen open and keep the ability for repeated longitudinal stretch.

Therefore, the present invention provides a solution by providing a lead extraction system comprising a flexible portion. The flexible portion comprising an articulated structure comprising a lumen, for example in a form of a spring or a metal hypo tube that has multiple bending axes while providing a connection configured to keep the lumen open. In some embodiments, during the bending, gaps are formed at the lateral side of the bending side of the structure. In some embodiments, gaps are shrunk on the inner side of the bending side of the structure. In some embodiments, by expanding gaps on the lateral side (and optionally shrinking gaps in the inner side), the structure can keep the lumen open without any substantial lateral stretch of the solid material, which is part of the structure. In some embodiments, the material used is configured to deform its structure to fit a greater length on the lateral curve rather stretching the material itself. In some embodiments, the insertion of gaps enables this substantial length change. In some embodiments, a potential advantage of this configuration is that it provides a device having a shaft that is configured to transmit forces and torques while maintaining open lumen at such small bending radius with such wide diameter of the shaft. This type of configuration is not found in prior art devices because there was no need to transmit forces and torques while maintaining open lumen at such small bending radius with such wide diameter of the shaft.

In an example of the present invention, in order to cover the gaps and to provide smooth surface, the structure is covered (e.g. reflow) with an elastic polymer, for example made of PBAX, silicone, polyurethane, PTFE, PVC, Nylon, or others, or a combination thereof, or a multilayers thereof. In some embodiments, the cover provides a smooth surface that can stretch. In some embodiments, the layer further provides mechanical support for the lumen or for force transmission. In some embodiments, a potential advantage of the combination of multi-layered properties, for example with a spring or articulated structure (for example metal or carbon), configured to form gaps while bending, and keeping the lumen open, while being covered with a stretchable material to form an external smooth surface, is to provide a unique (and possibly counter intuitive) approach, in relation to commonly used approaches of regular (braided, plastic) shaft, of just multi-coil based approaches for shafts, since those approaches apparently do not intentionally form substantial gaps while bending, thus showing that they were not planned or capable to withstand the forces and torque transmission, while keeping an open lumen (no break, tear or kink) even at bending radius as small as the diameter of the tube, reaching about 50% longer path in the lateral side versus the inner side (e.g. 8 mm bending radius, 8 mm outer diameter of the shaft, 90 degrees turn).

### Head Interface

In some embodiments, the head is integrated into the internal shaft by a “key feature” and fixed by laser welding.

### Handle Interface

In some embodiments, the handle is fixed to the internal shaft interface, which comprise of 5.3 OD. In some embodiments, the interface between the shaft and the handle comprises a collet 39l configured to act as interface between the handle 39m and the shaft 39n, on a location on the shaft that does not comprise a welding point. In some embodiments, a potential advantage of using a collet is that usually the shafts break at welding points, therefore, by moving the interface location to a location on the shaft that does not comprise a welding point, it potentially reduces the chances of the shaft to break. In some embodiments, the interface further comprises one or more gears configured to absorb extra pressure on the shaft.

### Torque Transmission Device—Tortuosity and “Power” Loss

Referring now to FIG. 4z, showing a schematic representation of a tortuously bending configuration. In some embodiments, the assembly is bended to the tortuous bending configuration as shown in FIG. 4u. In some embodiments, the total turning friction at the tortuously bending configuration should not exceed 1 [N*cm].

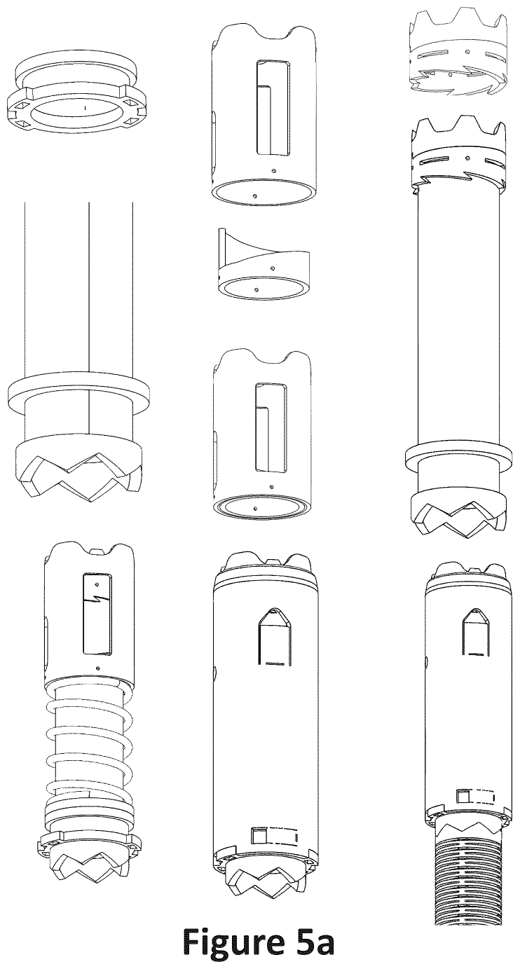

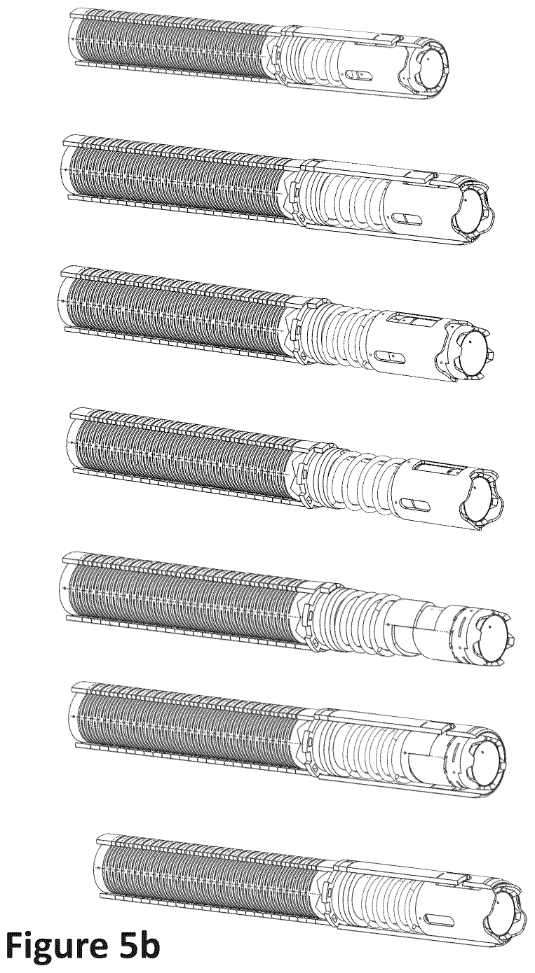

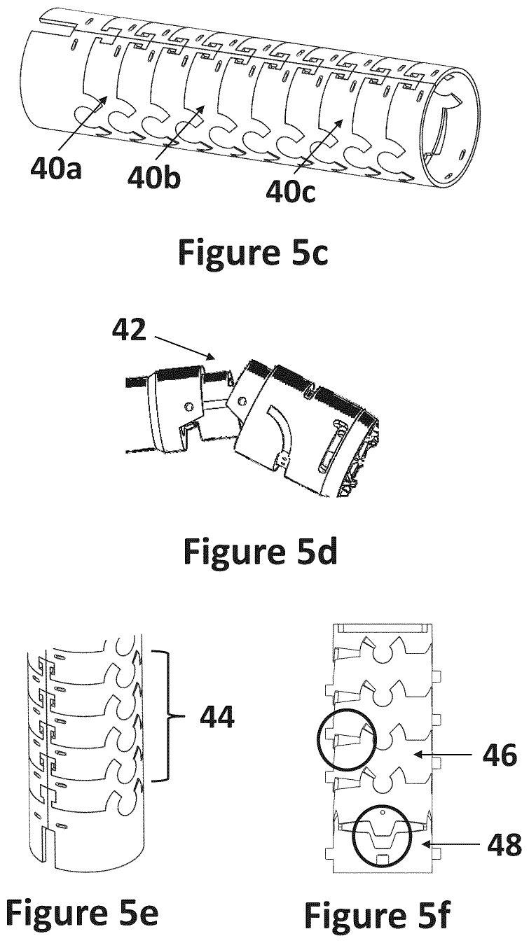

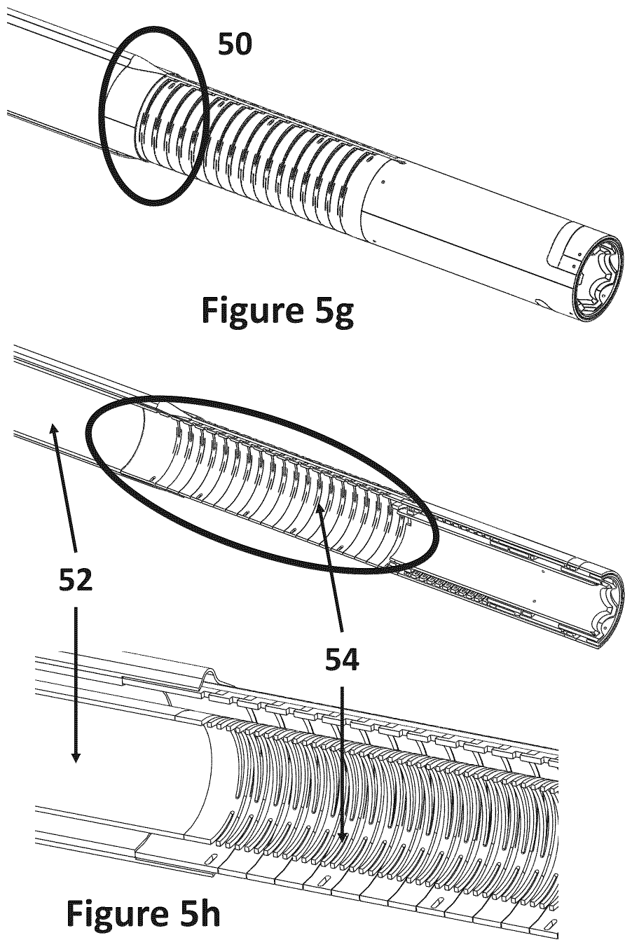

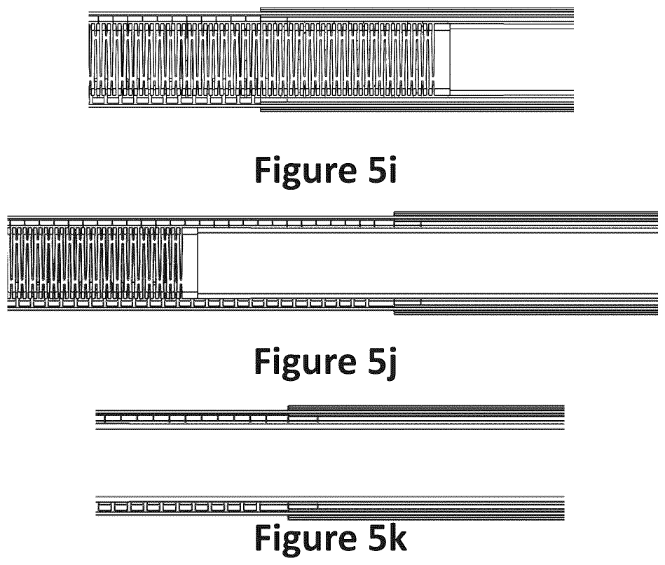

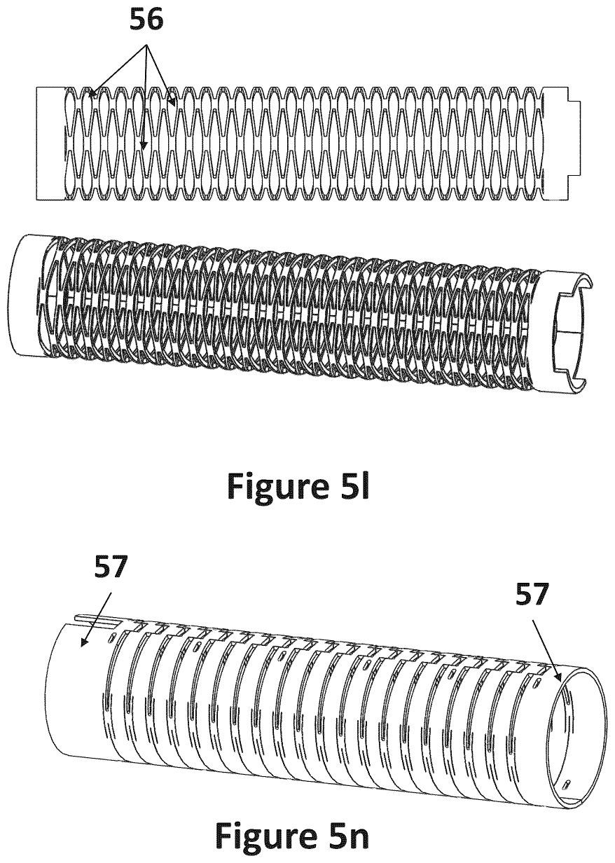

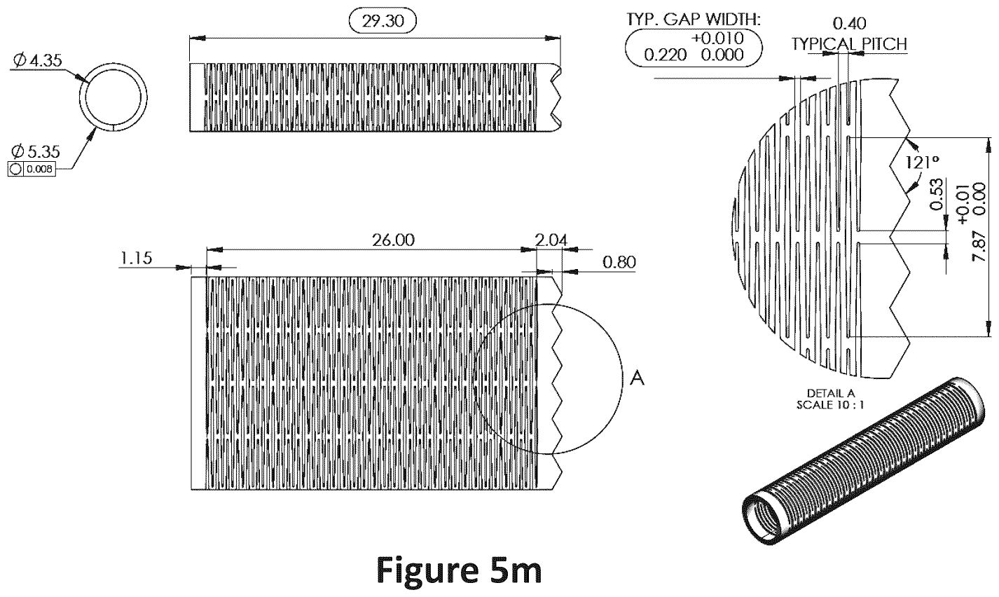

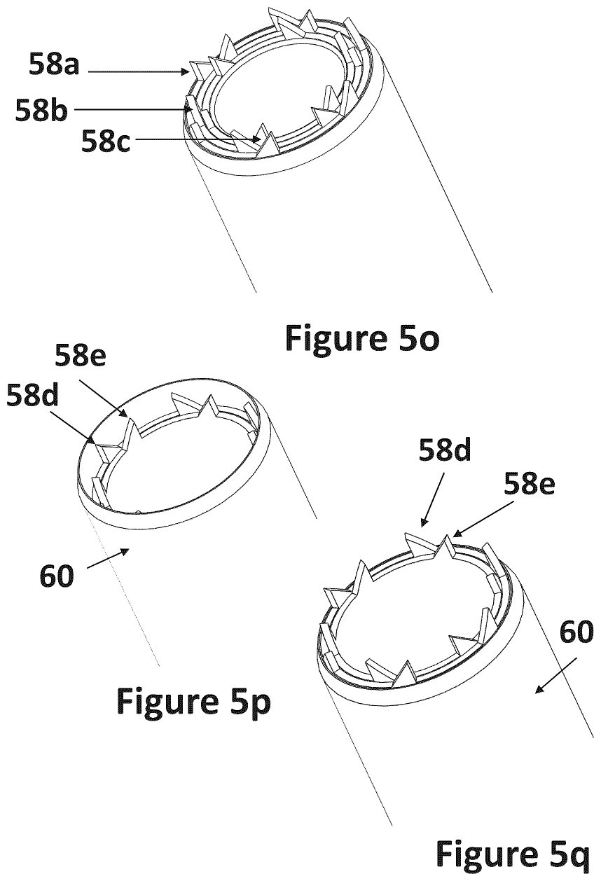

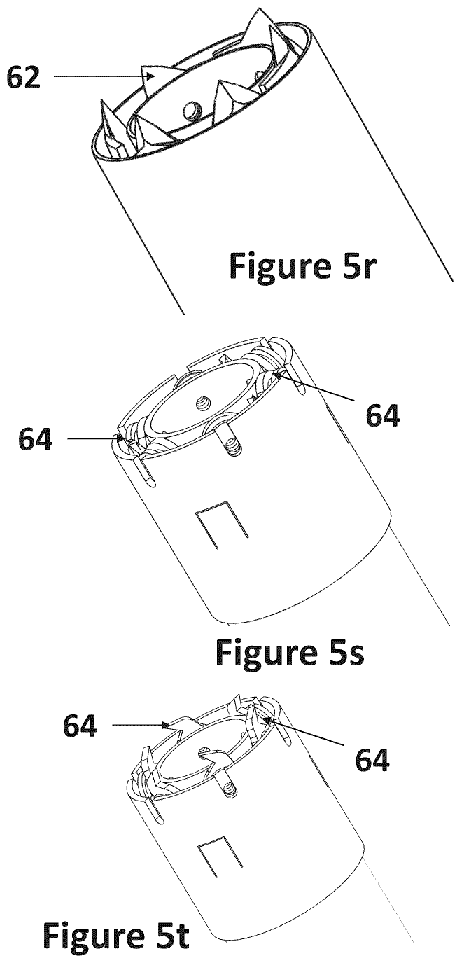

Laser welding and head assembly—as shown in FIGS. 5a and 5b, and further explained in Section 7.2, below.

### 4. Exemplary Distal Head 102

In some embodiments, the distal head of the lead extraction device includes one or more assistive tools in the extraction procedure of the cardiac lead. As mentioned above, in some embodiments, one or more of the following tools are located in the distal head of the lead extraction device: a tissue cutter tool, a tissue spreader tool, a tissue identification tool, a tissue ablation tool, a lead gripper tool, a lead cutter tool, a steering mechanism, a force measurement tool, a balloon device.

In some embodiments, the distal head includes a plurality of assistive tool fulfilling a plurality of functionalities, for example tissue/plaque cut/dissect by rotation movement of cylinder blade; tissue/plaque cut/dissect by hammering of cylinder blade/hammer; cutting edge extract/retract from/into housing to eliminate tissue damage through delivery; delivery along the lead extraction device while applying forward contact force (pushability, torquability and traceability).

In some embodiments, the length of the distal head, without the bending shaft (Hinge), is from about 2 mm to about 5 mm, for example 2 mm, 3 mm, 4 mm, 5 mm, or intermediate sizes. In some embodiments, a potential advantage of having a short distal head is that it provides the device with fewer areas that are not flexible.

In the following, some examples of each tool/mechanism will be described.

### 4.1 Exemplary Steering Tool of the Distal Head

As mentioned above, in some embodiments, the region configured for active deflection is located immediately proximal to a dilating tip portion of the sheath. In some embodiments, this configuration allows the user to actively direct the distal head of the lead extraction device towards the desired direction.

In some embodiments, a region proximal to the distal head includes a region which is significantly more flexible then other parts of the distal head and the sheath. Optionally, this region includes multiple highly-flexible regions along its length. These regions optionally include a hinge or multiple hinges (40a-c), for example as illustrated in FIG. 5c (showing multiple hinges).

Alternatively, or additionally the distal head at these locations is constructed differently and/or constructed of different material. For example, such highly flexible region may be located immediately proximal to the dilating tip portion of the sheath, as shown for example in FIG. 5d (showing a single hinge—42).

In some embodiments, the stiffness of a region of the distal head is actively controlled during use. For example, there may be a tension wire 34, which is configured to deflect the flexible region when the wire 34 is pulled, and/or there may be a tension wire, which is configured to straighten the flexible region when the wire 34 is pulled, as shown, for example, in FIGS. 3b and 3d, above. In some embodiments, the tension is adjusted by the user from the controls in the handle. In some embodiments, the tension is locked by the user allowing the distal head to remain at the selected level of tension. In some embodiments, the tension is locked using the mechanism as explained above and in FIG. 3e.

In some embodiments, active and/or passive deflection of the shaft and/or the tip of the sheath enables it to follow the curved path of the lead with reduced tension on the lead and/or less force on the sheath. In some embodiments, the hinge is capable of bending to a maximal angle during active deflection of the system while withstanding forces up to 3000 gf. In some embodiments, the hinge is capable of bending to a maximal angle during passive deflection of the system while withstanding forces up to 500 gf.

In some embodiments, the structure of the distal head of the lead extraction device is adapted to allow movement and steerability to the lead extraction device in order to enable directing the device in the right direction, especially at difficult points along the vessel where sharp turns are required.

In some embodiments, the steering mechanism is composed of multiple hinges interconnected to each other, which enable the movement of the distal head to at least one direction. In some embodiments, the hinges enable the movement of the distal head to at least two directions. In some embodiments, the hinges enable the movement of the distal end to at least three directions. In some embodiments, the hinges enable the movement of the distal end to at least four directions. In some embodiments, the hinges enable the movement of the distal head to any direction. It should be noted that, in some cases, the less directions comprise the distal head, the more torque the distal head can withstand.

In some embodiments, the bending shaft (Hinge) has a wall thickness of 0.2 mm, 0.3 mm, 0.35 mm, 0.4 mm, 0.45 mm or 0.5 mm, and optionally from about 0.1 mm to about 1 mm; optionally from about 0.2 mm to about 0.8 mm; optionally from about 0.4 mm to about 0.6 mm.

In some embodiments, the bending shaft (Hinge) is made of one cut (e.g.: laser cut) piece thereby creating in-body links. In some embodiments, the hinges are made of separate links attached and interlocked together. In some embodiments, the hinges are interlocked in the same direction 44, as shown for example in FIG. 5e. In some embodiments, the hinges are interlocked in different and/or alternate directions (46 and 48—circles), as shown for example in FIG. 5f.

In some embodiments, the bending shaft (Hinge) is connected 50 to the outer sheath and works as “counter-force” for the bending of the distal end, as shown for example in FIG. 5g, circled part. In some embodiments, the shaft is covered with a cover that extends from the proximal end, at the handle, until the distal end, comprising the hinge. In some embodiments, the cover only covers parts of the shaft. In some embodiments, a plurality of covers cover different parts of the shaft. In some embodiments, different covers having different mechanical characteristics cover different parts of the shaft thereby providing a shaft comprising different levels of stiffness along the shaft itself, as shown for example in FIG. 2i. In some embodiments, there is an inner layer 52 that rotates and\or makes the linear movement inside the sheath. In some embodiments, this inner layer has a smaller internal diameter than the outer sheath. In some embodiments, an internal layer that rotates and\or moves linearly under the bending shaft (Hinge), is called “inner bending shaft” 54, as shown for example in FIG. 5h, circled part and FIG. 5l. In some embodiments, the bending shaft is part of the inner shaft and in other option it can be a separate part that is connected to the rotating\impacting inner shaft (see further explanations in section 4.1.1). In some embodiments, the inner bending shaft holds the forces of push/torque/rotation/speed rotating/impacting/bending radius etc. In some embodiments, the inner bending shaft is smooth to reduce friction with the outer layer and the inner layer. In some embodiments, the inner bending shaft has no inner layer and in has minimum openings 56 and small dimensions for each opening to reduce friction with the lead/tissue that moves inside the device. In some embodiments, the inner bending shaft is made of stainless steel. Optionally, the inner bending shaft is a spring or multilumen. In some embodiments, the inner bending shaft is covered by a thin elastomer material, which makes the inner shaft waterproof while maintaining flexibility and reduces friction with the other parts.

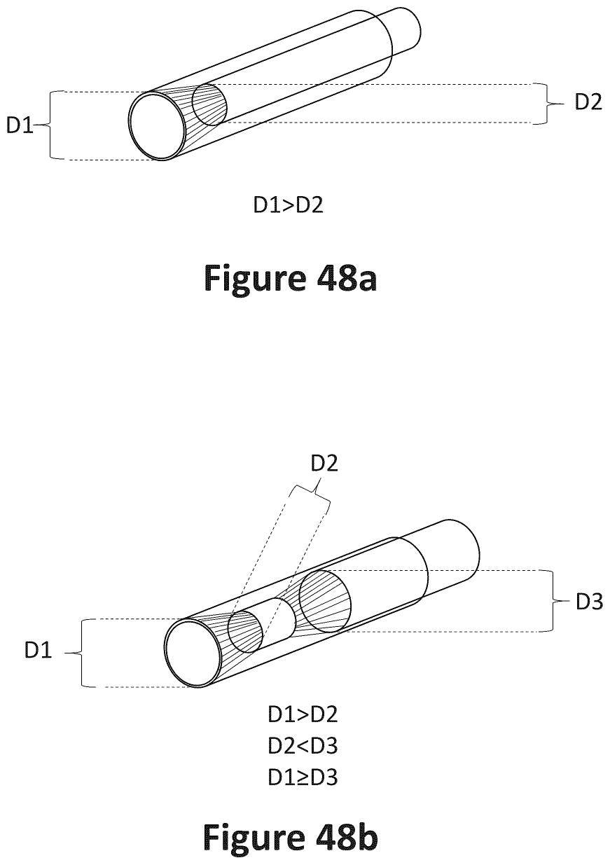

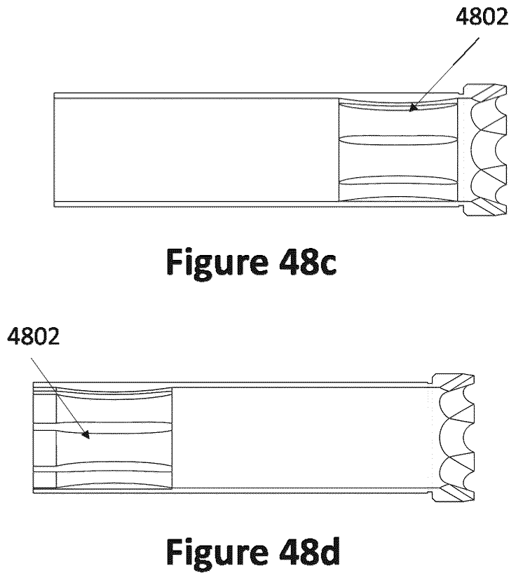

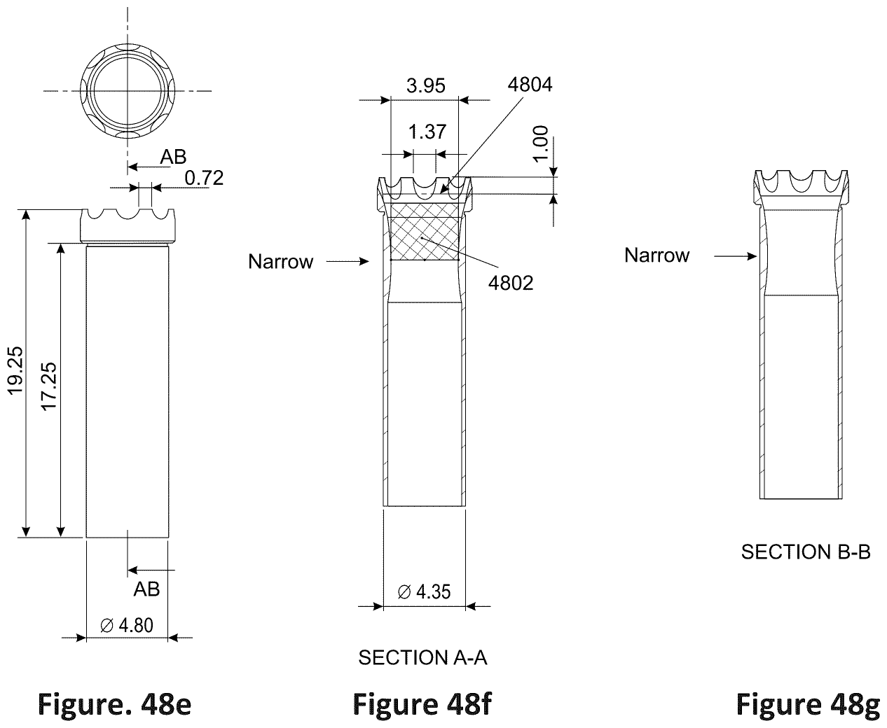

Referring now to FIGS. 48a-48g, showing exemplary distal end inner diameter configurations, according to some embodiments of the invention. In some embodiments, the most distal part of the distal end of the device comprises a certain first inner diameter D1. In some embodiments, moving proximally from that most distal part, the inner diameter of the distal end is gradually reduced to a second inner dimeter D2, thereby generating for example a conical entrance, as shown for example in FIGS. 48a and 48b. In some embodiments, D1>D2. In some embodiments, the second inner diameter D2 is kept all the way to the proximal end of the device, as schematically shown in FIG. 48a. In some embodiments, after the second inner diameter D2, the inner diameter is gradually increased to a third inner diameter D3. In some embodiments, D2<D3. In some embodiments, D1>D3. In some embodiments, a potential advantage of decreasing the inner diameter at the distal end of the device is that it potentially assists in reducing the friction in the shaft due to the lead/tissue moving inside the device. For example, only lead with or without tissue that are small enough to pass D2 will continue proximally and without exerting pressure and/or friction while passing the shaft lumen. In some embodiments, since the inner diameter is either kept as D2 or increased to D3, the friction is reduced due to the allowance of potential space within the device lumen. In some embodiments, the inner surface of the distal end, where the diameter is narrower than either the inner diameter of the entry point or the inner diameter of the rest of the device, comprises one or more indentations and/or protrusions and/or a rough surface, optionally blade-like indentations, configured to cut and/or smear and/or erode the tissue around the lead and assist in passing the lead with the tissue through the narrower inner diameter of the device. In some embodiments, D2 is smaller than D1 by a difference from about 1 mm to about 2 mm, optionally from about 0.5 mm to about 1.5 mm, optionally from about 0.1 mm to about 1 mm. In some embodiments, D2 is smaller than D3 by a difference from about 1 mm to about 2 mm, optionally from about 0.5 mm to about 1.5 mm, optionally from about 0.1 mm to about 1 mm, optionally from about 0.05 mm to about 0.5 mm, optionally by less than 0.4 mm. FIG. 48c shows that the narrower inner diameter is located at the most distal end of the device; while FIG. 48d shows that the narrower inner diameter is located at a more proximal position in relation to the opening of the device at the distal end. FIGS. 48e, 48f and 48g show exemplary dimensions of a distal end comprising a narrowing inner diameter at the most distal part of the distal end, where the indentations, in this case shown as a rough surface 4802, comprises a length of about 5 mm from the opening at the distal end, and the most distal part comprises a blade 4804 having a length of about 1 mm from the most distal part of the device. It should be noted that while the inner diameter of the distal end might change, the outer dimeter remains the same, as shown for example in FIG. 48e.

In some embodiments, the bending shaft (Hinge) is characterized by an outer diameter equal or less than the diameter of the sheath. In some embodiments, the length of the bending shaft (Hinge) is from about 6 mm to about 50 mm; optionally from about 10 mm to about 40 mm; optionally from about 20 mm to about 30 mm. In some embodiments, the bending shaft (Hinge) is characterized by a movement from 0 degrees (in the general orientation of the LE device) to about 180 degrees (in the opposite general orientation of the LE device), and in some options the bending shaft (Hinge) is characterized by a movement and bending only to one direction. In some embodiments, the minimum bending radius of the bending shaft (Hinge) is from about 2 mm to about 15 mm, optionally from about 4 mm to about 10 mm, optionally from about 6 mm to about 8 mm. Optionally less than 2.5 cmm, Optionally less than 1 cm. In some embodiments, the side movement radius of the bending shaft (Hinge) is from about 5 degrees to about 120 degrees; optionally from about 80 degrees to about 110 degrees; optionally from about 10 degrees to about 60 degrees; optionally from about 20 degrees to about 40 degrees.

In some embodiments, the minimum force required to bend the bending shaft (Hinge) is almost 0 g, since the bending shaft (Hinge) comprises a structure of multi hinges, as shown for example in FIGS. 5c, 5e, 5f, 5n. In some embodiments, the bending shaft (Hinge) is a tube that is cut to have a flexible structure, as shown for example in FIG. 5n. In some embodiments, the minimum force required to bend the bending shaft (Hinge) is from about 50 g to about 300 g; optionally, from about 100 g to about 250 g; optionally from about 150 g to about 200 g, to arrive at the maximum bending radius. In some embodiments, the bending shaft (Hinge) have 0% change in length or 1% or 3% change in length, and this almost lack of length does not affect the efficiency of the tissue cutting tool, which, in some embodiments, requires acceleration of the cutting part for a successful impact and cutting of the tissue and/or calcified tissue and/or plaque.

In some embodiments, the bending shaft (Hinge) comprises only links, meaning that at the distal end and/or at the proximal end there is no extra material that are no links, as shown for example by the arrows 57 in FIG. 5n.

In some embodiments, while the bending shaft (Hinge) mainly bends to one or more directions, the inner (named “inner hinge” or “inner bending shaft” or “tongue hinge shaft”) part of the bending shaft (Hinge) is able to rotate and expand. In some embodiments, the inner part is flexible and adapted to bend in all directions fast enough and with minimum required force while the flexible hinged part is bent. This enables the activation of the internal mechanisms while keeping to the minimum the general effects on the bending shaft (Hinge) that surrounds it. In some embodiments, the inner part is made of a cut stainless steel tube, which is flexible, as shown for example in FIG. 5l. In some embodiments, the inner part is made from a spring or from a braided polymer tube. In some embodiments, the length of inner bending shaft is similar to the bending shaft (Hinge) length and, in some embodiments, it can be shorter or longer to improve the radius of action of the bending shaft (Hinge).

In some embodiments, the bending shaft (Hinge) supports a bending radius of, for example, 2-10 cm, for example 3-5 cm, for example less than 4 cm. At this bending radius, the lumen is still open and not collapsed or kinked, so that the lead inside it can move freely.

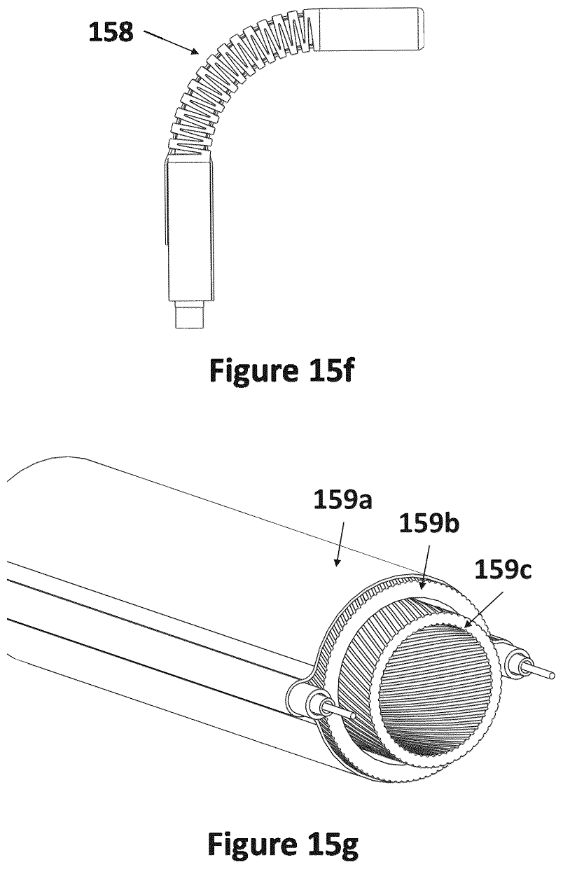

In some embodiments, the bending shaft (Hinge) comprises a small gap located between the outer-diameter (OD) of the inner bending shaft and the inner-diameter (ID) of the flexible hinged part that surrounds it. In some embodiments, this gap helps to protect the structure of the bending shaft (Hinge) from having a deformation at high torque forces, for example at forces between about 2N and about 20N; optionally between about 5N and about 15N; optionally between about 7N and about 10N; optionally the forces are at 5N, 7N, 8N, 10N, 12N, 15N or 20N. In some embodiments, the gap is from about 0.1 mm and about 0.4 mm; optionally from about 0.17 mm and about 0.3 mm; optionally from about 0.2 mm and about 0.25 mm; optionally the gap is 0.1 mm, 0.17 mm, 0.2 mm, 0.25 mm, 0.3 mm or 0.4 mm. In some embodiments, the bending shaft (Hinge) is flexible and adapted to bend in all directions. In some embodiments, the bending shaft (Hinge)'s structure is strong and adapted to hold high torque forces. In some embodiments, the ID of the bending shaft (Hinge) is similar to the ID of the inner shaft connected to distally to it, as shown for example in FIGS. 5h and 15b. In some embodiments, the ID of the bending shaft (Hinge) can be up to 1.5 mm bigger or 1.5 mm smaller from the ID of the inner shaft. In some embodiments, the lead runs through the bending shaft (Hinge), so the structure and manufacturing include elements to reduce unwanted wear or damage on the lead, for example, electro-polish after cutting the tube to lower the gaps from one strata to another. In some embodiments, the internal structure of the LE device is designed to decrease friction between the lead and the internal structure of the device. In some embodiments, this reduces the necessary force required by the user to extract the lead from the body.

In some embodiments, the bending shaft (Hinge) comprises another internal layer to reduce and/or avoid unwanted wear or damage or friction of small pieces due to the rotation of the parts.

In some embodiments, the bending shaft (Hinge) has a wall thickness of 0.2 mm, 0.3 mm, 0.35 mm, 0.4 mm or 0.45 mm or 0.5 mm. Optionally from about 0.2 mm to about 0.5 mm; optionally from about 0.3 mm to about 0.45 mm; optionally from about 0.35 mm to about 0.4 mm.

In some embodiments, the lead extraction device provides feedback to the user about the position or/and bending or/and the deflection or/and the curve of the shaft and/or the tip. In some embodiments, the deflection may be used to estimate the pressure of the tip on the tissue.

In some embodiments, the role of the bending shaft (Hinge) is to provide the maximum bending angle while keeping the lumen of the device with a round ID (and not ellipse) and avoiding the ID from collapsing or compressing or flatting or from deformation and/or while keeping the device functioning. In some embodiments, this is achieved by combining the bending shaft (Hinge) with an inner bending shaft, as will be further explained below.

In some embodiments, the bending part comprises the bending shaft (Hinge) and an inner bending shaft within, where the inner bending shaft is optionally a laser-cut inner bending shaft (see above FIG. 5h). In some embodiments, instead of the laser-cut inner bending shaft, a coiled body or a torque coil technology or braid technology is used (see below exemplary coils). In some embodiments, the coiled body is similar to that used in the elongated body (see point 3.2), but optionally having different mechanical characteristics, for example less rigid. In some embodiments, the coiled body that is used for the elongated body is also used as the inner bending shaft, meaning that the coiled body goes along the elongated body, all the way under the bending shaft (Hinge), to the distal head. In some embodiments, the connection point of the inner parts (internal coil to inner bending shaft, for example) are located at the same location of the connection point of the outer parts (bending shaft (Hinge) and outer shaft of the elongated body), for example, see 50 in FIG. 5g), as can be seen for example in FIGS. 5b, 5g and 5h. In some embodiments, the connection point of the inner parts (internal coil to inner bending shaft, for example) are located at a different location of the connection point of the outer parts (bending shaft (Hinge) and outer shaft of the elongated body, for example). Examples of this can be seen for example in FIG. 5i, showing the internal connection point is proximal to the external connection point; in FIG. 5j, showing the internal connection point is distal to the external connection point; and optionally, there is no internal connection point, as shown for example in FIG. 5k. In some embodiments, the connection point can be 10 mm to 250 mm proximally or distally from each other, either one distal from the other or the other way around, for example the distance between connection points can be 20 mm-50 mm or 40 mm-100 mm or 50 mm-250 mm for example 20 mm or 50 mm or 200 mm. In some embodiments, a potential advantage of having the connection point not at the same location is that it provides the device with less rigid parts (caused by the colocation of connection points), that will reduce the length of the rigid area, which is better when manipulating the device to follow the lead curves in the veins and into the heart at the LE procedure.

### 4.1.1 Exemplary Inner Bending Shaft

In some embodiments, an inner bending shaft is as disclosed, for example in FIGS. 5h-5l. In some embodiments, the inner bending shaft is located inside the sheath as shown, for example in FIG. 5h. FIG. 5l shows a schematic representation of the inner bending shaft. In some embodiments, the length of an inner bending shaft is, for example, from about 15 mm to about 55 mm, for example 15 mm, 25 mm, 28 mm or 35 mm. In some embodiments, the inner bending shaft is as long as the catheter. In some embodiments, the external diameter of an inner bending shaft is, for example, from about 3 mm to about 10 mm, for example 4-6 mm, 4 mm, 4.4 mm or 5 mm. In some embodiments, the thickness of the strati of an inner bending shaft is, for example, from about 0.1 mm to about 0.45 mm, for example 0.15 mm, 0.21 mm 0.5 mm or 0.25 mm.

Exemplary sizes for the inner bending shaft can be seen in FIG. 5m. It should be understood that the dimensions disclosed herein are examples only, which are provided to allow a person having skill in the art to understand the invention. While some of the examples are suitable for a 13 French scale catheter, for example, it is the scope of the invention to include either bigger or smaller sizes, for example 15 French, 11 French, 9 French, by reducing the size of the parts.

In some embodiments, the inner bending shaft is located between the distal end of the sheath and the proximal end of the distal head, inside the flexible hinged portion. In some embodiments, the inner bending shaft is interconnected, on its proximal end, to a transmission that runs inside the sheath and connected to, for example, a motor in the handle of the device; and on its distal end to an operational tool located at the distal head of the device.

### 4.2 Exemplary Tissue Cutting Tool

As explained above, in some cases, fibrous tissue surrounds parts of the lead. This can cause difficulty in the lead extraction procedure. In some embodiments, the distal head comprises a tissue-cutting tool adapted to cut the tissue surrounding the lead. In some embodiments, blades located at the distal end of the device are used as cutting tools. In some embodiments, the cutting action is linear (or axial), which means cutting the tissue when moving blades back and forth (proximally and distally). In some embodiments, the cutting action is rotational (or circumferential), which means that the blades rotate clockwise (CW) and/or counterclockwise (CCW) with one set of blades and/or by rotating one set of blades against another to shear tissue between them. In some embodiments, the cutting action is a combination of linear (or axial) and rotational (or circumferential). In some embodiments, the tissue-cutting tool comprises a protective cover, which protects from unwanted damage of the vessel walls. In some embodiments, the combination of two blades provides that one of the blades hold the tissue while the other cuts, which may prevent torque-induced damage to the vessel walls. In some embodiments, the cover mechanism comprises a distal end that is perfectly perpendicular to a longitudinal axis of the device. In some embodiments, the cover mechanism comprises a distal end that is angled in relation to the longitudinal axis of the device.

In some embodiments, the blades are smooth. In some embodiments, the blades comprise indentations and/or rough surface configured to assist in cutting the tissue. In some embodiments, a potential advantage of having indentations and/or rough surface is that it potentially helps against the “snow-plow” effect.

In some embodiments, the principals that guide the architecture of the tissue-cutting tool at the distal head are providing a tissue-cutting tool that cut the tissue to an outer-diameter (OD) as similar as possible to the outer tube of the distal head. As will be further explained below.

### 4.2.1 Exemplary Concentric Rotating Blades

In some embodiments, the distal end comprises a mechanical tip and/or rotating blades. In some embodiments, the rotating blades include, for example, two or more concentric tubes, which rotate relative to each other. Optionally, the concentric tubes have blades protruding from their distal ends, for example as illustrated in FIG. 5o. In this example, the concentric rotating blade tip comprises three concentric tubes with blades 58a-c. In some embodiments, the middle-bladed tube 58b optionally rotates with respect to the other two tubes. In some embodiments, relative rotation of the tubes optionally provides tissue disruption and/or facilitates the penetration of the tip into the fibrous tissue. Alternatively, or additionally, the tubes are not concentric. Alternatively, or additionally, the movement of the tips is non-continuous (for example a vibration and/or shaking). In some embodiments, the cutting action is rotational, which means that the blades rotate CW and/or CCW with one set of blades or by rotating one set of blades against another to shear tissue between them. In some embodiments, rotating one set of blades against another to cause a shearing effect may be an effective tool for cutting the lead, if necessary.

In some embodiments, the tissue-cutting tool includes a protective tube. In some embodiments, the protective tube is without blades. In some embodiments, the protective tube is located inside a cutting tube and/or inside a set of concentric cutting tubes. In some embodiments, the protective tube shields and/or protects the lead from the blades by physically separating between the two. Alternatively, or additionally, there is a protective tube outside the cutting tubes. In some embodiments, the protective tube shields and/or protects surrounding tissue from the blades of the cutting tubes by physically separating the blades from the surrounding tissue. In some embodiments, surrounding tissue includes walls of veins and/or the heart. An exemplary embodiment of tip having an outer protective tube is illustrated, for example, in FIGS. 5p-5q. In this example, a concentric rotating blade tip with two concentric bladed tubes and a non-bladed outer tube is shown. In some embodiments, for example, the outer tube shields surrounding tissue from the blades. In some embodiments, the blades move longitudinally relative to the outer shield. In some embodiments, the blades move distally in order to engage the fibrous tissue. In some embodiments, at the discretion of the user, the blades are extended distally and/or rotated when aggressive tissue cutting is necessary. In some embodiments, the protective tube is deployable when the blades are active. In some embodiments, the protective tube is deployable when the blades are not active.

In some embodiments, after the cutting tube moves distally, it rotates to apply a shearing momentum on the target. In some embodiments, the rotation after the cut completes the action of cutting. In the exemplary embodiment shown in FIG. 5p, the blades of a cutting tube 58d-e are shown covered by the outer tube 60 and do not extend beyond a distal surface of the outer tube. In some embodiments, the outer tube 60 comprises a distal end that is perfectly perpendicular to a longitudinal axis of the device. In some embodiments, the outer tube 60 comprises a distal end that is angled and/or beveled in relation to the longitudinal axis of the device. In some embodiments, covering the blades avoids damage to tissue and/or other leads, for example, when the sheath is moved. In some embodiments, the rotation of blades occurs only when the blades are extended beyond the outer tube, as shown for example in FIG. 5q. Alternatively, or additionally, blades rotate while within the outer tube. In some embodiments, rotating the blades while within the outer tube will free the device of tissue and/or material that is confined and/or lodged within the tube.

In some embodiments, the protective tube is deployed or retracted by the user, from the handle of the device. In some embodiments, the deployment mechanism runs from the handle to the distal end together with the motion mechanisms.

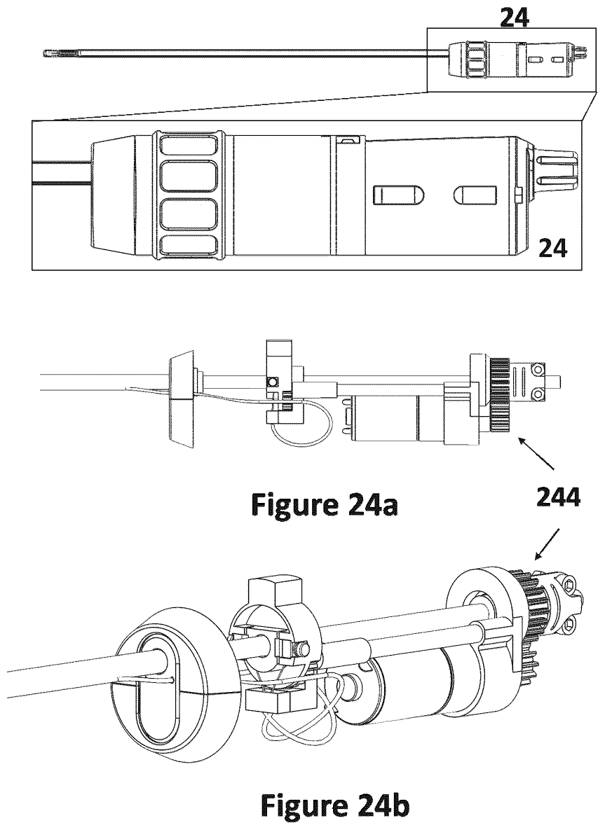

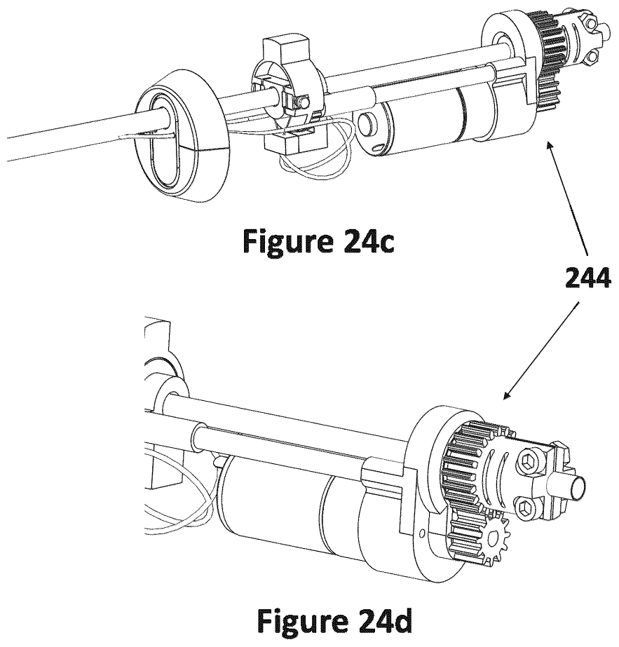

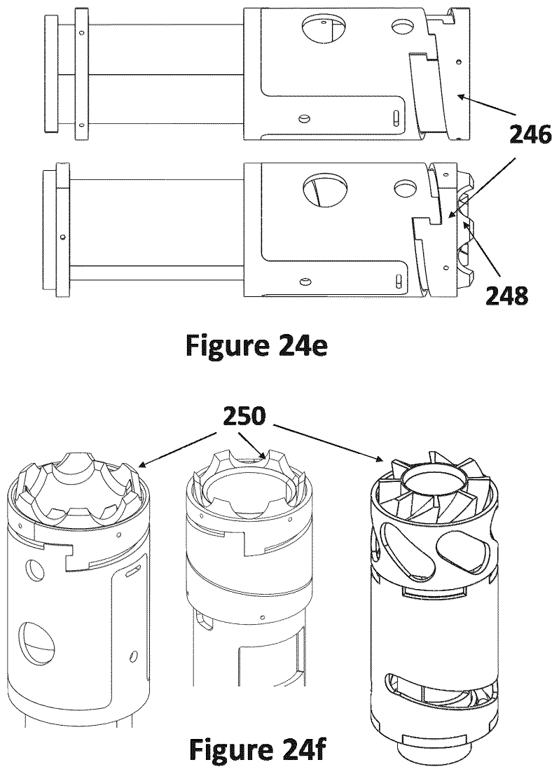

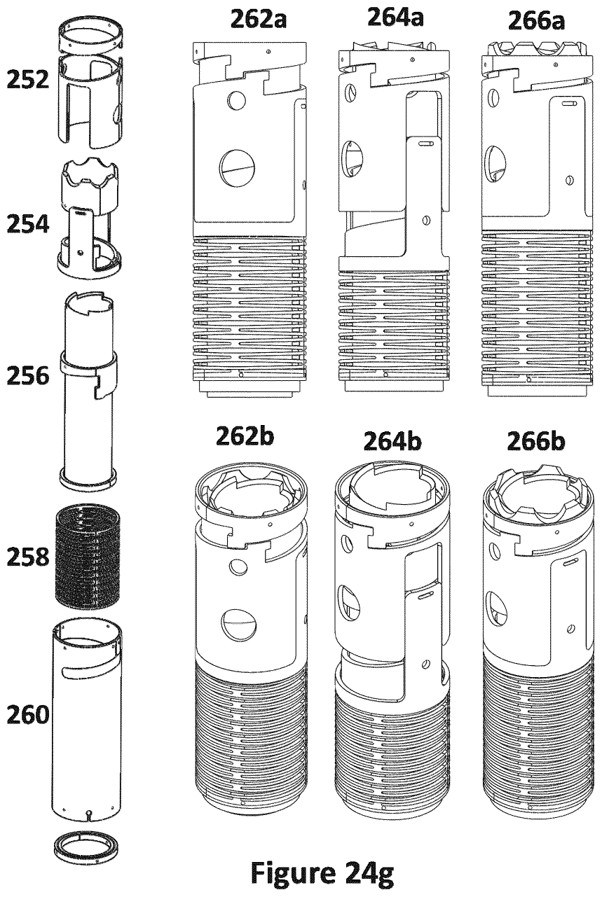

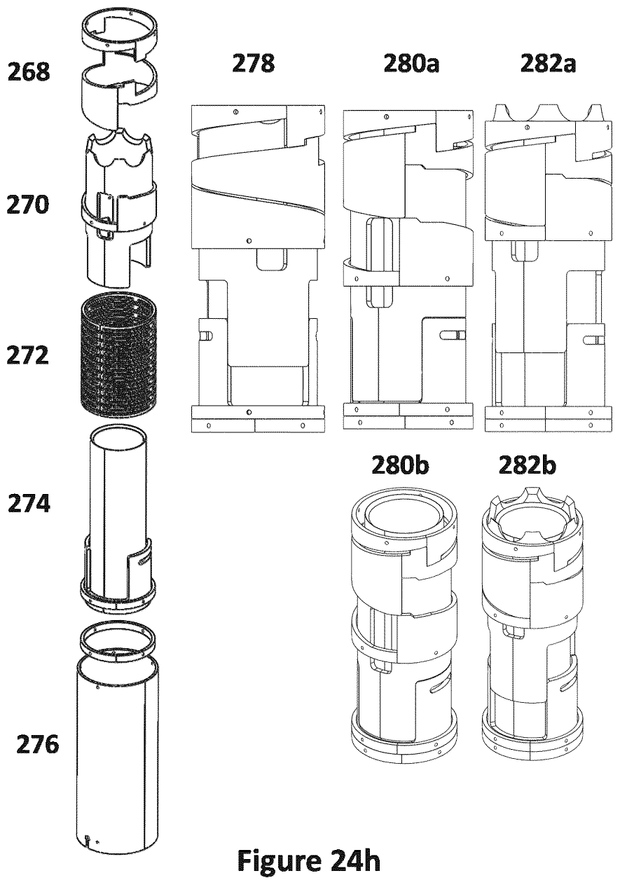

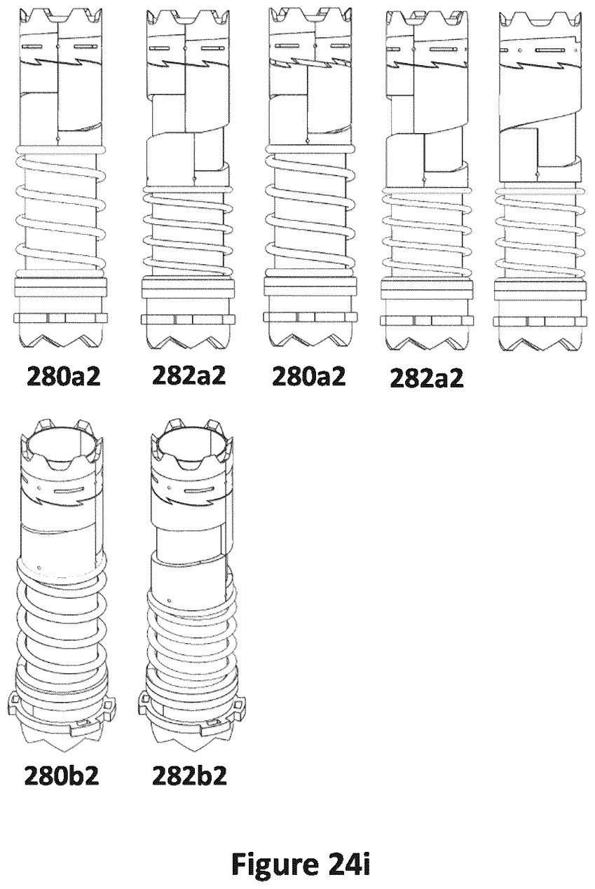

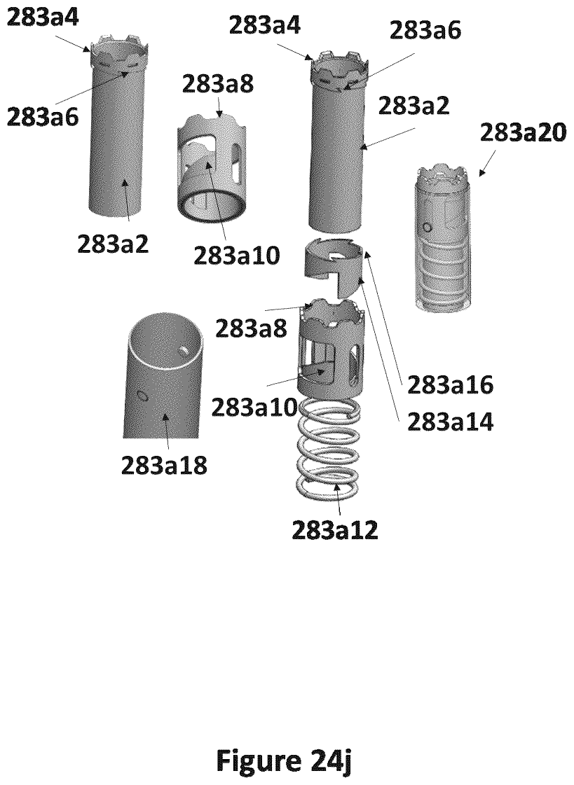

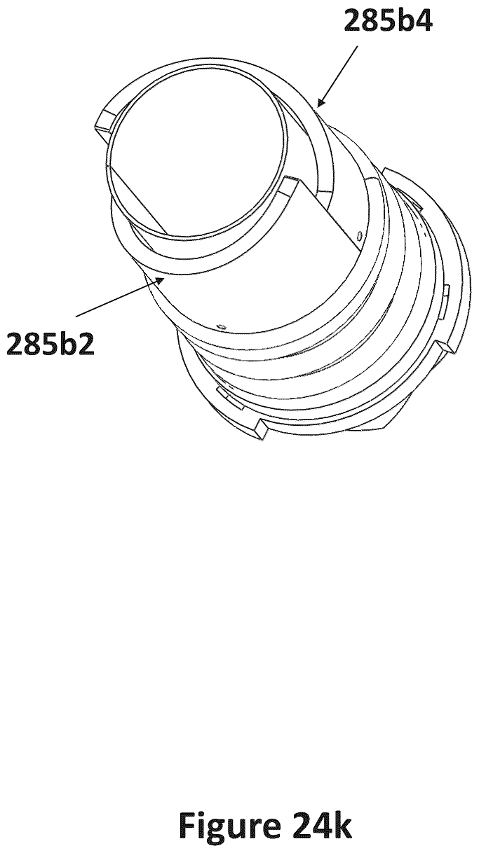

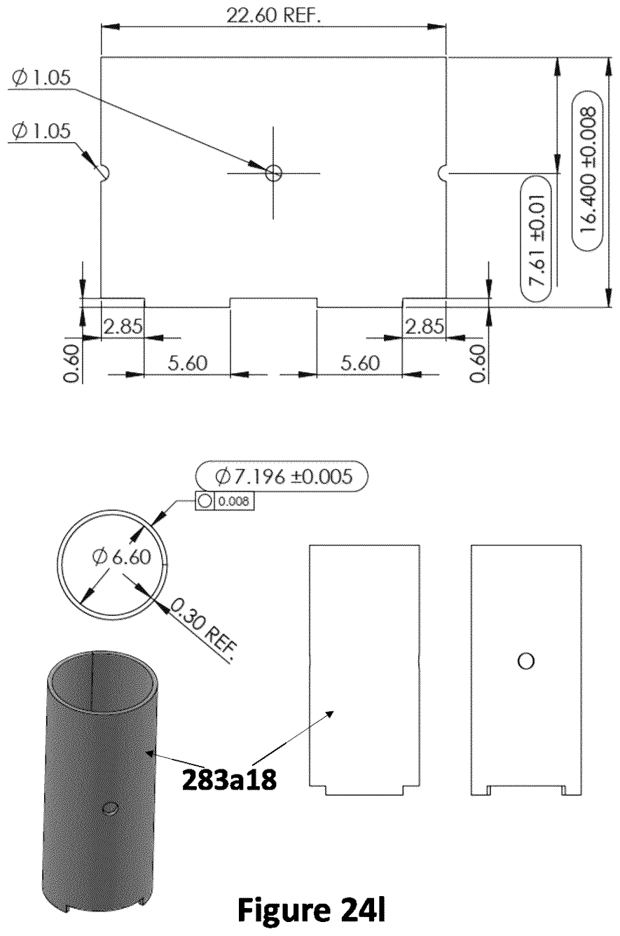

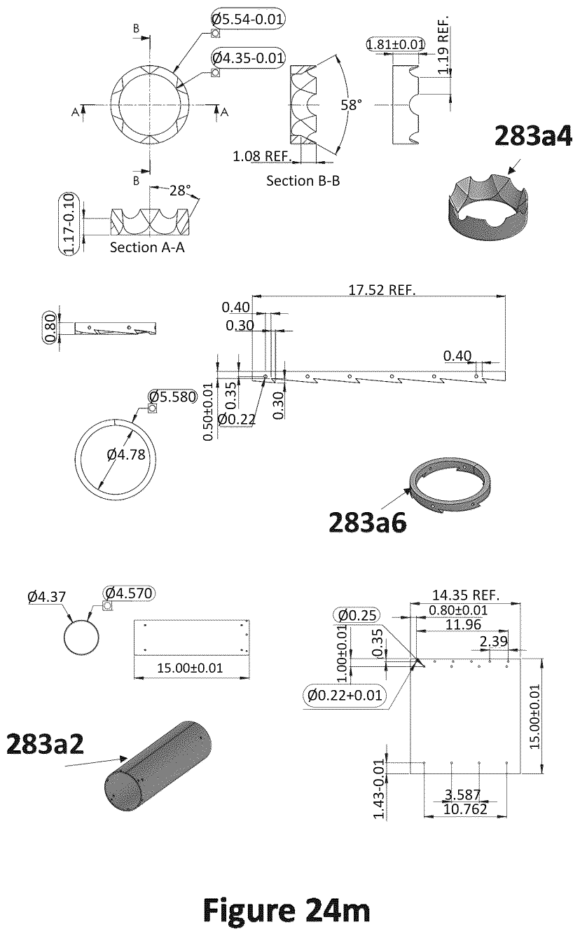

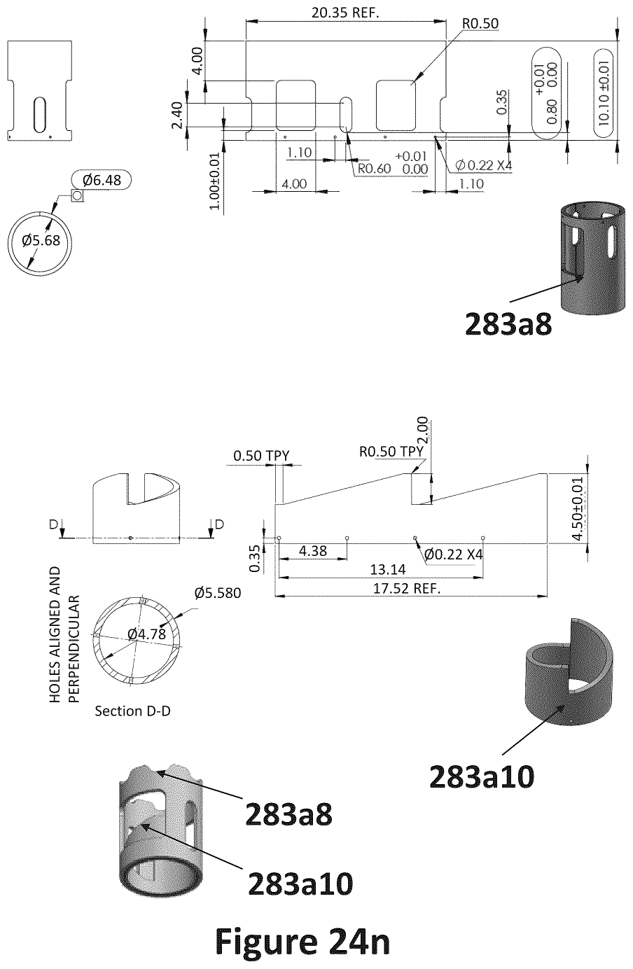

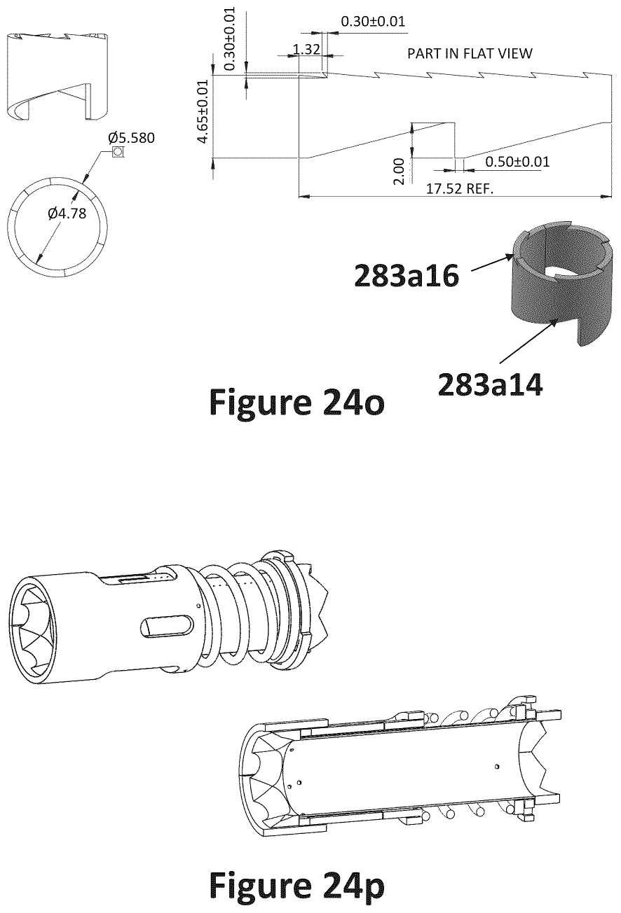

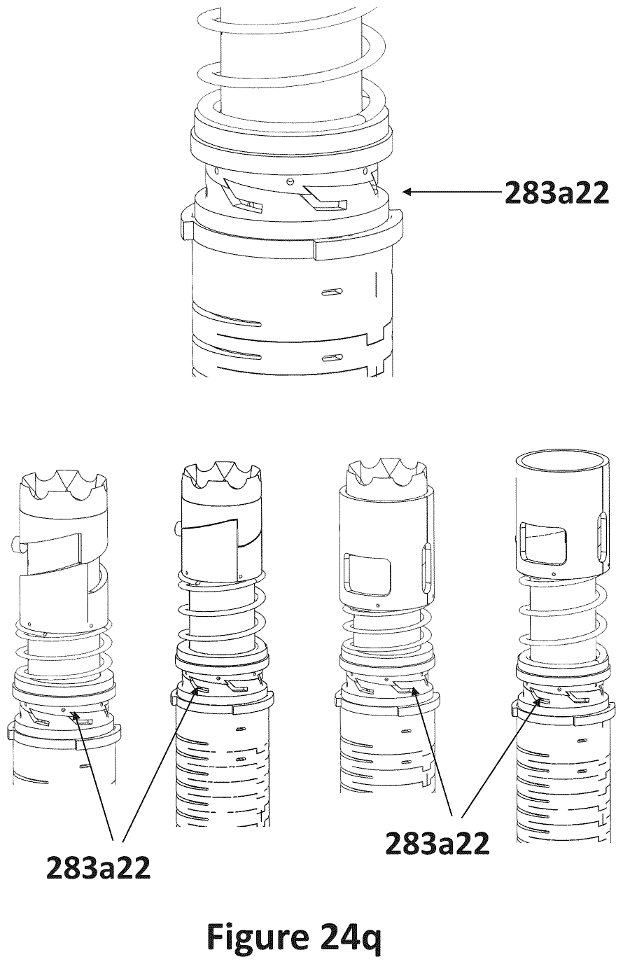

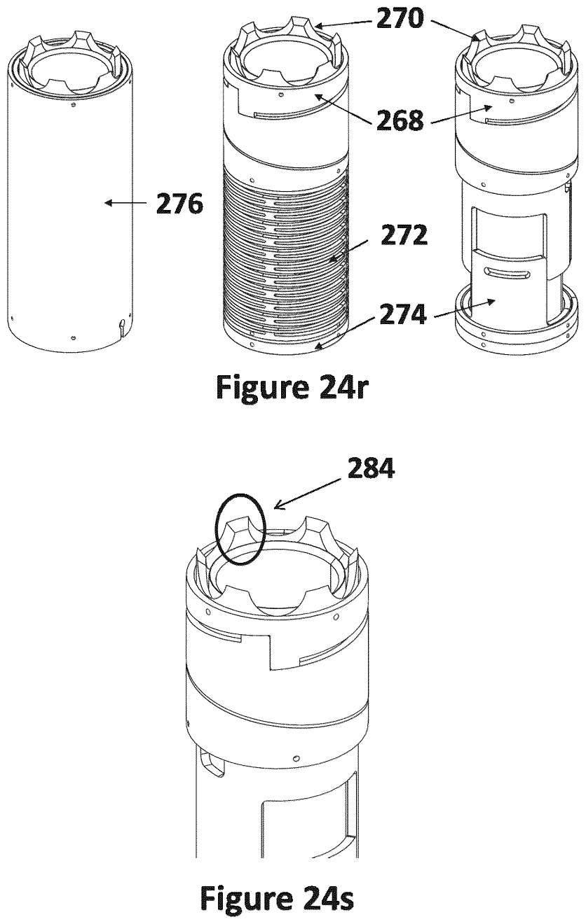

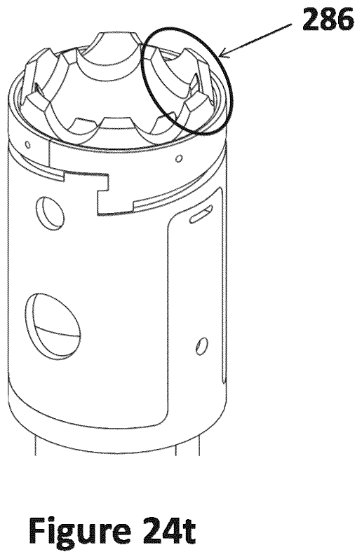

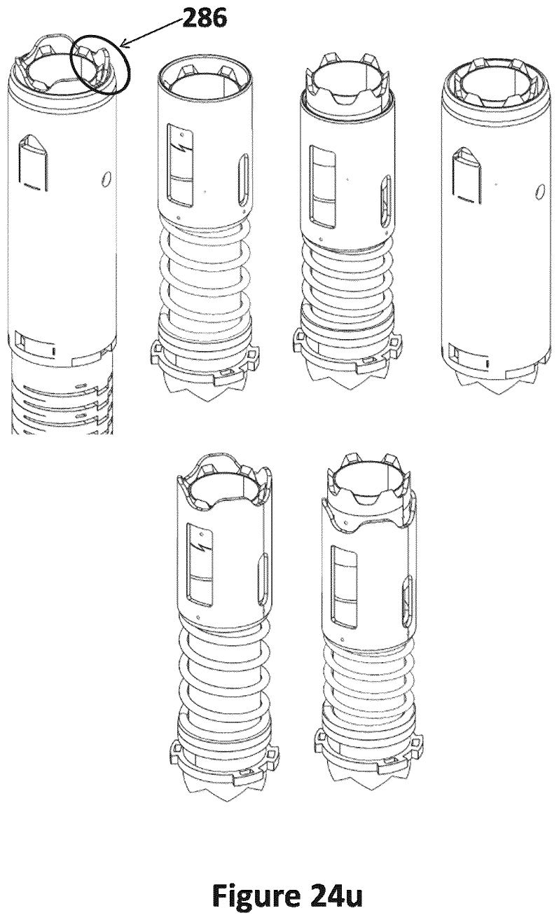

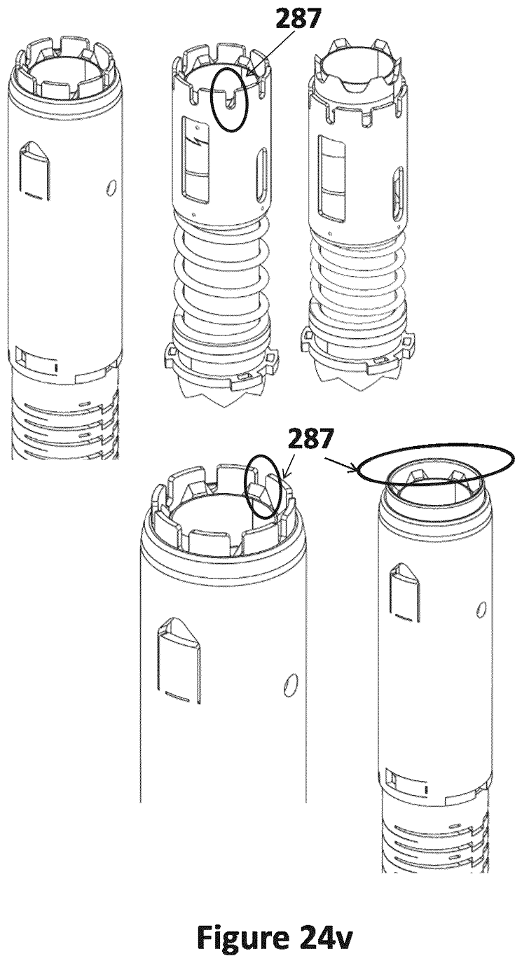

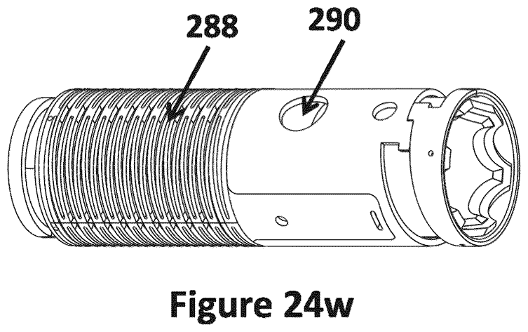

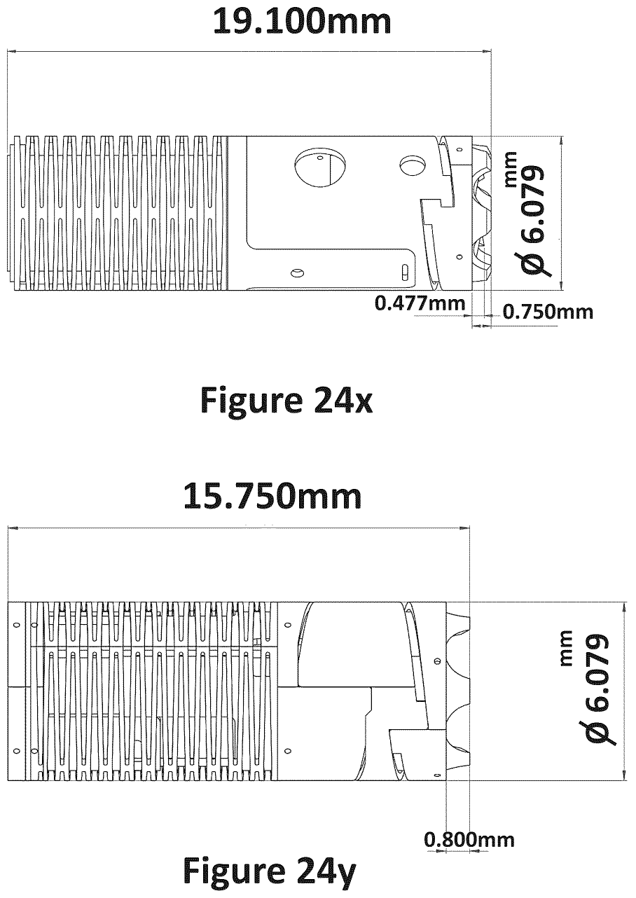

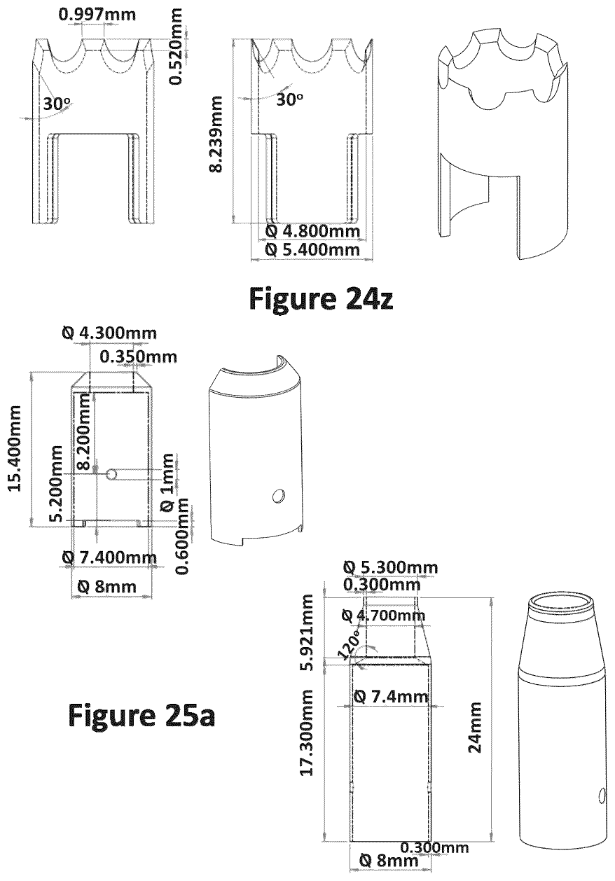

In some embodiments, the shape of the blades comprises a slope that helps in the cutting and in separating the lead from the tissue and/or from the vein. In some embodiments, the shape of the blades with the slope also avoids unwanted cutting of either the lead or surrounding tissue. In some embodiments, the blades comprise a triangular shape 62, as shown for example in FIG. 5r. In some embodiments, the blades comprise a scalloped shape with slope, as shown for example in FIG. 24f. In some embodiments, the blades comprise a scalloped shape with a slope inwards for the inner blade and with a slope outward for the outer blade. In some embodiments, the blades comprise a scalloped shape with a slope outward for the inner blade and with a slope inwards for the outer blade.

In some embodiments, the length of the blades is less than 2 mm, optionally in the range between about 0.1 mm to about 1.9 mm; optionally from about 0.2 mm to about 1.5 mm; optionally from about 0.5 to about 1.0 mm. Alternatively or additionally, a single tube and/or concentric tubes include blades of different lengths. In some embodiments, a single tube and/or concentric tubes include a number of blades from about 1 to about 50; optionally from about 4 to about 30; optionally from about 10 to about 20.

In some embodiments, the rotating blades are either inside the outer tube or slightly protruding beyond the end of the outer tube, for example, the distance between the distal end of the rotating blade in relation to the distal end of the outer is from about −0.1 mm to about 0.5 mm.

In some embodiments, the rotating blades are two concentric tubes with blades at their distal ends having different configurations. For example, in a first configuration the tubes rotate together in the same direction. In some embodiments, this configuration is used for less aggressive cutting and/or to gently dilate the fibrous tissue around the lead. In another example, in a second configuration, the tubes rotate relative to each other. In some embodiments, this second configuration is used for more aggressive cutting of the tissue.

In some embodiments, the two tubes rotate relative to each other by both tubes rotating. Alternatively, only one tube rotates. In some embodiments, a selection mechanism is supplied to enable a user to select one or a combination of various modes of blades motion. For example, modes of blade motion include one set of blades moving relative to a static (e.g. non-rotating) set of blades and/or one set of blades moving relative to one another set of moving blades and/or multiple sets of blades move together. Optionally, the various modes enable selection between more or less aggressive modes of cutting

In some embodiments, the rotational movement of the blades is provided by a motor located at the handle of the device or proximally to the user or at the pedal. In some embodiments, a transmission is connected to the motor, on one side, and to the rotating tubes on the other. In some embodiments, the user controls the action of the motor and thereby the action of the rotating tubes.

### 4.2.2 Exemplary Circumferential Rotating Blades

In some embodiments, a plurality of independent rotating blades are arranged circumferentially around a central lumen 64. Optionally, the blades are located at the tip of the LE device, as shown for example in FIGS. 5s-5t. In some embodiments, the rotating blades are sharp. For example, the blades may be configured to cut the tissue. Alternatively, or additionally there are blades that are configured to spread the tissue. Optionally, the blades are relatively blunt.

In some embodiments, rotating blades rotate continuously in one direction. Alternatively, or additionally a blade rotates a partial rotation and/or oscillate back and forth. Optionally, multiple rotating blades rotate altogether in the same direction. Alternatively, or additionally, some blades rotate clockwise while others rotate counter-clockwise. For example, alternating blades rotate in opposite directions. In some embodiments, some blades rotate clockwise or counter-clockwise for 360 degrees or 400 degrees or 500 degrees or 720 degrees or few turns in one direction and then to the other direction. In some embodiments, some blades rotate clockwise or counter-clockwise from about 15 degrees to about 1800 degrees; optionally from about 90 degrees to about 900 degrees; optionally from about 180 degrees to about 720 degrees. Alternatively, or additionally, some blades rotate clockwise or counter-clockwise and when the user turns off the rotation, the blades will turn the other way for about 360 degrees to about 720 degrees; optionally for about 90 degrees to about 180 degrees; optionally for about 120 degrees to about 160 degrees, to then pull-in the blades into the protective cover. In some embodiments, the cover will move forward to protect and cover the blades and prevent an injury of the vein or lead or other. In some embodiments, the cover comprises a distal end that is perfectly perpendicular to a longitudinal axis of the device. In some embodiments, the cover comprises a distal end that is angled and/or beveled in relation to the longitudinal axis of the device. Optionally, the rotation of blades in opposing directions is balanced to avoid twisting of the distal end of the device. For example, the balance results in a very small and/or negligible net rotational force on the tissue and/or the sheath. Additionally, or alternatively, each blade acts as a tissue anchor for another blade (not in the triangular blade embodiment), for example for the blade adjacent to it. In some embodiments, balancing rotation of different blades facilitates increased tissue spreading and/or cutting with reduced bulk tissue movement. In some embodiments, the blades always protrude from the tip of the device, for example as illustrated in FIG. 5r. Optionally, the blades only rotate when activated. In some embodiments, the blades are configured such that when they are not activated they do not protrude from the tip of the device. For example, the blades only protrude when they are activated, for example as illustrated in FIGS. 5s-5t. In some embodiments, there are between 4-12 blades. Alternatively, or additionally there are more or fewer blades.

In some embodiments, the rotating blades either are inside the outer tube or are slightly protrude beyond the end of the outer tube, for example, the distance between the distal end of the rotating blade in relation to the distal end of the outer is from about −0.1 mm to about 0.5 mm.

### 4.2.3 Exemplary Impact Element/Tip/Ring

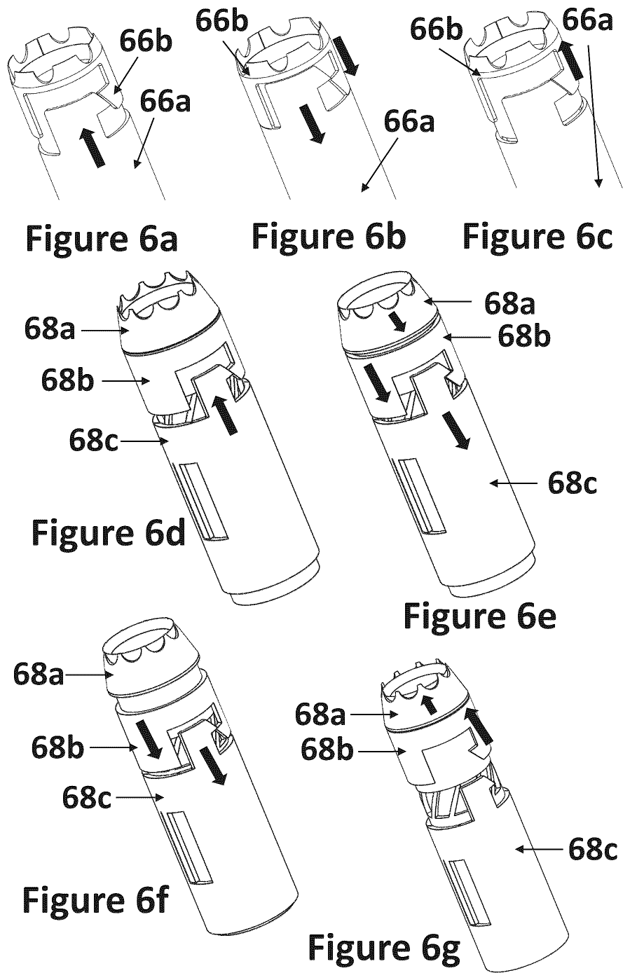

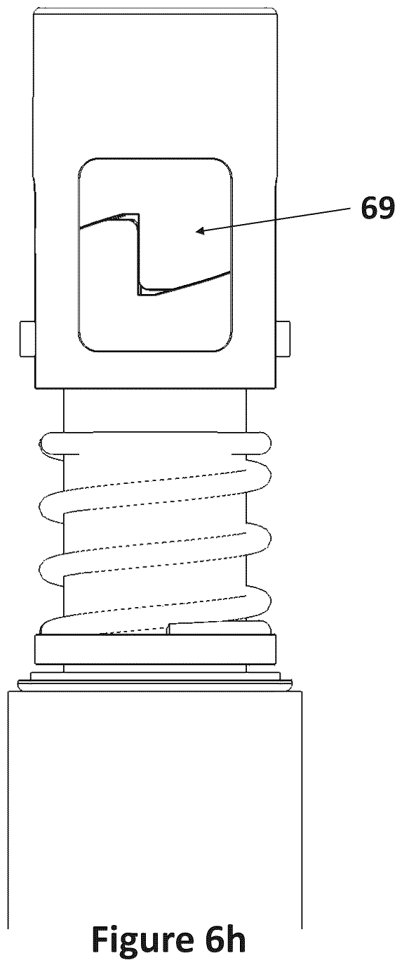

Removing tissue surrounding the lead can be difficult. In some cases, the tissue is strongly lodged around the lead and trying to separate them does not work. In these cases, a more aggressive procedure is required. In some embodiments, more aggressive procedures include hitting the tissue with something sharp and/or with something blunt. Contrary to prior art techniques, in which the force necessary to dislodge the tissue was apparently difficult to control, the following exemplary procedures disclose techniques that provide forces that are controlled, localized and having a range of motion of the impact element that is controlled as well. In some embodiments, the distal tip includes a mass, which is pulled proximally and/or pushed distally. In some embodiments, the mass is pulled against a spring and then released. In some embodiments, upon release, the mass optionally accelerates distally until it impacts the fibrous tissue, and/or impacts another component of the tip, which contacts the fibrous tissue. In some embodiments, the momentum of the accelerated mass enhances the penetration and/or dilation of fibrous tissue, for example as illustrated in FIGS. 6a-c. In this example, the impact tip is designed so the moving mass impacts directly on the tissue that it is intended to penetrate. FIG. 6a shows how the actuator 66a moves distally to engage the mass 66b. FIG. 6b shows how the actuator 66a moves proximally pulling the mass 66b with it against the spring (not shown). FIG. 6c shows how the actuator 66a releases the mass 66b, which accelerates distally until it impacts on the tissue. Other embodiments in relation to the impact ring and the tip are disclosed below. In some embodiments, impact actions are used to separate/cut hard tissue, while rotation actions are used to separate/cut soft tissue (see below paragraph regarding “differential interaction with tissue”).

In some embodiments, the mass is a tubular structure. In some embodiments, the tubular structure rides either inside and/or outside of a concentric tubular structure. In some embodiments, the mass is pulled proximally while the inner and/or outer tube remains stationary. In some embodiments, pulling the mass proximally optionally separates the mass from fibrous tissue. In some embodiments, after pulling the mass proximally, the mass comprises the space necessary to accelerate when it is released. In some embodiments, after accelerating, the mass optionally impacts fibrous tissue. In some embodiments, the mass impacts the tissue when it passes the distal end of the inner and/or outer tube.

In some embodiments, the mass impacts on a tissue-contacting component, thereby transferring its momentum to the tissue-contacting component. Optionally, the tissue-contacting component penetrates the fibrous tissue, for example as illustrated in FIGS. 6d-g. In these examples, an impact tip is design so the moving mass 68a impacts on a separate tissue-contacting component 68b that is intended to penetrate the fibrous tissue. In the exemplary configuration, the tissue-contacting component 68b may remain in contact with the tissue continuously, and/or the mass 68a may impact upon the proximal end of the tissue-contacting component 68b, transferring momentum to the tissue-contacting component 68b and/or causing it to penetrate the tissue. FIG. 6d shows how the actuator 68c moves distally to engage the mass 68a. FIG. 6e shows how the actuator 68c moves proximally pulling with mass 68a with it against a spring and/or allowing the tissue-contacting component 68b to move proximally. FIG. 6f shows how the actuator 68c continues to move proximally pulling with mass 68a with it against the spring (not shown) and/or separating the mass 68a from the tissue-contacting component 68b. FIG. 6g shows how the actuator 68c releases the mass 68a, which accelerates distally eventually impacting on the tissue-contacting component 68b and/or transferring momentum to the tissue-contacting component 68b and/or causing it to penetrate the tissue. Optionally, the accelerating mass may not contact the tissue. In some embodiments, this mode optionally enables a separation between the tissue-contacting surface and an accelerating mass. For example, the mass remains clear of the tissue and/or is free to run back and forth with reduced contact with the tissue and/or the mass accelerates with reduced friction. In some embodiments, the tissue-contacting component includes a tubular structure, which rides inside and/or outside of a concentric tubular structure. In some embodiments, the tissue-contacting structure is optionally limited in its longitudinal movement relative to an inner and/or outer tube. In some embodiments, the range of movement may be more than 2 mm, for example 2.1 mm, 2.5 mm, 3 mm. In some embodiments, the range of the movement may be less than 2 mm, for example in the range between about 0.1 mm to about 1.9 mm; optionally from about 0.2 mm to about 1.5 mm; optionally from about 0.5 to about 1.0 mm. In some embodiments, the range of the movement is between about 0.1 mm to about 5 mm. In some embodiments, the tissue-contacting component is in constant contact with tissue. For example, its distal end penetrates deeper into the tissue with each impact of the mass on its proximal end.

In some embodiments, the impact element/tip/rings are either inside the outer tube or slightly protrude beyond the end of the outer tube, for example the distance between the distal end of the rotating blade in relation to the distal end of the outer is from about −0.1 mm to about 1 mm. In some embodiments, the impact element/tip/rings protrude the same distance as the rotating blades. In some embodiments, the impact element/ring protrude about 0.5 mm more than the rotating blades.

In some embodiments, the proximal movement of one or more tension wires induce the mass to be pulled proximally and then released. In some embodiments, the proximal movement of one or more tension wires, which induces the mass to be pulled proximally and then released, is induced by the user by pulling a trigger. In some embodiments, the pulling of the trigger induces a single impact. In some embodiments, the pulling of the trigger induces multiple impacts. In some embodiments, the impacts are induced by an automated mechanism, which induces repeated impacts as long as the mechanism is activated.

In some embodiments, catch and/or pull and/or release components of the impact mechanism are located in the handle of the device. Optionally the catch and/or release components communicate with the mass and/or spring components of the impact mechanism via tension wires. For example, the wires run through the shaft of the device and/or from the handle to the distal end of the device where the mass and/or spring components are located. In some embodiments, the mass and/or spring components are located near the distal end of the device. In some embodiments, the mass and/or spring components are located proximal to another tissue cutting and/or tissue spreading mechanism. For example, in an exemplary embodiment, the handle of the device comprises a trigger interconnected to a tension wire that runs distally along the shaft and is connected to the actuator. Once the user presses the trigger, the actuator pulls back the mass, crunching the spring and loading the mass, which provides the momentum to the mass to move distally, hitting the tissue-contacting component, which then impacts the tissue.

In some embodiments, an impact mechanism is combined with other tissue cutting and/or tissue spreading mechanisms. For example, combining mechanisms may improve the effectiveness of tissue loosening, spreading and/or penetration of the device. For example, the impact mechanism is combined with a blade rotating mechanism.

Referring now to FIG. 6h, showing a schematic representation of an impact element, according to some embodiments of the invention. In some embodiments, the impact element comprises an opening 69 on the wall of the body of the impact element configured to allow fluids to freely flow in and out the impact mechanism and to potentially avoid the creation of vacuum inside the impact mechanism.

In some embodiments, the impact element comprises a distal end that is perfectly perpendicular to a longitudinal axis of the device. In some embodiments, the impact element comprises a distal end that is angled and/or beveled in relation to the longitudinal axis of the device.

### Exemplary Rounded Edges of Blades

In some embodiments, the ID and or the OD the blades is configured to be rounded. In some embodiments, a potential advantage of having rounded corners is that it reduces the chance to cause trauma to the tissue.

### 4.3 Exemplary Motion Mechanisms

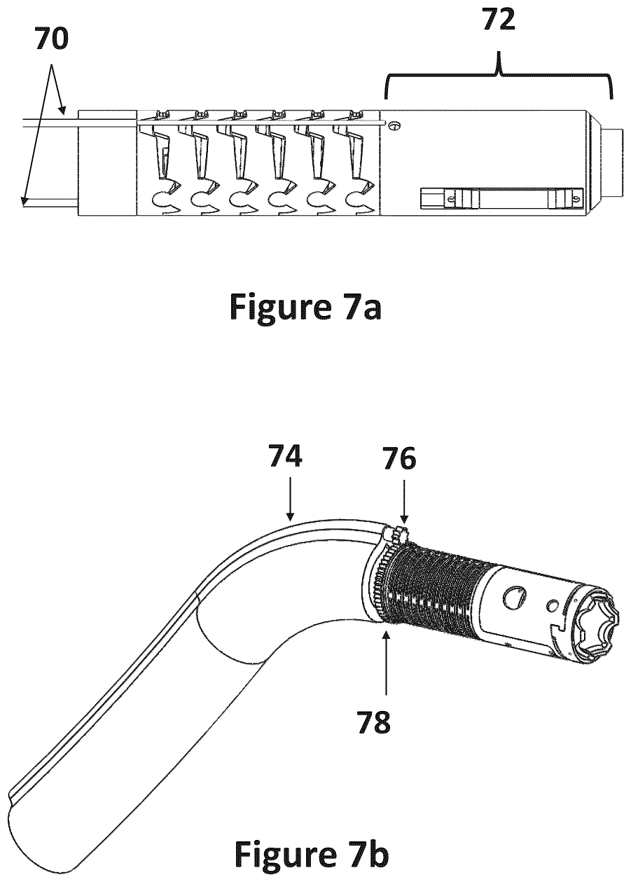

In some embodiments, the motion of one or more mechanisms of the distal tip are induced through flexible tension wires. For example, flexible tension wires may run from the handle of the device and/or through a flexible shaft of the device to the tip of the device. For example, as illustrated in FIG. 7a, pulling and/or pushing of the tension wires 70 may induce linear movement and/or rotational movement of the components of the tip 72. Some of the components of the tip may be configured such that they convert linear motion of the tension wire into rotational motion of the tip component. Optionally movement and/or changes of movement modes may be at different frequencies. In some embodiments, motion mechanism is used for steering and/or vibrating and/or impacting and/or cutting. In some embodiments, impact actions are used to separate/cut hard tissue, while rotation actions are used to separate/cut soft tissue (see below).

In some embodiments, the motion mechanism runs from the handle, for example, through the sheath, up to the distal head in a dedicated external lumen 74, as shown for example in FIG. 7b. In some embodiments, the motion mechanism is a linear mechanism. In some embodiments, the motion mechanism is a rotational mechanism 76, as shown for example in FIG. 7b. In some embodiments, the rotational mechanism engages a dedicated gear 78, which operates the tool located at the distal head. In some embodiments, an external motion mechanism may be advantageous since no movement is performed inside the lumen of the device, which may reduce the friction of the lead with the lodged tissues while passing through the device. In some embodiments, an external motion mechanism may be advantageous since delivery of the momentum from the handle to the tool is delivered by a wire, for example, using a very small radius. In some embodiments, the radius is from about 0.5 mm to about 3 mm; optionally the radius is from about 1 mm to about 2.5 mm; optionally from about 1.5 mm to about 2 mm. In some embodiments, an external motion mechanism may be advantageous since the OD of the device is smaller than a device with an internal motion mechanism.

### 4.4 Exemplary Vibration of the Distal Head

In some embodiments, the distal head is vibrated by means of the motion mechanisms described above. In some embodiments, vibration may help is loosening the tissue surrounding the lead since the resonant tends to react and/or break differently to vibrations. In some embodiments, vibration may help in the cutting action. In some embodiments, the distal head of the device includes one or more flexible regions, which are actively controlled. In some embodiments, the active regions are induced to bend or move laterally in one or more directions.

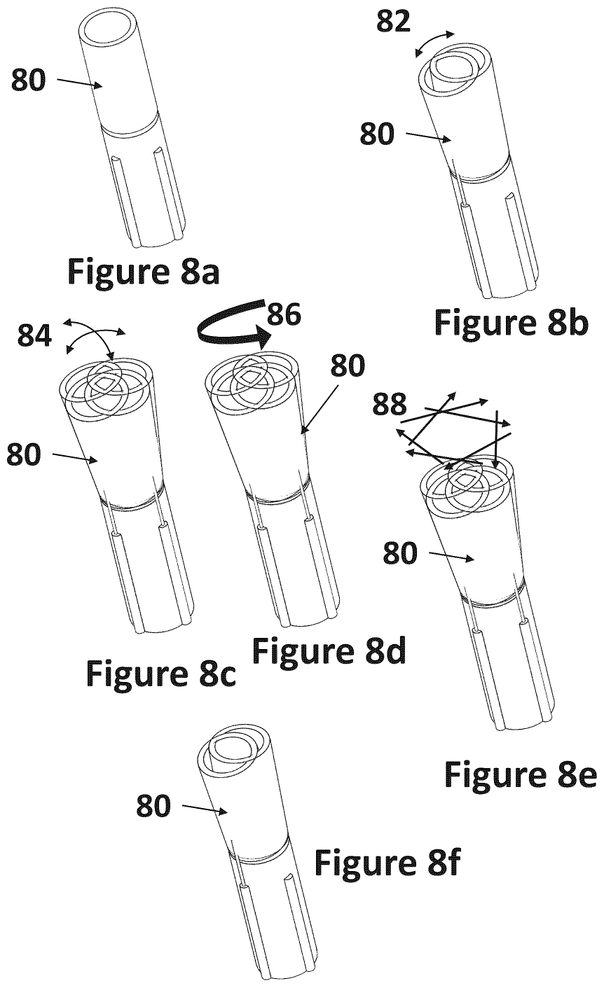

Optionally, the active regions are induced to bend back and forth repeatedly and/or to induce a vibration of the device. In some embodiments, vibration is induced in a distal portion and/or the distal end of the device. Optionally, vibrations soften, separate, disconnect and/or cause the device to penetrate the tissue. In some embodiments, the vibration affects fibrous tissue that is obstructing the movement of the lead by weakening the attachment of the tissue to the lead. This vibration is optionally induced by pulling alternatively on one or more pull-wires. In some embodiments, a pull wire induces the bending of an active region. In some embodiments, vibration is back and forth along a single axis of rotation. Alternatively, or additionally, vibration is cyclic and/or among multiple axes of rotation. In some embodiments, vibration is configured to induce a circular, random, and/or other pattern of motion of the distal tip, for example as illustrated in FIGS. 8a-e. FIG. 8a shows an example of a non-activated distal end 80. FIG. 8b shows an example on a one axis vibration 82 of the distal end. FIG. 8c shows an example of a two-axis vibration 84 of the distal end. FIG. 8d shows an example of a circular vibration 86 of the distal end. FIG. 8e shows an example of a random vibration 88 of the distal end. In some embodiments, the amplitude, frequency, and pattern of the vibration are adjusted according to the type of tissue that is being affected. For example, the type of tissue that is causing an obstruction.

In some embodiments, the amplitude of the vibration is in the range of, for example, between about 0.1 mm to about 4 mm; optionally between about 0.5 mm and 3 mm; optionally between about 1 mm and 2 mm. The frequency of vibration is, for example, in the range of between about 1 Hz to about 100 Hz; optionally between about 5 Hz to about 60 Hz; optionally between about 10 Hz to about 20 Hz. In some embodiments, the range of the movement is selected to distinguish tissue types. For example, 2 mm range of movement is compatible for use in the vein wall, but it is not compatible with calcified tissue. In some embodiments, different combinations of amplitude, frequency, and patterns of vibration are used on different types of tissue. For example, larger slower vibration is used to separate softer tissues. Alternatively, or additionally, smaller faster vibrations are used to break up harder tissues. Optionally, combinations of different amplitudes and frequencies are used to achieve the results for different tissue types and/or unknown tissue and/or combinations of different tissue. In some embodiments, the circuitry comprises a lookup table so when the user selects a desired effect and/or type of tissue, the parameters are ready and used. In some embodiments, vibrations are induced manually for example with a trigger activated device handle and/or automatically for example using an automated actuator. In some embodiments, an active vibrator includes a motor, solenoid, pneumatic and/or other type of automated mechanical actuator.

In some embodiments, a bending vibration mechanism is combined with fixed protrusions from the distal tip of the device and/or tissue cutting mechanisms and/or tissue spreading mechanisms at the tip of the device. In some embodiments, the combination increases the effectiveness of the tissue penetration. In some embodiments, cutting, spreading and vibration are synchronized. For example, different modes may be activated separately (for example to avoid uncontrolled damage) and/or different modes may be activated simultaneously (for example to cut more aggressively). In some embodiments, the circuitry comprises a lookup table so when the user selects a desired mode, the parameters are ready and used.

In some embodiments, the device includes a stiff tube. For example, an inner tube that is stiff. Alternatively, or additionally, the device includes a stiff outer tube. In some embodiments, the stiffness is defined as the stiffness necessary to transmit the vibrations in an amplitude of motion that is no more than half, or ⅓, or ⅕ or 1/10 of the amplitude of the vibrating tube. Optionally, the stiff tube does not bend significantly due to vibrations. Optionally, the inner tube holds the lead wire centered while the outer tube vibrates, moving the tissue relative to the lead wire, for example as illustrated in FIG. 8f.

In some embodiments, the stiff tube is semi-rigid. In some embodiments, the stiffness is defined as the stiffness necessary to transmit the vibrations in an amplitude of motion that is no more than half, or ⅓, or ⅕ or 1/10 of the amplitude of the vibrating tube, while also having a bending radius of about 10 cm without kinking. For example, the semi-rigid tube is flexible enough to bend slightly in order to navigate easily through the vasculature but does not flex easily or quickly enough to vibrate together with the outer tube during vibration. For example, the inner tube resists the movement of the lead with the vibration of the outer tube. In some embodiments, this resistance optionally causes the vibration to separate the tissue from the lead.

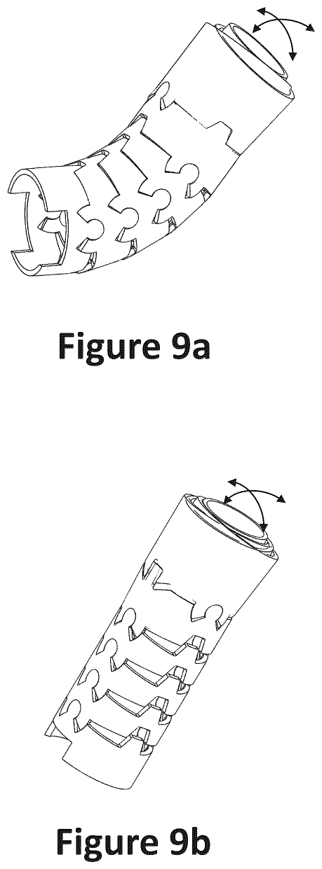

In some embodiments, a tube (for example an inner tube) bends in a controlled fashion. For example, controlled bending is achieved through the use of two tension wires on opposite sides of an axis of bending. For example, controlled bending of an inner tube may facilitate aligning a distal portion of the device with the orientation of the lead wire, as it bends within the vasculature, or with the orientation of the blood vessel itself. For example, during operation, the inner tube may be held at the appropriate bending angle, while the outer tube is vibrated around the inner tube. Optionally, vibration of the outer tube may loosen or detach the tissue from the lead wire, for example as illustrated in FIGS. 9a-b. FIG. 9a shows an example of a vibration about one axis with built-in hinge. FIG. 9b shows an example of a vibration about two-axes with built-in hinge.

### 4.5 Exemplary Eccentric Rings

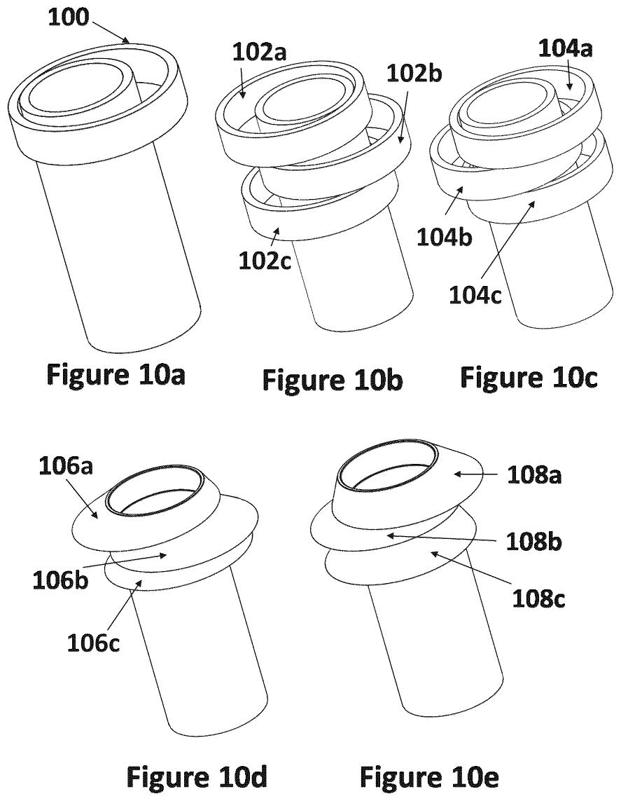

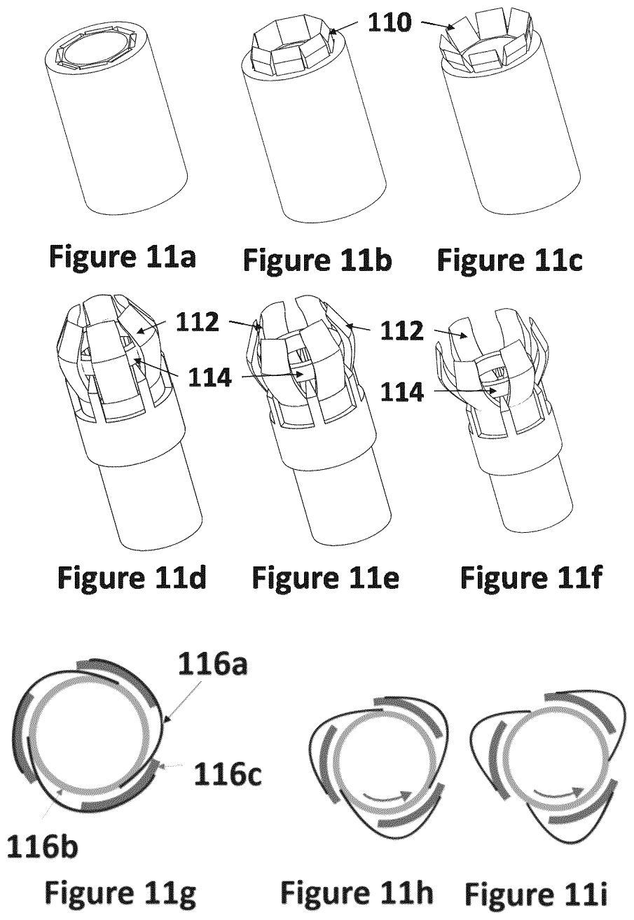

In some embodiments, the distal head of the device comprises a mechanism for breaking and/or separating and/or weakening the tissue surrounding the lead by exerting radial force. In some embodiments, the tissue breaking mechanism contains one or more eccentric rings. In some embodiments, the cross section of a ring may be circular, elliptical, egg shaped, or any other shape. In some embodiments, the ring is a complete ring. In some embodiments, the ring is a broken ring. Optionally, the position of the ring is at or near the distal end of the device. In some embodiments, rotation of the one or more eccentric rings causes them to exert force radially. Alternatively, or additionally, during rotation a ring exerts forces in different directions to induce stretching, breaking, tearing, loosening, and/or detachment of the tissue around the lead, as illustrated, for example in FIGS. 10a-c. FIG. 10a shows an example of an embodiment with one ring 100. FIG. 10b shows an embodiment with multiple rings 102a-c. FIG. 10c shows an embodiment of multiple ring of different sizes 104a-c.

In some embodiments, the device comprises more than one eccentric ring, and/or the largest radial extension of the rings increases with their distance from the distal end of the device to form a tapered form such that, as the device is advanced through the tissue, the opening in the tissue is enlarged. In some embodiments, adjacent rings rotate in opposite directions. In some embodiments, rotation in opposing directions may increase the effectiveness of the tissue separation.

In some embodiments, each eccentric ring is cone shaped, having a smaller radius at its distal edge and a larger radius at its proximal edge to make it easier for the device to advance into the fibrous tissue and the make it more effective at separating the fibrous tissue, as illustrated, for example in FIGS. 10d-e. FIG. 10d shows an embodiment of multiple cone shaped rings 106a-c. FIG. 10e shows an embodiment of multiple cone shaped rings of different sizes 108a-c. In some embodiments, there are gaps between the rings. In some embodiments, the connection between the rings provides a continuous slope.

### 4.6 Exemplary Tissue Spreaders

In some embodiments, the distal head of the device comprises a mechanism that enters between the tissue surrounding the lead and the lead. In some embodiments, the tissue is expanded locally, radially and in a limited manner. In some embodiments, the device comprises tissue spreaders at or near the distal end of the device. In some embodiments, tissue spreaders include components located at or near the distal end of the device that spread tissue in a radial direction.

In some embodiments, the tissue spreaders function by bending radially outwards. For example, they bend outward after they have penetrated the tissue or scrapped between the lead and the tissue. In some embodiments, the tissue spreaders are bent radially outwards. For example, when the spreaders are pulled into a tube of the device they straighten and/or when they are pushed distally to penetrate the tissue they at first protrude penetrating the tissue and/or when they protrude from the tip of the device they may bend radially outwards. In some embodiments, the radial protrusion optionally pulls the penetrated tissue radially outward away from the lead wire, for example as illustrated in FIGS. 11a-c.

In some embodiments, the tissue spreaders include round or flat wires (parallel to the device) and/or they may be flat and/or they may be significantly wider than they are thick. In some embodiments, the spreaders comprise a thickness from about 0.1 mm to about 1 mm; optionally from about 0.3 mm to about 0.8 mm; optionally from about 0.4 mm to about 0.6 mm. In some embodiments, the spreaders comprise a wideness from about 1 mm to about 5 mm; optionally from about 1.5 mm to about 4.5 mm; optionally from about 2 mm to about 4 mm. Optionally, the tissue spreaders are distributed around the circumference of the distal tip of the device. In some embodiments, there may be between three to 20 or 30 spreaders. Optionally, the spreaders are rigid enough to penetrate and/or push away tough fibrotic tissue. Optionally, the spreaders are strong enough, for example 0.01 Newton, or 0.1 Newton, up to 1 Newton, to spread and/or tear the tissue radially outwards. In some embodiments, the force of spreading may be due to the bending forces in the spreader. In some embodiments, the force of spreading is due an inner tube that presses the spreaders radially. In some embodiments, the spreaders may be made of Nitinol or another super-elastic material. In some embodiments, the spreaders are straightened while inside the tip of the device and bend forcefully outwards to spread the tissue. FIG. 11a shows an example of an embodiment of 8 tissue spreaders retracted. FIG. 11b shows an example of an embodiment of 8 tissue spreaders 110 partially extended. FIG. 11c shows an example of an embodiment of 8 tissue spreaders 110 fully extended and bent outwards.

In some embodiments, the spreaders extend distally from the distal end of a tube and are arranged around the circumference of the distal end of the tube, as illustrated for example in FIGS. 11d-e. In some embodiments, the spreaders are formed from longitudinal cuts in a tube. In some embodiments, the spreader flaps formed by the cuts are forced to bend radially outward by a ring located inside of the flaps and pulled proximally. In some embodiments, a number of connecting ribs, which pass between the flaps, connects the ring to a puller tube located proximal to the ring, which slides along the outside of the cut tube. In some embodiments, the flaps have a defined living hinge, which is more flexible than the rest of the flap. In some embodiments, this hinge point is made more flexible by cuts in the tube at the location of the living hinge. In some embodiments, the flaps are bent such that they bulge radially outwards around the ring and then bend back inwards distal of the ring. FIGS. 11d-f show an example of an embodiment of a device tip with optional spreaders 112 formed from cuts in a tube, which are pushed radially outwards by a ring 114 located inside the flaps which is pulled proximally.

In some embodiments, the spreader includes a circumferential band around the circumference of a distal portion of the device. In some embodiments, the circumferential band is optionally expanded radially outward by pushing or pulling the ends of the band along the circumference of the distal portion such that a portion of the band bulges radially outward. In some embodiments, the band optionally cover a portion of the circumference of the tip region, such as a portion (for example one third, one-half, and/or two thirds of the circumference or may cover the entire circumference of the tip region of a tube). Optionally, there are multiple bands. In some embodiments, the ends of the bands are located at different points around the circumference, whereby pushing or pulling the ends of the multiple bands induces bulging radially outward at multiple regions around the circumference. In some embodiments, an end of each band 116a is connected to an outer tube 116c and the other end of each band may be connected to an inner ring 116b. For example, a portion of the band may pass through a window in the outer tube 116c. In some embodiments, rotation of the inner ring 116b relative to the outer tube 116c optionally causes the band 116a to bulge radially outwards, for example as illustrated in FIGS. 11g-i. FIG. 11g shows an example of an embodiment of a “bulging band” 116a spreader mechanism in a contracted configuration. FIG. 11h shows an example of an embodiment of a “bulging band” 116a spreader mechanism in a partially expanded configuration. FIG. 11i shows an example of an embodiment of a “bulging band” 116a spreader mechanism in a fully expanded configuration.

In some embodiments, the spreader mechanisms are combined with a rotational movement and/or with a longitudinal movement and/or with an impact mechanism. For example, the combined mechanisms aid in the loosening and/or spreading of the tissue and/or with the tissue penetration of the device.

### 4.7 Exemplary Lead Wire Grasping

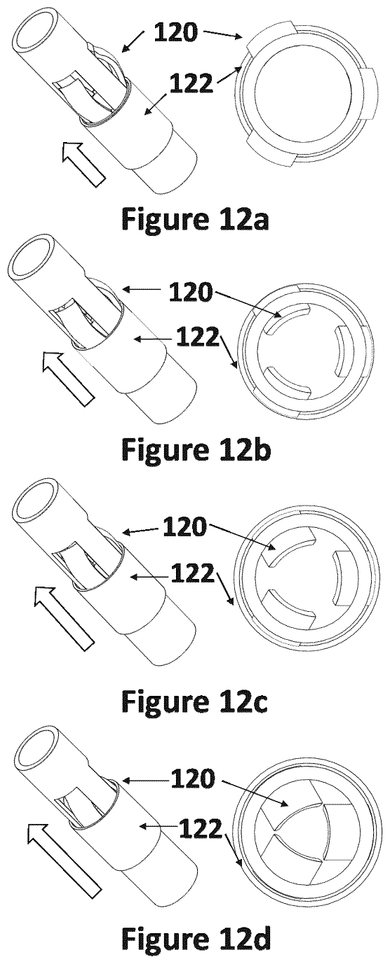

In some embodiments, the device includes a mechanism to grasp a lead wire within an inner lumen of the device. For example, the wire is grasped near the distal end of the device. For example, grasping occurs during actuation of the distal tip of the device. In some embodiments, the grasping mechanism includes an inflatable component that reduces the inner diameter of the inner lumen of the device. In some embodiments, the grasping device includes a mechanical component that protrudes inward from the wall of the device into the inner lumen reducing the diameter of the inner lumen of the device. In some embodiments, the grasping component comprises bent flaps. For example, the flaps may be formed by cutting the wall of a tube. Optionally, the flaps 120 are pushed inwards to contact the lead, for example, by sliding an outer tube 122 over the protruding portions of the flaps 120, as illustrated for example in FIGS. 12a-d. FIGS. 12a-d show an exemplary lead grasping mechanism made from 3 flaps 120 cut in the wall of a tube and bent to form grasping components that are pushed inwards to contact the lead by the movement of an outer tube 122 over the protruding portion of the flaps. Left: oblique view, Right: top view. In some embodiments, for example, the use of the lead wire-grasping tool may ensure that the user applies force to the lead at the place where lead is lodged in the tissue.

In some embodiments, a mechanism to grasp the lead wire within an inner lumen of the device is combined with other mechanisms such as cutting blades, circumferential expansion, tissue spreading, or any other mechanism. For example, the other mechanisms may apply forces on tissue in order to loosen it and/or separate it from the lead wire and/or the vessel and/or heart wall. In some embodiments, separating the lead from the tissue comprises sideways steering the tip to assist in the separation of the lead from the tissue and/or from other leads, rather than only advancing and/or dilating.

### 4.8 Exemplary Tissue and Binding Site Assessment

In some embodiments, during the lead extraction procedure, the lead extraction device comprises the ability to distinguish, in real time, between different types of matter that the distal end of the device encounters during the procedure. In some embodiments, this ability assists in the intra-procedural decision-making and increase safety. In some embodiments, for example, an indication that the LE device cutting or ablating head is facing a blood vessel tissue, rather than plaque or blood fluid, may suggest to the clinician to steer the device head, if possible, or at least to stop activating the device in that direction. In some embodiments, for example, the device comprises the ability to classify a binding site based on the chemistry of the plaque, and judge whether it is more fibrotic or more calcified. In some embodiments, this ability aids in the selection of the appropriate tool (for example: some laser LE devices are more suitable for cutting through fibrotic plaque than for penetrating a calcified plaque).

### 4.9 Exemplary IR (Infrared) Spectroscopic Classification of Matter Distally to the Device Head

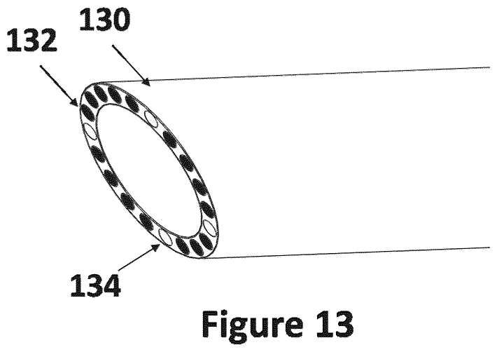

In some embodiments, the device comprises integration of spectroscopy components with ablation components, either within the lead extraction (LE) device structure or as part of an add-on or accessory device (see below—section 12), to assemble a spectroscopy system for the classification of objects distally and around the device head (FIG. 13). In some embodiments, for example, the classification is between blood fluid, blood vessel tissue, fibrotic plaque, calcified plaque and the lead itself. In some embodiments, the system provides a feedback to the clinician in the form of a score or a color scale, to distinguish between the possible objects. In some embodiments, the system provides indication that the device head is in proximity to the object in front of it, and, in some embodiments, comprises an alarm feature, to warn the clinician from further advancement. In some embodiments, alarms can be actuated together with other feedback features, like LED, vibrations, buzzers, etc.

In some embodiments, the system includes a single or multiple light emitting components, such as optical fiber tip or a light emitting diode (LED). In some embodiments, these components are mechanically positioned to radiate in a direction aligned with the LE device head. In some embodiments, depending on the light emitting technique, the signal to be radiated is carried in, to the tip of the device, by optical fibers or electrical wires along the length of the catheter. In some embodiments, the reflected signal is collected by a lens and transmitted either to an optical fiber (to be carried outside the body) or to a photodiode to convert the light to an electrical signal (to be carried outside the body by an electrical wire). In some embodiments, the system comprises a control unit used to induce either light or electrical signal, and to analyze reflected signal, whether optical or electrical. In some embodiments, the system comprises dedicated software and algorithms with examples of functions, lookup tables, activation/deactivation rules, machine-learning models, neural network models, other models, and/or ranges to classify tissue based on spectroscopic values.

In some embodiments, the fibers used for spectroscopy are integrated as part of the fibers 130 that perform the ablation functionality—as can be seen, for example, in FIG. 13. FIG. 13 shows an example of an embodiment of a laser lead-extraction device comprising ablating fibers 130 (ablating fibers are ‘black’ circles 132, spectroscopy fibers are ‘white’ circles 134).

### 4.10 Exemplary Ultrasonic Classification of Matter Distally to the Device Head

In some embodiments, the system comprises an ultrasound system for the assessment of mechanical properties of a matter, for example, based on echo analysis. In some embodiments, the generated sound waves by the transducers, propagates through the matter and is reflected according to its acoustic or mechanical properties.

In some embodiments, this modality is used to classify between blood vessel tissue, blood fluid, fibrotic plaque, calcified plaque or the lead itself, based on their acoustic properties.

In some embodiments, ultrasound transducers are embedded and/or incorporated on the head of the LE device for the purpose of matter classification. In some embodiments, the ultrasonic transducer is designed as a single piezoelectric transducer that mechanically rotates several thousand times per minute around the LE device head and thus creates a beam that is centered on and around the catheter head and projects the region ahead of it. In some embodiments, an electronic phased array of transducers are stationary placed around the device head and sequentially activated to create a focal point—of ultrasonic energy in a process known as beam forming. In some embodiments, the element 130 in FIG. 13 can be a piezo electric transmitter and/or receiver. In some embodiments, the ultrasonic system is also used to detect device proximity to the object and provides alerts on being at close proximity (e.g. 1-2 mm) to the blood vessel wall or the lead itself.

### 4.11 Exemplary Lead Cutter

In some cases, during the lead extraction procedure, the user arrives at the conclusion that the lead cannot be taken out from the tissue without causing too much damage. In these cases, it may be preferable to cut the reminder of the lead instead of forcing it out.

In some embodiments, the distal end of the LE device comprises a lead cutter. In some embodiments, the lead cutter works on a lead that is located at the lumen of the device. In some embodiments, the cutting of the lead is done by bending the lead where a blade, along the tube, can cut it. In some embodiments, the bending of the lead is done within the tube or outside the tube. In some embodiments, the lead is actively bent towards the blade. In some embodiments, the blade is a dedicated blade for lead cutting. In some embodiments, the blade is a tissue-cutting blade.

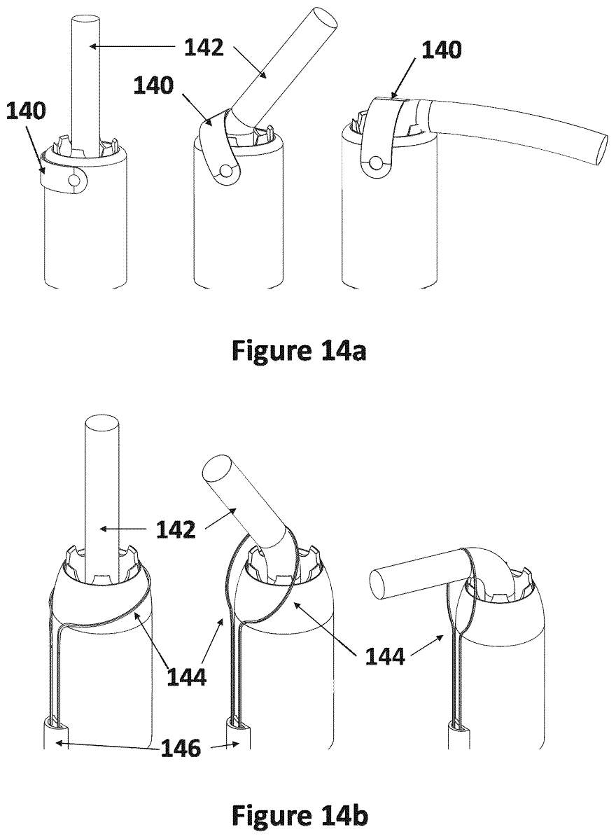

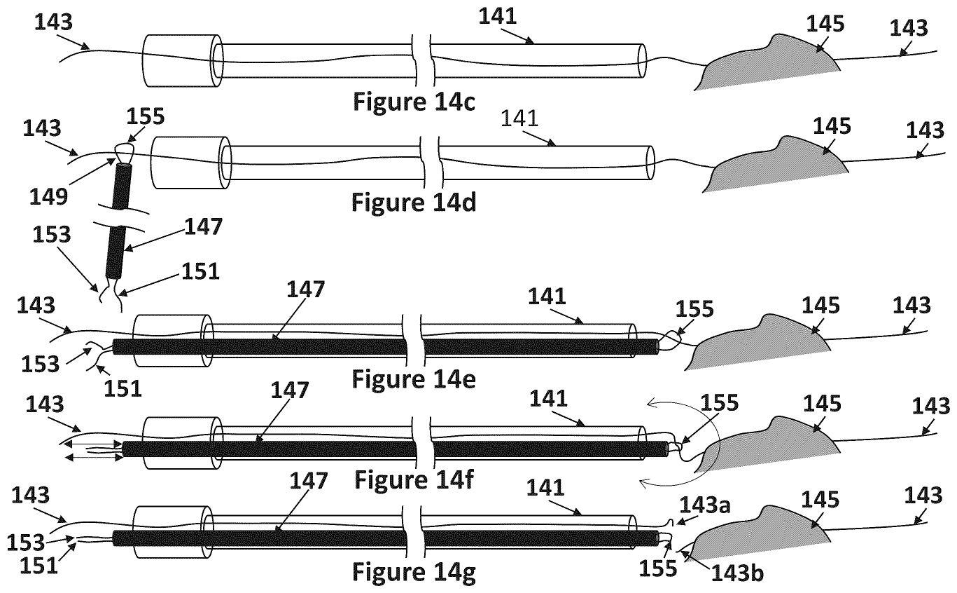

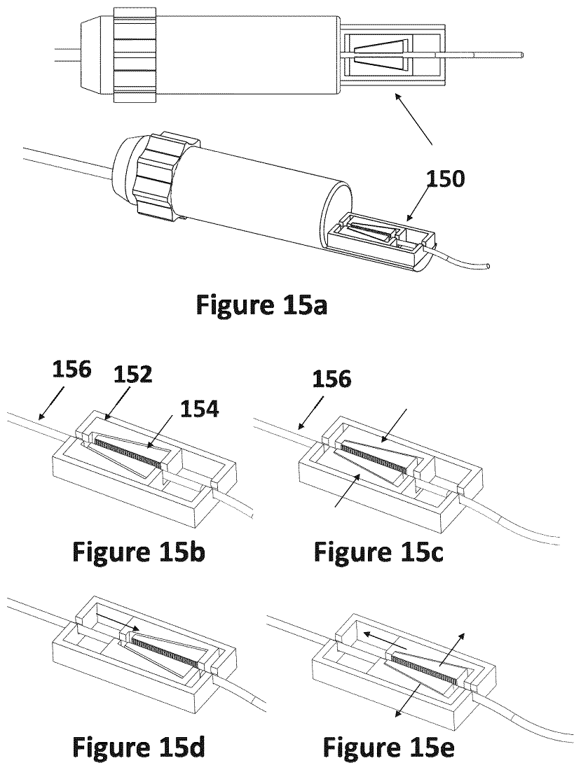

In some embodiments, the lead cutter comprises a small moving part 140, optionally as an add-on or accessory (see below—section 12), that slides and engages the lead 142 when the extractor is out or while the extractor is still in position where a cut is needed by the user, as shown for example in FIG. 14a.

In some embodiments, the lead cutter can be redrawn and/or reloaded after a cutting attempt was done for relocating or replacing a tool, according to the user decision.

In some embodiments, the lead cutter comprises a wider device, optionally as an add-on or accessory (see below—section 12), that goes around the extractor, as shown for example in FIG. 14b. In this embodiment, a wire-like 144 is shown to exit from an external additional elongated tube 146 running parallel to the LE device. In some embodiments, the wire-like 144 is made, for example, of nitinol or any other material. In some embodiments, the wire is in a non-deployed state hugging the LE device (left side). In some embodiments, a dedicated groove (not shown) is used to keep the wire in its non-deployed state. In some embodiments, the groove is perpendicular to the LE device. In some embodiments, the groove is non-perpendicular to the LE device, having a diagonal orientation. In some embodiments, the wire “natural” memory state is in an opposite orientation related to the non-deployed state. This means that, once deployed, the wire will try to return to the “natural” memory state, which is moving apart from the LE device.

In some embodiments, the external additional elongated tube 146 running parallel to the LE device and containing the wire is irreversibly attached to the LE device. In some embodiments, the external additional elongated tube 146 running parallel to the LE device and containing the wire is reversibly attached to the LE device. In some embodiments, the external additional elongated tube 146 running parallel to the LE device and containing the wire is adapted to move forward and backwards in relation to the LE device.

In some embodiments, the lead is cut by using the existing deployable blades in the LE device. Since the lead is attached on its distal part to the heart and is being pulled from its proximal part by the user, a tension is created on the lead. In some embodiments, a steering movement of the distal part of the LE device while maintaining the tension created on the lead induces a sharp bending radius to the lead and forces the lead to “lean” on the edge of the distal end where the rotating blades are located and deployed. The steering mechanism is strong enough to provide the force necessary to cut the lead by means of the blades and the tension created on the lead itself.

### Exemplary Lead Cutter Wire “Lasso” Configuration

Referring now to FIGS. 14c-14g, showing a schematic representation of an exemplary use of a wire as a lead cutter, according to some embodiments of the invention. It is known in the art that sometimes it is difficult to separate a lead from the tissue in which it is trapped. In these cases, the operator might choose to cut the lead instead of continuing extracting it. The action performed today is to cut the lead at the proximal side because that is the closest an operator can reach the lead. Then, the rest of lead is left inside the vessel, which is known to possibly cause problems in the future, for example, due to calcifications and/or tissue growth, which naturally accumulate on the lead left inside the vessel over time. FIG. 14c schematically shows the situation where the lead extraction device 141 has reached a point within the vessel where the lead 143 is trapped in hard tissue 145. In some embodiments, the lead extraction device 141 of the invention allows the user to utilize a dedicated wire lead cutter. In some embodiments, an exemplary wire lead cutter device comprises a hollowed elongated body 147, in which the cutting wire 149 is inserted within the lumen of the hollowed elongated body 147. In some embodiments, the cutting wire 149 is inserted within the lumen of hollowed elongated body 147 so the two ends 151/153 of the cutting wire 149 exit from a proximal end of hollowed elongated body 147, while a looped side 155 (or just loop 155) of the cutting wire 149 exists the distal end of hollowed elongated body 147, as shown for example in FIG. 14d. In some embodiments, the proximal end of the lead 143 is inserted within the looped end 155 when the wire 149 has already been inserted within the hollowed elongated body 147. In some embodiments, insertion of the wire 149 within the hollowed elongated body 147 and preparation of the lead 143 for cutting is performed as following: one end of the cutting wire (either 151 or 153) is inserted at a proximal end of hollowed elongated body 147. In some embodiments, then it is brought to the distal end of hollowed elongated body 147. In some embodiments, then it is looped around the lead 143 at a proximal end of the lead 143 (usually done outside the body, where the lead exists the lead extraction device) and then reinserted within the distal end of hollowed elongated body 147 until is brought out through the proximal end of hollowed elongated body 147, to bring the device ready to continue, as shown for example in FIG. 14d. In some embodiments, at this point, the user inserts the hollowed elongated body 147 inside the lumen of the lead extraction device 141, until it reaches the location where the user wants to perform the cutting, as shown for example in FIG. 14e. In some embodiments, the cutting of the lead can be done anywhere along the lumen of the lead extraction device (not shown in FIGS. 14c-14g). In some embodiments, the cutting of the lead can be performed anywhere outside the lead extraction device at any distal location within the vessel, as shown for example in FIG. 14e. In FIG. 14e, the wire lead cutter is brought the close to the tissue 145 in which the lead 143 is trapped. In some embodiments, then the user pulls the two ends 151/153 of the wire 149 to generate tension between the wire 149 and the lead 143 at the location of the loop 155. In some embodiments, the user then actuates the two ends 151/153 by moving one end of the wire 149 forwards while moving the other end of the wire backwards, thereby generating the rotating/cutting movement of the loop 155 at the desired cutting location, as shown for example I FIG. 14f. In some embodiments, the user actuates the wire 149 until the lead 143 is cut, as schematically shown in FIG. 14g, showing the cut location at points 143a and 143b on the lead 143.

### 5. Exemplary General Mechanisms/Characteristics of the Device

### 5.1 Exemplary Motion Repetition

In some embodiments, a movement of one or more of the components is repeated. In some embodiments, repetitions are due to manual repetition. In some embodiments, repeated motions are motor driven. In some embodiments, the user controls the rate of repetition. In some embodiments, a repetition rate may range between 0.1 Hz to 300 Hz; for example 1-100 Hz, for example 25-80 Hz, for example 50 Hz, for example 60 Hz, for example 24 Hz, for example 1-10 Hz. In some embodiments, the frequency is selected among several predefined frequency modes, alternatively or additionally, a frequency is selected over a continuous range of frequencies. Alternatively or additionally, a frequency is adjusted automatically. In some embodiments, a combination of multiple frequencies is used. In some embodiments, the frequency regime is chosen to achieve a clinical goal. In some embodiments, the clinical goal may be related to the tissue type and/or breaking of the tissue from the lead and/or separating the tissue from the lead. In some cases, it is preferred that a lodged segment of 1 cm of the lead, be breached in less than 1 minute, better in less than 30 seconds, better in less than 10 seconds. In some embodiments, the device makes a forward progress of at least 0.1 mm, better 0.2 mm, better 0.3 mm per each activation cycle of the device (e.g. per hit, or cut motion, or vibration, or a combination of these). In some embodiments, this progress is achieved with minimal force applied by the user from the proximal end of the device or in pulling the lead, for example with a force less than 10 Newton, for example less than 5 Newton, for example less than 3 Newton, for example less than 2 Newton. In some embodiments, a repetition rate of 3-10 cycles per second should provide, for example, a total progress rate of 3 mm-1 cm in 10 seconds. Optionally, the frequency regime is adjusted depending on tissue types and/or with different challenges and/or for selecting a speed of progression. For example, the frequency ranges between 5 Hz to 10 Hz. For example, the frequency is less than 70 Hz, and/or less than 30 Hz and/or less than 20 Hz. Optionally, the frequency ranges between 5 to 20 Hz. In some embodiments, higher frequency ranges are selected for one or more of the components. In some embodiments, one or more of the components, having an interface with and/or in proximity to the tissue, is activated with repeated motion at one or more frequencies of above 100 Hz. In some embodiments, the frequency ranges between 500 Hz to 2 KHz and/or between 2 KHz to 5 KHz and/or between 5 KHz to 10 KHz and/or between 10 KHz to 15 KHz, and/or between 15 KHz to 20 KHz or above. In some embodiments, the one or more frequencies may be supersonic and/or ultrasonic. In some embodiments, the system comprises dedicated software and algorithms with examples of functions, lookup tables, activation/deactivation rules, machine-learning models, neural network models, other models, and/or ranges to activate frequency regime based on the type of tissue.

In some embodiments, the one or more frequencies and/or one or more frequency controls for one component of the catheter differs from another component. In some embodiments, frequencies of different components may be independent. Alternatively or additionally, frequency of one component may be dependent on a frequency of another component. In some embodiments, bending may be repeated at one or more frequencies in one axis while repeated in a different one or more frequencies in a second axis. In some embodiments, bending is repeated in one or more frequencies while the impact is at another one or more frequencies. In some embodiments, the bending is at one or more frequencies while cutting blades and/or spreading mechanism act at one or more other frequencies.

In some embodiments, one or more of the components of a catheter have power control for regulating a force being applied to it or by it to another component and/or by it to tissue. In an example, the catheter may include one or more force limiters. In some embodiments, a force limiter might be, for example, a spring with a spring constant k, which is large. In some embodiments, the spring lies in series with the force-applying element. In some embodiments, when the force applied is close to the designed limit, the spring starts to respond and compress, taking some of the force instead of the target. In some embodiments, the lead extraction device comprises a steering limiter configured to prevent excessive steering force to be applied, either to the device, to the tissue or to the lead. In some embodiments, the force limiter is located in the handle, for example, using springs that do not allow transmitting steering forces over the pulling wires beyond a certain limit, for example, of 5 Kg. In some embodiments, a potential advantage of the steering limiter is that the applicable torque of the steered tip is not more (is configured to be not more) than 500 grams (meaning this is the maximal force applied on the vein by the steering). In some embodiments, if an external sheath is passed forward over the steered-bending segment then the steering is not forcefully applied against the external sheath (the external sheath overcomes the steering).

In some embodiments, one or more of the components of a catheter may have motion magnitude control for regulating the extent of motion being applied to it or by it to another component and/or by it to tissue. For example, a catheter may include one or more motion limiters.

In some embodiments, the one or more frequency and or one or more power controls for moving one or more of the components is controlled-based, at least in part on input from a sensor. For example, the sensor relates to force applied to the tissue. Optionally or additionally, for example, the sensor relates to the power needed to move a component of the catheter. For example, the sensor relates to magnitude of motion.

In some embodiments, one or more motion limiters and/or force limiters is controlled, at least in part by a sensor.

In some embodiments, a system provides to an operator (for example a physician) one or more indications of the force and/or the motion and/or the location, and/or the bending angle of one or more of the components of the catheter. In some embodiments, the operator receives information based on a sensor. In an example, the handle includes an indicator of the position of wires and their extension. For example, from the indicator, the operator may observe the motion of a certain component. In some embodiments, the system provides information about bending (e.g. angle) of the catheter and/or its tip. In some embodiments, the system provides information about a lateral forces and/or a longitudinal force and/or a pressure on tissue and/or a friction applied on a portion of the catheter by tissue and/or by a vein and/or the system may provide information on a central lead and/or other leads.

### Exemplary Velocities

In some embodiments, the device is configured to work at at least two velocities, one fast and one slow. In some embodiments, the fast velocity is from about 300 RPM to about 500 RPM. For example, 400 RPM, 420 RPM, 500 RPM, or any RPM in between. In some embodiments, the slow velocity is from about 50% to about 75% of the fast velocity. For example 55% of the fast velocity, 65% of the fast velocity, 70% of the fast velocity, or any percentage in between. In some embodiments, a potential advantage of providing a fast velocity and a slow velocity is that it allows the user to choose the velocity in specific cases, for example, if the user feel that he is entering a sensible zone during the extraction, he can choose to work at slow velocities to ensure higher safety.

### 5.2 Exemplary Modifiable Mechanical Properties

In some embodiments, the catheter comprises a mechanism that adjusts mechanical properties of the catheter. For example, the catheter's shaft includes one or more lumens, which are used for the insertion of property adjusting elements. For example, a property-adjusting element may include stiffening rods. In some embodiments, stiffening rods are made of stainless steel, nitinol, polymers having various mechanical properties, or any material that has advantageous mechanical properties to modify the mechanical properties of the catheter shaft. In some embodiments, the stiffening rod is made of nitinol and is configured to make the catheter shaft more pushable without significantly increasing the stiffness. In some embodiments, the stiffening rod is made of stainless steel and is intended to significantly increase the stiffness of the catheter shaft. In some embodiments, the stiffening rods are coated with PTFE or another highly lubricious material to aid in insertion into the lumen. In some embodiments, the one or more lumens are lined with PTFE or another highly lubricious material to aid in insertion of the rods into the lumens. In some embodiments, the stiffening rods are inserted into and/or removed from the catheter shaft without having to remove the device from the patient. In some embodiments, the modification of mechanical properties is controlled manually or automatically. In some embodiments, the user manually modifies the mechanical properties from the handle of the device. In some embodiments, sensors located on the shaft and/or on the distal head receive inputs that modify automatically the mechanical properties. In some embodiments, the system comprises dedicated software and algorithms with examples of functions, lookup tables, activation/deactivation rules, machine-learning models, neural network models, other models, and/or ranges to activate the modification of the mechanical properties based on the input received by the sensors and/or from the user.

### 5.3 Exemplary Combinatorial Use of Components/Embodiments

In some embodiments, one or more components and/or subcomponents and/or embodiments and/or sub-embodiments described therein are used and/or included once or more than once within an embodiment. For example, one or more components are combined with other one or more component and/or a subcomponent and/or embodiment and/or sub-embodiment described in the invention and together their combination forms an embodiment described therein.

### 5.4 Exemplary Characteristics of the Pull-Wires and Lumens of the Device

In some embodiments, the device comprises pull-wires, which run through the catheter shaft to actuate a distal portion of the catheter. In some embodiments, a pull wire runs inside a sleeve, which passes through a lumen in a multilumen catheter shaft. In some embodiments, a lumen is larger than the outer diameter of the sleeves so that the sleeve is free to bend slightly within the lumen. In some embodiments, bending of a sleeve within a lumen allows the sleeve to compensate for bending of the shaft. In some embodiments, the compensation does not change the total length of the lumen within the catheter shaft. In some embodiments, a sleeve is rigidly connected to the catheter shaft at both ends and/or is free to move within the lumen along the length of the shaft, thereby maintaining the same pull-wire length independent of the shaft bending. In some embodiments, a multilumen shaft twists along its length, doing one full revolution every 20-100 cm, optionally there is a twist over fixed and or varying intervals ranging between 20 to 30 cm and/or between 30 to 50 cm and/or between 50 to, 75 cm and/or between 75 to 100 cm. In some embodiments, each braid may increase the flexibility of the shaft when pull-wire and pull-wire sleeves are passed through the lumens. In some embodiments, the pull wires, which go through the sleeve, shrink or stretch when the catheter is bent. In some embodiments, controlling the tension in the pull wires during the shrinking or stretching is done, for example, by twisting the multilumen shaft, which causes a wire in one segment to shrink and to stretch in another, with the total canceling out.

In some embodiments, the pull-wires or pull-cable hold forces between about 20N to about 100N, optionally between about 30N and about 80N; optionally between about 40N and about 60N; optionally the forces are at 40N, 45N, 47N, 50N or 54N.

In some embodiments, the pull-wires comprise a total diameter of about 0.21 mm. Optionally, from about 0.15 mm to about 0.3 mm. Optionally from about 0.19 mm to about 0.25 mm.

In some embodiments, the pull-wires or pulling cable are coated to increase the breaking load of the puling wires and to lower the friction.

In some embodiments, tensile elements (for example wires and/or cables) run through the flexible shaft of the device. For example, tensile elements run from a handle of the device to a distal tip of the device. In some embodiments, wires are optionally connected and/or grouped. In some embodiments, one wire running along the shaft may connect to multiple wires near the tip. In some embodiments, grouping may reduce the number of tensile elements running through the flexible. In some embodiments, grouping may reduce the number of tensile elements connected to a handle. In some embodiments, the tensile elements will run independently and/or separately through the flexible shaft. In some embodiments, some tensile elements are used to adjust properties of the device and/or some tensile elements may be for feedback and/or some tensile elements will be used for control.

In some embodiments, the device includes a mechanism to compensate the tension of the tensile elements due to curves in the flexible shaft or/and in a hinge or/and during vibration. In some embodiments, the compensation is controlled. In some embodiments, the compensation is controlled automatically, using a spring in the tip or/and in the handle.

### 5.5 Exemplary Tension Control and Movement Limiting Mechanism

In some embodiments, incorporating an automated tension control mechanism into a lead extraction sheath may increase ease of use and/or safety of the device. In some cases, applying the appropriate tension to the lead as the extraction sheath is inserted, manipulated, and/or activated, may be complex and/or require more than two hands. In some embodiments, an automated lead tensioning mechanism is supplied. Using an automatic lead tensioning mechanism may facilitate performance of the procedure by a single operator. Alternatively, or additionally, the lead tensioning mechanism provides increased control over the procedure. In some embodiments, an automated lead tensioning mechanism limits the tension to a level at which such complications (for example breakage of the lead and/or tearing of tissue) are less likely to occur.

In some embodiments, when a single operator operates the device, the following exemplary combinations can be applied to use the device:
Holding the shaft and the wire.
Holding the handle and the wire.
Holding the handle and the shaft.

In some embodiments, in each configuration, there is an accessory that is configured to do something, for example, the pedal can be used to activate and deactivate the device when the user is not holding the handle. For example, a wire holder and/or an automatic wire puller, when the user holds the handle and the shaft.

In some embodiments, the system is configured to work efficiently while pushing at forces of higher than 500 gr, higher than 800 gr, higher than 1 Kg, higher than 1.5 Kg, higher than 2 Kg, while maintaining no pulling at all and/or no tension at all and/or minimal tension, for example lower than 200 gr, lower than 300 gr, lower than 500 gr, lower than 600 gr, lower than 800 gr.

In some embodiments, the device handle includes an automated controlled lead tensioning mechanism 150, as shown for example in FIG. 15a (upper—top view, bottom—perspective view). Referring now to FIG. 15b, showing a schematic representation of the lead tensioning mechanism 150. In some embodiments, the lead tensioning mechanism 150 comprises a body 152, gripping teeth 154 and a motor (not shown) adapted to move the gripping teeth. In some embodiments, the lead tensioning mechanism force will pull in between the force of 50 gr to 1500 gr or between the force of 150 gr to 1000 gr or between the force of 200 gr to 600 gr or between the force of 250 gr to 400 gr or between the force of 300 gr to 400 gr. Referring now to FIG. 15c, showing the gripping teeth sliding inwards in the direction of the arrows and gripping the lead 156. Referring now to FIG. 15d, showing the gripping teeth sliding back (proximally), following the arrow, and pulling the lead in the proximal direction. Referring now to FIG. 15c, showing the gripping teeth sliding outwards, following the arrows, and then sliding forwards (distally) to the initial position ready to re-engage the lead. Optionally, the automated controlled lead tensioning mechanism grasps a proximal portion of the lead. Optionally, the mechanism includes a locking stylet that applies a controlled tension to the lead in relation to the LE device. For example, the stylet pulls in the proximal direction. For example, the stylet maintains the lead in constant tension as the sheath is inserted, manipulated, and/or activated.

Another potential source of complications is uncontrolled movement of the lead, for example, when the lead is under tension and abruptly freed. For example, this may result in the tension being suddenly applied to a new location in a vein or heart. Sudden changes in tension may result in a tear in a vein or heart wall. In some cases, this type of complication may result from elasticity of the lead and/or from the uncontrolled movement of a tension producing mechanism (for example the hand of the operator). For example, uncontrolled movement may occur under the sudden release of tension. In some embodiments, a locking stylet is used.

In some embodiments, the locking stylet reduces the elasticity of the system. In some cases, a locking stylet is used while manually holding the lead. In some cases, another possible complication may be the uncontrolled movement of the hand of the user. In some embodiments, the device includes a mechanism that limits the movement and/or the velocity of a lead and/or a stylet. The limitation mechanism is optionally independent of the tension applied. For example, the limiting mechanism prevents sudden movements. For example, sudden movements are prevented when a very high tension is applied and/or when the lead is suddenly released.

In some embodiments, the device includes a mechanism for limiting the movement and/or the velocity of the lead. Optionally, the limiting mechanism inhibits sudden movements of a stylet and/or lead. In some embodiments, the limiting mechanism includes a clamp that attaches to a stylet and/or lead. For example, the clamp may have a limited motion range and/or limited velocity.

### 6. Exemplary Characteristics of Force Measurements in the Device

In some embodiments, the LE device comprises elements which allow the sensing of force and/or pressure applied by the device tip and/or segments along the catheter length. In some embodiments, the basic approach common to all methods is the translation of the force or pressure applied by the device, into a mechanical displacement and/or material deformation, which is translated to a sensible signal that is captured and processed to provide force or pressure indication. In some embodiments, the methods provide indication on force applied in 1, 2 or 3 dimensions. In some embodiments, the dimensions may be independent or relative to the axes of the catheter position.

### 6.1 Exemplary Force Transducer in the Distal Portion of the Device

In some embodiments, the device comprises a force sensor. For example, the sensor may include an axial force sensor, which measures the force that the device is exerting in the distal direction upon tissue, for example at the distal tip of the device. In some cases, difficulties in performing lead extraction are due to variable and/or unknown flexibility and/or friction along the path that the lead extraction device takes from the user's hands to the distal tip. These factors may make it difficult to judge the amount of force that the distal tip is applying on the tissue based on the force being applied by the user. In some embodiments, a force sensor near the distal tip of the device provides information about the amount of force being applied by the distal portion of the device on the tissue. In some embodiments, this information aids in the safe and effective performance of the lead extraction procedure.

In some embodiments, the force sensor is comprised of a mechanically weakened region of the wall of the shaft of the device and/or a sensor, which senses the force-dependent distortion of the wall of the shaft of the device near the weakened region. In some embodiments, the weakened region includes cuts in the wall of the shaft of the device. In some embodiments, the sensor includes a strain gauge.

### 6.2 Exemplary Model and Shape Based Force Estimation

In some embodiments, the estimation of forces applied by the LE device is performed without the need for integration of sensors on the device itself or any other device add-on (such as an additional outer sheath). In some embodiments, the estimation of forces is performed by the activation of an external imaging or tracking system to track the shape and position of catheter inside the body. In some embodiments, for example, such a system, which is also in common use in LE procedures, is the X-RAY system, used for tracking and navigation of the device inside the body. In some embodiments, access to the raw data of the X-RAY machine provides the necessary and sufficient data for this method. In some embodiments, radio opaque markers may be incorporated along the length of the LE device for easier and more accurate extraction of data on the catheter's shape. In some embodiments, a force displacement model is developed per each LE device type intended to be used with this method. In some embodiments, the force estimation is performed by force-displacement modeling of special mechanical structures within the device such as: an articulated structure that is intended for catheter steering (see FIG. 15f—Articulated structure 158 of an LE catheter) or a multi-luminal structure designed for instance to support rotational force transfer from the proximal end of the catheter to distal end (see FIG. 15g—Multi-luminal structure 159a-c (lumens) of a LE catheter). In some embodiments, the model allows for a shape-based force estimation equation to be solved, with real-time coordinates of the catheter as inputs, for the force applied by the tip or by every point along the length of the catheter.

### 6.3 Exemplary Opto-Mechanical Methods

In some embodiments, opto-mechanical methods for force and/or pressure estimation are used and may have the advantage of being free of electrical currents inside the patient's cardiovascular system and hence are possibly safer than electro-mechanical methods. In some embodiments, in addition, sensing is not influenced by electromagnetic fields or RF power that may exist in the environment.

In some embodiments, laser LE devices are based on catheter advancement through binding sites, by laser ablation, where light is emitted on the target by an array of optical fibers. In some embodiments, for such devices, opto-mechanical methods for force sensing at the distal end of the device are based on taking up some fibers from the array and using them for force sensing, for example, in one of the methods described below.

### 6.3.1 Exemplary Optical Methods Based on Reflective Intensity of Light

In some embodiments, light is transmitted at a reflector and the reflected light intensity is modulated by the applied force, using a mechanical force-to-displacement translation unit, such as a flexure, a diaphragm or similar.

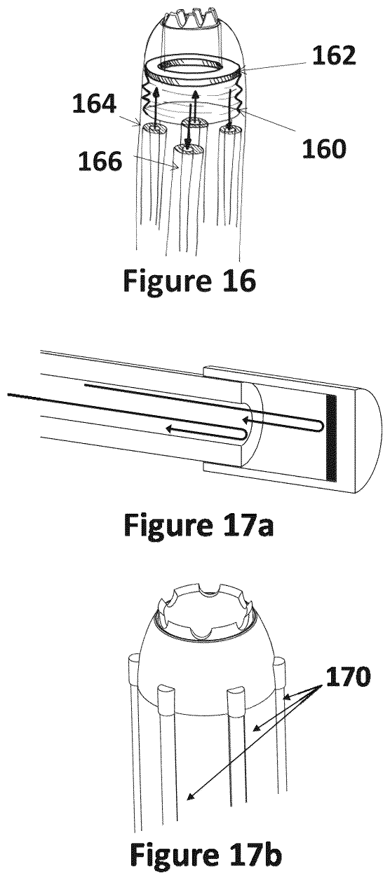

In some embodiments, a flexure 160 is used to convert force to displacement, to be sensed by light reflectance. In some embodiments, the flexure is an integral part of the catheter head, as shown in FIG. 16. In some embodiments, the flexure 160 is structured within the articulated structure designed for a steering capacity, in a steerable catheter. In some embodiments, the flexure 160 is part of an add-on sheath, separated from the LE device. In some embodiments, the flexure 160 holds a reflecting structure—the reflector 162, designed to enable force sensing, with no interference to the penetrating mechanism of the catheter head. In some embodiments, the reflector is shaped as a ring 162, positioned above the tips of the light transmission medium, as shown for example, in FIG. 16. In some embodiments, light is transmitted along the length of the catheter by a single or a multiple of optical fibers 164, and is emitted towards the reflector 162. In some embodiments, a single or multiple of optical fibers 166 are used to receive the reflected light, and carry it to a control unit outside the body. In some embodiments, the control unit includes the light source of the transmitting fibers, and a reception sub-unit in charge of translation of light intensity into force indication.

In some embodiments, light interference patterns are sensed by a Fabry-Perot interferometer, based on the principle of interferometry. In some embodiments, a single fiber is used for transmission of emitted and reflected light. In some embodiments, a cavity is located on the tip of a single-mode optical fiber and enclosed by a miniature glass diaphragm. In some embodiments, light is reflected both from the end face of the fiber and from the diaphragm. In some embodiments, the two reflected signals interfere with each other and have a phase difference, as shown for example in FIG. 17a (Illustration of the Fabry-Perot effect), since light from the diaphragm has traveled an extra distance through the cavity. In some embodiments, the phase difference depends on the diaphragm distance from the fiber tip. In some embodiments, the intensity of the light penetrating back into the fiber is a function of the phase difference between the interfering signals and hence related to the diaphragm displacement resulting from pressure applied on it. In some embodiments, a single fiber with a cavity and diaphragm is used. In some embodiments, multiple of fibers 170 are used to sense forces in multiple directions and orientations, as shown for example in FIG. 17b (Exemplary optical force sensor based on Fabry Perot interferometer, embedded on LE device). In some embodiments, a control unit is in charge of providing the light source for emission and for receiving the Fabry-Perot interference signal and deducting force indications based on the intensity.

### 6.3.2 Exemplary Fiber Bragg Grating Methods Based on Wavelength Shift

In some embodiments, Fiber Bragg Grating (FBG) structure is constructed by creating a periodic variation in the refractive index of the fiber core. In some embodiments, when created in a short segment of an optical fiber, FBG reflects particular wavelength and transmits all the others and therefore can be used as an inline optical filter. In some embodiments, the wavelength at which high reflectivity occurs is determined by the periodicity of the gratings. In some embodiments, when the FBG segment in the fiber is stretched or compressed, the dimensions of the grated area are shifted, resulting in a shift in the reflected wavelength. In some embodiments, this property is used for sensing the pressure applied on the fiber.

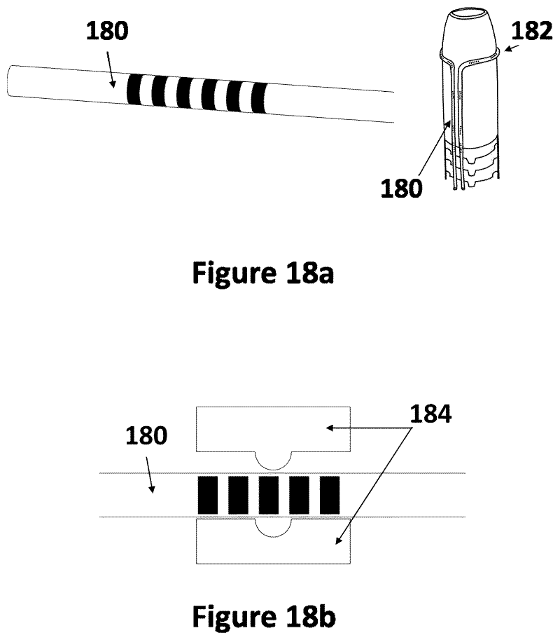

In some embodiments, FBG sensors are integrated on the device. In some embodiments, an optical fiber embedded with FBG segments 180 is spread along the length of the catheter and wrapped in a ring shape 182 around the device head. In some embodiments, each FBG segmented is formed to reflect a different wavelength, this way frequency analysis of the reflected and transmitted signals at a control unit can estimate the forces sensed at different locations along the length and around the head of the device, as shown for example in FIG. 18a (FBG segmented fiber integrated in an LE device.). In some embodiments, for the FBG segments along the length of the catheter, bending and deformation as a result of contact and friction with the blood vessel wall, are translated directly to a wavelength shift that can be sensed. In some embodiments, for the ring shaped section, wrapping the head of the catheter, a mechanical unit for the translation of longitudinal forces to FBG segment strain or bending, might be needed. In some embodiments, for example, a mechanical structure composed of a top circular plane with protuberances 184 positioned above the FBG segments in the fiber ring and bottom circular plane with sockets located straight under the FBG segments and the protuberances, as shown for example in FIG. 18b (Force to FBG bending mechanical translation unit.). In some embodiments, such structure allows for forces on the head of the catheter to translate into FBG segment bending and therefore to a reflected wavelength shift.

In some embodiments, the measurement of the shift in the reflected wavelength is performed, for example, by laser interferometry. In some embodiments, the sensor mounted in the LE device produces a signal with a wavelength varying depending on the stress acting on the device. In some embodiments, this signal is compared to a nearly-identical sensor that is left outside and/or inside the catheter in a part that do not deforms, in an external analysis unit. In some embodiments, when the LE device is not under any strain, both sensors provide a nearly identical wavelength. In some embodiments, this is calibrated as the reading “zero”.

In some embodiments, when the LE device sensor experiences any stress, its wavelength shifts and this is measured by the interferometer.

### 6.4 Exemplary Electro-Mechanical Methods

### 6.4.1 Exemplary PVDF Force Sensing

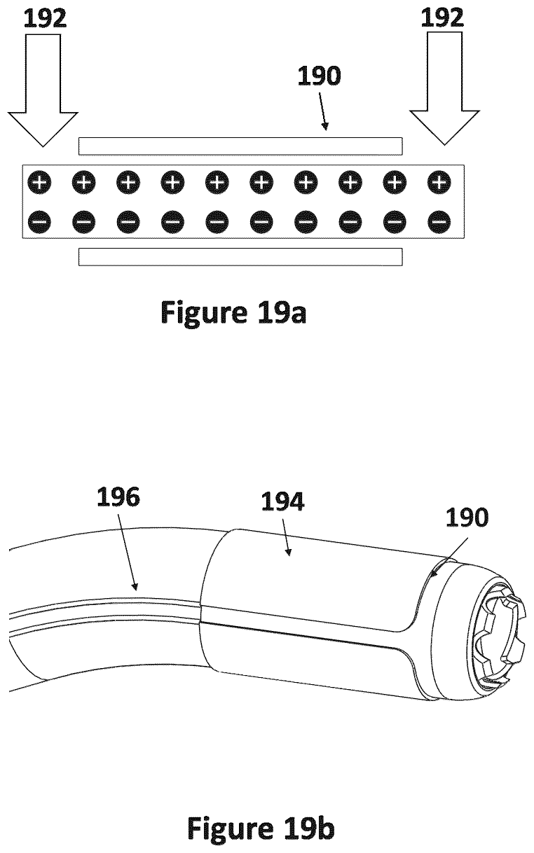

Polyvinylidene Difluoride (PVDF) is a chemically stable piezoelectric polymer with high piezoelectric properties. PVDF films have been used as force sensors in various applications. When a load is applied on the top of a PVDF film 190, the polymer accumulates electric charge on both sides of the material, which has equal number and opposite polarity. This charge is proportional to the applied force 192 and can be sensed electrically, as shown for example in FIG. 19a (Piezoelectric principle in PVDF).

In some embodiments, PVDF film segments are used to wrap parts of the catheter where force sensing is desirable, for instance: the LE device head, for the purpose of spatially continuous sensing of force in all directions. In some embodiments, the PVDF sensor is composed of PVDF film 190, an outer coating of insulated and damp proof rubber film 194 and 2 conductive wires 196 attached to electrodes on both sides of the PVDF film. In some embodiments, the wires are spread along the catheter length and serve as inputs to a control unit, where measured voltage is analyzed and translated to force indication, as shown for example in FIG. 19b.

### 6.4.2 Exemplary Capacitive-Inductive Force Sensing

An electric circuit made up of a capacitor and an inductor is called an LC circuit, and is characterized by a resonance phenomenon at a frequency Fr=1/[2pi*sqrt(L*C)], with L being the inductance in Henry, and C being the capacitance in Farad.

In some embodiments, such a design is used as a pressure sensor, when the capacitor is made up from two electrode plates with a soft dielectric material between them. In some embodiments, when an electrode plate is pressed, the capacitance increases and with it the resonance frequency of the circuit.

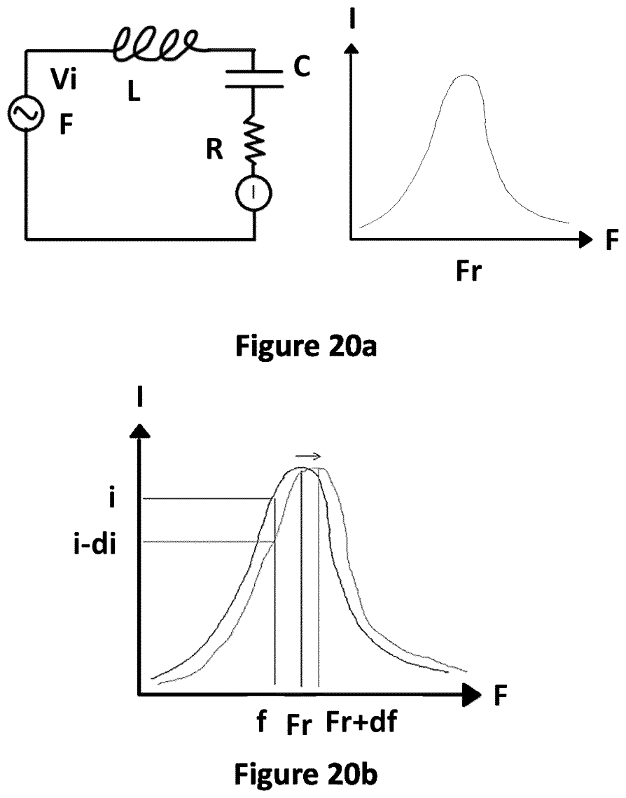

In some embodiments, if the circuit is driven at a frequency f close to the resonance frequency, and the transmitted amplitude is measured, then it is highly sensitive to a change in resonance frequency, as shown for example in FIG. 20a.

$$i = Vi / sqrt [ R \hat{} 2 + ( wL - 1 / wC ) \hat{} 2 ] \\ w = 2 * pi * f$$

In some embodiments, when the capacitance changes, the resonance frequency shifts. As a result, the attenuation of the signal at frequency f changes. In the example in FIG. 20b, the electric current decreases (if the capacitance increases and f<Fr).

In some embodiments, the circuit can be very sensitive to a change in current, if the quality factor is high (the parasitic resistance low).

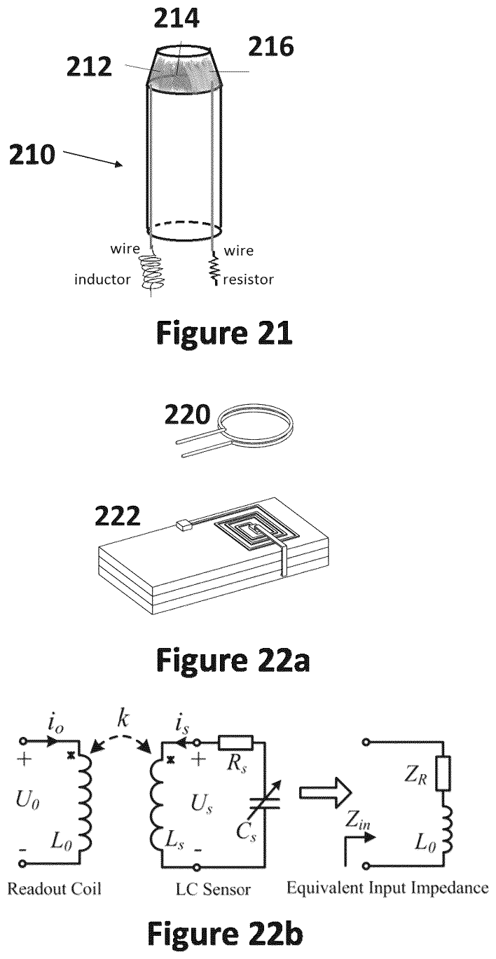

In some embodiments, an LC based force sensor is integrated in the device. In some embodiments, current is measured by a 4-wire current probe, using a series resistance low enough not to disrupt the quality factor of the circuit too much. In some embodiments, for use on a catheter, the sensor 210 is made up of an inner electrode 212, thin and soft dielectric foam 214, and an outer electrode 216, as shown for example in FIG. 21.

In some embodiments, the electrodes are connected by wires to the catheter handle, were the inductor and 4-wire current probe resistor are located. In some embodiments, the electrodes are located along the sides of the tip, along the length of the shaft, as patches on a hinge (see FIG. 19b) or any other location on the exterior of the catheter. In some embodiments, a sensor may also be located in the interior of the device, as a reference sensor against which the external sensors' signal can be compared (in an electronic differential manner).

In some embodiments, force is sensed by the capacitive-inductive resonance method described above and shift in resonance is detected and measured wirelessly, through resonant inductive coupling. In some embodiments, the sensor circuit is made up of a small capacitor as described above, with the inductor arrayed next to it or around it. In some embodiments, there are no wires connected to it.

In some embodiments, the sensor is probed by an external read-out coil 220, inductively coupled to the sensor inductor, as shown for example in FIGS. 22a-b. In some embodiments, the effective impedance of the readout coil is affected by the resonance frequency of the sensor circuit.

In some embodiments, the readout coil 220 is positioned externally to the patient, and the LC pressure sensor 222 is integrated as part of the LE device. In some embodiments, the capacitor dielectric material is soft and compressible, thus when it is pressed the capacitance increases. In some embodiments, the coil is insulated from the adjacent capacitor plate.

### 6.5 Force Analysis Unit—Exemplary Feature

In some embodiments, in each one the force sensing methods, an external control and analysis unit that processes the received optical or electrical signal and produces a force indication for the user is included.

In some embodiments, the analysis unit comprises an algorithmic signal processing capacity to filter out measurement noises and artifacts produced by any mechanical feature of the device such as: a motorized mechanism, cutting head rotation, inner lumen periodic friction etc. In some embodiments, such signal processing algorithmic capacity considers spectral and temporal properties of the device activation mechanism and reduces their impact on the quality of the force measurement, by algorithmic methods such as time domain windowing or frequency domain filtering.

In some embodiments, the force sensing features are used additionally for educational purposes, for example during training, to teach the user how to properly use the device.

### 6.6 Lead Centering Detection Unit—Exemplary Feature

In some situations, the physician requires to understand the 3D orientation of the lead, of the vein and of the device, in order to determine how to rotate and steer the lead extraction device in the most effective manner and in a manner that is safe to the veins, such that the forces or energy is not aggressively applied to the vein wall. In some embodiments, the device comprises a sensor, which monitors the tissue type or content of matter in different orientations of the tip (e.g. right-left, up/down in the steering orientations). In some embodiments, the device comprises a sensor that displays the lead orientation relative to the center of the lumen of the device, e.g. whether the lead is in the center, or trending towards the right or left side of the catheter, or towards the up (outer curve) or down (inner curve) of the steerable segment. In some embodiments, the display is an interface that provides clear and simple feedback to the user, for example, about power level, steering direction, rotation direction and impact mode. In some embodiments, the information provided by the lead centering detection unit, may be used by the physician to decide to turn the catheter in the direction of the lead, and preferably away from the venous wall. In some embodiments, the device is connected to an external 3D navigation system.

### 7. Handle of the Device and Motion

### 7.1 Exemplary Linear/Impact Element/Ring Motion of a LE Device

In some embodiments, the LE device comprises an impact generator to provide pulsating strokes at the distal end of the LE device. In some embodiments, the mechanical part provides an additional rotation movement for the cutting, spreading and hammering tip. In some embodiments, the mechanical part enables a controlled, linear movement of blades in a forward-backward (distally-proximally) manner. In some embodiments, the movement of the blades provides a precise and controlled cutting of the tissue in front of the distal end of the LE device. In some embodiments, the linear motion mechanism is a motorized mechanism. In some embodiments, the motorized mechanism is activated by a controller on the handle of the LE device. In some embodiments, the motorized mechanism is activated by a dedicated pedal (or similar mechanism) located in close proximity to the user. In some embodiments, motion mechanisms are divided between the handle of the device and an adjacent unit. In some embodiments, all the motion mechanisms are located outside the handle and they are delivered into the handle from an external connector. In some embodiments, for linear or for radial LE device the speed of the motor can be in the range from about 1 Hz to about 100 Hz for example, and/or from about 1 Hz to about 100 Hz, and/or from about 20 Hz to about 70 Hz, and/or from about 15 Hz to about 80 Hz, and/or from about 10 Hz to about 80 Hz, and/or from about 3 Hz to about 90 Hz, and/or from about 35 Hz to about 60 Hz, and/or from about 1 Hz to about 15 Hz, and/or from about 2 Hz to about 20 Hz.

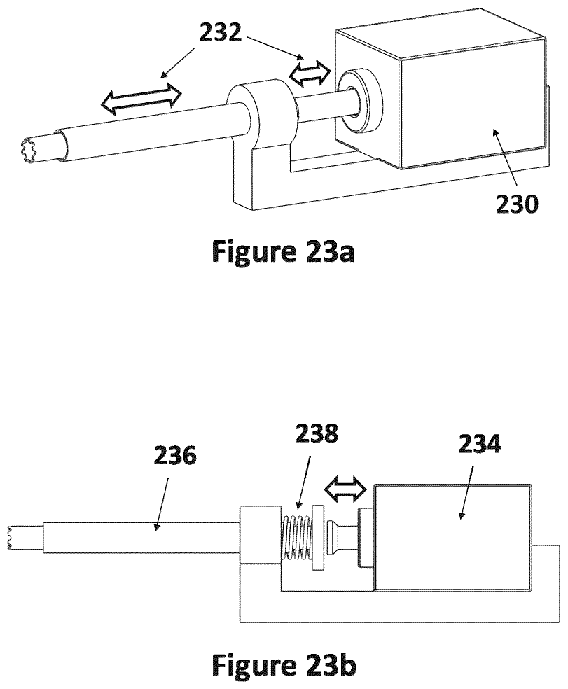

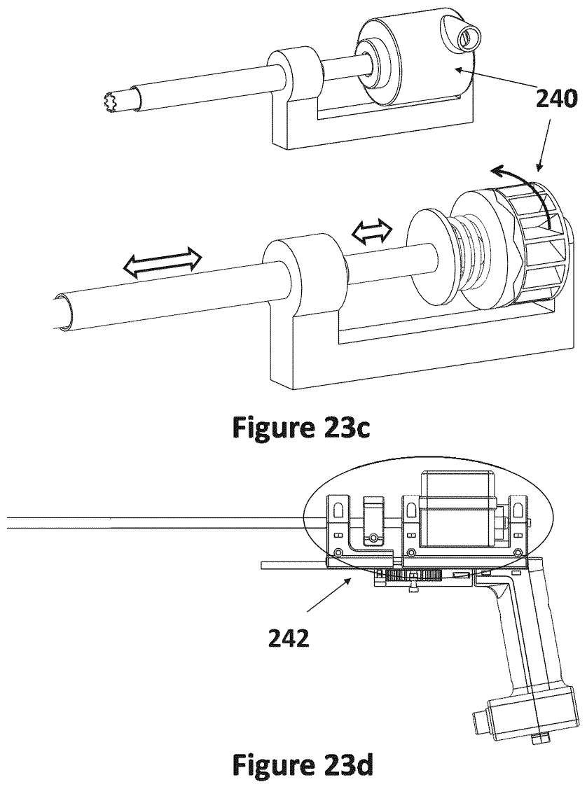

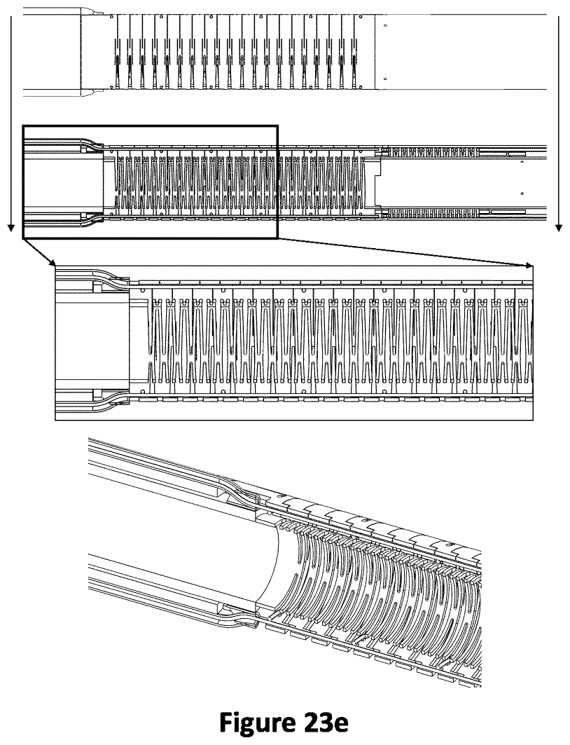

In some embodiments, a pedal or a button in the handle activates the linear motion mechanism in an on/off manner or PWM controlled (speed controlled). In some embodiments, a pedal or a button in the handle activates the linear motion mechanism in an incremental motion controlled manner. In some embodiments, a pedal or a button in the handle activates the linear motion mechanism using pneumatic mechanism. In some embodiments, the movement of the blades created by the linear motion mechanism is performed mainly inside the LE device (i.e.: internally—without an external manifestation of the movement) and at the end of the forward cycle the blades protrude from the distal end of the LE device. In some embodiments, the linear motion motor mechanism is located at the LE device's handle. In some embodiments, the linear motion motor mechanism is located outside the LE device. In some embodiments, the linear movement of the cutting mechanism is performed inside the LE device/catheter, therefore protected from the outside environment of the LE device. In some embodiments, the linear movement comprises a catheter structure as shown for example in FIG. 23e, and/or is done with a multilumen sheath or Fort Wayne HHS® or similar material that provides the bending radius/torque/pushability parameters or a combination of materials.

Several possibilities of linear motion mechanisms are shown, for example, in FIGS. 23a-c. As shown in FIG. 23a, the linear motion is created by a motor 230 inside the box and transduced into linear motion 232. FIG. 23b shows an example of a motor 234, which pushes the central shaft core 236, which is adapted to return due to the spring 238. FIG. 23c shows an example of a device in which an external source of energy (not shown) rotates a gear engine 240. The gear engine transforms rotational motion into linear motion.

In some embodiments, the motion mechanisms 242 (circled) are incorporated into the handle as shown, for example in FIG. 23d.

In some embodiments, when external pressure, for example of about 400 gr, or 600 gr, or 800 gr, or 1200 gr, is applied on the hitting blades, the hitting blades slightly retract thereby providing a less aggressive stroke, due to the fact that it has less trajectory for acceleration.

### 7.2 Exemplary Dual Motion Cutting Mechanism—Rotating Hammer

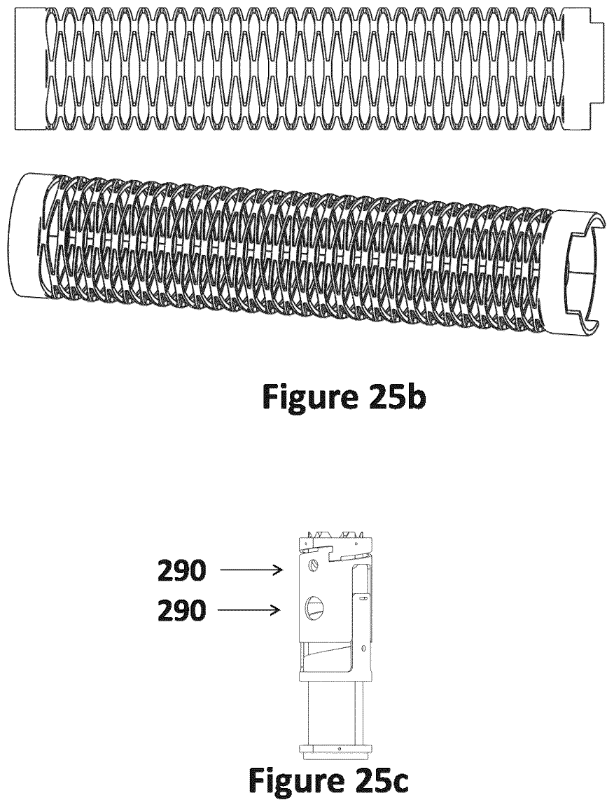

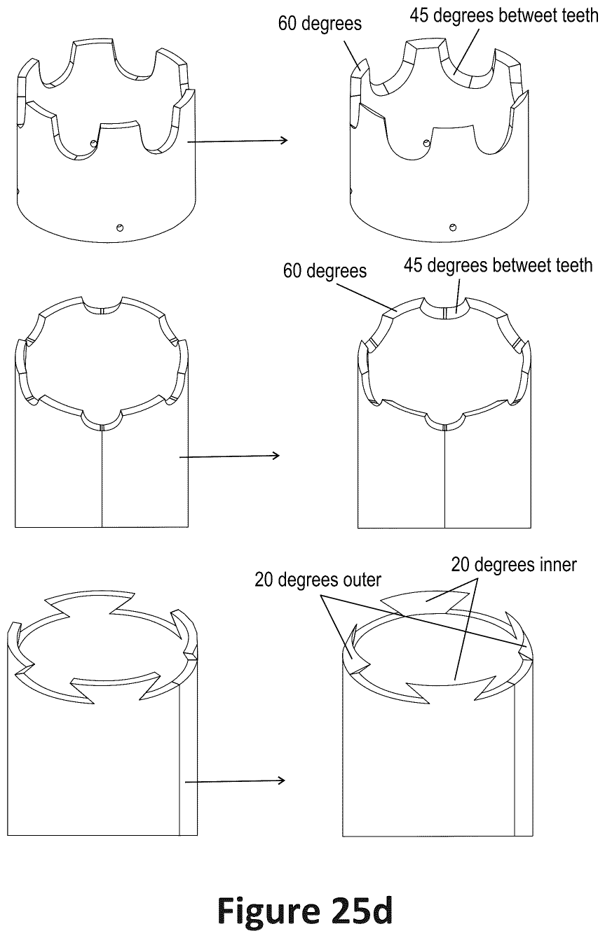

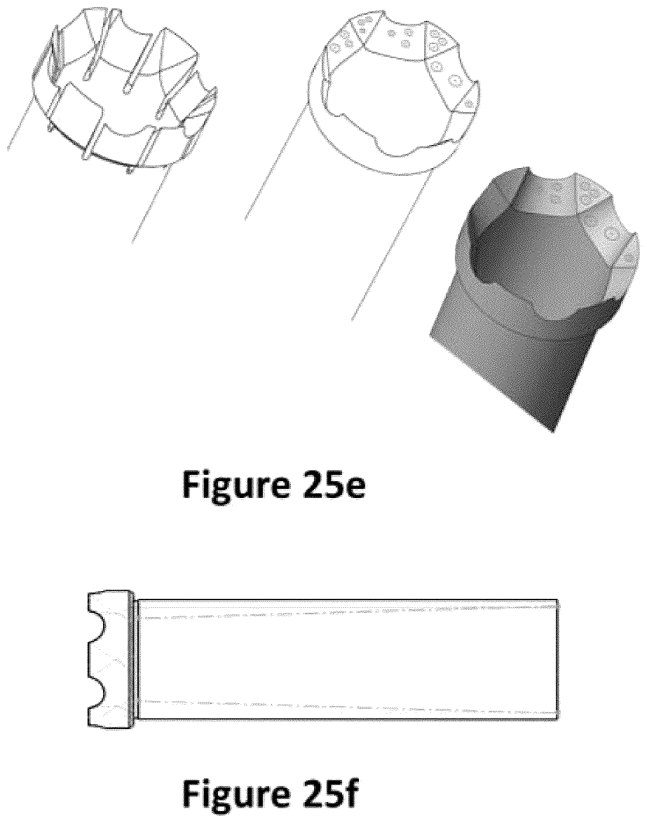

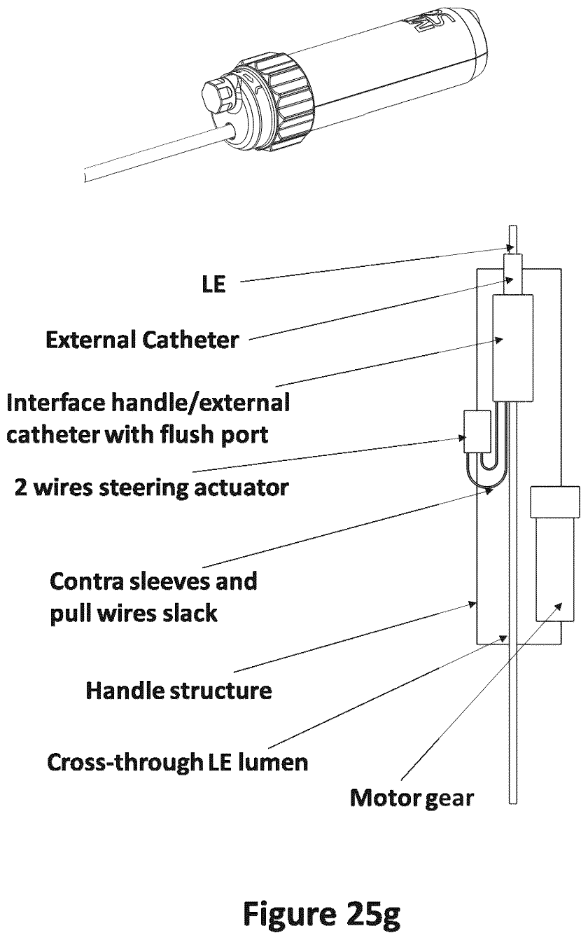

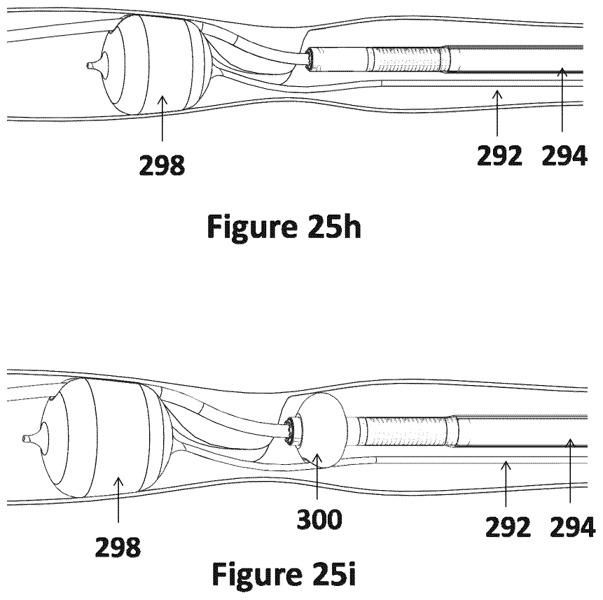

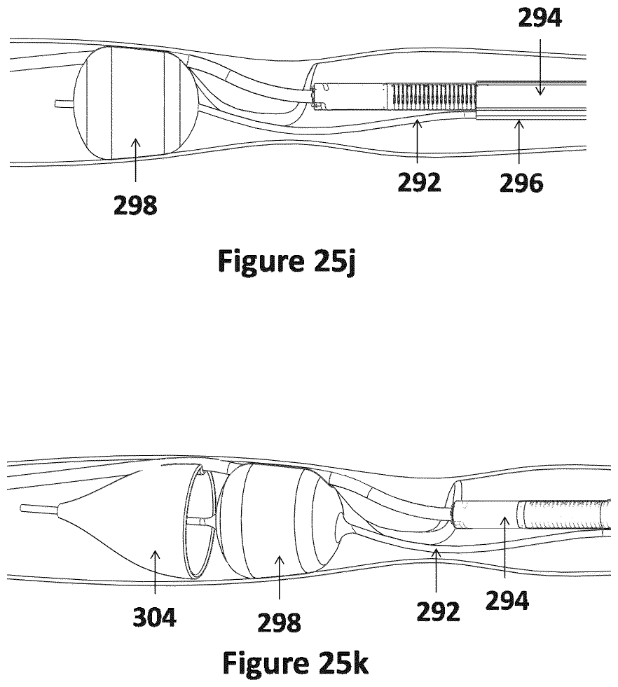

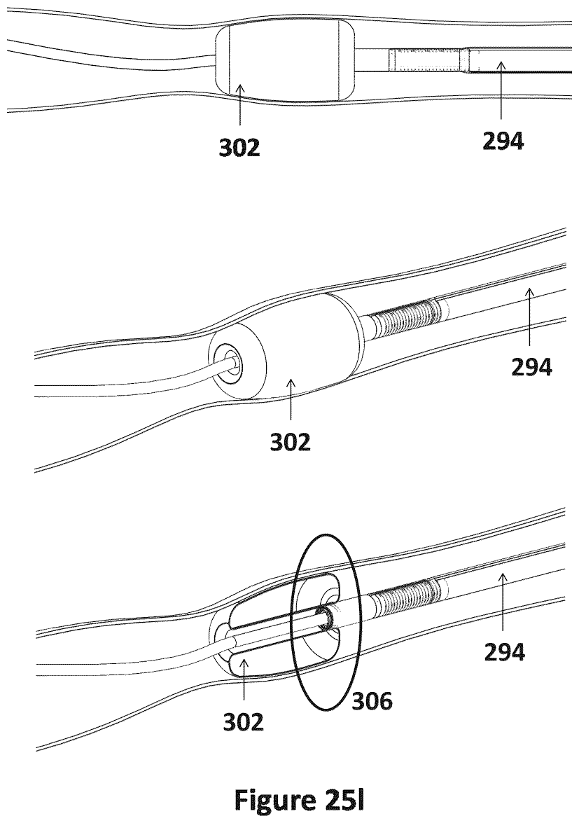

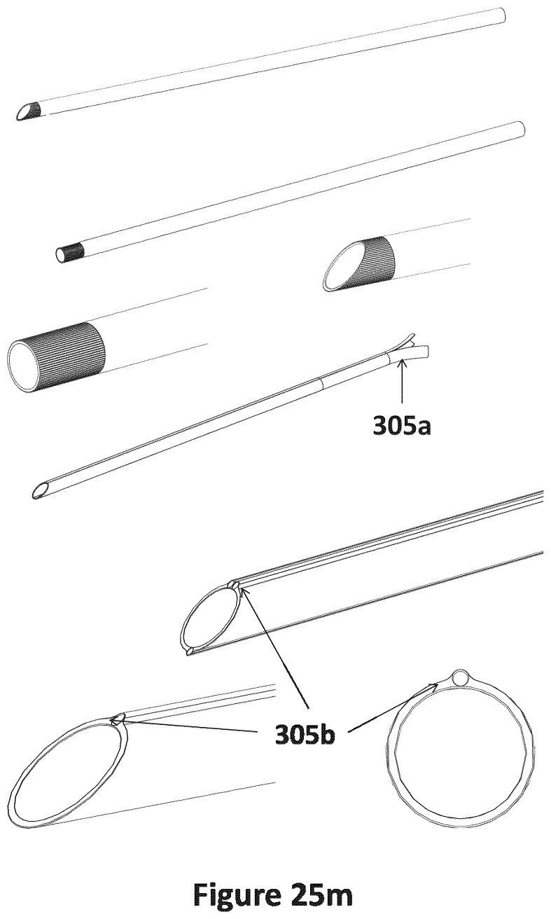

In some embodiments, a rotating motion mechanism is used in addition to the linear motion mechanism, as shown for example in FIGS. 24a-25c. FIG. 24a shows a close-up of the handle 24 of the device and an example of a rotating motion mechanism 244 inside it. FIGS. 24b-d show perspective views of the example of the rotating motion mechanism showed in FIG. 24a. In some embodiments, the cutting of the tissue is performed by the linear movement of the first blades together with the rotating movement of the second blades, as will be further explained below. In some embodiments, the rotating mechanism rotates from right to left and/or from left to right. In some embodiments, blades located at the distal end of the device are used as cutting tool. In some embodiments, the cutting action is linear, which means cutting the tissue when moving blades back and forth (proximally and distally). In some embodiments, the cutting action is rotational, which means that the blades rotate CW and/or CCW with one set of blades or by rotating one set of blades against another to shear tissue between them (similar to scissors).

In some embodiments, the blades 246 are inserted or a cover 248 moves forward to protect from cutting the vein, as shown for example in FIG. 24e.

In some embodiments, the cutting or separating blades 250 are configured for example as shown in FIG. 24f, or other structure of blades or a combination thereof. In some embodiments, the rotating cutting mechanism and/or linear mechanism are as shown, for example in FIGS. 24g-h.

In some embodiments, the rotating cutting mechanism with the linear mechanism comprises, for example, the following parts: upper cradle and cradle 252 impact element/ring, impact element/ring bridge and CAM 254, rotating inner shaft and shaft cam 256, spring 258 and outer tube 260. In some embodiments, the CAM is the element that takes the impact element/ring into the free run area and the CAM is the part that will start the free run and stops the hit of the impact element/ring according to the CAM structure. As can be seen in FIG. 24g, 262a shows the configuration where the rotating blades and the impact element/ring are inside, therefore inactive and guarded from cutting anything; 262b shows the same configuration but in perspective—showing the blades inside. 264a shows the rotating blades outside while in action. In some embodiments, the rotational action of the blades causes the CAM to move and to push backwards the impact element/ring. It can be seen that the spring is being pulled backwards as well, therefore loading the impact element/ring. 264b shows the same configuration in perspective. 266a shows the impact element/ring outside, after it was fired and the rotating blades outside as well. 266b shows the same configuration in perspective. In some embodiments, the mechanism repeats itself with the rotation of the rotating blades. In some embodiments, the slope of the CAM might be linear, or quadratic or exponential to compensate for forces. In some embodiments, the slope translates directly to the force the shaft CAM applies on the CAM. In some embodiments, the more slope there is, there is less force but larger amplitude. In some embodiments, because it is driven by a spring, the force applied depends on the spring compression, and changes during the motion as the spring is released. In some embodiments, the compensation is performed by changing the angle of the slope, for example quadratically, to match the degree of release of the spring, to achieve constant force along the motion. In some embodiments, the CAM can be a structure of two slopes in order to have better aligned and centered linear movement of the impact element/ring, when excoriating and moving forward to achieve the hit. In some embodiments, when the blade is rotating back, in case of retraction of the blades, the two cams rotate until they meet.

In some embodiments, the mechanism of friction at the lower CAM helps the device to move in correctly synced steps. In some embodiments, when the blades move forward to perform the hit, they first have to be exposed from the protective cover. In some embodiments, once the blades are exposed and have a clear path to the target, the CAM starts its rotation, loading the spring for the hit. In some embodiments, this order is ensured by the friction mechanism, which is a little ‘step’ in the two parts of the CAM right before the slope begins. In some embodiments, these matching steps make the two parts move together, pushing each other, until the blades are exposed. In some embodiments, only then they dislodge from one-another and start to slide, with the slope increasing the distance between them and loading the spring. This can be seen, for example, in FIGS. 24g and 24h.

Exemplary Calculations when Choosing a Spring

In some embodiments, the following calculations are used when choosing a spring:

| Spring A | Spring B |
|---|---|
| ID = 5.1 mm | ID = 4.98 mm |
| OD = 6.1 mm | OD = 6.1 mm |
| $t = \frac{( OD - ID )}{2} = 0.5 mm$ | ${t}^{′} = \frac{( OD - ID )}{2} = 0.56 mm$ |
| L₀ = 10 mm (unloaded) | L₀′ = 7.87 mm (unloaded) |
| L₁ = 8.6 mm (preloaded) | L₁′ = 5.9 mm (preloaded) |
| L₂ = 6.1 mm (loaded) | L₂′ = 3.9 mm (loaded) |
| \|L₂ − L₁\| = 2.5 mm (loading travel) | \|L₂′ − L₁′\| = 2 mm (loading travel) |

Calculating the spring constant (Hooke's Law) and the spring travel of each spring, we can calculate the impact speed and the impact force of each spring, which are as follows:

| Spring A | Spring B |
|---|---|
| $Vf = \sqrt{Ks * \frac{\vert L {2}^{2} - L {1}^{2} \vert}{M}} = 9.489 \frac{m}{s}$ | ${Vf}^{′} = \sqrt{Ks * \frac{\vert L {2}^{′2} - L {1}^{′2} \vert}{M}} = 8.358 \frac{m}{s}$ |
| Impact Speed | Impact Speed |
| $\begin{matrix}Im = M * Vf = 3.606 \frac{gm * m}{s} \\ Impact Force\end{matrix}$ | $\begin{matrix}{Im}^{′} = {M}^{′} * {Vf}^{′} = 5.182 \frac{gm * m}{s} \\ Impact Force\end{matrix}$ |

When comparing the two results we get:

$$( \frac{{Vf}^{′} - Vf}{Vf} ) * 100 = - 11.912 \% Decrease in Impact Speed \\ ( \frac{{Im}^{′} - Im}{Im} ) * 100 = 43.722 \% Increase in Impact Force$$

In some embodiments, according to the requirements of the spring, using the above calculation, the user can choose which spring to use.

In some embodiments, the cutting action is a combination of linear and rotational. In some embodiments, the linear mechanism and the rotating mechanism are synchronized. In some embodiments, the rotating motion mechanism further comprises a “hammer-drill like” mechanism. In some embodiments, the “hammer-drill like” mechanism enables to “hammer” (controlled strong forward strokes) while rotating the rotating blades. In some embodiments, the movement of the rotating blades together with the linear blades provides a scissor cutting effect. In some embodiments, both cutting mechanisms can be retracted inside the LE device/catheter. In some embodiments, the linear mechanism and the rotating mechanism are activated independently of each other.

In some embodiments, the device applies impact force to the target tissue. In some embodiments, the impact is generated by hitting the target. In some embodiments, in order to hit the target effectively the (one or more) impact element(s) have a (relatively) “free run” path. In some embodiments, the path in which it accelerates comprises a region with friction, which is lower than the friction force and deceleration that is caused by the impact with the target tissue. An example of this can be seen, for example, in FIGS. 24h-r—showing the different layers of the mechanism: upper cradle and cradle 268, impact element/ring, impact element/ring bridge and CAM 270, spring 272, rotating inner shaft 274, and outer tube 276. In some embodiments, the CAM is the element that takes the impact element/ring into the free run area and the CAM is the part that will start the free run and stops the hit of the impact element/ring according to the CAM structure. 278 shows the impact element/ring mechanism in the inner safe position. 280a and 280b (perspective) show the impact element/ring mechanism fully loaded. 282a and 282b (perspective) show the impact element/ring mechanism after being activated. In some embodiments, the impact element runs within the encapsulated range (the “free run”/acceleration path) a distance, for example, of 5 mm, 3 mm, 2.5 mm, 2 mm, 1.5 mm, 1.2 mm, 1 mm or 0.7 mm; optionally from about 0.2 mm to about 7 mm; optionally from about 0.7 mm to about 5 mm; optionally from about 1 mm to about 3 mm.

Referring now to FIG. 24i, showing the different layers of the mechanism of another embodiment, similar to those described in FIG. 24h. While in FIG. 24i the impact element/ring cannot be seen, the changing position of the cam can be seen. Furthermore, in this embodiment, the position of the rotating blade is fixed. In 280a2 and 280b2 the impact element/ring is out, in 282a2 and 282b2 the impact element/ring is in.

### Exemplary Ratchet Mechanism

Referring now to FIG. 24j, showing an exemplary rotating cutting/linear mechanism, including a ratchet mechanism, together with an exploded view of the different parts, according to some embodiments of the invention. In FIG. 24j, the following parts are shown: inner shaft 283a2 comprising the rotating blades 283a4 and the teeth of the upper part of the ratchet mechanism 283a6, the hitting blades 283a8 with the lower cradle 283a10 (also named lower cradle/hitting blade complex), spring 283a12, the upper cradle 283a14 comprising the teeth of the lower part of the ratchet mechanism 283a16, and the outer tube 283a18. All the parts are shown together in 283a20. In some embodiments, when the inner shaft 283a2 is rotated, for example, CW, the teeth of upper part of the ratchet mechanism 283a6 lock with the teeth of the lower part of the ratchet mechanism 283a16 thereby causing the rotation of the upper cradle 283a14. In some embodiments, the rotation of the upper cradle 283a14 causes the interaction with the lower cradle 283a10 causing the lowering of the lower cradle/hitting blade complex, which due to the spring 283a12, will accelerate back forward at the end of the rotation of the cradles, thereby linear movement of the hitting blades 283a8. Therefore, in some embodiments, providing at the same time, the rotational movement of rotating blades 283a4 and the linear movement of the hitting blades 283a8. In some embodiments, when the inner shaft 283a2 is rotated, for example, CCW, the teeth of upper part of the ratchet mechanism 283a6 slide on the teeth of the lower part of the ratchet mechanism 283a16 thereby providing a very short very fast linear movement to the hitting blades 283a8. The movement is also ensured due to the spring 283a12, which constantly pushes forward the lower cradle/hitting blade complex.

In some embodiments, the cradle comprises two helixes 285b2-285b4, which provides two hits per rotation, as shown for example in FIG. 24k.

In some embodiments, the ratchet mechanism comprises between 4 to 8 teeth, optionally 12 teeth, optionally 24 teeth. In some embodiments, the amplitude of each tooth is from about 0.3 mm to about 1.5 mm, for example, 0.3 mm, 0.5 mm, 0.7 mm, 1.0 mm. In some embodiments, the angles of the teeth, at the top and at the base, are rounded. In some embodiments, a potential advantage of rounded angles is that it avoids unwanted shock during the locking of the teeth.

An example of the performance of the ratchet mechanism is as follows: at an exemplary rotation of the inner shaft in a CW direction of about 420 RPM causes about 7 rounds per second. In an embodiment having double helix cradle, it will cause 14 hits per second. In the case the inner shaft will rotate in a CCW direction, in a 6-teeth ratchet mechanism, it will cause 6(teeth)×7(rounds)=42 short hits of the hitting blades. In some embodiments, when external pressure, for example of about 400 gr, or 600 gr, or 800 gr, or 1200 gr, is applied on the hitting blades, the hitting blades slightly retract thereby providing a less aggressive stroke, due to the fact that it has less trajectory for acceleration.

In some embodiments, optionally, when high pressures are applied on the impact element, the ratchet mechanism disconnects thereby leaving only the movement of the rotating blades.

In some embodiments, pressures are applied on the impact ring, due to one or more of the following: the pressure of the tissue on the impact ring when pushing the device and/or over the lead when trying to achieve a contact between the tip of the device (or the impact ring, or the cutting blade) with the tissue, or when trying to penetrate through the tissue and disconnect it from the vein. In some embodiments, the pressures on the impact ring will cause the ratchet mechanism to disconnect, optionally giving the physician an option to activate the mechanism while the impact ring is not active (without impact effect) and the rotating blade is active and rotating. In some embodiments, the pressure to detach the ratchet mechanism is determent by the impact spring (as shown in FIG. 24j, part 283a12) and can be between 200 gr to 2000 gr or between 350 gr to 1000 gr or between 500 gr to 850 gr or around 500 gr or 800 gr or 1000 gr or 1200 gr.

In some embodiments, the pressures on the impact ring will not cause the ratchet mechanism to disconnect, due to a different mechanism, as shown for example in FIG. 24q. In this options, the ratchet 283a22 is in the lower side of the mechanism, it is located proximally to the impact ring and the edge of the tip. In FIG. 24q, the ratchet mechanism will not disconnect due to high pressure on the impact ring. In some embodiments, when pressure is applied and increases due to the pressure of the device or the tip of the device on the tissue, the impact ring will be pressed on the lower side of the complete mechanism of the tip, and will continue to fully function and achieve the impact on tissue by the impact ring even over 2000 gr of pressure.

As mentioned above, in some embodiments, the principals that guide the architecture of the tissue-cutting tool at the distal head are providing a tissue-cutting tool that cut the tissue to an outer-diameter (OD) as similar as possible to the outer tube of the distal head. For example, using the parts as shown in FIG. 24j, the scope is to cut the tissue at least as big as the outer-diameter (OD) of the outer tube 283a18. Therefore, the lower cradle/hitting blade complex comprises a certain ID (internal diameter) and a certain OD. Since the idea is to provide a cut as close to the OD of the outer tube, the bladed part of the hitting blade is located on the OD of the part. In some embodiments, potential advantages of having the bladed part in the OD side is that it provides a cutting very similar to the OD of the outer tube. Another advantage is that, since there is no bladed portion on the ID of the hitting blade, the distance between the hitting blade and the rotating blade is the shortest, thereby increasing the scissor effect between the two blades. Therefore, in some embodiments, the outer tube will be manufactured to be very thin, therefore diminishing the difference between the OD of the outer tube and the size of the cutting made by the hitting blade.

In some embodiments, the location of the hitting blade and the cutting are interchanged, meaning attached to the outer tube there is first the cutting blade and then the hitting blade, according to the needs of the system.

Exemplary sizes of the parts are shown in FIGS. 24l, 24m, 24n, 24o and 24p as follows: Exemplary sizes for the outer tube 283a18 can be seen in FIG. 24l. Exemplary sizes for the inner shaft 283a2 comprising the rotating blades 283a4 and the teeth of the upper part of the ratchet mechanism 283a6 can be seen in FIG. 24m. Exemplary sizes for the lower cradle/hitting blade complex comprising the hitting blades 283a8 with the lower cradle 283a10 can be seen in FIG. 24n. In this example, the hitting blade is 283a8 circular and continuous. Exemplary sizes for the upper cradle 283a14 comprising the teeth of the lower part of the ratchet mechanism 283a16 can be seen in FIG. 24o. It should be understood that the dimensions disclosed herein are examples only, which are provided to allow a person having skill in the art to understand the invention. While some of the examples are suitable for a 13 French scale catheter, for example, it is the scope of the invention to include either bigger or smaller sizes, for example 15 French, 11 French, 9 French, by reducing the size of the parts. FIG. 24p shows the mechanism without the outer tube and a cross section of the mechanism, were the hitting blade is a circular ring.

In some embodiments, the distance outside of which the impact element/ring extends from the distal end of the catheter, as shown for example in FIG. 24s—284) is, for example, 0.3 mm, 0.5 mm, 0.8 mm, 1 mm or 1.3 mm; optionally from about 0.1 mm to about 3 mm; optionally from about 0.3 mm to about 2 mm; optionally from about 0.5 mm to about 2 mm. In some embodiments, the impact element/ring extends from the second blade that rotates, as shown for example in FIGS. 24t and 24u—286, for example, 0.3 mm, 0.5 mm, 0.8 mm or 1 mm; optionally from about 0.1 mm to about 3 mm; optionally from about 0.3 mm to about 2 mm; optionally from about 0.5 mm to about 2 mm. In some embodiments, the impact element is also rotating when returning into the “free run” area until it gets to the beginning of the acceleration path. Then, the impact element, runs within the encapsulated range (the “free run”/acceleration path) a distance, for example, of 5 mm, 3 mm, 2.5 mm, 2 mm, 1.5 mm, 1 mm or 1.3 mm; optionally from about 0.1 mm to about 3 mm; optionally from about 0.3 mm to about 2 mm; optionally from about 0.5 mm to about 2 mm. In some embodiments, the structure of the impact element is characterized by and internal diameter in range of the sheath. In some embodiments, the structure of the impact element is characterized by an internal diameter from about 0.5 mm to about 7 mm; optionally from about 1 mm to about 5 mm; optionally from about 2 mm to about 4 mm, for example 1.5 mm, 2.5 mm, 3 mm, 3.5 mm, 4 mm, 4.4 mm, 5 mm, 6, or 7 mm. In some embodiments, the structure of the impact element is characterized by a teeth number in the range of 0 to 8 units for example 0, 3, 6, 8. In some embodiments, the structure of the impact element is characterized by a depth between teeth in the range from about 0 mm to about 3 mm; optionally from about 0.1 mm to about 2 mm; optionally from about 0.5 mm to about 1.5 mm, for example 0 mm, 0.5 mm, 0.8 mm, 1 mm. In some embodiments, the structure of the impact element is characterized by a width of blade in the range of from about 0.1 mm to about 2 mm; optionally from about 0.2 mm to about 2 mm; optionally from about 0.5 mm to about 1 mm. In some embodiments, the structure of the rotating element is characterized by and internal diameter in range of the sheath. In some embodiments, the structure of rotating element is characterized by an internal diameter from about 0.5 mm to about 8 mm; optionally from about 1 mm to about 6 mm; optionally from about 1.5 mm to about 4 mm, for example 1.5 mm, 2.5 mm, 3 mm, 3.5 mm, 4 mm, 4.4 mm, 5 mm, 6 mm, or 7 mm. In some embodiments, the structure of the rotating element is characterized by a teeth number in the range of 0 to 8 units for example 0, 3, 4, 6, 8. In some embodiments, the structure of rotating element is characterized by a depth between teeth in the range from about 0.1 mm to about 3 mm; optionally from about 0.3 mm to about 2 mm; optionally from about 0.5 mm to about 2 mm, for example 0 mm, 0.5 mm, 0.7 mm, 0.9 mm 1 mm. In some embodiments, the structure of the rotating element is characterized by a width of blade in the range of from about 0.1 mm to about 2 mm; optionally from about 0.2 mm to about 2 mm; optionally from about 0.5 mm to about 1 mm.

Referring to FIG. 24u, it is shown an embodiment where the impact element/ring comprises a wave form (upper left side). Also shown, an embodiment having an even\strait and blunted impact element/ring (upper side, second from left), the impact element/ring is out, hitting. In this mode, it protects the rotating sharp blades from being traumatic. Also shown, an embodiment having an even\strait and blunted impact element/ring. The impact element/ring is charged in and under (in the free zone) before hitting. In some embodiments, when the impact element/ring hits, it protects the tissue or the vein from contacting the sharp rotating blades. In some embodiments, the same thing happens when the device is stopped; the impact element/ring comes out and stays out to protect the tissue and/or the vein, allowing the doctors to move forward the device without activating it. In some embodiments, the device is activated to run in the opposite direction, the cutting blades rotate on the opposite direction but under the protection of the impact element/ring. In some embodiments, a vibration (˜0.4 mm) is activated. In some embodiments, when and if a cut is done, it will be performed by the rotating blade. In some embodiments, the rotating blade will be located inside the ID of the shaft. This embodiment is especially relevant for all impact element/ring shapes. In these embodiments, all will be blunt. In some embodiments, if it comprises a waveform, then there will be a rotating knife “picking” out in the low points of the wave. In these embodiments, the movement cut like scissors, and removes the tissue. In some embodiments, the rotating blades do not change position, they can only rotate.

Referring to FIG. 24v, showing variations of embodiments having different variations of impact element/rings, according to some embodiments of the invention. In some embodiments, the tip comprises a narrow notch 287. In the bottom part of the FIG. 24v, it can be seen a comparison between an impact element/ring having a narrow notch and one without. In some embodiments, a number of notches is provided, for example between 1 notch and 13 notches. In some embodiments, the form of the notch can be rounded having a “U” shape, or can be sharp having a “V” shape. In some embodiments, a mix of shaped notches are provided. In some embodiments, each notch can have an opening of from about 0.1 mm to about 3 mm, for example, 0.5 mm, 0.7 mm, 1.0 mm, 1.5 mm, 2.0 mm. In some embodiments, a potential advantage of providing notches is that they increase the scissor effect between the moving parts.

In some embodiments, the source for generating the impact is, for example, as follows:

The linear motion approach: the force is generated at the handle: wherein the generation of the impact motion is at the handle side and is transmitted forward by a coupling item along the catheter, such as a compression coil, or push-able stiff wires (e.g. made of Stainless Steel or NiTi). In some embodiments, the motion is contained within the inner shaft and affecting mostly the inner parts, and not the whole device. This may be advantageous because impact motions will not cause the whole device to move while inside the patient.

The “rotational cam with a spring at the tip” approach:
a. In some embodiments, the force is generated by a rotational sheath (e.g. HSS) that surrounds the lead;
b. In some embodiments, the force is generated by a side wire/cable/NiTi structure, HSS, which does not surround the lead, and rotates and transfers the momentum to the tip;
c. In some embodiments, the force is generated by a combination in which part of the path comprises a rotational sheath (e.g. HSS) that surrounds the lead and part of the path comprises a side wire/cable/NiTi structure, also HSS, which does not surround the lead.

The “linear charging spring at the tip” approach: wherein the spring is at the tip and the tip is being pulled/compressed to “charge” the spring (either compress it or stretch it relative to its rest condition). The tip is then abruptly released. The loading of the spring can be performed by pulling or pushing a side wire/cable/NiTi structure, HSS, that transfers the pulling or pushing force to the tip.

In some embodiments, the impact is characterized by the characteristics, for example, of a spring. In some embodiments, the impact element/ring spring when pressed from starting length of about 8 mm to about 5.5 mm it will give a force from about 150 gf to about 3000 gf; optionally from about 300 gf to about 2000 gf; optionally from about 500 gf to about 1000 gf. For example, it can be 250 gf, 350 gf, 380 gf, 400 gf, 450 gf, 5000 gf, 650 gf, 850 gf, 1000 gf, 1,500 gf, 2000 gf, 2,500 gf or 3000 gf when the spring is pressed by the cam in the head, as shown for example in FIG. 24g—252a-b. In some embodiments, when pushing the blades into a surface, neither the impact element/ring nor the rotating blade retracts due to counter forces. In some embodiments, the system withholds forces up to 250 gf, 350 gf, 5000 gf, 650 gf, 850 gf, 1000 gf, 1,500 gf, 2000 gf, 2,500 g or 3000 gf. In some embodiments, even if the user is pushing the handle, the system will not move since it comprises a “stopper” at the distal head. In some embodiments, the spring comprises a length of from about 2 mm to about 12 mm; optionally from about 4 mm to about 10 mm; optionally from about 6 mm to about 10 mm, optionally 9 mm or 8 mm. In some embodiments, the spiral spring comprises wire diameter in the range of from about 0.1 mm to about 5 mm; optionally from about 0.5 mm to about 3 mm; optionally from about 1 mm to about 2 mm. In some embodiments, the complex spring structure comprises struts of from about 0.05 mm to about 0.45 mm; optionally form about 0.08 mm to about 0.40 mm; optionally from about 0.1 mm to about 0.2 mm; for example 0.1 mm, 0.15 mm, 0.21 mm, 0.25 mm. In some embodiments, a complex spring structure is manufactured by cutting a stainless-steel tube or Niti, as shown for example in FIG. 24w (288). In some embodiments, the length of the non-engaged spring will be from about 5 mm to about 10 mm; optionally from about 6 mm to about 9 mm; optionally from about 7 mm to about 8 mm; for example: 6 mm, 7 mm, 7.5 mm, 8 mm. In cases where a preload effect is desired, the non-engaged spring will from about 8 mm to about 20 mm; optionally from about 10 mm to about 18 mm; optionally from about 12 mm to about 16 mm; for example: 9 mm 10 mm 13 mm or 14 mm. In some embodiments, the length of the engaged spring will be from about 2 mm to about 10 mm; optionally from about 3 mm to about 8 mm; optionally from about 4 mm to about 7 mm; for example: 3.5 mm, 4 mm, 5 mm, 5.5 mm, 6 mm, 8 mm or 9 mm.

In some embodiments, as examples, the head of the device comprises the following dimensions as disclosed in FIGS. 24x-24y. FIG. 24x shows a distal head with two blades—one rotating and one impact element/ring. FIG. 24y shows a distal head with rotating and impact element/ring in the same blade.

In some embodiments, the rotating blade (the lower diameter blade) has a phase of 30 degrees facing to the inner diameter. In some embodiments, the rotating blade comprises a phase of from about 20 degrees to about 90 degrees; optionally from about 30 degrees to about 80 degrees; optionally from about 40 degrees to about 70 degrees; for example 60 degrees, 50 degrees, 30 degrees. In some embodiments, the dimensions of the rotating blade are as shown, for example, in FIG. 24z.

In some embodiments, the impact element/ring blade (the higher diameter blade) has a phase of 50 degrees facing to the outer diameter of the tube. In some embodiments, the impact element/ring blade comprises a phase of from about 20 degrees to about 90 degrees; optionally from about 30 degrees to about 80 degrees; optionally from about 40 degrees to about 70 degrees; for example 60 degrees, 50 degrees, 30 degrees.

In some embodiments, the dimensions of the head comprises an arrow-like shape, as disclosed, for example in FIG. 25a, arrowhead (right lower corner) compared to non-arrow head (middle).

In some embodiments, the cutting element is characterized by a combination of a rotational set of teeth, so to generate a cutting effect of a dual tooth-saw (as opposed to stretching or single tooth saw approach). In some embodiments, the combination includes one set moving predominantly longitudinally while the other predominantly rotates (which acts similar to a wiper that removes blocked tissue and avoids congestion, often called “snow plowing effect”).

In some embodiments, the device is characterized by a combination of a steering of the head, with a metal-based (articulated) bending structure (i.e. inner bending shaft), so that pushing forces up to 10 Kg or up to 8 kg up to 5 kg up to 2 kg up to 1 kg up to 800 kg up to 500 kg or less up to 300 gr and than moments up to 150 Ncm or up to 70 Ncm or up to 40 Ncm or up to 20 Ncm (in some test setups the moments can get up to 200 Ncm or less) are transferred to the tip without causing unwanted movements of the bending structure and without blocking of the rotational component or linearly moving components. In some embodiments, the bending component is made of a spring, or a cut tube or an articulated structure as shown for example in FIG. 25b (inner bending shaft—showed in two different perspectives). In some embodiments, the inner component (sheath/HSS) that rotates or moves linearly is made of a spring. In some embodiments, the springs can be an extension coil for example OD 5.5 mm WT 0.4 mm-1 mm length can 2 mm to 15 mm of example 5 mm to 10 mm or 6 mm to 9 mm or other to have the impact as in the calculation shown later, made of metal for example Nitinol for example stainless steel (example: manufacture “febrotec” part number 0T49060, or 0T49030 or from other manufacture). In some embodiments, the spring is designed to transfer the motion in/out or rotation, but not to stretch, e.g. by bonding along its inner radius in case of linear motion.

### 7.3 Exemplary Fluid Dynamics and Forces

Movements of parts often behave differently in an air-based environment and in a liquid-based environment. While parts move freely in an air-based environment, due to lack of resistance, in a liquid-based environment they do not. This can disturb the correct function of parts of the LE device.

In some embodiments, the LE device is characterized by having dedicated holes on parts of the device configured to allow movement/displacement of liquids during activation of moving parts of the device, thereby reducing the resistance of the liquid on the moving parts. Exemplary holes are shown, for example, in FIG. 24w (290), and FIG. 25c (290). In some embodiments, the holes provide access from the outside of the device to the inside of the device, and vice versa, allowing the flow of liquids to and from the two zones. It should be understood that even if not all the drawings show the dedicated holes, the scope of the invention is to include said holes in any variation of the parts of the device.

Referring now to FIG. 25d showing more examples of blades, according to some embodiments of the invention. Referring now to FIG. 25e showing an exemplary blade for engaging calcifications during the lead extraction procedure, according to some embodiments of the invention. In some embodiments, the blades are configured to reduce the cut tissue to the dimension of the inner shaft to potentially easy the passage of the materials through the device. In some embodiments, the blades comprise grooves and/or imperfections on the surface of the blades to further help in the cutting of the tissue, as shown for example in the images on the right on FIG. 25e.

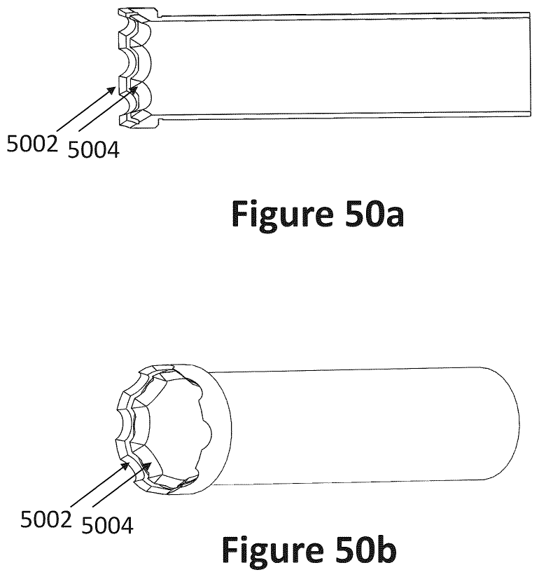

Referring now to FIGS. 50a and 50b, showing an exemplary double blade configuration, according to some embodiments of the invention. In some embodiments, the blades comprise a first set of external blades 5002 and a second set of internal blades 5004. In some embodiments, the double blade configuration potentially performs better cutting actions, than a single blade configuration since it provide a grinding effect on the tissue. In some embodiments, each one of the two blades is configured to rotate independently from the other. In some embodiments, the two blades rotate together. In some embodiments, the blades can rotate at the same direction. In some embodiments, the blades rotate at opposite directions. In some embodiments, the blades are aligned to each other. In some embodiments, one blade is located more distally than the other blade. In some embodiments, blade 5002 is located more distally than blade 5004. In some embodiments, blade 5004 is located more distally than blade 5002. In some embodiments, the blades can rotate at the same velocity. In some embodiments, the blades rotate at different velocities in relation to each other.

In some embodiments, the internal space of the inner shaft, adjacent to the blades, comprises a tapered form, being narrower at the distal end, close to the blades, and widening towards the proximal side, as shown for example in FIG. 25f. In some embodiments, a potential advantage of having a tapered form of the internal space of the inner shaft is that only matter that is big enough to pass the narrow opening created by the tapered shape will enter the internal space of the device, and after that, the matter can easily slide inside the inner shaft since the area is greater due to the widening of the tapered shape.

### 7.4 Exemplary General Description of the Handle Assembly

In some embodiments, the handle of the cardiac lead extractor contains a plurality of controls for the operational features of the device. Referring now to FIG. 25g, showing a schematic representation of the mechanisms included in the handle. In some embodiments, the handle contains the following components: a motor gear, a power source-battery, a transmission gear, a control PCB, indicators, switches, mechanical controls, a steering mechanism, a housing and a cardiac lead lumen.

In some embodiments, the shaft is attached to the handle. In some embodiments, the interface definition between the shaft and the handle delivers torque of 75 [N*cm] and 10 [N] axial force. In some embodiments, additional strain relief between handle and shaft is provided to help with the pushability of the shaft and to prevent kink. Furthermore, in some embodiments, the shaft is stiffer in the proximal end to improve the pushability. In some embodiments, “Contra sleeves” and “steering wires” slack are routed inside the handle.

### 8. Exemplary Balloon Embodiment

In some embodiments, the LE device comprises an inflatable system (balloon) adapted to be inflated and deflated by the user. In some embodiments, the inflatable balloon is applied in a variety of uses. In some embodiments, the inflatable balloon is used as a tissue separator. In some embodiments, the inflatable balloon is used for isolating specific zones from the blood flow (see below). In some embodiments, the inflatable balloon is used as anchorage for the LE device. In some embodiments, the inflatable balloon comprises built-in canals, which allow blood flow to run in them. In some embodiments, one or more balloon components is used to form a support (relative to one or more of a vein, muscle, tissue and/or lead) for assisting the catheter to be pushed against, and/or to generate an anchoring point for the device within the vessel, and/or to assist in directing the device by centralizing/positioning the device within the vessel using balloons as a pushing mechanism, and/or to achieve dilation by separating tissue from and/or surrounding the lead, and/or to use the balloon as a rescue balloon, for example, in case of bleeding and/or vein rupture, and/or to use the balloon as a blood blocker to allow the medical personnel to intervene in case of bleeding. Referring now to FIG. 49, showing a schematic representation of an exemplary lead extraction device with two balloons and drainage system, according to some embodiments of the invention. In some embodiments, the device 4902 comprises two balloons 4904/4906 located at a distal end of the device, with a certain separation S from each other. For example, with a separation of from about 1 cm to about 5 cm. Optionally from about 0.5 cm to about 7 cm. Optionally from about 0.1 cm to about 10 cm. In some embodiments, the lead extraction device comprises a drainage system 4908 located between the two balloons 4904/4906. In some embodiments, a user inflates the two balloons, thereby isolating a specific portion S (equal to the separation distance between the two balloons) of the vessel 4910 from the rest, and then the user activated the drainage system, thereby evacuating the contents within the isolated portion. In some embodiments, more than two balloons are used.

In some embodiments, the inflatable system runs inside the LE device, alongside the lead. In some embodiments, the inflatable system 292 runs outside the LE device 294, as shown for example in FIGS. 25h-i. In some embodiments, the inflatable system 292 runs outside the LE device, inside a dedicated elongated canal 296 attached to the LE device 294, as shown for example in FIG. 25j. In some embodiments, the balloon 298 of the inflatable system 292 is deployed distally ahead of the LE device when the user encounters a place where the lead has been encapsulated by fibrous tissue, as shown for example in FIGS. 25h-j. In some embodiments, the inflatable system 292 can be deployed circumventing the fibrous tissue, as shown for example in FIG. 25j. In some embodiments, the inflatable system 292 can be deployed traversing through the fibrous tissue.

In some embodiments, the deployment of the balloon distally of the fibrous tissue, and inflating the balloon 298 as to put the fibrous tissue between the inflated balloon 298 and the LE device 294 is used as a method for providing further support to the LE device. In some embodiments, once the balloon 298 is inflated, the user pulls proximally the inflation system 292 cord thereby providing a stable counter support for the LE device 294, which needs to move forward in a distal direction.

In some embodiments, the inflation system 292 comprises a built-in inflatable ring-like balloon 300 around the head in the distal end of the LE device 294, as shown in FIG. 25i. In some embodiments, the ring-like balloon 300 is compartmentalized and each compartment is inflated independently. In some embodiments, the inflated ring-like balloon 300 is used an anchorage to the LE device by pressing the vein walls.

In some embodiments, the ring-like balloon 300 is inflated and also a forward distally balloon 302 is inflated distally of the fibrous tissue. The two inflated balloons (300, 302) create a closed space, which, in some embodiments, can be filled with saline (or other transparent liquid) and enable visibility for microcameras located at the distal end of the LE device 294, for example. This is shown, for example in FIG. 25i.

In some embodiments, the inflation system 292 further comprises a deployable net 304, as shown for example in FIG. 25k. In some embodiments, the deployable net 304 is configured to allow the passage of blood and to block the passage of debris caused by the elimination of fibrous tissue around the lead during the activation of the LE device. In some embodiments, the deployable net 304 is deployed by itself, regardless of the inflation system 292.

In some embodiments, the inflation system 292 is built-in 306 (circle) in the outside of the head of the LE device 294, as shown in FIG. 25l. In some embodiments, the built-in inflation system extends partially along the circumference of the head of the LE device. In some embodiments, the head of the LE device comprises more than one built-in inflation system along its circumference. In some embodiments, the deployed inflated balloon works as a tissue separator. In some embodiments, the deployed inflated balloon surrounds the lead, providing space for the lead to move, while gently pushing the vein walls, as shown for example in FIG. 25l. In some embodiments, this method is complementary to the cutting method of the LE device. In some embodiments, this method is substitute to the cutting method of the LE device.

In some embodiments, the balloon, when inflated, can support a force of 1 Newton, or 10 Newton, up to 50 Newton without collapsing or undergoing deformation. In some embodiments, the balloon has a defined form when inflated, and does not stretch and increase in volume under inflation pressure of 2 atmospheres or less. In some embodiments, the balloon can withstand contact with the blades located in the distal end of the device.

In some embodiments, one or more balloon components is used to form a support (relative to one or more of a vein, muscle, tissue and/or lead) for assisting the catheter to be pushed against, and/or to generate an anchoring point for the device within the vessel, and/or to assist in directing the device by centralizing/positioning the device within the vessel using balloons as a pushing mechanism, and/or to achieve dilation by separating tissue from and/or surrounding the lead, and/or to use the balloon as a rescue balloon, for example, in case of bleeding and/or vein rupture, and/or to use the balloon as a blood blocker to allow the medical personnel to intervene in case of bleeding.

### 9. Additional Information

In some embodiments, the device is used as a lead extraction (LE) device, as an atherectomy device, as an object extraction device, and/or as any device for extracting an object from one or more veins and/or a heart chamber and/or the cardiovascular system and/or any tubular structure/lumen in the body (including GI tract). In some embodiments, the device is used for separating an object and/or tissue from surrounding vascular/lumen tissue and/or to separate and/or dissect fibrous/calcified tissue/plaque. In some embodiments, separating the lead from the tissue comprises sideways steering the tip to assist in the separation of the lead from the tissue and/or from other leads, rather than only advancing and/or dilating. In some embodiments, the object is an implantable pacing or a defibrillation lead. In some embodiments, the lumen is a cardiovascular lumen inside the body. In some embodiments, the lumen is a vein inside the body. In some embodiments, the lumen is a heart chamber inside the body.

In some embodiments, the steering is controlled by: 2 pulling wires and/or one or more pulling wires with one or more springs to straighten the catheter and/or one or more springs to keep the wire tight. In some embodiments, the device includes an extension coil to maintain length while the catheter is flexible along its path. In some embodiments, the steering tool comprises modes: free—to maintain flexibility of the catheter and allow it to freely respond to path curvatures and/or to counter force applied by the tissue or leads; or sets a certain force/pressure/moment but responds (bends/stretches) to changes in the curvature of the path and/or responds to the counter forces applied by the tissue or by the leads; or sets fixed elongation/stretch/bending at the tip. In some embodiments, the modes can be changed manually, for example, by engaging the pulling wires and holding them firmly to set a fixed bending, or letting them loose to have the bending angle free to be changed by the path it is in.

In some embodiments, the device provides controlled steering of the tip to control the orientation of the force or applied energy to the desired target tissue and reduce the likelihood of applying the energy to the vein wall.

In some embodiments, the controlled steering mechanism is integrated with extraction tool and forms a single device with steering of its head, which applies cutting, sawing, and/or impact forces to the tissue by its tip. The steering provides control of the orientation of the forces.

In some embodiments, the device is an outer sheath with steerable bending capabilities that provides control over an extraction tool (whether mechanical or laser or thermal or ultrasound, or balloon based, or others) that passes through (internally) the steerable outer sheath device. In some embodiments, the steerable outer sheath is made of stainless steel or plastic. In some embodiments, the steerable outer sheath bends up to 90 degrees or less over a radius of up to 20 mm or larger and length of 100 mm up to 1400 mm; for example 30 mm, 48 mm, 90-1200 mm. In some embodiments, the steering device slides over the extraction tool and can control the location of the bending along the path of the extraction tool.

In some embodiments, the steerable sheath has a circular lumen with a length of at least 5 cm (or up to 10 cm, or up to 20 cm, or at least 20 cm, or 20-30 cm, or 25 to 55 cm or 90-140 cm) through which the extraction tool is passed. In some embodiments, the steerable sheath has a side opening, such that it can be attached to or fitted over an extraction tool from the side of the extraction tool without having to pass the tip of the extraction tool through the steerable sheath. In some embodiments, the steerable sheath has a separator component that opens the side opening to enable passage of the extraction tool through its side into the steerable sheath, and enable closure (whether full or partial) of the steerable sheath over the extraction tool.

In some embodiments, the LE device is made of materials suitable for being passed within the vasculature and that support lightweight and electrically isolated device qualities (e.g. Plastic). In some embodiments, other components' materials provide a device suitable for being passed within the vasculature and with high and easy usability, easy cleaning, easy handling, high safety and medical device standard requirements, including biocompatibility and corrosion resistance.

In some embodiments, the LE device is electrically powered from external sources (e.g. directly connected to the power grid). In some embodiments, the LE device comprises an internal power source (e.g. lithium batteries). In some embodiments, the internal power source is sufficient to power the LE device for more than 20 minutes and at least 3 hours accumulated. Optionally 2 hours accumulated.

Exemplary Integration of the LE Device with Imaging Devices

In some embodiments, the LE device is configured to be used with a variety of imaging devices, for example, XRAY devices, ultrasound devices, etc. In some embodiments, the device comprises a plurality of radio-opaque markers that are used to help the user identify the tridimensional orientation of the device using bi-dimensional means.

### Exemplary Ports for Insertion of Liquids

In some embodiments, the LE device comprises one or more entry ports for the insertion of liquids (e.g. saline) through the device into the zone in the body where the lead is being extracted. In some embodiments, the port is connected to a dedicated channel that runs along the device. In some embodiments, the dedicated channel runs in parallel to the contra sleeves of the wires. In some embodiments, the dedicated channel runs inside the lumen of the device from which the lead is being pulled. In some embodiments, the dedicated channel runs between the inner torque lumen of the device, from which the lead is being pulled, and the outer lumen surrounding the inner lumen, then the liquid will flow between the static outer lumen and the rotating lumen that transfer the lead and the torque the to the mechanism at the tip, the liquid will be delivered to the bending shaft (Hinge) and the tip area. In some embodiments, a potential advantage of having ports is that flushing liquids during the procedure helps to reduce friction, it cleans the vessels, and it clears the view when cameras are used in the device.

### Exemplary Additional Outer Sheath

Referring now to FIG. 25m, showing exemplary additional outer sheath, according to some embodiments of the invention. In some embodiments, the device can be equipped with an additional outer sheath. In some embodiments, the additional outer sheath is as long as the device itself. In some embodiments, the additional outer sheath is short and it is configured to work as a port for the insertion of the device and protection of the patient during the procedure. In some embodiments, the additional outer sheath is stiffer than the device itself. In some embodiments, the length of the additional outer sheath is from about 5 cm to about 30 cm, for example, 10 cm, 15 cm, 20 cm, 25 cm. In some embodiments, the shorter outer sheath, for example half of the length of the device, is configured to be inserted and/or removed during the procure itself. In some embodiments, a potential advantage of this is that it allows flexibility during the procedure. In some embodiments, the outer sheath also allows a reduction of friction at the entry point into the body. In some embodiments, the length of the additional sheath when used during femoral insertion is from about 40 cm to about 50 cm, optionally from about 35 cm to about 60 cm, optionally from about 30 cm to about 70 cm, for example 43 cm, 45 cm, 47 cm. In some embodiments, the additional outer sheath is a split 305a outer sheath. In some embodiments, the additional outer sheath comprises one or more channels 305b configured to be used for the delivery of liquids, the passage of cables and/or electronics. In some embodiments, the additional outer sheath comprises one channel. In some embodiments, the additional outer sheath comprises two channels. In some embodiments, the additional outer sheath comprises three channels. In some embodiments, the additional outer sheath comprises between one and eight channels. In some embodiments, the additional outer shaft comprises an internal diameter of from about 7.5 mm to about 8.5 mm, for example, 7.8 mm, 7.9 mm, 8.1 mm, 8.2 mm. In some embodiments, the outer diameter of the additional outer sheath is from about 0.01 mm to about 0.1 mm bigger than the internal diameter. In some embodiments, the thickness of the wall of the additional outer diameter is from about 0.5 mm to about 1.0 mm, for example, 0.7 mm, 0.75 mm, 0.8 mm, 0.6 mm, 0.9 mm. In some embodiments, the internal diameter of the one or more channels is from about 0.3 mm to about 1.1 mm, for example, 0.3 mm, 0.4 mm, 0.5 mm, 0.6 mm, 0.7 mm.

In some embodiments, the sheath comprises a certain level of stiffness. In some embodiments, the stiffness is being measured by using a setup as shown, for example, in FIG. 4g when L=180 mm-220 mm and deflection is 70 mm to 80 mm, then it will have the force in the range of F=(0 gr to 500 gr); nominal forces can be in the range of F=(45 gr to 130 gr) or F=(30 gr to 50 gr) or F=(60 gr to 110 gr) or (100 gr to 250 gr) or (150 gr to 250 gr) or for example F=60 or F=80 or F=100.

In some embodiments, by adding materials on the inside walls of the lumens, for example stainless steel metal and/or Nitinol and/or any other material and/or other device like one of the sensors (as described here), the outer sheath will be less flexible and stiffer. In some embodiments, the addition of materials as disclosed above, will be used when the user will be interested in changing the stiffness of the shaft during the procedure and depending on the vessel and/or lead structure (path\orientation).

In some embodiments, the additional outer sheath is an accessory comprising one or more elements that enhances the capabilities of the LE device, for example: tissue identification mechanism, sensors, steering (when the LE device does not have steering capabilities), cutting mechanism, lead cutting mechanisms, cameras, or any of the mechanisms described herein.

### Exemplary Additional Inner Sheath

In some embodiments, an additional inner sheath is used. In some embodiments, the additional inner sheath is sized to be inserted inside the lumen of the inner shaft and long enough to reach the distal end of the device. In some embodiments, the additional inner shaft is used for example, to help cutting tissue, help pushing tissue, help reducing radial forces over the inner shaft by allowing the tissue to pass through it.

### Exemplary Sensors

In some embodiments, the device comprises one or more sensors on the device itself and/or on the outer sheath and/or on the steerable sheath and/or on any of the exemplary accessories as disclosed herein. In some embodiments, the sensors are configured to measure, for example, one or more of sensing viability and/or tissue type and/or 02 concentration and/or local temperature and/or heat dissipation. In some embodiments, this is done to differentiate between one or more of blood, vessel, muscle, adhesion, substantial calcification, lead, metal, etc. In some embodiments, any of the abovementioned or the foregoing measured data and/or one or more measurements are used to generate one or more images and/or other visualization modalities for the user. In some embodiments, one or more 2D images, one or more 3D images, one or more circular images, one or more linear images, and/or one or more scattered images.

In some embodiments, data and/or measurements are obtained in plurality of points around the perimeter of the catheter in the vicinity of the tip of the catheter. In some embodiments, said plurality of points is displayed along a circular shape indicating of a cross section of the catheter and its vicinity. In some embodiments, at least two data points are obtained indicating information along the left-right directions of the catheter's tip. In some embodiments, at least two data points are obtained indicating of information along the lateral-medial directions of the catheter's tip.

In some embodiments, any of the abovementioned or the foregoing one or more measurements and/or data and/or images are displayed on a standalone display. In some embodiments, the data and/or one or more images are integrated to be displayed as part of one or more of the operation room display systems and/or as part of other imaging modalities, for example fluoroscopy display and/or ultrasound display.

In some embodiments, the device includes one or more light sources, for example, in visible light range, or in infra-red range. In some embodiments, the light source includes one or more fiber optics and/or LED and/or laser sources. In some embodiments, LED indicators are located on the handle, the pedal or both.

In some embodiments, the device includes one or more irrigation channels and/or ports to convey fluids or gas to the area of imaging and/or sensing. In some embodiments, saline and/or water and/or CO2 and/or contrast media and/or a marker/stain (that enhances the detection e.g. of adhesion, vein, lead, muscle, other tissue). In some embodiments, irrigation is performed at the tip of the device near the region of interaction of the lead extraction tool with the adhesive tissue and/or vein and/or muscle and/or lead. In some embodiments, such irrigation is used to enhance visualization clarity and or sensing capabilities.

In some embodiments, the device includes one or more photo diodes and/or photo detectors and/or array of visual light detectors, and/or array of infrared detectors, and/or one or more fibers optics and/or one or more cameras. In some embodiments, such measurements are used to generate an image or other visualization for the user.

In some embodiments, the device includes one or more RF sources and/or antennas at the vicinity of the tip. In some embodiments, both transmission of RF energy and receiving and/or measurement and/or detection are performed at the vicinity of the tip.

In some embodiments, the device includes one or more of heating element and/or cooling element and/or irrigation of heated or cooled fluid at the vicinity of the tip. In some embodiments, both temperature measurement and or heat dissipation measurement are performed at the vicinity of the tip. In some embodiments, such measurement is used to evaluate tissue response and/or tissue thermal properties and/or blood flow properties and/or tissue heat dissipation properties.

In some embodiments, the device includes one or more ultrasonic energy sources (transducer, ceramic and/or polymer) at the vicinity of the tip. In some embodiments, both transmission of ultrasound energy and receiving and/or measurement and/or detection are performed at the vicinity of the tip. In some embodiments, such measurement enables evaluation of mechanical properties of the tissue, including one or more of thickness, density, calcium content, elasticity, fragility and mechanical resistance.

In some embodiments, the device includes one or more force transducers and/or pressure sensors at the vicinity of the tip. In some embodiments, such measurement enables evaluation of mechanical properties of the tissue, including one or more of elasticity, fragility and mechanical resistance.

In some embodiments, the device includes one or more low power electrical power (current and/or voltage) sources at the vicinity of the tip. In some embodiments, such electrical power is delivered in frequencies of above 1 KHz or above 3 KHz above 10 KHz. In some embodiments, such electrical power is delivered in frequencies of below 1 GHz or below 100 MHz or below 10 MHz or below 1 MHz. In some embodiments, such electrical power is delivered in one or more pulses having an effective pulse width of less than 300 microseconds, or less than 100 microseconds, or less than 10 microseconds, or less than 1 microsecond. In some embodiments, both transmission of electrical energy and receiving and/or measurement and/or detection are performed at the vicinity of the tip. In some embodiments, tissue electrical impedance is evaluated. In some embodiments, tissue resistance, absorption, and/or reflection properties are evaluated. In some embodiments, these are used to produce one or more values and/or to produce an image or an array of values indicating the tissue and/or target properties. In some embodiments, pacing and/or defibrillation electrodes are located at the vicinity of the tip. In some embodiments, the device is capable of delivering pacing or defibrillating signals. In some embodiments, any of the abovementioned channels and/or ports and/or sensors and/or energy sources are positioned along the device. For example in the vicinity of the device's distal end.

In some embodiments, any of the abovementioned channels and/or ports and/or sensors and/or energy sources are positioned along an outer sheath that is located over the lead extraction catheter. For example, near the outer sheath's distal end.

In some embodiments, any of the abovementioned channels and/or ports and/or sensors and/or energy sources are positioned along a parallel catheter that is applied along the venous path and or in the heart next to the lead extraction catheter. For example, near the parallel catheter's distal end.

In some embodiments, one or more of a tension sensor, and/or a pressure sensor and/or a force sensor and/or a lead impedance sensor and or lead to device impedance sensor are applied to evaluate lead integrity and completeness, and/or to alert on lead break, and/or alert on lead unfolding, and/or alert on lead over-stretch, and/or partial entry or capture of a locking stylet.

In some embodiments, the system includes 3D position sensors, such as electromagnetic position sensors (e.g. coils measuring RF or magnetically induced field relative to electromagnetic field produced in the vicinity of the patient) or impedance balance sensors (e.g. using multiple stickers/electrodes on the patient's skin or electrodes within the patient body that measure the impedance from each such sticker/electrode to a sensor/electrode in a target location). In some embodiments, such one or more 3D position sensors are located near the device distal end, and/or along the bending shaft (Hinge), and/or along the shaft, and/or along an outer sheath. In some embodiments, the one or more position sensors are used to create a 3D map of the path catheter. In some embodiments, measurement of impedance or induced field is used to evaluate the location and path of the lead. In some embodiments, the 3D map displays the path of the lead and/or the position of the lead relative position and orientation of the CLE device. In some embodiments, the location and orientation of the lead in the vicinity of the device tip is displayed relative to the device tip location and orientation. In some embodiments, such relative location and/or orientation is used to guide the direction towards which it is desired to orient the tip of the extraction device.

In some embodiments, the system provides inputs to the user regarding any information on progression of the device and alerts. For example, if the motor consumes high current, if a lot of force is applied but there is no progress, if high tension is applied to the lead/locking stylet, if any of the abovementioned sensors indicated a value above or below a predetermined/configurable threshold. In some embodiments, the system will then provide feedback (including for example by sound, display, light, vibration, etc.), possibly suggesting to stop and try again or change direction, or reposition, etc. In some embodiments, such feedback includes auto stop of the device operation immediately, or after a certain preconfigured delay, or after crossing a certain threshold of time in which certain data crossed a threshold. In some embodiments, the device is configured to alert when there is progress of less than 3 mm over 30 seconds of operation.

In some embodiments, the device includes one or more sensors of blood pressure and/or blood flow and/or velocity of blood stream and/or spectral analysis of blood flow (e.g. Doppler shift and/or plurality of velocities and/or turbulence). In some embodiments, such one or more sensor are located near of the extraction device′ distal end. In some embodiments, such one or more sensors are located in the vicinity of the outer sheath′ device distal end. In some embodiments, such sensors is used to detect local blood leakage. In some embodiments, such sensors is used to detect venous tear. In some embodiments, such sensors is used to detect muscle perforation. In some embodiments, an event is detected by one or more of temporary blood pressure drop and/or change in spectral properties of blood flow, or detection of turbulence. In some embodiments, a detected event is used to alert the user. In some embodiments, a detected event is used for auto-stop activation of the device.

### 10. Exemplary Pulling/Grapping Device 6

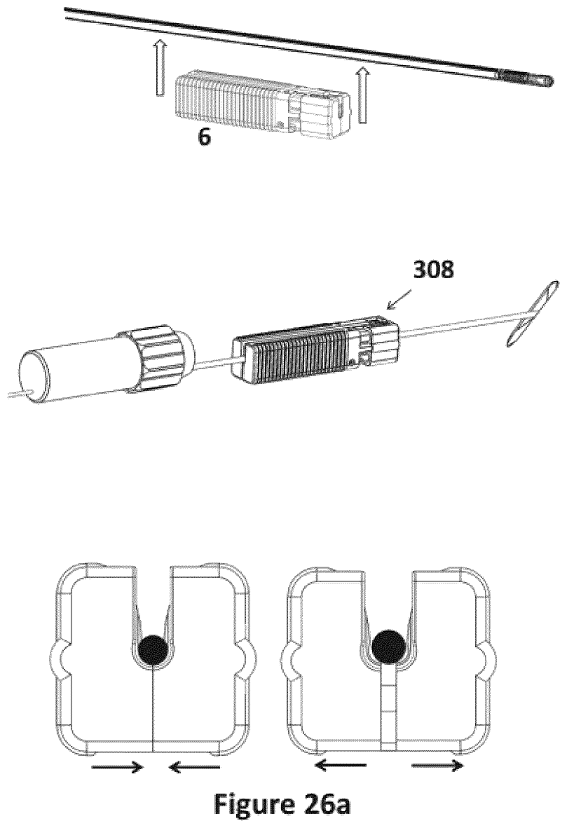

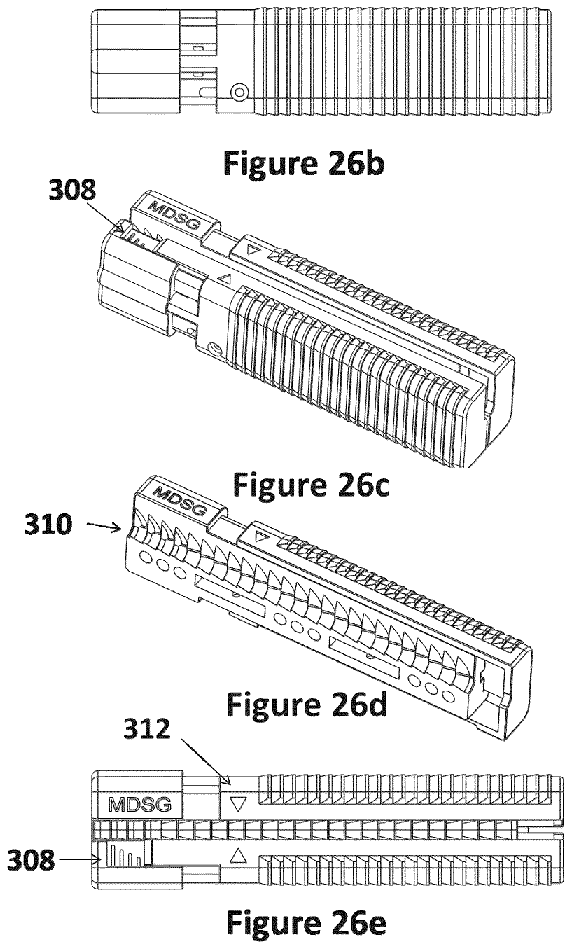

In some embodiments, a dedicated handle 6 is attached to a LE device, which provides an ergonomical grapping of the sheath, said dedicated handle is held by the free hand of the user for wrapping, catching and pulling the sheath. In some embodiments, the handle is reversibly attached to the LE device at a location of choice of the user, as shown for example in FIG. 26a. In some embodiments, the handle comprises a longitudinal aperture, which allows the mounting and dismounting of the handle from the LE device during the procedure performed by the user. In some embodiments, the dedicated handle is adjustable to grab the sheath or be loose to slide on the sheath according to the pressure that the user applies. In some embodiments, the handle comprises a force indicator 308, which enables the user to be aware of the force applied when pushing/pulling, as shown for example in FIG. 26a. In some embodiments, the indicator is a meter, a screen showing colors, a sound, or any other suitable system (e.g. to be shown on displays, on fluoro, etc.). Some examples of architecture of the handle can be seen, for example in FIGS. 26b-e. In some embodiments, the handle is configured to be attached to a variety of sizes and lengths of LE devices/catheters.

In some embodiments, a dedicated handle will have additional lock (not shown) to prevent from unwanted dismounting.

In some embodiments, the inner side of the handle is designed to fit the sheath of the LE device, and in some embodiments, it includes a rubber or radial shaped configuration 310 or other to increase the friction between the handle and the sheath and to reduce damage to the sheath.

In some embodiments, the handle comprises a manual stepper 312 of pulling, so the user does not apply force by hand, but rather determines either the force or the distance of progress relative to the handle, as shown for example in FIGS. 26a-e. In some embodiments, the handle comprises a force/distance/velocity limiter, so no pulling becomes too abrupt (when an obstacle is gone, the force of pushing and pulling makes the catheter run with less control that might impact the vein).

In some embodiments, the handle includes an option to stay locked on the sheath, so the user can push\pull the handle without having any concern of maintaining the grab on the handle but just for pushing or pulling in along the sheath.

### 11. Exemplary Pulling Device 4

The extraction of the lead from the patient is typically performed by pulling the lead from the patient. During the extraction, the user usually coils the pulled lead on his own hand in order to continue pulling the lead. The coiled lead hurts the user hand.

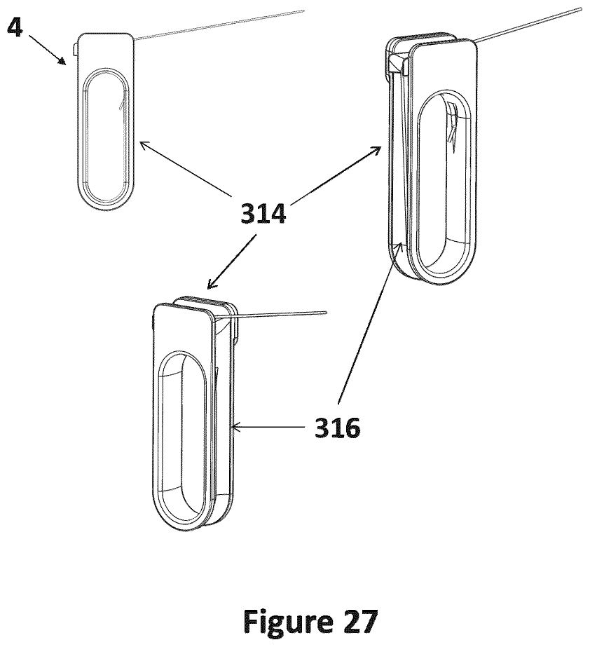

In some embodiments, a pulling accessory device is used. In some embodiments, the pulling accessory comprises a body 314, adapted to be held by the user and surrounds the hand of the user. In some embodiments, the pulling accessory comprises a canal or groove 316 on its external periphery where the extracted lead is collected. In some embodiments, the lead is firmly attached to the pulling accessory device. In some embodiment, the user coils the stylets/wires/lead around the pulling accessory device therefore not damaging the user's hand. In some embodiments, the pulling accessory device comprises a force indicator. In some embodiments, the pulling accessory device is as shown, for example, in FIG. 27.

### 12. Exemplary Accessories

In some embodiments, add-on's and/or accessories, for example, lead cutter, sensors, steering, force measure, etc., are adapted to be either an integral part of the lead extraction device or an add-on as separate tools or combined add-on's. In some embodiments, accessories are used without requiring taking the extractor out from the patient. For example, wires or other cutting tools can be mounted externally to the existing extraction tool and pushed in the body along the said device till reaching the distal end and preform the cutting action.

### 12.1 Steerable Sheath (for LE Device)

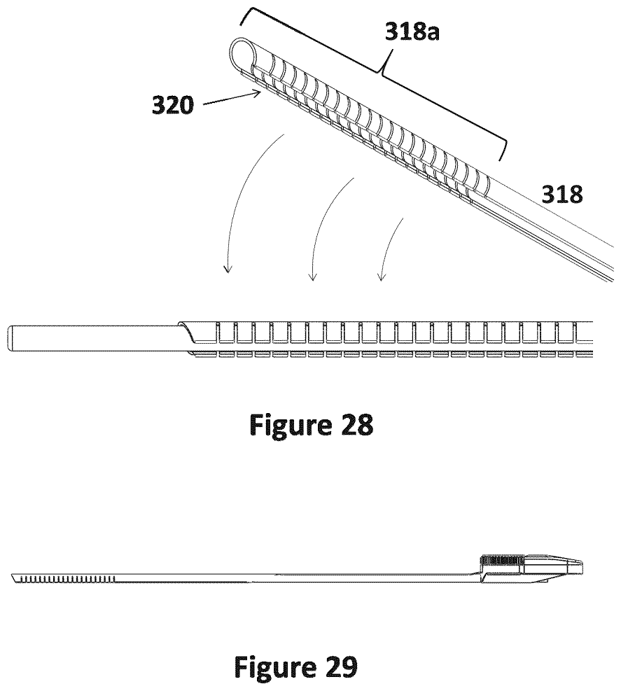

In some embodiments, when the user already has an LE device without steerable capabilities, it would be an improvement to enable said LE device with steerable capabilities. In some embodiments, a steerable sheath is used to provide LE devices with steerable capabilities. In some embodiments, the steerable sheath 318 is reversibly attachable to the LE device, as shown for example in FIG. 28. In some embodiments, the distal part of the sheath 318a is the steerable part. In some embodiments, the steerable part can be made in different dimensions and different materials from the sheath, like plastic or metal or other material according to the existing LE device. In some embodiments, the end or tip of the distal side can be smooth with low friction. In some embodiments, the tip can be from the same material as the shaft. In some embodiments, the tip is made from other material like plastic or metal. In some embodiments, the tip is as shown for example in FIG. 25m.

In some embodiments, the bending shaft (Hinge) (part of the steering mechanism), the sheath and the active parts which maintain the needed pushing and pulling forces, includes one or more channels as shown in FIG. 25m. In some embodiments, the characteristics of the steerable part are the same as disclose above for the built-in steerable mechanisms.

In some embodiments, the bending shaft (Hinge) (part of the steering mechanism), the sheath and the active parts which maintain the needed pushing and pulling forces, are all part of an unified device, as shown for example in FIG. 29. In some embodiments, the user pulls and pushes as needed in the procedure but the shape of the bending shaft (Hinge) stays fixed as the user tuned, due to the internal mechanisms inside the bending shaft (Hinge) and the extractor, which ensure the stability of the steered end albeit the forces. In some embodiments, the device and the extruder are adapted to sustain the strong forces due to the materials chosen and the design, which enable them to hold the torque and pulling, and pushing forces.

In some embodiments, the sheath comprises a longitudinal aperture or sideway insertion with hooks 320, as shown for example in FIG. 28, which enables the attachment and removal of the steering sheath from the LE device during the physician regular procedure.

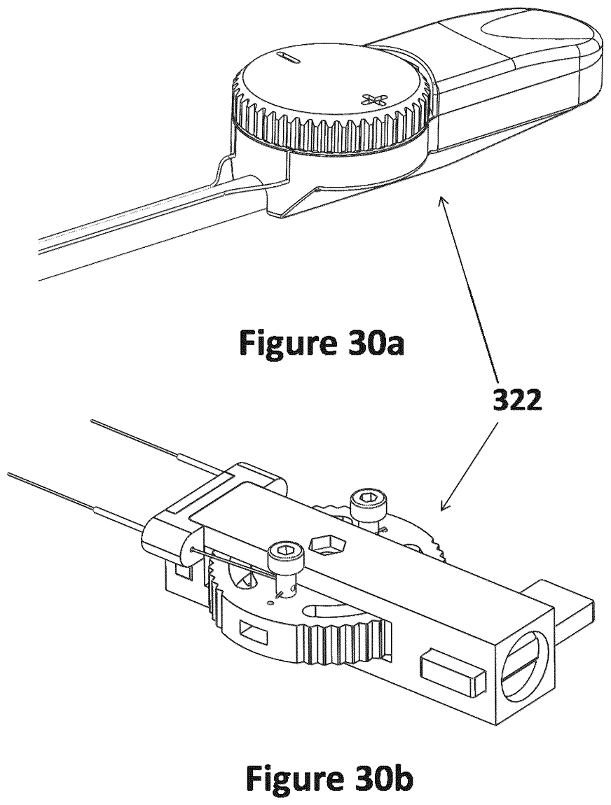

In some embodiments, a manual controller of the steering movement 322 is located on the proximal end of the steerable sheath, close to the user, as shown for example in FIGS. 30a-b. FIG. 30b showing the internal mechanism of the manual controller shown in FIG. 30a. In some embodiments, it can be relocated during procedure.

In some embodiments, the length of the sheath can be from 10 cm to 1.4 meter.

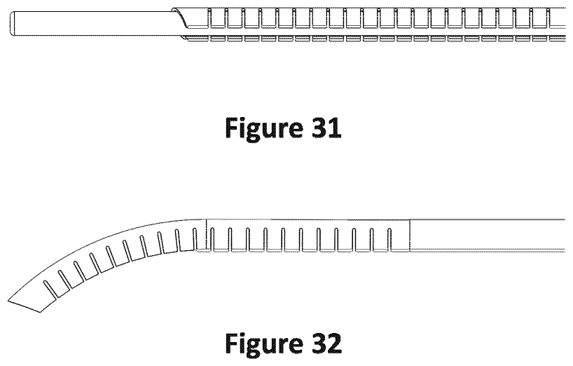

In some embodiments, once the steerable sheath is mounted on the LE device (as shown in FIG. 31), the user actuates the controller which steers the distal end of the LE device, as shown for example in FIG. 32. In some embodiments, the steering is performed from left to right and/or to right to left and\or axial rotating on the sheath or handle.

In some embodiments, the steering movement is adapted to be loose. In some embodiments, the steering movement is adapted to be stiff. In some embodiments, the steering movement is adapted to be manipulated according to the physician request.

In some embodiments, the handle of the steerable device is ergonomically designed longitudinally to the LE device, as shown for example in FIG. 30a. This feature enables the user to control the steering of the LE device while holding the proximal end of the LE device, potentially leaving the second hand of the user free for other roles during the procedure.

In some embodiments, the proximal end of the steerable sheath is configured to be firmly attached to the LE device, permitting the user to use it for pulling and/or pushing actions. In some embodiments, the proximal end of the steerable sheath is configured to be attached to a variety of sizes of LE devices/catheters.

In some embodiments, the handle of the steerable sheath comprises a force indicator, which enables the user to be aware of the force applied when pushing/pulling. In some embodiments, the indicator is a meter, a screen showing colors, a sound, or any other suitable system (e.g. to be shown on displays, etc.).

In some embodiments, the handle of the steerable sheath comprises an indicator showing how much the distal head of the LE device is bent by the vein, and how much resistance the bending head has when trying to bend. In some embodiments, these indicators are used as indicators of obstacles.

### 12.2 Exemplary Attachment Ring for LE Device

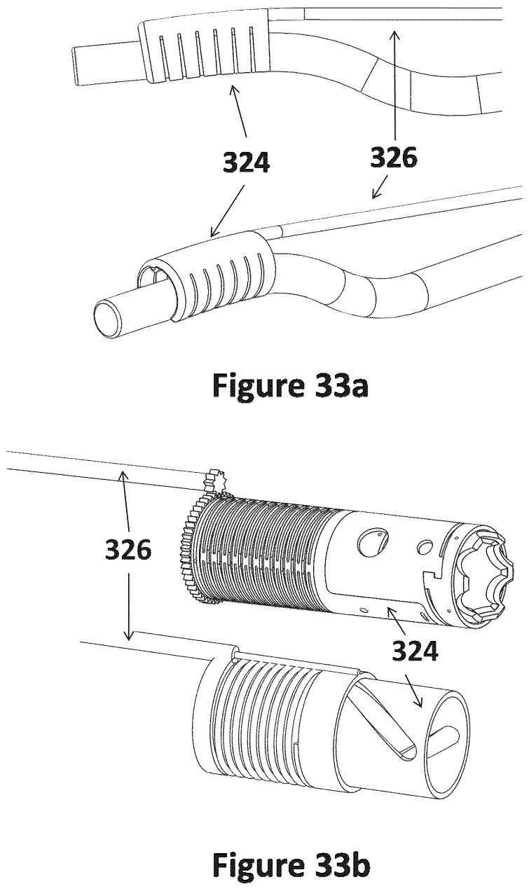

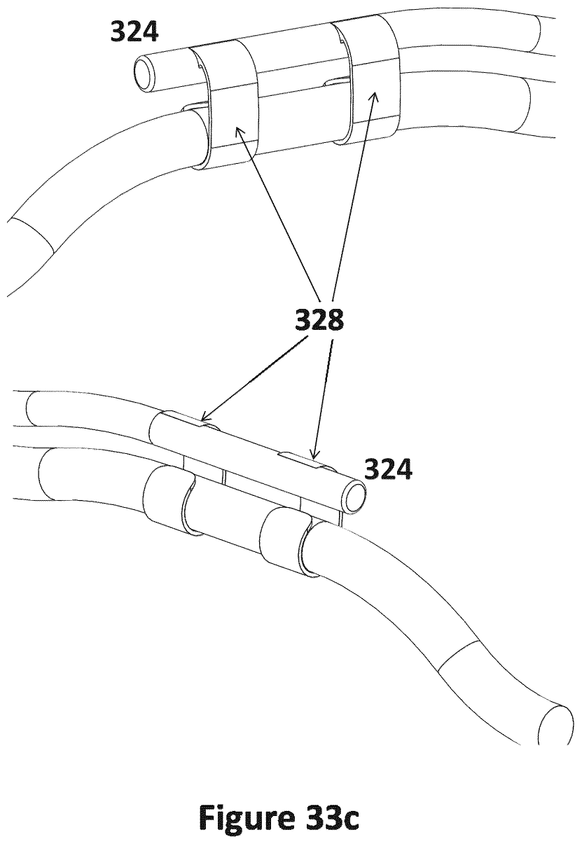

In some embodiments, the mechanisms disclosed (e.g.: steering mechanism, cutting mechanism, etc.) are configured in a single “head unit” which is attachable to an existing LE device deprived of said mechanisms or to the lead itself, as shown for example in FIGS. 33a-b. In some embodiments, the head unit 324 is connected to an elongated body 326, which comprises on its proximal end the hand controller of the mechanisms (not shown). In some embodiments, the elongated body 326 comprises inside all the required machinery for activating the mechanisms in the head unit. In some embodiments, the head unit is reversible attachable to an existing LE device or the lead itself, as shown for example in FIG. 33a. In some embodiments, the head unit is not directly attached to a LE device or the lead, rather it utilizes a ring-like attachment 328, as shown for example in FIG. 33c. In some embodiments, the ring-like attachment “hugs” the LE device or the lead, and this attachment is used for guiding and following the head unit to the path of the lead. In some embodiments, the total diameter of the LE device and the head unit connected to the elongated body are from about 5 mm to about 8 mm. In some embodiments, the characteristics as disclosed above concerning the distal end apply here.

### 12.3 Exemplary Pulling/Grapping Accessory Device

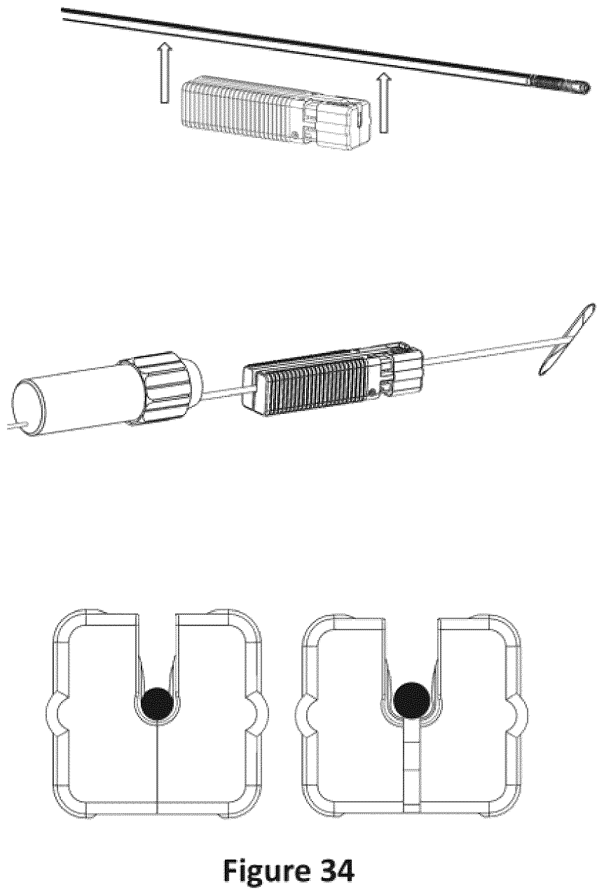

In some embodiments, a dedicated accessory handle is attached to a LE device, which provides an ergonomical grapping of the sheath, said dedicated handle is held by the free hand of the user for wrapping, catching and pulling the sheath. In some embodiments, the handle is reversibly attached to the LE device at a location of choice of the user, as shown for example in FIG. 34. Exemplary characteristics related to the handle accessory are disclosed above in section 10.

### 12.4 Exemplary Pulling Device Accessory

The extraction of the lead from the patient is usually performed by pulling the lead from the patient. During the extraction, the user usually coils the pulled lead on his own hand in order to continue pulling the lead. The coiled lead hurts the user had.

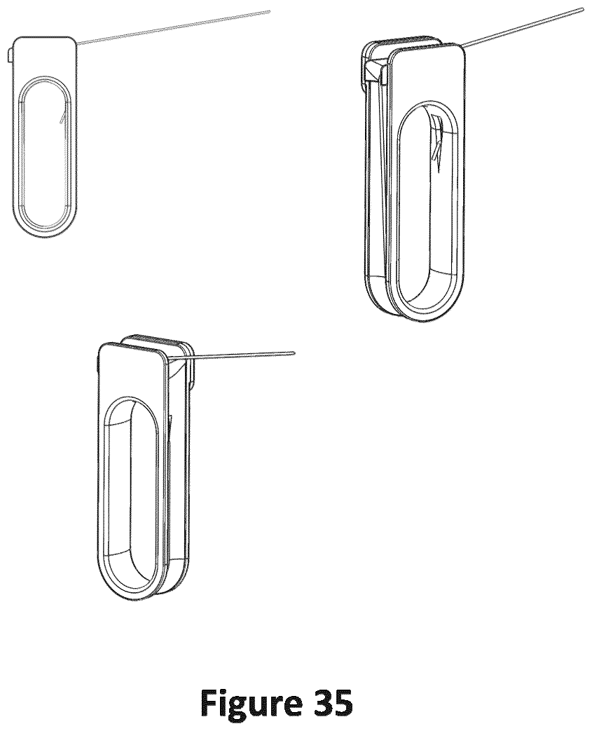

In some embodiments, a pulling accessory device is used. In some embodiments, the pulling accessory device is as shown, for example, in FIG. 35. Exemplary characteristics related to the handle accessory are disclosed above in section 11.

### 12.5 Exemplary Tissue and Binding Site Assessment Accessory

In some embodiments, accessories allowing to classify matter distally of any LE device are provided. In some embodiments, the accessories work in the same manner as described above in sections 4.9 and 4.10.

### 12.6 Exemplary Lead Cutter Accessory

As mentioned above, in some cases, during the lead extraction procedure, the user arrives at the conclusion that the lead cannot be taken out from the tissue without causing too much damage. In these cases, it may be preferable to cut the reminder of the lead instead of forcing it out.

In some embodiments, a lead cutter accessory is provided. In some embodiments, the lead cutter accessory slides around the lead when the extractor is out or while the extractor is still in position, where a cut is needed by the user.

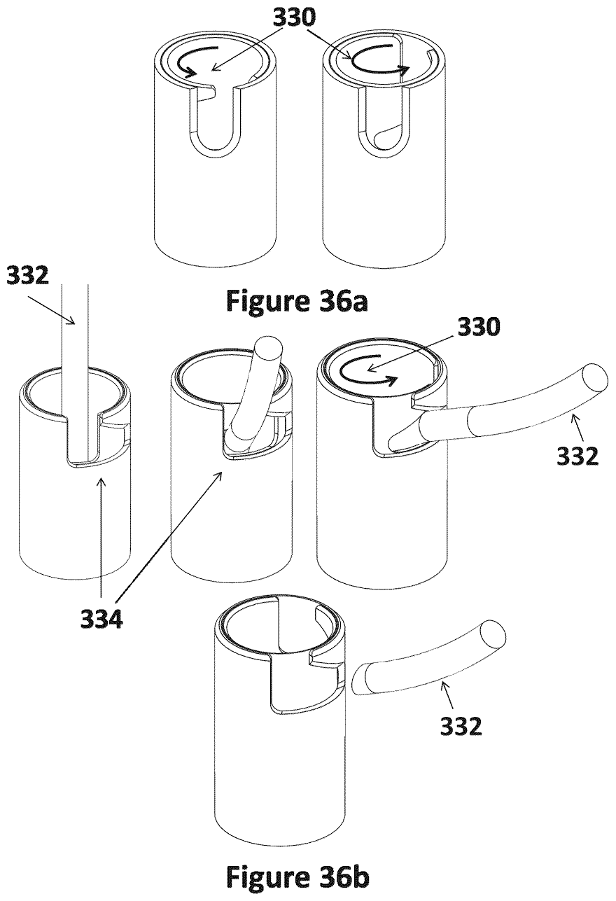

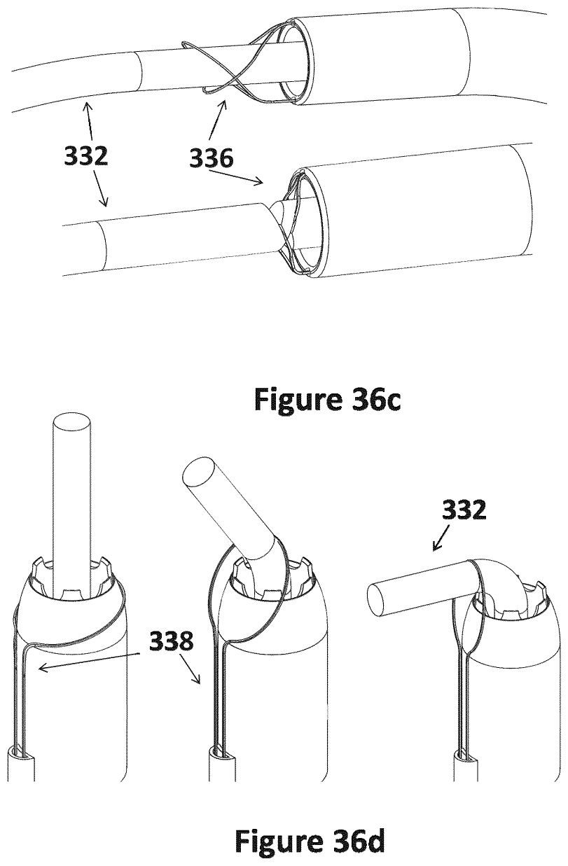

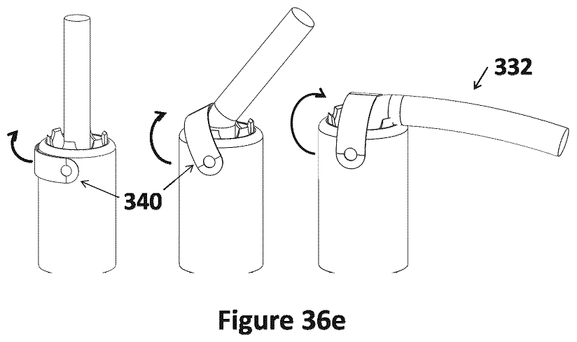

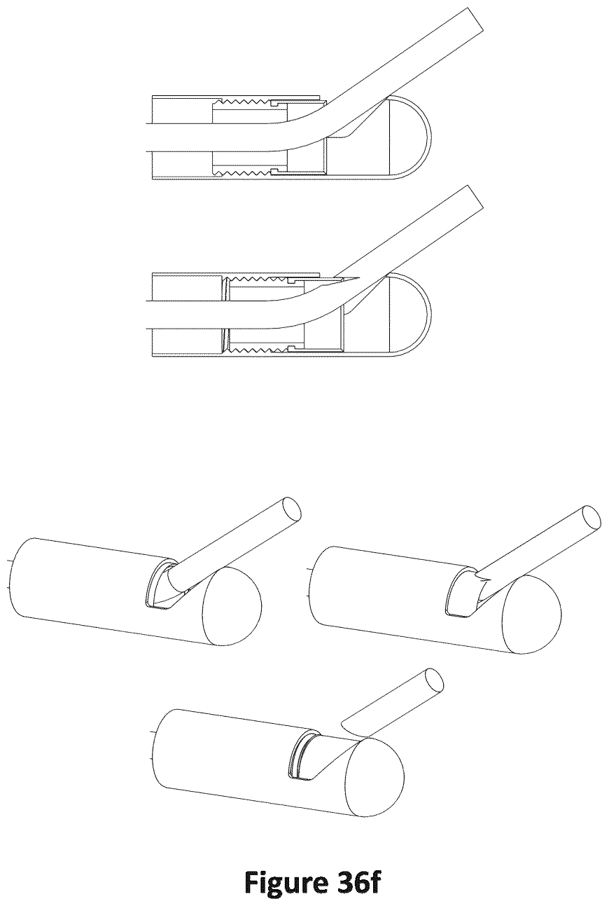

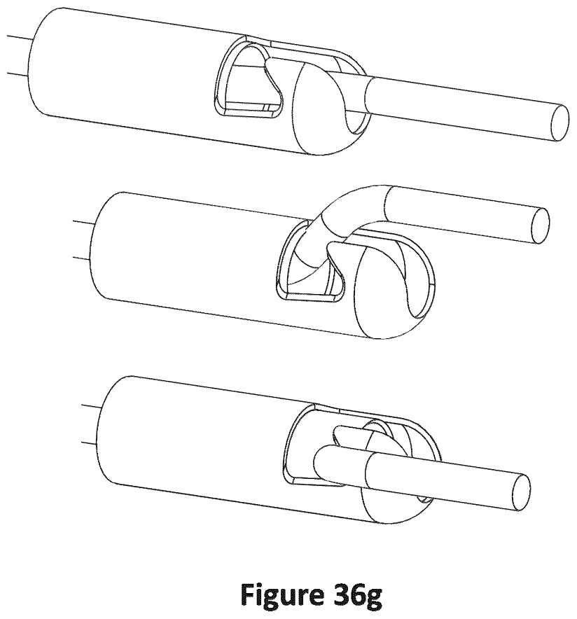

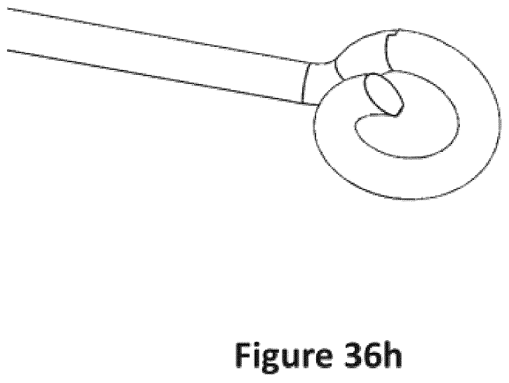

In some embodiments, the lead cutter comprises at least one rotating/sliding plate 330, as shown for example in FIG. 36a. In some embodiments, the action of cutting the lead is as shown in FIG. 36b, and it is, for example, as follows:

The lead 332 passes through the lead cutter accessory. When the user brings the device to the desired point where the lead 332 needs to be cut, the user moves the distal end of the device so as to insert the lead 332 into the groove 334. Once the lead 332 is in the groove 334, the user activated the rotating plate 330, which cuts the lead 332.

In some embodiments, the distance between blades in the cutting mechanisms is zero. In some embodiments, the distance between blades in the cutting mechanisms is negative and at least one blade is made of flexible metals or other materials, which adapts to the second blade during the cutting action. In some embodiments, the blades at the distal end are made of different metals, for example, one blade made of hard metal and one made of soft metal.

In some embodiments, the lead cutter accessory comprises different mechanisms, which ensure that the lead does not move, or escape from the cutting zone. In some embodiments, the sliding of the rotating plate is from right to left and/or to left to right. In some embodiments, where the cutting edge is sharp, the cutting mechanism will be forced to have a phase to make a cut. In some embodiments, the edge is designed to be safe for use in the internal organs. In some embodiments, the lead cutter can be redrawn and/or reloaded after a cutting attempt was done for relocating or replacing a tool, according to the user decision.

In some embodiments, the lead cutter accessory comprises at least 2 wires 336 which choke the lead 332, and by using pressure, and/or by pulling the wire or wires back and forward or in one direction, the wires cut the lead, as shown for example in FIG. 36c.

In some embodiments, another mechanism of a lead cutter accessory comprises a wider device 338 that goes around the extractor, as shown for example in FIG. 36d. In this embodiment, a wire-like 338 is shown to exit from an external additional elongated tube running parallel to the LE device. In some embodiments, the wire-like is made, for example, of nitinol or any other material. In some embodiments, the wire is in a non-deployed state hugging the LE device. In some embodiments, a dedicated groove is used to keep the wire in its non-deployed state. In some embodiments, the groove is perpendicular to the LE device. In some embodiments, the groove is non-perpendicular to the LE device, having a diagonal orientation. In some embodiments, the wire “natural” memory state is in an opposite orientation related to the non-deployed state. This means that, once deployed, the wire will try to return to the “natural” memory state, which is moving apart from the LE device, as shown in the right upper corner of FIG. 36d. In some embodiments, the external additional elongated tube running parallel to the LE device and containing the wire is irreversibly attached to the LE device. In some embodiments, the external additional elongated tube running parallel to the LE device and containing the wire is reversibly attached to the LE device. In some embodiments, the external additional elongated tube running parallel to the LE device and containing the wire is adapted to move forward and backwards in relation to the LE device.

In some embodiments, another mechanism of a lead cutter accessory comprises a wider device 340 that goes around the extractor, as shown for example in FIG. 36e. In this embodiment, the wider device comprises a shutter or band 340 attached to the distal end at two points. In some embodiments, when activated, the band moves distally towards the distal end pushing the lead towards the cutting blades of the tissue cutter, until the pressure is enough to hold the lead against the blades. In some embodiments, the lead is cut by pulling the lead itself. In some embodiments, the lead is cut by the pressure applied by the band against the lead on the blades.

In some embodiments, the lead cutter comprises a linear cutting mechanism, as shown for example in FIG. 36f. In some embodiments, this mechanism comprises a channel and a side looking window through which the lead enters. In some embodiments, the sliding cutting part is configured with a cutting angle. In some embodiments, the fixed cutting part is configured with a cutting angle. In some embodiments, the internal part is the movable part in the cutting action. In some embodiments, the external part is the movable part in the cutting action. In some embodiments, the cutting mechanism is a screw rotating mechanism, as shown for example in FIG. 36f, upper figures. In some embodiments, the screw mechanism provides further force to the cutting action.

In some embodiments, the specialized groove is located near the distal end of the lead cutter accessory, as shown for example in FIG. 36g. In some embodiments, the groove enables the movement of the accessory without damaging the lead or any tissue on its way to the cutting site. In some embodiments, as shown in FIG. 36g, once the lead cutter accessory arrives at the cutting site, the lead in maneuvered into the groove where similar mechanisms to those explained in FIG. 36f are activated to cut the lead.

In some embodiments, the lead cutter comprises a shutter blades mechanism. This mechanism comprises a several blades configured to produce a round shape that closes against the lead and cut it.

### 12.7 Exemplary Pulling Rod Accessory

In some embodiments, an atraumatic pulling rod is used to catch a loose lead, from one or more of the internal lumen of the device, from the lumen of the blood vessel distally to the device, as shown for example in FIG. 36h. In some embodiments, the pulling rod is inserted within the lead extraction device. In some embodiments, the pulling rod is inserted between the external sheath and the outer shaft of the device. In some embodiments, other catching and/or holding devices can be used and inserted in the device to capture lead and/or other materials located inside the blood vein.

Therefore, in some embodiments, a pulling rod is used together with the lead extraction device. In some embodiments, the pulling rod is used as a lead-wire extender for convenient pulling. For example, the pulling rod has a round snap, which is a-traumatic and does not have sharp edges, therefore, it can pass through the inner lumen of the extraction device while the device is activated and the inner lumen rotates. In some embodiments, the snap allows easy connection to pulling wires or suture wires of the lead. In some embodiments, the pulling rod and snap allows use of short or long locking stylets, without a need to extend them separately.

In some embodiments, the pulling rod is used as a stylet within the lumen of the lead extraction device while the lead is within the lumen. In some embodiments, the pulling rod is used to release and/or fix conditions in which the lead and adhesive tissue might be stuck or form high friction. In some embodiments, the pulling rod may be pushed forward all the way to the tip of the device, to assist from within in clearing the entrance of the device and handle “snow-plow effect” of bulky tissue or materials at the entrance of the device. In some embodiments, the rod may be even longer, with ability to capture, for example by snap, a loose end of a lead in order to pull it into the lumen of the device.

### 13. Exemplary Methods

In some embodiments, the device is configured to be operated using only two hands, without the need of another user to assist (therefore requiring “more hands” to finish the procedure). In some embodiments, the user can hold different parts of the device in order to operate the device successfully. In some embodiments, the user holds the lead and/or wire located behind the handle and the other hand holds the shaft. In this embodiment, the handle is not held. The mechanisms that allow the correct functioning of the device is the weight of the handle, which is lower than 300 grams, preferably lower than 200 grams. In some embodiments, the weight of the handle is more than 300 gr. In some embodiments, the weight of the handle is from about 300 gr to about 400 grams, optionally from about 250 gr to about 450 gr, optionally from about 200 gr to about 500 gr. In some embodiments, the weight of the handle is 290 gr±30 gr. In some embodiments, the weight of the handle is measured without the alimentation cord. In some embodiments, the weight of the handle with the alimentation cord is from about 550 gr to about 650 gr, optionally from about 500 gr to about 700 gr, optionally from about 450 gr to about 750 gr, for example 600 gr.

In some embodiments, the handle comprises a 4 m±0.2 m alimentation cord (cable) un-detachable with a connector at the tip. In some embodiments, the foot pedal unit has a 10-pin push-pull connector to match the handle (with cable) connector.

Furthermore, the handle is configured to stop any rotational momentum due to the actions of internal mechanisms. In some embodiments, the user holds the handle with one hand and the shaft with the other hand. The mechanisms that allow the correct functioning of the device is the lead tension mechanism, which pulls the lead proximally, according to the activation of the lead tension mechanism by the user. In some embodiments, the user holds the handle with one hand and the wire and/or lead with the other hand. The mechanisms that allow the correct functioning of the device is the mechanism that allows to change the rigidity of the shaft. The user changes the rigidity of the shaft, thereby allowing the user to not hold the shaft. In some embodiments, the user hold the handle with one hand while the other hand is free. The mechanisms that allow the correct functioning of the device are the lead tension mechanism and the mechanism that allows to change the rigidity of the shaft.

In some embodiments, during the use of the device, one hand pulls the lead or the locking stylet and the other hand will be on the shaft to push the device as needed. In some embodiments, during the use of the device, one hand will pull the lead or the locking stylet and the other hand will be on the handle to push the device as needed. In some embodiments, during the use of the device, one hand will be on the handle to push the device as needed and an automated controlled lead tensioning mechanism 150, as shown for example in FIG. 15a, will pull the lead or locking stylet.

In some embodiments, the following methodology is performed when using a lead extraction device, for example, as disclose above:

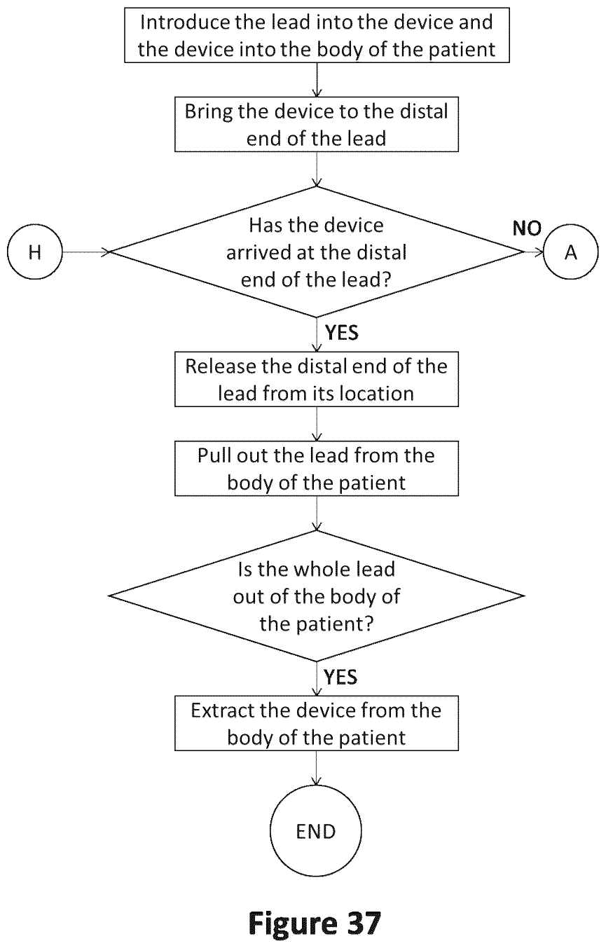

Reference is now made to the flowchart shown in FIG. 37: once the leads are exposed from the chest cavity, the user inserts the lead inside the device, and then the device inside the body of the patient. In the ideal conditions, the user is able to bring the distal end of the device to the distal end of the lead, near its place in the heart. Then the user detaches the lead from the heart, extracts the whole lead through the device outside the body of the patient. The procedure ends by extracting the device from the body of the patient.

As explained above, ideal conditions are difficult to find, especially in patients where the lead has been inside the patient for more than six months. In these cases, lead is usually entrapped by tissue.

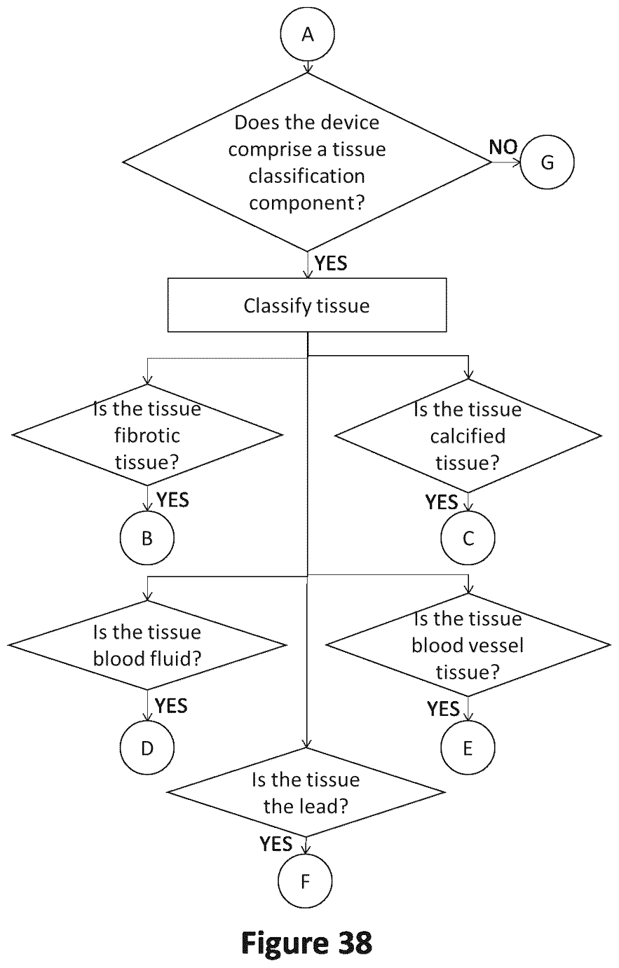

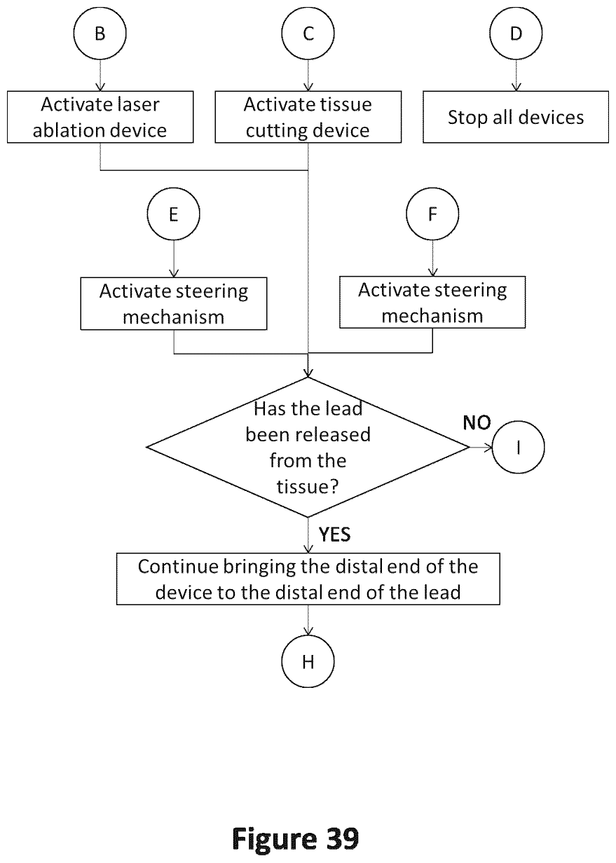

Therefore, once the user feels that the device cannot continue to follow the lead (following the letter “A” to FIG. 38), the user optionally activates the tissue classification component, if the device is equipped with one. The component can identify what kind of tissue is found in front of the distal end of the device. In the case where fibrotic tissue is found (following the letter “B” to FIG. 39), the user can choose, in some embodiments, to activate the laser ablation device. Optionally, he can also choose to activate the tissue cutting device. In the case where calcified tissue is found (following the letter “C” to FIG. 39), the user can choose, in some embodiments, to activate the tissue cutting device. Optionally, it can also choose to activate the laser ablation device. In the case where blood fluid is found (following the letter “D” to FIG. 39), the user can choose to stop all devices and assess if damage was caused to the blood vessel. In the case where blood vessel tissue is found (following the letter “E” to FIG. 39), the user can choose, in some embodiments, to activate the steering mechanism in order to direct the device towards the path of the lead. In some cases, the tissue classification component can identify that is the lead in front of the device and that is the lead itself that is not allowing the device to continue. In this case (following the letter “F” to FIG. 39), the user can choose, in some embodiments, to activate the steering mechanism in order to align the device in the direction of the lead, centralizing the lead, as much as possible, within the device to avoid damaging the lead. In any of the above cases, once the lead is released from the tissue, the user will continue bringing the device to the distal end of the lead to release it (following the letter “H” to FIG. 37).

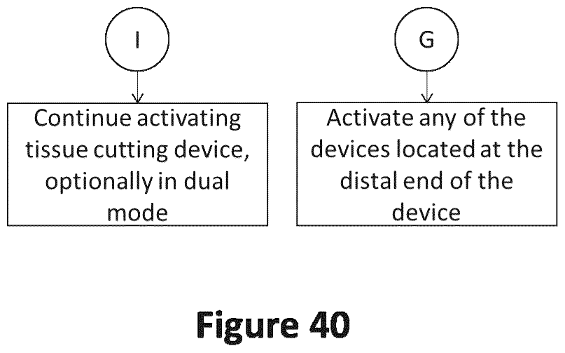

In some embodiments, where the lead is strongly buried in the tissue, the user can choose (following the letter “I” to FIG. 40), in some embodiments, to activate from the handle the dual cutting mechanism comprising linear cutting movement and circular cutting movement, including the “hammer-like” strikes, on the tissue.

In some embodiments, where the device is not equipped with a tissue classification component (following the letter “G” to FIG. 40), the user can choose to activate from the handle any of the components located in the distal end of the device, for example the tissue spreaders, the tissue cutting devices, the laser ablation device, and any combination thereof, depending on his professional assessment of the situation. For example, switching between laser ablation and tissue cutting devices if the one in use does not provide efficient progress. In another example, the user may choose a cautious approach when in doubt, choosing a device less likely to damage the lead or blood vessel, as the situation is understood by the user.

Exemplary Diameters, Frictions, Forces, Ability to Withstand Curves and Forces while Steering

In some embodiments, the device comprises a tip, including a bending shaft (Hinge), configured to be inserted in blood vessels and perform curves, either passively or actively, in order to perform a lead extraction without forcing the user to apply substantial pulling/pushing forces, for example forces of less than 750 gr or less than 500 gr or less than 300 gr. In some embodiments, exemplary curvatures are:

The junction between left cephalic vein (through the innominate vein) to the SVC, which comprises a curvature higher than 60 degrees and up to about 90 degrees, with a bending radius of about 2-4 cm.

The junction between right cephalic vein to the SVC, which comprises a curvature higher than 60 degrees and up to about 120 degrees, with a bending radius of about 2-3 cm. The path (turn) between SVC through the RA to the RV.

### Exemplary Femoral Approach

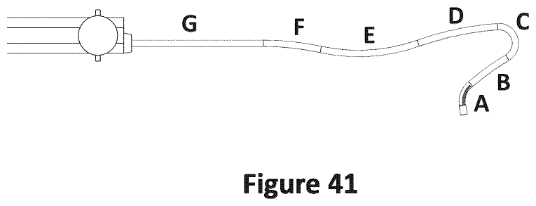

Referring now to FIG. 41, showing an exemplary embodiment of a lead extraction device configured to perform a lead extraction using a femoral approach, according to some embodiments of the invention. In some embodiments, the lead extraction procedure is performed using a femoral approach. In some embodiments, during this procedure, the device passes through the junction between femoral and iliac vein to the IVC (inferior vena cava). In that trajectory, there are curves of about 30-80 degrees (e.g. 45 degrees) while still needing to be able to maintain catheter pushability along the path towards the heart, which has a length of about 40-90 cm. In some embodiments, the device comprises a shaft having portions comprising different stiffnesses in relation to other portions within the same shaft, for example, starting from the handle, the shaft comprises a portion from about 400 mm to about 500 mm, optionally from about 300 mm to about 400 mm, optionally from about 500 mm to about 600 mm, of stiffer shaft. Optionally, it could be section G, or sections G+F, or sections G+F+E that are stiffer that the rest of the shaft (see FIG. 41). In some embodiments, the stiffness can be homogeneous along the shaft, or variable when measuring the different segments. In some embodiments, the stiffness of the shaft is measured by using a setup as shown for example in FIG. 4g, where L is from about 180 mm to about 220 mm, and the deflection is from about 70 mm to about 80 mm. In some embodiments, the sections marked in FIG. 41 as G or G+F or G+F+E will have a force of F=(40 gr to 50 gr) or (30 gr to 40 gr) or (45 gr to 60 gr) or (50 gr to 70 gr) or (60 gr to 90 gr) or any number from F=35 gr to F=100 gr.

In some embodiments the stiffest part of the shaft is followed by a less stiffer part, for example sections marked as D+C+B, and, in some embodiments, it includes the bending shaft (Hinge) marked as section A. In some embodiments, the flexible part does not includes the bending shaft (Hinge). In some embodiments, the less stiffer part comprises a length of from about 50 mm to about 230 mm, for example from about 100 mm to about 200 mm, for example about 150 mm. In some embodiments, the stiffness of the sections marked in FIG. 41 as B or B+C or B+C+D are measured by using a setup as shown for example in FIG. 4g, where L is from about 180 mm to about 220 mm, and the deflection is from about 70 mm to about 80 mm. In some embodiments, the sections will have a force of F=(15 gr to 25 gr) or F=(20 gr to 35 gr) or F=(40 gr to 50 gr) or (45 gr to 60 gr) or for example F=15 or F=20 or F=25 or any number from F=10 gr to F=60 gr. Similarly, the stiffness of the sections are measured where L is from about 90 mm to about 110 mm, and the deflection is from about 70 mm to about 80 mm. In some embodiments, the sections will have a force of F=(40 gr to 70 gr) or F=(70 gr to 100 gr) or F=(100 gr to 135 gr) or (120 gr to 150 gr) or (140 gr to 180 gr) or for example F=60 or F=80 or F=100 or F=120 or any number from F=30 gr to F=200 gr or less than 250 gr or less than 150 gr or less than 60 gr or less than 50 gr. In some embodiments, the less stiffer parts of the shaft are configured to take curves with radius as minimal as from about 3 mm to about 45 mm, for example from about 5 mm to about 10 mm, for example from about 7 mm to about 12 mm, for example from about 10 mm to about 20 mm, for example from about 15 mm to about 30 mm, for example from about 25 mm to about 40 mm, for example from about 40 mm to about 60 mm. In some embodiments, at any of the abovementioned radiuses, the device continues to work properly albeit of the bending.

In some embodiments, the bending shaft (Hinge) is able to take curves with radius as minimal as from about 3 mm to about 20 mm. Optionally, from about 5 mm to about 10 mm.

Optionally from about 7 mm to about 12 mm, as the radius of the minimal curve. In some embodiments, the bending shaft (Hinge) comprises a length of from about 30 mm to about 50 mm. Optionally, from about 40 mm to about 55 mm. Optionally, from about 50 mm to about 65 mm. Optionally, from about 65 mm to about 150 mm. For example, 55 mm, 50 mm, 40 mm. In some embodiments, the bending shaft (Hinge) is able to form bending angle of about 170 degrees. Optionally, from about 135 degrees to about 175 degrees, in one or more axis, thereby allowing the device to follow the path from the IVC into the RA, along the lead, into the RV or the CS. In some embodiments, the device will then follow the path (turn) between IVC through the RA to the RV, and then will enter to the coronary sinus, all this following the cardiac lead.

In some embodiments, the device configured for the femoral approach comprises a high flexible shaft having a one-way bending direction, for example from 0 degrees to any positive angle, for example 90 degrees. Optionally having a two-way bending direction, for example from −90 to +90. In some embodiments, the shaft also comprises one or more dimensional axis of bending, for example, X-axis bending, XY-axis bending, XYZ-axis bending. In some embodiments, a potential advantage of this is to provide a device that can follow the pathway to the room that it might be tortuous. In some embodiments, the device comprises one or more sections comprising steering capabilities.

It should be understood that all the disclosure regarding the stiffness, the forces and the utilization of the device by one user alone with relation to the short device apply the same to the above mentioned device for the femoral approach and vice versa.

In some embodiments, for any approach as disclosed above, and specifically for the femoral approach, the lead extraction device allows the use of additional instruments to be used together during the procedure. For example, the lead extraction device having an inner lumen of from about 9 F to about 20 F, for example from about 13 F to about 16 F, can used together with a Snare device, either straight snare device or needle eye snare device, of any other tool needed to assist in the extraction of the lead.

In some embodiments, the lead extraction device allows the user to follow the path of the vein and lead without pulling the lead. In some embodiments, this means that the lead extraction device, characterized by having a combination of pushability and flexibility of the tip, together with steering when needed, allows following the path even if no locking stylet is applied and/or if the lead is fragile and cannot be pulled (i.e., without basing the passage of the lead extraction device on the pulling force of the lead). In some embodiments, this is important especially when performing a in right-side approach.

In some embodiments, a hemostatic valve can be used together with the lead extraction device. For example, a hemostatic valve can be connected either to the external sheath or to the back-end of the device. In some embodiments, the hemostatic valve is a plug-in device and/or an add-on device connectable at the back-end of the device.

In some embodiments, a dilatator can be used together with the lead extraction device. For example, to assist in the entry into the femoral vein.

### Exemplary Use of the Rotational Inner Shaft to Release Leads

In some embodiments, the system is configured to utilize the rotational inner shaft with control of rotation orientation, for example Clockwise or Counter-Clockwise, for assisting in unscrewing the tip of the lead from the tissue (or the leadless pacemaker—see below), in the case of active fixation leads that have corkscrew tips. In some embodiments, for example, using the lead wire-grasping tool (FIGS. 12a-d), the user grabs the lead and/or a distal end thereof and activates the rotational inner shaft to release it.

Exemplary Use of the Rotational Inner Shaft to Avoid Curl Leads and/or to Uncurl Leads

In some embodiments, the system is configured to use the selectable rotation and orientation for minimizing lead curling within the shaft. In some embodiments, in case of high friction within the lumen of the device the lead might curl when the shaft rotates. In some embodiments, the system is configured to allow the user to use alternating rotation and orientation to reduce lead curling. In some embodiments, additionally, selecting one directional rotation may be used to uncurl the lead in case a curling has already occurred.

### Exemplary Dimensions of Parts of the System

In some embodiments, the different parts of the device will comprise the following exemplary dimensions when a 13French device is described. It should be understood that these should not limit the scope of the invention and they are provided to allow a person having skills in the art to understand the invention.

### Exemplary Coils:

Inner coil

| Parameter | Dimension | |
|---|---|---|
| OD | 5.3 mm | (±20%) |
| ID | 4.6 mm | (±20%) |
| Length | 500 mm | (±20%) |
| Distal welding length | 2 mm | (±20%) |
| Proximal welding Length | 4 mm | (±20%) |
| Torque | 40 | N*cm |

Outer coil

| Parameter | Dimension | |
|---|---|---|
| OD | 6.3 mm | (±20%) |
| ID | 5.7 mm | (±20%) |
| Length | 450 mm | (±20%) |
| Distal welding length | 2 mm | (±20%) |
| Proximal welding Length | 4 mm | (±20%) |
| Torque | 14 | N*cm |

Inner distal coil

| Parameter | Dimension | |
|---|---|---|
| OD | 5.3 mm | (±20%) |
| ID | 4.6 mm | (±20%) |
| Length | 50 mm | (±20%) |
| Distal welding length | 2 mm | (±20%) |
| Proximal welding Length | 2 mm | (±20%) |
| Torque | 40 | N*cm |

Exemplary general parts

| | ID (mm) | OD (mm) | length (mm) | technology |
|---|---|---|---|---|
| TIP | 4.3 (13F) | 7.1 | 9 to 16 | hypo-tube laser cut/CNC SS/ |
| Mechanism | (±20%) | (±20%) | (±20%) | |
| Inner Shaft | 4.3-4.7 | 5.3 | 280-490 | coil/braid/hypo-tube laser cut |
| | (±20%) | (±20%) | (±20%) | |
| Outer Shaft | 5.7 | 7.7/6.5 | 420-480 [1100 | coil/braid/hypo-tube laser cut |
| | (±20%) | (±20%) | femoral] (±20%) | |
| Hinge | 5.68 | 6.48 | 30-80 | coil/braid/hypo-tube laser cut |
| | (±20%) | (±20%) | (±20%) | |
| Inner bending | 4.3 | 5.35 | 30-200 | coil/braid/hypo-tube laser cut |
| shaft | (±20%) | (±20%) | (±20%) | |

Exemplary distal tip parts

| | | part length for one part | | | |
|---|---|---|---|---|---|
| ID mm | OD mm | number | part name at the tip | | |
| 4.360 | 4.570 | 14 mm | inner tube | | |
| (±20%) | (±20%) | (±20%) | | | |
| 4.780 | 5.580 | 1.5 mm, 5 mm, 5 mm | ratchet | body cam | hammer |
| (±20%) | (±20%) | (±20%) | | | cam |
| 4.330 | 5.350 | 30 mm, 3 mm | bending shaft | bending | |
| (±20%) | (±20%) | (±20%) | | shaft | |
| | | | | connector | |
| 6.660 | 7.200 | 18 mm | outer tube | | |
| (±20%) | (±20%) | (±20%) | | | |
| 5.680 | 6.480 | 10 mm, 30 mm | Impact | hinge | |
| (±20%) | (±20%) | (±20%) | element | | |

### Exemplary Models

In order to assess the performance of the device of the invention, several models were created, as will be further explained below.

### Exemplary Animal Models

### Sheep:

### Preclinical Lead Extraction Model:

Implantation of at least 2 leads (>1) in the same vessel and at least 3 in total (typically 3-4) leads in a sheep for >3 months, typically >4 months, typically 4-12 months, for example 2-9 months. Unlike exiting models in which it is common to keep an animal implanted for 1-2 years, or longer, until fibrosis is formed. The purpose of using multiple leads in an implanted model is for enabling to evaluate the effects of lead-to-lead interaction in relation to fibrosis, and to possibly accelerate the formation of fibrosis in the veins. It also enables to assess improvement in the efficiency of the device by evaluating multiple extractions in the same case. It also enables to improve the efficiency of the evaluation of similar devices by shortening the time for fibrosis formation (as higher friction is hypothesized to accelerate fibrotic conditions). It also enables extraction of one lead at one occasion, while leaving the other leads for a later, thus enabling to evaluate extraction capabilities and challenges as they are formed over time in the same case/subject. Similarly, it enables to evaluate veins ability to withstand extraction after some chronic local damages or fibrosis formation due to a certain period from a prior extraction procedure.

In some embodiments, at least one lead of defibrillation lead is placed in a heart chamber. In some embodiments, at least 2 of the leads are defibrillator. In some embodiments, at least one of the defibrillator leads to be implanted in the model is a dual coil lead that has a coil segment positioned in the SVC.

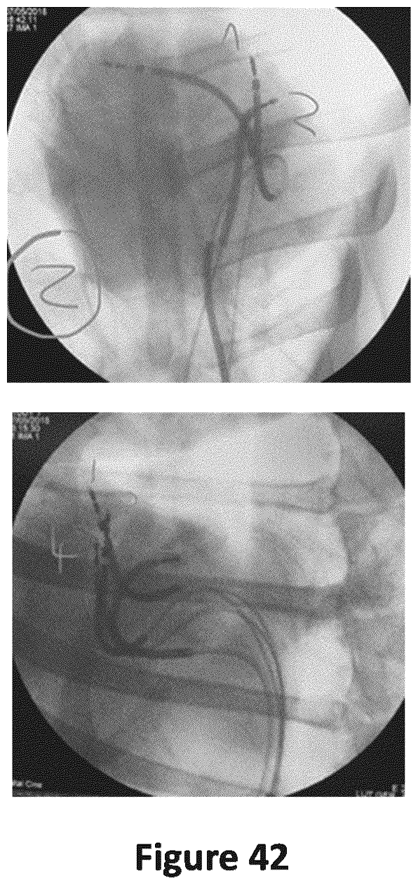

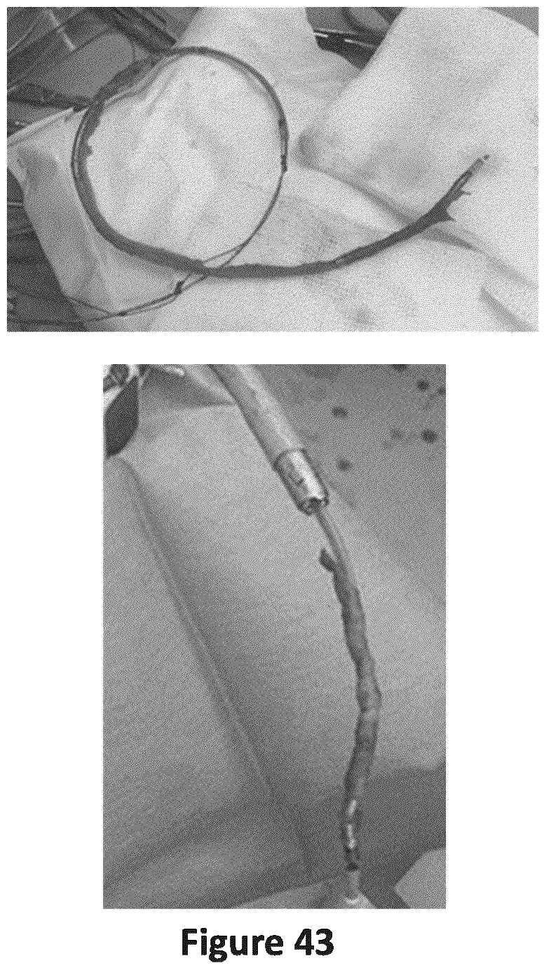

In an exemplary experiment, 4 defibrillator leads were implanted in each sheep (2 from right jugular vein and 2 from left jugular vein), as shown in FIG. 42, and kept for periods of 4-5 months. After 4 months, when extracting the leads it was clearly shown that the leads were substantially covered with fibrotic, possibly calcified, tissue, which required complex lead extraction procedure using extraction tools, as shown in FIG. 43.

### Subcutaneous Implantation in Swine

Swine subcutaneous model: one or more leads are implanted during an acute procedure, and left inside for at least 1 month, or at least 1 year to achieve a good fibrosis formation period to create a model for difficult pathways, and/or for pocket and near-bone and near-cartilage pathways, and for a lead extraction of a subcutaneous ICD lead. In an example, the lead is positioned under the skin in a curly path, or in a zig-zag path, or in a straight path. In an example, the lead is passing through tissue adjacent to the ribs. In an example, the lead is captured or sutured or otherwise affixed at its distal end either during the implant and/or during the extraction procedure, so that it enables mimicking adhesion or substantial resistance while pulling the lead or applying extraction forces or extraction tools.

### Exemplary EX-VIVO Models for Evaluating Performance of a Lead Extraction System

Referring now to FIG. 44 showing an exemplary model for evaluating performance of a lead extraction system, according to some embodiments of the invention. The model is made of a Silicone, Polyurethane or PVC (or similar stiffness or other transparent material), and represents the junction between left Cephlic vein (through the Innominate vein) to the SVC (it may be a J-shaped model, or include the connection to the upper portion of the SVC and right jugular and cephalic veins). The model has 2 segments. The first segment is a long arc or a long straight segment. The second segment is a relatively challenging angle (about 80-100 deg), turning over a path of about 4 cm and a target would be placed in its remote lateral side or inner medial side. The devices are tested by going over the lead and getting to the end without pulling the wire, or just slightly pulling to maintain wire non-curled. This model is aimed to demonstrate cases where some procedures become complex, thus evaluate the flexibility and ability to take tough curves without applying substantial forces to the vessel wall or to the lead itself, and without directly impacting or cutting (and potentially damaging) the vessel wall or the lead.

### Exemplary Model for Demonstrating Adhesion on the Inner and Outer Curves

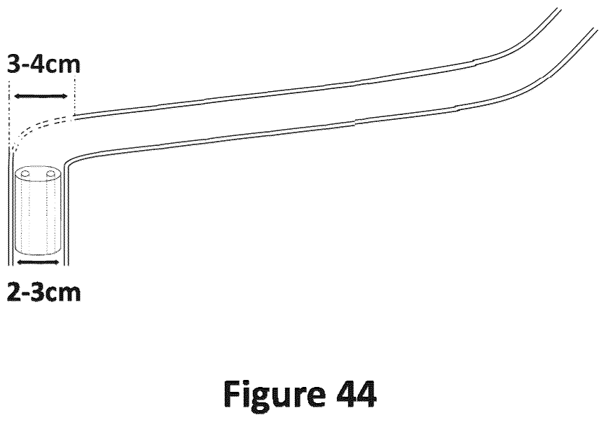

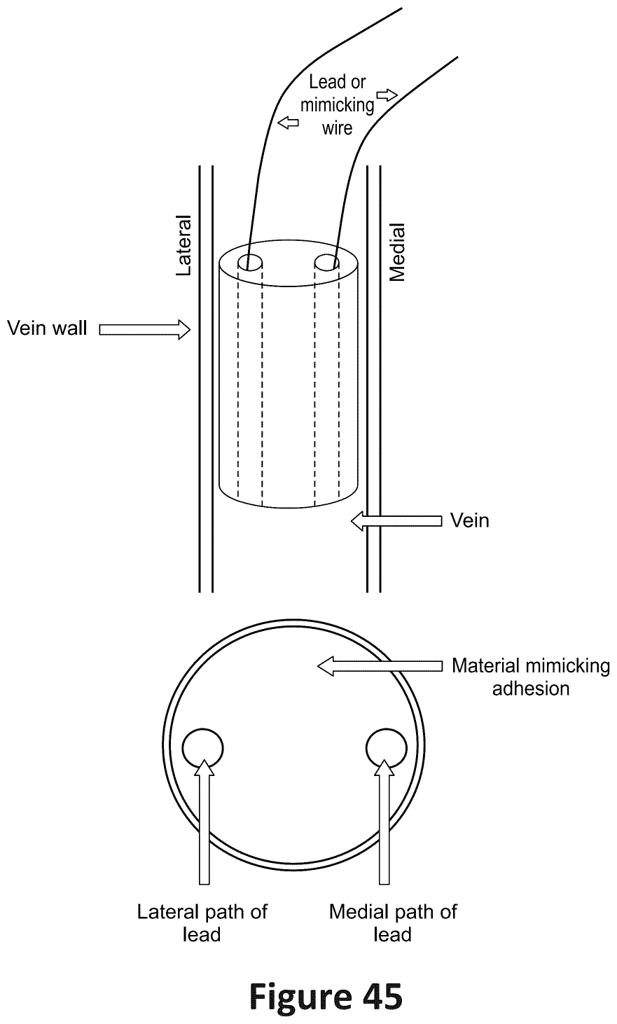

The scope of the model is to position the lead (or lead mimicking wire or illustrator) at the lateral location of the target area in order to demonstrate possible path of the leads over which the lead extraction device needs to go through (not in all cases the lead will be at the middle of the vessel in the target area). An exemplary element of the model which allows the mimicking of adhesion and positioning of a lead or lead mimicking wire in a lateral or medial path along the vein model is shown in FIGS. 45a and 45b. Setting the adhesion site of the lead laterally or medially (and not in the middle of the vein) is used to evaluate effectiveness and safety of the flexible portions of the shaft. While a straight lead extraction tools goes straight through the curved area and might cause either a cut of the lead (if the adhesion is in the inner curve of the vessel) or lead extraction tool cutting against the vein's wall, a flexible and further steerable lead extraction tool is capable of adapting the orientation to the proper direction while protecting the vessel wall and the lead, without necessarily pulling the lead and/or the vein, and without substantial push of the tool. This difference is shown in the following Figures:

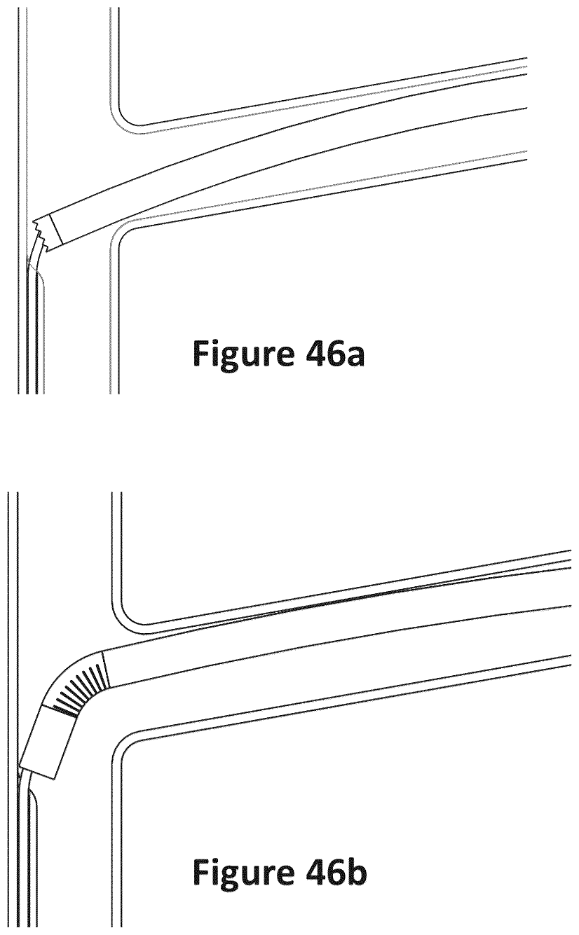

FIG. 46a: Adhesion on outer curve (lateral vein wall), a non-bending device (unless lead and vein are firmly pulled) might apply its forces and cutting to the vein wall with a risk of venous tear.

FIG. 46b: Adhesion on outer curve (lateral vein wall), a device with a flexible tip (no need for substantial pulling or any substantial tension in order to reach a desired orientation for extraction).

FIG. 46c: Adhesion on inner curve (medial vein wall), a device with flexible tip naturally fits the orientation of the lead with no (or no substantial) lead and vein tension.

FIG. 46d: Adhesion on inner curve (medial vein wall), a stiff device might either need substantial lead and vein tension to align the vein and lead to the device path (thus risk the vein and lead completeness), or apply cutting forces against the lead which might risk the lead completeness.

Exemplary materials that are used to test lead extracting in accordance with embodiments of the present invention

| Target | | Mfg. details | |
|---|---|---|---|
| Material | use | and notes | Rationale |
| Polyethylene | bench tests | Stratocell | Homogeneous and repetitive material, |
| foam | | Polyethylene | therefore the material does not add noise into |
| | | foam 16 | the experiment. This material has very little |
| | | kg/m3 | resistance and so much can be learned about |
| | | | the device. (the target length is about 80 mm) |
| iclay | demonstrations | AMOS | Homogeneous material that demonstrates |
| | and training, | Korea, SM70 | fibrotic flexible tissue. It has high friction, |
| | simulating | ITVU | which mimics high friction scenarios. In an |
| | fibrotic tissue | | example, it is being used in 5-20 mm |
| | | | thickness, for example about 10 mm |
| | | | thickness. |
| iclay mixed | demonstrations | AMOS | Demonstrated fibrotic flexible tissue, with |
| with drywall | and training, | Korea, SM70 | presence of calcium deposition. In an |
| particles | simulating | | example, mixing of approximately 80% iclay |
| | calcified tissue | | with 20% drywall is useful. In an example, |
| | | | particle of the dry wall are grained to small |
| | | | particles, for example of up to 0.3 mm, to |
| | | | form piecewise homogenous texture. |
| Acrylic glue | bench test vs | Soudal | This is a white matter which is useful for |
| | laser devices | plasto-elastic | evaluating cases of tough adhesive target, in |
| | | waterborne | which mechanical tools are typically usually |
| | | acrylic | needed (as laser energy is typically not very |
| | | sealant | effective in such matter). In an example, this |
| | | | material can be useful for reproducible and |
| | | | comparative testing of tough and |
| | | | homogeneous target. |
| Acrylic glue | bench test vs | Soudal | A white matter which is useful for evaluating |
| mixed with | laser devices | plasto-elastic | cases of tough adhesive target, in which |
| boiled | | waterborne | mechanical tools are typically usually |
| eggshell | | acrylic | needed with calcium; approximately 80% |
| particles | | sealant | iclay with 20% eggshell is useful. In an |
| | | | example, particle of the dry wall are grained |
| | | | to small particles, for example of up to |
| | | | 1.5 mm, to form piecewise homogenous |
| | | | texture. Resived very goodfidfack for its |
| | | | similarity to the human fibrotic fiber tissue. |
| | | | This is a mixture with organic material. |
| Acrylic glue | bench test vs | Soudal | A white matter which is useful for evaluating |
| mixed with | laser devices | plasto-elastic | cases of tough adhesive target, in which |
| drywall | | waterborne | mechanical tools are typically usually |
| particles | | acrylic | needed with calcium; approximately 80% |
| | | sealant | iclay with 20% drywall is useful. In an |
| | | | example, particle of the dry wall are grained |
| | | | to small particles, for example of up to |
| | | | 0.3 mm, to form piecewise homogenous |
| | | | texture. material with synthetic addition, |
| | | | easier to manufacture |
| Cubic dry | bench test vs | HW store | A homogenous synthetic matter which is |
| wall | laser devices | | useful for evaluating cases of tough adhesive |
| | | | target, in which mechanical tools are |
| | | | typically usually needed with calcium |
| Boiled | bench test vs | | Organic material that demonstrates calcium |
| eggshell | laser devices | | tissue |
| Green Apple | demonstrations | | Organic; Designed for demonstration; Not |
| | and marketing | | homogeneous |
| Turkey | simulating | | organic. Fibrous tissue is stiff and less |
| breast | fibrotic tissue | | tearing than the chicken. More homogeneous |
| (fresh) | | | because the same large tissue can be used in |
| | | | many experiments, organic. Demonstrate a |
| | | | very soft, easily ruptured fibrotic tissue; Less |
| | | | homogeneous since each chicken breast can |
| | | | only be used for a single experiment |
| Chicken | simulating | | Organic. A soft portion that simulates a very |
| breast | fibrotic tissue | | soft, easily ruptured fibrotic tissue; Less |
| (fresh) | | | homogeneous since each chicken breast can |
| | | | only be used for a single experiment |
| Rump top | simulating | | Tissue that sticks very well to leads; Solid |
| beef meat | fibrotic tissue | | meat, dry (slightly fat) and therefore stick |
| (fresh) | | | very well to the lead and to itself. Simulates |
| | | | organic fibrotic tissue |
| Beef | simulating | | Hard organic fibrotic tissue with a large |
| Achilles | fibrotic/calcified | | amount of collagen |
| Tendon | challenging | | |
| (fresh) | tissue | | |

### Exemplary Button Operations

### Exemplary Footswitch Activation Button

In some embodiments, when pressing the footswitch activation button, the device's motor is activated (optionally only) after making a beep sound and a performing a short delay. In some embodiments, optionally it then starts a 12 sec timer as a running period.

In some embodiments, when pressing the handle activation button, the device makes a beep sound and then waits a predefined period of time (for example between 2 to 5 secs) then it starts the motor, optionally it also starts to run a 12 sec timer on the background before automatic stopping.

In some embodiments, the device auto stops after 12 sec. In some embodiments, re-pressing the activation button on handle or footswitch reset timer.

In some embodiments, the device applies a short delay for predefined time, then performs a beep sound before motor starts turning when pressing the activation button on handle or footswitch.

In some embodiments, by pressing MODE Button, the device changes a select mode. In some embodiments, any press shall cycle between three motor states: MODE 1—motor will turn Clockwise; MODE 2—motor will turn Counterclockwise; MODE 3—motor will toggle motor direction (default).

In some embodiments, by pressing the SPEED Button, the Device shall toggle between two states—MOTOR speed HIGH/LOW. Speed button toggles between full speed according to PRD or 70% [270 RPM] for high speed and 60% [230 RPM] for low speed. Speed will be controlled by adjusting the PWM signal (Pulse Width Modulation).

In some embodiments, by pressing the ENABLE Button, the Device Shall toggle between two states—Disable/Activate Handle button to run motor.

In some embodiments, after 12 Sec termination there is a Wait of 2 sec.

In some embodiments, after 20 min of activation (motor runs total of 20 min) an indication of sound and LED will appear. Optionally, it will repeat for the user every 2 min.

In some embodiments, high current of motor will indicate of high forces by user. In some embodiments, the motor stops if current is above 1.1 A. In some embodiments, additionally a buzzer 3 BEEPS will be sounded and wait 2 Sec before allowing next activation. In some embodiments, this method (algorithm) potentially helps give feedback to the user when using high pulling forces or high pushing forces, in some case low radius of shaft combined with high forces. In some embodiments, a buzzer is located in the handle, the pedal or both. In some embodiments, LED indicators are located on the handle, the pedal or both. In some embodiments, the system comprises a dedicated software configured to monitor the correct functioning of the parts of the device, for example, monitoring the motor current and the predetermined motor activation limits.

Exemplary Information about the Battery

In some embodiments, the controller checks the battery state every 10 seconds. In some embodiments, at battery voltage lower than 25%, the controller triggers 4 Beeps and blink pedal orange LED. In some embodiments, at battery voltage lower than 15%, the controller triggers 4 Beeps every timeout or stop activation, and no less than 60 sec from last beeps. In some embodiments, at battery voltage lower than 10%, the controller enters error state while pedal red LED light and 4 error beeps triggered every 5 seconds. In some embodiments, the controller checks the battery state at startup.

In some embodiments, the battery is a rechargeable battery. In some embodiments, the battery is a reusable battery. In some embodiments, the battery is positioned in a location that is easy to access and replace it, if necessary.

### Exemplary Stop Activation by User or by Internal Process

In some embodiments, when device in impact mode turn the motor to opposite direction (dilation mode) so the impact ring will be distal to the knife and will “cover” the knife.

In some embodiments, the controller checks the last motor direction. In some embodiments, this is designed to make sure that there are the same number of turns in each direction even if the user stops in the middle of a certain direction. In some embodiments, the device is configured to change direction each time it stops so that at the end of the procedure there was not too much rotation in the same direction.

In some embodiments, if CW, then the controller turns the motor CCW for 450 mSec of rise and another 450 ms, always in HIGH speed, to advance the impact ring over the Blade.

In some embodiments, turn CCW after CW will take place even if the motor start the rise of the CCW and disabled only after the rise of the CCW

In some embodiments, if current is below 0.2 A when motor is in running mode save—error. In some embodiments, this shows that the current or forces are not too weak, protects the risk of disconnection or its power is too weak in the cave, used for failure detection algorithms.

### Exemplary Motor Profile

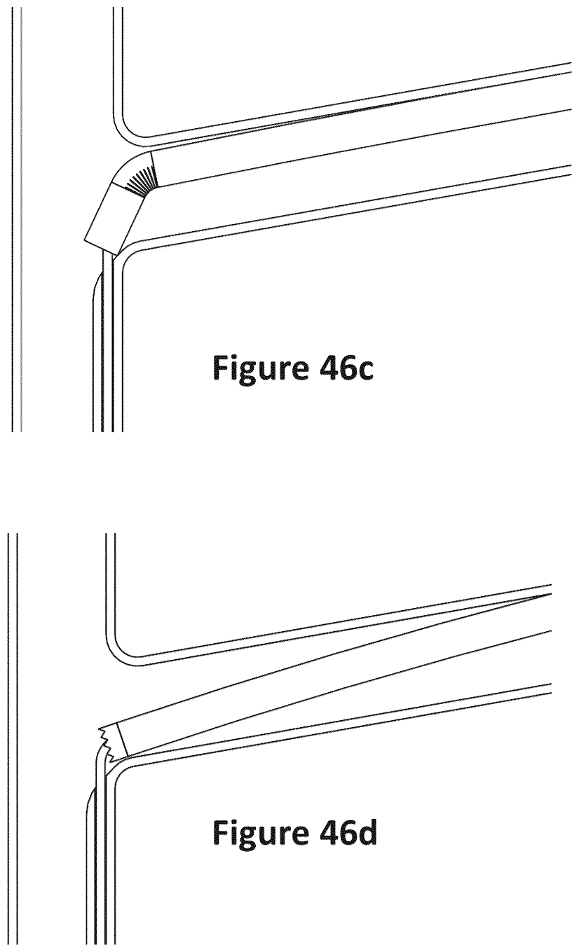

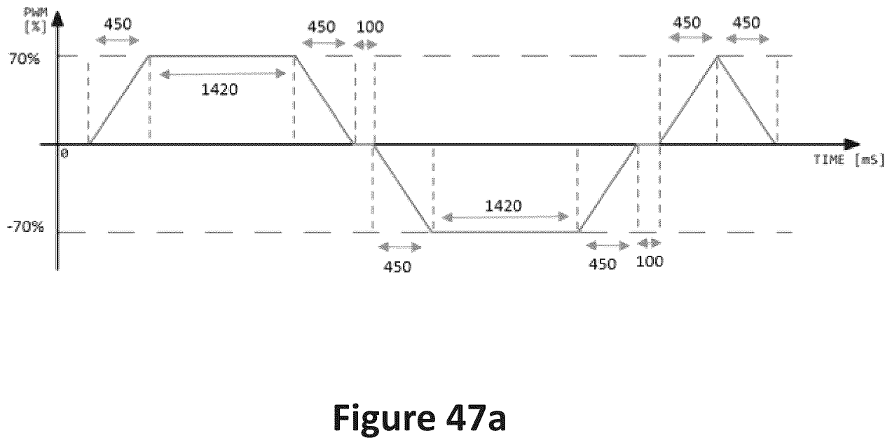

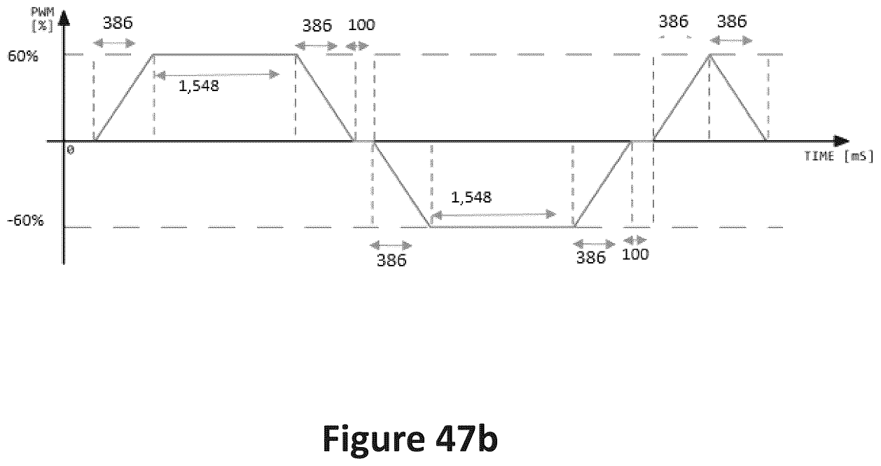

Referring now to FIGS. 47a and 47b, showing graphs presenting exemplary motor activation profiles, according to some embodiments of the invention. In some embodiments, the system is configured to maintain performance and durability of the operation of the motor by monitoring and/or controlling the current provided to the motor. In some embodiments, additionally, the system is configured to perform continuous measurement of loads applied to the motor, which are coupled to real-time alerts (visual, sound or both) and/or amendments in the motor activation parameters (for example: pause, stop, star, slow down, change voltage profile, etc.), and when the measured load exceeds (either above or below) certain predetermined parameters.

In some embodiments, the system is configured and programmed to manage the motor-operating regime in order to avoid sharp shocks and transitions. In some embodiments, in order to avoid said sharp shocks and/or sharp transitions, the system is programmed to perform one or more of the following exemplary actions:

1. In some embodiments, the system is programmed to reach a desired motor speed in a gradual manner. For example, the motor will gradually increase the current and load over a predetermined period of time, for example over a period of time of 450 mSec, until reaching a designated speed, as shown for example in FIG. 47a. In some embodiments, the time window for increasing the current and load is from about 300 mSec to about 500 mSec. Optionally from about 200 mSec to about 750 mSec. Optionally from about 100 mSec to about 1000 mSec.

In some embodiments, during the gradual increase the system monitors that there is no deviation from a predetermined current/range/profile, for example a predetermined or programmable current threshold. In an exemplary motor, a current threshold of about 1.1 ampere may correspond to a torque of about 9N*cm. Optionally, the monitoring of deviations is performed during the entire time that the motor is operated. In some embodiments, the predetermined current from which the motor should not deviate, or at least the measurements show no deviation from, is a current of from about 0.8 ampere to about 1.3 ampere. Optionally from about 0.6 ampere to about 1.5 ampere. Optionally from about 0.3 ampere to about 3 ampere. In some embodiments, a motor high current value is from about 1000 mA to about 3000 mA, for example 1100 mA, 1150 mA, 1200 mA, 1250 mA, 1300 mA, 1500 mA, 1700 mA, 1900 mA, 2000 mA. In some embodiments, optionally, the motor of the system is configured to provide, for example, 4 N*cm torque at 500 mA, optionally 6 N*cm at 750 mA, optionally 9 N*cm at 1000 mA, optionally 9.5 N*cm at 1100 mA, optionally 12 N*cm at 1280 mA, optionally 15 N*cm at 1500 mA, and/or optionally 20 N*cm at 2000 mA.

The graph in FIG. 47a shows that the motor maintains the desired speed for 1420 mSec. This is an exemplary time window of activation of the motor provided to allow a person having skills in the art to understand the invention, other times can be applied, for example below 1420 mSec, above 1420 mSec. For example from about 1000 mSec to about 2000 mSec. Optionally from about 750 mSec to about 2500 mSec. Optionally from about 500 mSec to about 4000 mSec.

2. In some embodiments, the system is programmed to decrease the motor speed in a gradual manner. For example, the motor will gradually decrease the current and load over a predetermined period of time, for example over a period of time of 450 mSec, until reaching a zero speed, as shown for example in FIG. 47a. In some embodiments, the time window for decreasing the current and load is from about 300 mSec to about 500 mSec. Optionally from about 200 mSec to about 750 mSec. Optionally from about 100 mSec to about 1000 mSec. In some embodiments, the decrease is performed until reaching a full stop of the motor.

3. In some embodiments, the system is configured to perform a standby time before performing the next activation of the motor. FIG. 47a shows an exemplary standby time of 100 mSec. In some embodiments, the standby time window is from about 75 mSec to about 150 mSec. Optionally from about 50 mSec to about 200 mSec. Optionally from about 25 mSec to about 500 mSec.

In some embodiments, optionally: in order to change the rotational speed of the motor or in order to save motor speed when reached to “high Current limit”, the device is configured to not allow an immediate stop of the motor. For example, the motor will continue to work (does not stop immediately), and it will slow down as long as there is a torque/friction sensed by the measured current. For example, about 4 Vdc will give less than 1 A. In some embodiments, for example, motor activation profile (similar to those shown in FIGS. 47a and 47b), when looking at time line will stay the same (450 mSec-200 mSec-450 mSec-100 mSec). In some embodiments, this actuation profile is similar to that of an electric screwdriver that tries to keep turning when it has resistance, but does not actually rotate.

In some embodiments, the motor is configured to provide a speed of from about 1 Hz (or revolutions per second (rps)) to about 5 Hz. Optionally from about 0.5 Hz to about 10 Hz. Optionally from about 0.1 Hz to about 50 Hz.

4. In some embodiments, the system is configured to begin a new cycle of activation after the standby period. In some embodiments, the reactivation is performed by rotating the motor to the same direction. In some embodiments, the reactivation is performed by rotating the motor to the opposite direction in relation to the previous activation.

In some embodiments, the system is configured to periodically change the polarity of the operation of the motor, which changes the direction of the rotation of the motor.

In some embodiments, the device is configured to actuate the motor in a “combined mode”, which means that the device actuates the linear impact mechanisms and the rotational cutting mechanism. In some embodiments, optionally, when high current is reached a change in the direction of the motor is performed automatically, and optionally, while avoiding sudden full stop, for example, by reducing speed gradually, for example, having a 450 mSec fall time or 400 mSec or 200 mSec or 100 mSec or 0 mS fall time.

### Exemplary Controlling and Monitoring Parameters

In some embodiments, as mentioned above, the system is configured to perform monitoring, optionally continuous monitoring, of the performance of the motor. For example, the system performs monitoring by applying real-time control and monitoring of the current. In some embodiments, monitoring comprises one or more of: measuring current; receiving feedback on gravity, thrust, torque and engine load;

In some embodiments, controlling comprises voltage control. For example, mandating the system to provide a specific waveform current/voltage that is desired. Additionally, in some embodiments, the system is configured to automatically stop the motor in case the measured parameters exceed (either above or below) a predetermined threshold, and optionally, provide an alarm to the user (sound, visual or both). In some embodiments, controlling comprises performing an automated pause at determined time-periods, for example, every 10 seconds, or 12 seconds or 15 seconds. In some embodiments, a potential advantage of provided automated predetermined pauses is that it provides the user with an opportunity to review the situation and optionally reposition and advance again.

In some embodiments, controlling comprises performing an automated stop and alert the user that the motor has been activated beyond a cumulative motor-operation time (set in advance). In some embodiments, examples of cumulative motor-operation time are for example 15 min or 20 min or 25 min or 30 min. In some embodiments, performing an automated stop is done when an integral of current/accumulated load surpasses a predetermined value.

In some embodiments, to calculate the high current, the algorithm comprises instructions to sample the current, for example, every 10 mSec; and after each 10 samples, the system will calculate an average. In some embodiments, the samplings and/or the results of the averaging of the sampling are saved in an internal memory log. In some embodiments, the data that is saved in the log is the averages alone. In some embodiments, the measured current is a running average consisting of 10 samples that are saved every 100 mSec. In some embodiments, the decision to stop (either suddenly complete stop or gradual stop) are performed every 10 mSec. It should be understood that the timings of performing the sampling, the number of samplings used to make an average, the total of averages used to make a decision to stop, could be different from those stated above. For example, average will be calculated over 200 mSec using 20 samples, or over 300 mSec using 30 samples, and so on. In some embodiments, additionally or alternatively, a median average can be used instead of the “regular” average of the 10 samples.

It should be noted that the times provided in FIG. 47a can be changed, for example as shown in FIG. 47b, where instead of having an increment over 450 mSec, the increment is performed over 350 mSec. This is true for each of the phases in the motor profile.

Exemplary Sheath with Additional Components/Materials

In some embodiments, the sheath comprises additional components and/or materials added therein. For example, the sheath comprises one or more of antibiotics and drugs. For example, anti-thrombosis drugs, anticoagulants, etc. In some embodiments, the additional components/materials are added to a specific location in the sheath, for example, within the sheath, at the tip of the sheath, or any combination thereof. In some embodiments, the effect of the additional components and/or materials on the patient is configured to be localized to the location of the additional components and/or materials on the sheath

### Exemplary Leadless Pacemaker Removal

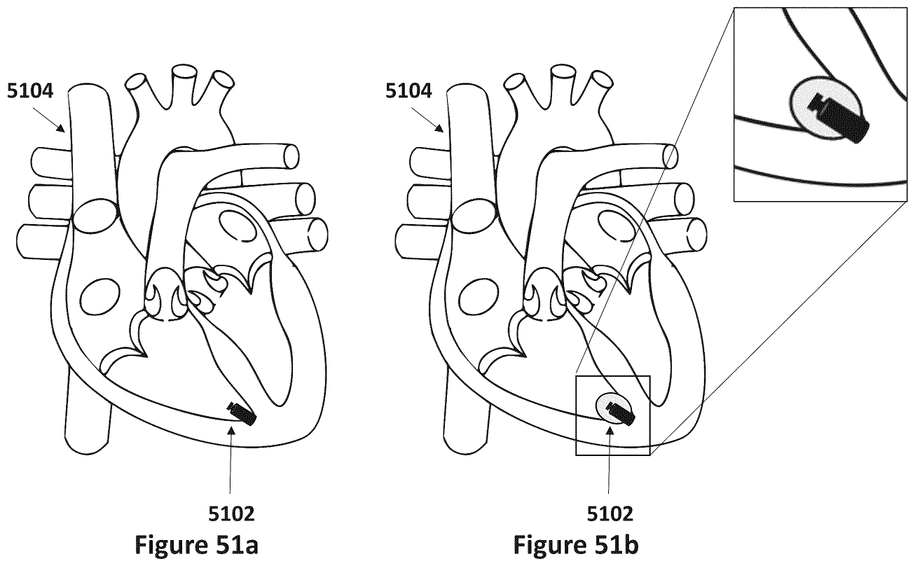

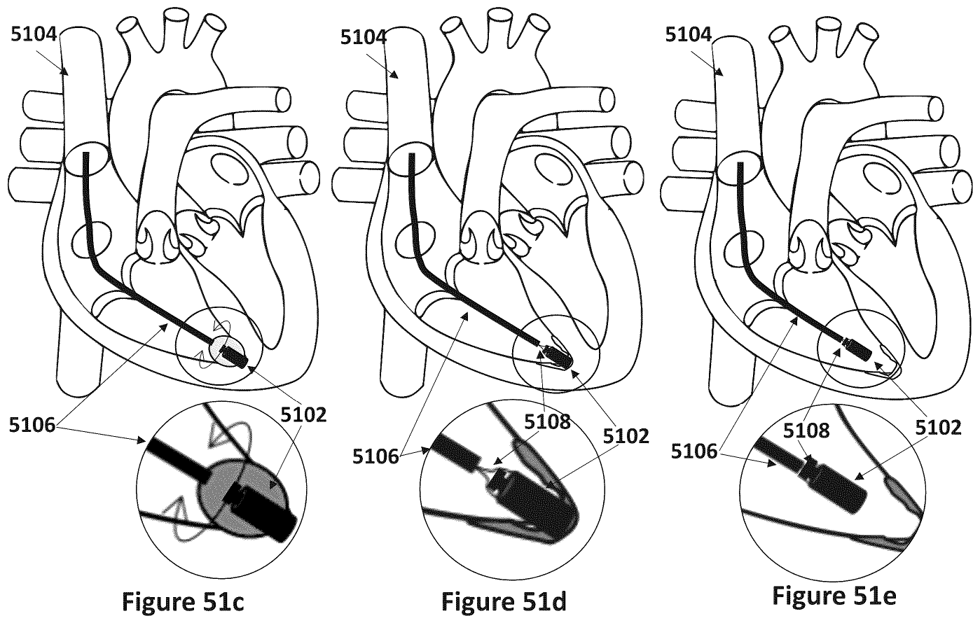

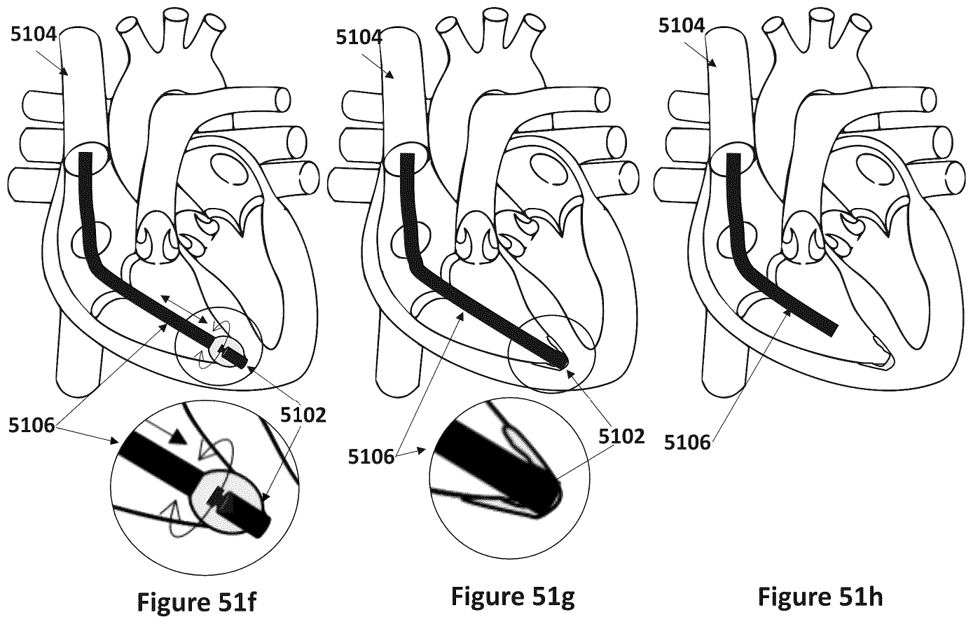

Referring now to FIGS. 51a-h, showing schematic representations of exemplary use of the lead extraction device for assisting and/or extracting leadless pacemaker, according to some embodiments of the invention. Without being bound to theory, a leadless pacemaker is a small, battery-powered device 5102, configured to send mild, painless electrical signals to the heart 5104, which potentially keep the heart beating normally, as shown for example in FIG. 51a. In some embodiments, the leadless pacemaker comprises two sides. In some embodiments, a first side is configured to be attached on the wall of the heart, which comprises one or more mechanical elements (for example one or more screws) configured to reversibly attach the leadless pacemaker to the heart's wall. In some embodiments, the other side comprises a holding element configured to allow a user to manipulate the leadless pacemaker during insertion and/or extraction. In FIGS. 51a-h, the holding element is the small head protruding from the leadless pacemaker. In some cases, the leadless pacemaker is needed to be removed and/or changed. One of the main issues with the removal of a leadless pacemaker is that the device can be encased in tissue that grew over time, making the extraction of the leadless pacemaker extremely difficult, as schematically shown for example in FIG. 51b.

In some embodiments, the lead extraction device 5106 as disclosed herein is used for the extraction and/or for assisting in the extraction of leadless pacemaker from a heart of a patient.

Exemplary Scenario 1—Diameter of the Lead Extraction Device being Smaller than the Diameter of the Leadless Pacemaker

In some embodiments, the device is brought to the location where the leadless pacemaker is located within the heart, as shown for example in FIG. 51c. In some embodiments, the cutting and/or impact tools are activated to remove the tissue that grew over the leadless pacemaker, thereby exposing the leadless pacemaker, as shown for example in FIG. 51c-51d. In some embodiments, since the diameter of the lead extraction device is smaller than the diameter of the leadless pacemaker, it would be highly possible and correct to assume that the tissue enclosing the leadless pacemaker will also be bigger than the diameter of the lead extraction device. Therefore, in some embodiments, the cutting and/or impact actions are performed on the tissue, for example, following a circular movement, as shown in FIG. 51c. In some embodiments, the cutting and/or impact action is performed until the leadless pacemaker is liberated from the tissue, as shown for example in FIG. 51d. In some embodiments, removal of the tissue is performed to expose the holding element of the leadless pacemaker. In some embodiments, it is enough just to expose the holding element in order to allow release of the leadless pacemaker from the wall of the heart. In some embodiments, additional tissue around the leadless pacemaker can be removed to further assist in the detachment of the leadless pacemaker from the wall of the heart. In some embodiments, the user then activates a grabbing tool 5108 configured to firmly hold the holding element of the leadless pacemaker, optionally rotate the leadless pacemaker to unscrew the leadless pacemaker from the heart's wall, and then extract the device from the heart, as shown for example in FIG. 51e.

Exemplary Scenario 2—Diameter of the Lead Extraction Device being Bigger than the Diameter of the Leadless Pacemaker

In some embodiments, the device is brought to the location where the leadless pacemaker is located within the heart, as shown for example in FIG. 51f. In some embodiments, the cutting and/or impact tools are activated to remove the tissue that grew over the leadless pacemaker, thereby exposing the leadless pacemaker, as shown for example in FIG. 51f-51g. In some embodiments, since the diameter of the lead extraction device is bigger than the diameter of the leadless pacemaker, the cutting and/or impact actions are performed on the tissue, for example, following a circular movement, as shown in FIG. 51f, and, once a part of the leadless pace maker is found, the device is moved straight into the grown tissue so as to insert the leadless pacemaker into the lead extraction device. In some embodiments, the cutting and/or impact action is performed until the leadless pacemaker is liberated from the tissue, as shown for example in FIG. 51g. In some embodiments, removal of the tissue is performed to expose the holding element of the leadless pacemaker. In some embodiments, it is enough just to expose the holding element in order to allow release of the leadless pacemaker from the wall of the heart. In some embodiments, additional tissue around the leadless pacemaker can be removed to further assist in the detachment of the leadless pacemaker from the wall of the heart. In some embodiments, the user then activates a grabbing tool 5108 configured to firmly hold the holding element of the leadless pacemaker, while inside the lead extraction device, and optionally rotate the leadless pacemaker to unscrew the leadless pacemaker from the heart's wall, and then extract the device from the heart, as shown for example in FIG. 51h.

Exemplary Principle of Differential Interaction with Tissue

In some embodiments, as disclosed above, the lead extraction device is provided with dedicated mechanisms to allow separation of the lead from tissue that has grown around it. In some embodiments, as mentioned above, the dedicated mechanisms actuate two distinct actions: cutting action and impact action. In some embodiments, the impact action is characterized by a forward-backward movement of an element, for example a ring, which optionally comprises a sharp edge or optionally at least part of the edge, is sharp. In some embodiments, the cutting action is characterized by a circular movement of one or more blades, optionally rotating in opposite directions. In some embodiments, as mentioned above, the impact element can also act as a protector for the blades, to avoid unnecessary expose the blades to non-target tissue (for example: the blood vessel). In some embodiments, the impact element is passively held, for example, by a spring mechanism, which allows retraction of the impact element when pressure is applied on the impact element, thereby allowing exposing of the blades. In some embodiments, as mentioned above, the impact element comprises a distal end that is perpendicular to a longitudinal axis of the device or is angled or beveled in relation to a longitudinal axis of the device.

For commodity of the explanations these two distinct actions, will be referred as “cutting” and “impact”, are characterized by having a differential interaction with the tissue.

Without being bound to any specific theory, one can assume that cutting blades could be configured in having a sharp shape and motion for effectively cutting tissue (for example, as a sharp surgical blade), while being less effective on hard matter, for example on calcified bulk of tissue. On the other hand, impact motion, even with a blunt surface, may be effective on hard matter (for example, by braking, reshaping or displacing it), while being less effective on soft matter (e.g., only temporarily elastically stretching it), for example, a blunt surface may have little cutting effect on soft stretchable tissue. In some embodiments, the lead extraction device of the present invention comprises a mechanism having dual action that provides a potential advantage of effectively, and possibly selectively, performing the right action on the right type of tissue. Known lead extractor devices include tubes (sheaths) and rotational blades that affects soft tissue. In addition, some systems include a tube (sheath) with additional energy that is provided at the tip of the device, for example by the use of lasers, however, such additional energy is useful for affecting soft tissue (which is a relatively easy task which can also be handled by means of the sheath or blades), while on the other side, do not resolve the need to affect hard tissue. Therefore, such energy-based systems provide “stronger” solution for the “easy” task (e.g. soft fibrous tissue), while not improving the solution for the “harder” task (e.g. hard calcified tissue). In contrast, a potential advantage of the lead extraction system of the present invention is the ability to provide “stronger” solution (rotating blades plus effect impact) for the “harder” task (tissue which is composite of fibrous and calcification), while remaining with “softer” solution (rotating blades) for the “easier” task (soft fibrosis). The present invention potentially provides such a differential interaction mechanism, which comprises a solution for both problems, being gentle on easier targets, and being more powerful on the harder targets.

### Exemplary Specification of the Lead Extraction Device

In some embodiments, the specifications of an exemplary lead extraction device are as follows:

In some embodiments, the following paragraphs describe and define exemplary details for the ‘lead extraction device specifications’ as described in an exemplary ‘user, customer and marketing requirements for Cardiac Lead extractor device’ hereafter ‘device’, as well as exemplary intended use and exemplary operational use of its key users. In some embodiments, it provides an exemplary framework against which the device shall be validated.

Definitions, acronyms and abbreviations

| | |
|---|---|
| SVC | Superior Vena Cava |
| OR | Operating Room |
| CIED | Cardiac Implantable Electrical Devices |
| TLE | Transvenous Lead Extraction (procedure) |
| ICD | Implantable Cardioverter Defibrillator |
| DI | Design Input |
| ID | Industrial Design |

### Exemplary Device Description

In some embodiments, the device is a single use sterilized device designed to be placed over a cardiac lead. In some embodiments, the device is designed to be an electro-mechanical motorized tool that is introduced over the lead and follows its path, using a flexible and steerable shaft connected to a control handle. In some embodiments, the proximal section of the shaft maintains pushability while the distal section is very flexible, thus to promote an easy passage through vein curves and junctions. In some embodiments, the device distal tip is designed to cut the fibrous binding tissue and engage through calcified binding sites along the implanted lead (for example by combined rotational and impact movements). In some embodiments, impact actions are used to separate/cut hard tissue, while rotation actions are used to separate/cut soft tissue. In some embodiments, the device control handle, which is connected to the shaft, contains a steering mechanism for the device maneuverability, and a motorized power transmission mechanism to activate the device tip. In some embodiments, the device is visible under fluoroscopy during the procedure.

A detailed exemplary explanation of each device component and operational information is provided below.

Steerable Shaft: In some embodiments, the shaft is the only part of the device, which is inserted into the venous system. In some embodiments, it is in direct contact with blood vessels, through which the cardiac leads are placed. In some embodiments, the distal part of the shaft provides high flexibility while maintaining a clear passage for the lead (and lead capturing accessories). In some embodiments, the shaft has a biocompatible hydrophilic coating for smooth insertion of the device through the vessels. In some embodiments, the shaft consists of two longitudinal intercalated components, and a distal tip. In some embodiments, the design of the shaft (materials and structure) the device main characteristics, such as pushability, flexibility, steerability, radiopacity and its overall performance. In some embodiments, the control mechanism at the handle intuitively reflects to the user the angulation of the distal flexible part.

Actuation tip: In some embodiments, the distal part of the shaft includes a tip, which consist of a cutting mechanism for fibrous tissue dilation and detaching calcification from the implanted lead. In some embodiments, it provides with both rotational and longitudinal impact motion. In some embodiments, impact actions are used to separate/cut hard tissue, while rotation actions are used to separate/cut soft tissue.

Control handle: In some embodiments, the device control handle is connected to the shaft at one end, and to an electric cable on the other end. In some embodiments, it contains a steering mechanism which allows maneuverability, an electronic DC motor and a transmission mechanism which delivers rotary power to the device tip, two (2) operation ON/OFF buttons, and a set of four (4) LEDs as visual indications to the device operating mode. In some embodiments, a steering rotator knob enables steering from both sides of the handle. In some embodiments, it comprises a symbol that indicates the bending direction.

Control Pedal: In some embodiments, the control pedal is a multi-use component, used for an easy foot operating control over the device. It contains the following: A rechargeable battery pack which provides energy to the device; The main electronic parts; LEDs for visual indications; A buzzer for audio indications; An ON/OFF power button (to activate by hand at the begin and at the end of procedure); Three (3) cyclic mode select buttons (activate by foot during procedure); A foot pedal for device motor activation (while stepped on the motor is activated); A dual-purpose electrical connector: to connect the device cable from the control handle to the pedal and/or to connect the charger to the pedal to recharge the battery pack.

### Exemplary Device Requirements (Device Level)

### Exemplary Materials

In some embodiments, the device materials support the lightweight and electrically isolated device qualities. In some embodiments, the device handle shells and steering material are PA2200. In some embodiments, the shaft jacket material is Pebax. In some embodiments, the device is biocompatible. In some embodiments, the elements that are in contact with the body (direct/indirect) are made of biocompatible material to match the user and the patient. In some embodiments, the materials of the handle and the shaft are made of materials that comprise a certificate of biocompatibility and pass biocompatibility tests. In some embodiments, the device is configured as a single use device. In some embodiments, the device is corrosion resistant. In some embodiments, the device's stainless steel/metal elements show no signs of corrosion throughout the lifecycle of the device. In some embodiments, the device is designed for practical pushing, controlling and navigating along the veins into the heart's apex inserted through the subclavian veins (right or left side). In some embodiments, the device is designed to match left and right-handed surgeons for controlling and navigation, as well as working from left and right side of patient. In some embodiments, the device is a steerable device with ability to steer the shaft distal segment for the device handle. In some embodiments, the device comprises a steerable shaft controlled by a handle for distal tip one-side navigation using pull wires. In some embodiments, the maximum pulling force of wires is about 6000 gr±500 gr. In some embodiments, the “pully” mechanism of the steering comprises a breaking-prevention mechanism over tension feature and the spring in the “pully” comprises a role to keep the wire from tear in a case of wire over tension. In some embodiments, the device is configured to provide the ability to control a steering mechanism from the handle. In some embodiments, the steering mechanism is located in the handle and it controls the distal segment of the device for a one-side bend. In some embodiments, the device comprises a rotating cutting head that rotate over the lead. In some embodiments, the device has a bidirectional rotating cutting blade at the “Tip” (“head”) in the distal part of the shaft. In some embodiments, the device has unidirectional rotating blade. In some embodiments, the device is configured to provide the ability to control over the inner shaft rotating speed. In some embodiments, the rotation speed-changing button is located on the pedal to changing the rotating speed of the inner shaft. In some embodiments, the device is configured to provide the ability to control on over rotating modes. In some embodiments, the pedal has a button for changing the rotating mode of the shaft affecting the blade at the Distal Tip. In some embodiments, the device (handle+shaft+tip) is single use, disposable and sterile. In some embodiments, all the components of the handle and shaft of the device are disposable for single use and compatible with ETO sterilization process. In some embodiments, the pedal is multi use unit and does not require to be sterile. In some embodiments, the pedal comprises a lifetime of more than 2 years and/or 500 battery recharge cycles.

### Exemplary Mechanical Performance

In some embodiments, the device is lightweight (handle of less than 0.6 Kg) and comprises an ergonomic shape that allows to comfortably hold by hand, work easily and fit right-handed and left-handed users. In some embodiments, the device is configured to fit right side and left side patient procedures. In some embodiments, the weight of the device without the cable is about 290±30 gr. In some embodiments, the device is configured with an ingress protection. In some embodiments, the device is protected from external fluids and materials. In some embodiments, the device is protected from blood or Contrast material, water, at any part of the device (e.g. handle, shaft or pedal). In some embodiments, the device and shaft are safe to be in contact with Contrast material, and other liquids, electronic PCB in the handle are protected, all electro-mechanic parts on handle or pedal, as user interface is waterproof. In some embodiments, the device is configured to withstand medical catheter mechanical performance requirements. In some embodiments, the device is configured to meet the requirements in terms of Strength, bending Etc.

### Exemplary Electrical Performance

In some embodiments, the device power is sufficient to cumulative work time of 30 min. In some embodiments, it is preferable that the battery would be sufficient for at least 1.5 hours by a single battery charge. In some embodiments, the battery pack capacity in the pedal unit is 33000 mAh sufficient to 3 hours working time. In some embodiments, the device pedal contains integrated power source-rechargeable battery. In some embodiments, a rechargeable 5 cell (18V) lithium ion battery is integrated in the pedal unit. In some embodiments, the device is configured to have the ability of electrical connection to pedal unit, which control over the motors and device UI. In some embodiments, the device has electric cable with 8 electric wire and connector to pedal unit that control over the motors (and feedback) of the device. In some embodiments, the device comprises a cable having a length of about 4 m. In some embodiments, the rechargeable power source for the pedal is provided—220V and 110V power source for target countries. In some embodiments, a rechargeable power source for the pedal provided from dedicated charger—220V and 110V [50-60] Hz power source for target countries, including outlet to match power inlet of destination country.

### Exemplary Functionality/Usability

In some embodiments, the device is configured to allow working as and with common existing procedures. In some embodiments, the device properties, balance and weight are configured to allow working as common existing procedures. In some embodiments, the device includes a flexible distal part to enable fitting into vein curvatures. In some embodiments, the device includes Flexible and steerable distal segment of the shaft and allow movement to single side angulation. In some embodiments, the device includes a motor for activation of the device operation on the tissue. In some embodiments, the device comprises an integrated DC motor in the device handle. In some embodiments, the device includes steering to affect the orientation of the distal part. In some embodiments, the steering mechanism at the distal segment of the shaft is operated from the handle using for example a 0.2 mm stainless steel pull wire. In some embodiments, the steering mechanism is configured to cause bending towards a certain orientation that corresponds to the handle orientation, for intuitive control. In some embodiments, the steering mechanism at the distal segment of the shaft that is operated from the handle provides a one direction bending orientation, provides a feedback to the user about the forces and the steering angle. In some embodiments, the device includes an impact force generation at the tip to improve passage through stiff binding sites. In some embodiments, the device comprises an impact force at the tip distal side due to the Tip mechanism and a spring. In some embodiments, the device is configured to provide the ability to operate of the steering and rotation power transmission button using a single hand. In some embodiments, the device has On/Off switch and steering on the Handle Intuitively to the user. In some embodiments, the device may include a pedal for enabling control of some features and/or device activation. In some embodiments, the pedal has 4 operation buttons: Activation foot switch (ON when pushed); Operation mode switch; Operation speed switch; and Enabling handle activation switch. In some embodiments, the device controls are configured to be intuitive and with operation feedback to user. In some embodiments, the device has indicator LEDs, buzzer, and icon prints, which give the use the option for ease and intuitive controlling as well as indication for operation modes. In some embodiments, a buzzer is located in the handle, the pedal or both. In some embodiments, LED indicators are located on the handle, the pedal or both. In some embodiments, the system comprises a dedicated software configured to monitor the correct functioning of the parts of the device, for example, monitoring the motor current and the predetermined motor activation limits. In some embodiments, the device is configured to potentially enable some of the tasks to be performed in some cases by a single user. The procedure may be performed in some cases by a single user (one hand holds the wire and the other slides the device over the wire, without necessarily need a second operator)—pulling the lead, In some embodiments, steering, device activation and operation mode changing are controlled by single user. In some embodiments, the locking stylet, the extension wires, and the lead are to be passed though the lumen of the device, and held the operator. In some embodiments, the puller has the ability to pass through the device from proximal side to the distal side, then connect for example easily to the locking stylet or the extension wires so it could be pulled back through the device.

In some embodiments, the device comprises additional components, for example an external sheath in the sterile package of the device. In some embodiments, the external sheath comprises a length of from about 15 cm to about 40 cm, optionally a preferred length of 25 cm. In some embodiments, the external sheath provides and includes minimal friction with the outer shaft and with the anatomy. In some embodiments, the external sheath is configured to be flexible according to the anatomy and less flexible than the shaft. In some embodiments, it is optional to add a hemostatic valve at the proximal side of the sheath, for injection of contrast material between external sheath and outer sheath. In some embodiments, the external sheath dimensions and parameters are as follows: ID 7.90+−0.05 mm; OD 9+−0.05 mm; Length 264.5+\−2 mm; Tip shape on one side: round; Tip shape on second side: ellipse; Materiel: PTFE—10-12% Bismuth; Low friction like the shaft of the device or lower and radiopaque material.

In some embodiments, the device is configured to be visible at fluoroscopy (X-ray). In some embodiments, the device is configured to be compatible with additional imaging modalities that are commonly operated during procedure: TEE, vital signs, arterial pressure, ECG, etc. In some embodiments, the shaft material meets the requirements in terms Radiopacity standard.

### Exemplary Interface Requirements

In some embodiments, the device is configured to allow to be inserted into the SVC. In some embodiments, the device is configured to have a bending radius of the distal segment 17 mm+/−3 mm. In some embodiments, the device is configured to allow to be inserted over common leads that are in use, in the range of 5 to 10 French. In some embodiments, the inner diameter of the device is 13Fr (4.3 mm+−0.1 mm). In some embodiments, the device is configured to provide an interface that is clear, simple and comprises an intuitive feedback. In some embodiments, the device comprises a plurality of feedback LED and sound of operational device, for example: LED Power condition on charger for battery feedback while charging; LED battery condition on pedal+sound for low battery power; LED on handle to show working mode selected (dilation or impact or combination); LED on handle for selected Speed mode; LED on handle for enabling the activation button on the handle; Alerts by LED and sound on handle and\or on pedal; Steering orientation by handle structure and steering knob; Printed clear labeling and icons on pedal and handle cover.

In some embodiments, the device comprises a user interface that is simple and minimal. In some embodiments, the device has ergonomic handle to match variety of users, intuitive knob for steering that can be operated optionally in during fluoroscopy, minimal mode changing are needed (if any) with LED feedback on handle. In some embodiments, the device is configured to allow free movement of steering to follow the lead, that gives the user the option to operate with less steering activation “free movement of tip” to keep trackability. In some embodiments, the pedal is used to increase the usability and different users' habits. In some embodiments, the device comprises audio alerts and LED indication for the user.

### Exemplary Device Requirements Table: Shaft Assembly

### Exemplary Configuration

In some embodiments, the device comprises a distal cutting tip blade with geometry and sharpness. In some embodiments, the device is configured with the ability of one-way directional steering of >90 deg. In some embodiments, the device comprises a steering mechanism comprising a one-way steering ability >100 deg. In some embodiments, the device is configured to supply trackability, flexibility and pushability needs to be sufficient inside the procedure anatomy. In some embodiments, the flexible distal section and proximal section are semi rigid and are divided to 4 Pebax sections. In some embodiments, the Pebax jacket lengths are for example as following: Pebax 55D—314+/−3 mm; Pebax 40D—120+/−3 mm; Pebax 35D—100+/−3 mm; Pebax 25D—40+/−3 mm. In some embodiments, the device is configured to provide the ability to transfer axial and rotational forces. In some embodiments, the device comprises a proximal semi rigid end that allows transmission of axial and torque forces.

### Exemplary Mechanical Performance

In some embodiments, the device's shaft dimensions are configured to fit to average anatomical dimensions, for example: shaft working length is about 540 mm; External diameter: 7.75 mm max; Internal Diameter: 4.3+/−0.1 mm; Distal bending section length: Pebax 35D—100+/−3 mm; Pebax 25D—40+/−3 mm. In some embodiments, the shaft and tip are designed for low friction. In some embodiments, the device comprises Hydrophilic Coating that covers the shaft. In some embodiments, the external sheath material is PTFE. In some embodiments, the device inner shaft is configured to have the ability to transfer torque for cutting tissues. In some embodiments, the device inner torque coil and/or braided shaft and/or hypotube component has torque transmission ability of more than 35 [N*cm] both directions. In some embodiments, the device comprises the following stability of bending/steering geometry: bending of shaft due to handling proximal segment or due to anatomy will not affect steering of distal tip and will not cause unintended stress on anatomy. In some embodiments, the device comprises a “Contra sleeve” for the steering mechanism. In some embodiments, the device comprises a tensile rigidity of 15 [N] and compression rigidity of 50 [N]. In some embodiments, the Shaft fixturing over the handle withstand the following tensile and compression forces: Tensile rigidity: >15 [N/mm]; compression rigidity: >50[N/mm]. In some embodiments, the device comprises a Surface finish that does not have sharp edges, bumps and has smooth finish to eliminate tissue trauma. In some embodiments, the surface is smooth with Pebax jacket over it.

### Exemplary Interface Requirements

In some embodiments, the outer shaft is configured to support the inner shaft while rotating at up to 400 RPM, CW and CCW. In some embodiments, the outer shaft inner surface, design and gap from the inner shaft, support the inner shaft while rotating at 400 RPM. In some embodiments, the tip mechanism is integrated into the shaft. In some embodiments, all moving parts in the tip are lubricated.

### Exemplary Handle Assembly

### Exemplary Configuration

In some embodiments, the device is configured to have the ability to steer the distal tip of the shaft and move freely with the hinge movement from the device handle. In some embodiments, the steering is configured to support one or more of the following steering movements: Idle Mode the distal segment is passive and free to move as head is being bend by external forces such as tissue force during devices delivery; handle's lever is manually operated to manipulate the distal segment angle; contra sleeve to support the pulling wire, that will give the option of free move of the hinge. In some embodiments, the device's features are controlled by the handle. In some embodiments, the shaft maneuvering (push/pull, torque) is performed by steering activation by lever over the handle for controlled steering and/or by motor activation. In some embodiments, the device comprises physical and visual indications for steering direction. In some embodiments, the handle section is not symmetric and comprises marking indications for shaft direction over the steering.

### Exemplary Mechanical Performance

In some embodiments, the device is small, lightweight and has an ergonomic shape that allows to comfortably hold it by hand, work easily and safely. In some embodiments, exemplary sizes of the device are: Total Length: 792 mm max (handle & shaft); Max width: 62 mm. In some embodiments, exemplary weights of the device are: 290 gr+/−30 gr (not including cable); 515 gr+/−50 gr (including cable). In some embodiments, the motor unit is located inside the handle. In some embodiments, the motor is located inside handle and electrically connected to control unit (pedal).

### Exemplary Electrical Performance

In some embodiments, the device comprises sufficient motor power transmission for the tip mechanism. In some embodiments, the motor power is about 11.4 W. In some embodiments, the handle comprises 2 symmetric operation on/off switches to activate the motor. In some embodiments, the device is configured to have the ability of electrical connection to the pedal unit. In some embodiments, the device comprises one cable connector 2+8 conductors wire (22 AWG+28 AWG). In some embodiments, the device comprise a cable having a length of about 4-meter.

### Device Requirements Table: Pedal

### Exemplary Configuration

In some embodiments, the pedal comprises a main motor operation on\off switch. In some embodiments, the pedal comprises a Speed mode selection button. In some embodiments, the pedal unit has momentary button that user can push to change by foot and change the motor speed to produce nominal tip speed between High: 235 RPM+−20 PRM and Low: 200 RPM+−20 RPM. In some embodiments, the pedal comprises a device operation mode button. In some embodiments, the pedal comprises a handle on/off switch enable switch button. In some embodiments, the pedal is configured to be covered during the procedure for keeping it clean. In some embodiments, the pedal-device connection located in the rear of the pedal that able to cover the pedal during the procedure.

### Exemplary Device Description

In some embodiments, the Lead Extraction device is an intra-operative device designed for percutaneous dilation of tissue surrounding leads during transvenous lead extraction. The lead extraction device is operated by the physician, by holding and advancing it with one hand and gently pulling (applying minimal traction to) the electrode lead with the other hand. Its unique features enable the device to potentially advance over the lead through the vasculature with relatively low forces, while dilating the surrounding tissue and facilitating the removal of the lead.

In some embodiments, the device consists of a sterile single use component, which is partially inserted intravenous and is visible under fluoroscopy.

In some embodiments, the lead extraction single use unit has a motorized handle, having a steering control mechanism and a tip mechanism for tissue dilation. The unit is designed for use with potentially low pushing and pulling forces and includes a steerable shaft to assist in following the lead path with minimal tension.

In some embodiments, the unit includes a steering knob for assisting in orienting the tip, and an activation button on each side of the handle, for activating the motor. The steering is one directional, to enable straightening of the tip orientation or bending the flexible part towards a predefined orientation as marked on the handle. The handle's activation buttons may be enabled or disabled. The handle includes LED indicators related to the device mode. A detailed description of the device buttons and LED indicators is provided in the “Exemplary Button Operations” section.

In some embodiments, the unit includes a flexible outer shaft and an inner shaft, having a 13 Fr lumen for passage of a pacemaker/defibrillator lead. The shafts are connected to a control handle having a miniature motor, capable of mechanically passing motion through the inner shaft to a tip mechanism. The tip mechanism is designed to provide dilation/rotating motion (by a ring that rotates within the shaft's lumen, designed to facilitate dilation and fibrosis separation) and impact motion (by a longitudinally moving ring, designed for impacting/hitting fibrotic and calcified binding sites) to facilitate tissue dilation around the lead at the adhesion site.

In some embodiments, device is characterized by a distal and or distal portion or distal segment that is flexible and allows steering. In some embodiments, the flexibility is bi-directional bi-axial, while the steering is single directional (or straight). In some embodiments, a potential advantage of this combination of features potentially allows the device to fit a 3D structured vein, where the passage will adapt in 3D to be flexible in all directions (up/down/right/left) while steering may be, in one example, possible in all directions, or in another example limited to one direction only.

In some embodiments, while propagating through the vein, the rotating ring is typically covered by the impact ring (to minimize interaction with the vein wall), and their operation is controlled using a button on the handle, or by the pedal which controls their activation and includes buttons to control the dilation/impact modes and speed. While activated, the rings extend by less than a millimeter for interacting with and separating fibrosis in the local target tissue. The shaft is flexible and intended to follow the vein and lead path, and the operator can control its orientation by the handle and the steering mechanism using a knob on the handle. The device flexibility and steerability is intended to assist the user in orienting the tip towards the direction of the lead and potentially away from the vein wall.

In some embodiments, an external telescopic polymer sheath is provided and can be optionally used to support the shaft and for facilitating tissue dilation. The external sheath is relatively shorter than the full length of the main shaft, and is designed for optional use, potentially in the proximal part of the venous entry and path. Being shorter than the full shaft of the device enables advancing the distal part of the device without necessarily advancing the external sheath.

In some embodiments, the single-use unit is controlled by using a multi-use non-sterile foot operated pedal, which provides power to the motorized handle and is rechargeable (using a charger).

In some embodiments, the package includes a wire pulling-rod accessory, optionally used for convenient passage through the device′ lumen and assisting the passage of typical puling wires and/or locking stylet through the device′ lumen.

In some embodiments, the pedal includes a power-on button, a socket for connecting the handle's cable, a foot switch for activating the handle's motor, a button for enabling or disabling the handle's activation buttons, a speed control button (high/normal speed), and mode selection button (impact mode, dilation mode, combined mode). The pedal includes a rechargeable battery, a status indication LED, and a buzzer. The pedal package includes a charger accessory.

In some embodiments, the Lead Extraction device may be used in conjunction with lead locking and pulling devices, pulling wires/sutures and wire extension, in accordance with their Instructions For Use (IFU).

EXEMPLARY DIMENSIONS

| | | | | | | | External Sheath Inner | | |
|---|---|---|---|---|---|---|---|---|---|
| | Inner diameter | | | Outer diameter | | | diameter | | |
| | (Fr) | (In.) | (mm) | (Fr) | (In.) | (mm) | (Fr) | (In.) | (mm) |
| Diameter | 13 | 1.7 | 4.33 | 23.3 | 3 | 7.75 | 23.7 | 3.1 | 7.9 |

| | Shaft Length | | End-to-End Length | | External Sheath Length | |
|---|---|---|---|---|---|---|
| | (In.) | (cm) | (In.) | (cm) | (In.) | (cm) |
| Length | 21.3 | 54 | 31.1 | 79 | 10.2 | 26 |

All dimensions mentioned herein are subject to changes and to production tolerance.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A cardiac lead extraction system, comprising:
a. a handle;
b. an elongated body in communication with said handle;
c. a bendable flexible portion in communication with said elongated body, said bendable flexible portion comprising a first lumen sized and shaped to fit over a cardiac lead; said bendable flexible portion being more flexible than said elongated body;
d. an operational distal end in communication with said bendable flexible portion;
wherein said bendable portion is configured to bend to a bending radius of less than 4 cm while keeping said first lumen open; and
wherein said operational distal end comprises at least one lead extraction assistive tool, said operational distal end comprising a second lumen sized and shaped to fit over a cardiac lead, said second lumen being in communication with said first lumen, and said first lumen comprises an inner diameter of from about 1 mm to about 8 mm.

2. The system of claim 1, wherein said system further comprises a controllable steering mechanism configured to orient said operational distal end.

3. The system of claim 1, wherein said bendable portion is configured to perform one or more of the following:
a. bend to a minimum bending radius of from about 2 mm to about 15 mm;
b. bend to a maximal angle of from about 35 degrees to about 150 degrees;
c. change in length from about 0% to about 10% during said maximal angle;
d. perform a movement from 0 degrees to about 180 degrees;
e. bend to said maximal angle during active deflection of the system while withstanding forces up to 3000 gf; and
f. bend to said maximal angle during passive deflection of the system while withstanding forces up to 500 gf.

4. The system of claim 1, wherein said bendable portion comprises at least one articulated structure configured to maintain said first lumen open.

5. The system of claim 1, wherein a size of said inner diameter is selected from the group consisting of:
a. from about 2 mm to about 8 mm;
a. from about 2 mm to about 5 mm; and
b. from about 4 mm to about 6 mm.

6. The system of claim 1, wherein the outer diameter of said cardiac lead extraction system is from about 5 mm to about 8 mm.

7-10. (canceled)

11. The system of claim 1, wherein:
a. said elongated body comprises a first proximal end, a first distal end, and a third lumen extending from said first proximal end toward said first distal end, said third lumen sized and shaped to fit over a cardiac lead; and
b. said bendable flexible portion comprises a second proximal end, a second distal end and said first lumen extending from said second proximal end toward said second distal end, said second lumen sized and shaped to fit over a cardiac lead.

12. The system of claim 1, further comprising one or more of a motor and a pedal; and wherein at least one of the following is true:
a. said motor is located at said handle;
b. said motor is located at said pedal;
c. said pedal is used to activate and control said at least one lead extraction assistive tool.

13-16. (canceled)

17. The system of claim 1, wherein said handle is used to activate and control said at least one lead extraction assistive tool.

18. The system of claim 1, wherein at least one lead extraction assistive tool comprises one or more components configured to perform repeatable movement at a repetition rate of from about 1 Hz to about 100 Hz; and wherein said repetition rate is from about 5 Hz to about 60 Hz.

19. (canceled)

20. The system of claim 1, wherein said lead extraction assistive tool comprises a tissue cutter; and wherein said tissue cutter comprises one or more of:
a. at least one movable blade;
b. at least one transmission attached to said motor; said transmission adapted to transfer motion from said motor to said at least one movable blade;
wherein at least one of the following is true:
i. said motion of said at least one movable blade is linear;
ii. said motion of said at least one movable blade is circular;
iii. said movement of said transmission is configured to provide said at least one movable blade with a linear movement comprising an impact force to apply on the tissue;
iv. said motion of said at least one movable blade is a combination of linear movement and circular movement;
v. said motion of said at least one movable blade is characterized by a frequency from about 0.5 Hz to about 100 Hz;
vi. said motion of said at least one movable blade is characterized by a frequency from about 1 Hz to about 15 Hz;
vii. said at least one movable blade comprises a retracted state where said at least one movable blade is not exposed thereby minimizing said at least one movable blade from damaging tissue;
viii. said at least one movable blade exits distally said operational distal end from about 0.15 mm to about 2 mm;
ix. said tissue cutter comprises at least two movable blades;
x. a relative movement of said at least two movable blades provides cutting by shearing.

21-32. (canceled)

33. The system of claim 1, wherein said bendable portion comprises at least one internal structure configured to transmit motion from said handle to said operational distal end through said elongated body.

34. The system of claim 1, wherein said lead extraction assistive tool comprises a lead cutter.

35. The system of claim 2, wherein said controllable steering mechanism comprises at least one wire that runs from said handle to said operational distal end, and wherein said at least one wire runs inside a counter sleeve on said elongated body.

36. The system according to claim 12, wherein said pedal comprises one or more of a rechargeable battery, a reusable battery and a replaceable battery.

37. The system according to claim 12, wherein said system comprises one or more of a rechargeable battery, a reusable battery and a replaceable battery.

38-42. (canceled)

43. A cardiac lead extraction system configured to be operated by a single operator, comprising:
a. a handle;
b. an elongated body in communication with said handle;
c. a bendable flexible portion in communication with said elongated body, said bendable portion being more flexible that said elongated body;
d. an operational distal end in communication with said bendable flexible portion, said operational distal end comprises at least one lead extraction assistive tool;
wherein said system comprises at least one selected from the group consisting of:
e. an automatic lead tensioning mechanism configured to automatically pull said lead, thereby allowing a single operator to operate said system;
f. a controllable steering mechanism configured to orient said operational distal end;
g. a motor;
h. a lead cutter assistive component;
i. an operational distal end accessory, instead of said operational distal end, said operational distal end accessory comprising:
I. a body configured to be mounted on a distal end of said elongated body;
II. said at least one lead extraction assistive tool; and
III. a hand controller configured to control said at least one lead extraction assistive tool.

44-53. (canceled)

54. The system according to claim 1, wherein said at least one lead extraction assistive tool comprises:
a. a hollow elongated body sized to fit in a blood vessel and having a lumen sized to fit a lead of an electrical stimulator, said body having a hollow distal tip and said elongated body defining an axis;
b. an impactor having a first axial position and a second axial position, said first axial position being distal than said second axial position;
c. a local energy store coupled to said impactor and localized at said tip; and
d. a trigger which selectively releases said energy into said impactor, causing said impactor to move distally and apply axial force to tissue adjacent said tip.

55. The system according to claim 54, wherein at least one of the following is true:
a. said impactor moves within a lumen/cover/outer tube of the apparatus;
b. said local energy store is a spring;
c. a distance between said first axial position and said second axial position is from about 2 mm to about 2.5 mm;
d. said impactor comprises a blade;
e. said axial force is applied directly by a head of said impactor;
f. said axial force is applied indirectly via the tip of said apparatus;
g. said local energy store is charged by rotating said impactor in relation to said tip;
h. a blade located at said tip cuts when said impactor is periodically activated;
i. said impactor comprises a non-uniform proximal side matching a non-uniform geometry at said tip, said non-uniform comprises different circumferential locations at different axial positions;
j. said impactor impacts between one time to three times per rotation.

56-82. (canceled)
